SEQUENCE LISTING (45 sequences)

SEQ ID NO: 1 - protein, 12 aa, Artificial Sequence
  Synthetic Construct
AAVLLPVLLAAP

SEQ ID NO: 2 - protein, 15 aa, Artificial Sequence
  Synthetic Construct
KKAAAVLLPVLLAAP

SEQ ID NO: 3 - protein, 16 aa, Artificial Sequence
  Synthetic Construct
KKKAAAVLLPVLLAAP

SEQ ID NO: 4 - protein, 15 aa, Artificial Sequence
  Synthetic Construct
KKKAAVLLPVLLAAP

SEQ ID NO: 5 - protein, 20 aa, Artificial Sequence
  Synthetic Construct
KKKAAAVLLPVLLAAPREDL

SEQ ID NO: 6 - protein, 15 aa, Artificial Sequence
  Synthetic Construct
KKAAAVLLPVLLAAP

SEQ ID NO: 7 - protein, 16 aa, Artificial Sequence
  Synthetic Construct
KKKAAAVLLPVLLAAP

SEQ ID NO: 8 - protein, 15 aa, Artificial Sequence
  Synthetic Construct
KKKAAVLLPVLLAAP

SEQ ID NO: 9 - protein, 20 aa, Artificial Sequence
  Synthetic Construct
KKKAAAVLLPVLLAAPREDL

SEQ ID NO: 10 - protein, 11 aa, Artificial Sequence
  Synthetic Construct
ACDCRGDCFCG

SEQ ID NO: 11 - protein, 9 aa, Artificial Sequence
  Synthetic Construct
HWSYKLRPG

SEQ ID NO: 12 - protein, 15 aa, Artificial Sequence
  Synthetic Construct
KKAAAVLLPVLLAAP

SEQ ID NO: 13 - protein, 15 aa, Artificial Sequence
  Synthetic Construct
KKAAAVLLPVLLAAP

SEQ ID NO: 14 - protein, 11 aa, Artificial Sequence
  Synthetic Construct
ACDCRGDCFCG

SEQ ID NO: 15 - protein, 23 aa, Artificial Sequence
  Synthetic Construct
GLFGAIAGFIENGWEGMIDGWYG

SEQ ID NO: 16 - protein, 18 aa, Artificial Sequence
  Synthetic Construct
GLFEALLELLESLWLLEA

SEQ ID NO: 17 - protein, 8 aa, Artificial Sequence
  Synthetic Construct
HHHHHWYG

SEQ ID NO: 18 - protein, 30 aa, Artificial Sequence
  Synthetic Construct
WEAALAEALAEALAEHLAEALAEALEALAA

SEQ ID NO: 19 - protein, 30 aa, Artificial Sequence
  Synthetic Construct
WEAKLAKALAKALAKHLAKALAKALKACEA

SEQ ID NO: 20 - protein, 22 aa, Artificial Sequence
  Synthetic Construct
CPCILNRLVQFVKDRISVVQAL

SEQ ID NO: 21 - protein, 16 aa, Artificial Sequence
  Synthetic Construct
RQIKIWFQNRRMKWKK

SEQ ID NO: 22 - protein, 16 aa, Artificial Sequence
  Synthetic Construct
RQPKIWFPNRRKPWKK

SEQ ID NO: 23 - protein, 10 aa, Artificial Sequence
  Synthetic Construct
PLSSIFSRIG

SEQ ID NO: 24 - protein, 18 aa, Artificial Sequence
  Synthetic Construct
RGGRLSYSRRRFSTSTGR

SEQ ID NO: 25 - protein, 11 aa, Artificial Sequence
  Synthetic Construct
YGRKKRRQRRR

SEQ ID NO: 26 - protein, 7 aa, Artificial Sequence
  Synthetic Construct
RRRRRRR

SEQ ID NO: 27 - protein, 9 aa, Artificial Sequence
  Synthetic Construct
HHHHHHHHH

SEQ ID NO: 28 - protein, 16 aa, Artificial Sequence
  Synthetic Construct
KKKAAAVLLPVLLAAP

SEQ ID NO: 29 - protein, 15 aa, Artificial Sequence
  Synthetic Construct
KKKAAVLLPVLLAAP

SEQ ID NO: 30 - protein, 17 aa, Artificial Sequence
  Synthetic Construct
KKKKAAAVLLPVLLAAP

SEQ ID NO: 31 - protein, 15 aa, Artificial Sequence
  Synthetic Construct
KKAAAVLLPVLLAAS

SEQ ID NO: 32 - protein, 16 aa, Artificial Sequence
  Synthetic Construct
SKKAAAVLLPVLLAAP

SEQ ID NO: 33 - protein, 15 aa, Artificial Sequence
  Synthetic Construct
KKAAAVLLPVLLAAP

SEQ ID NO: 34 - protein, 15 aa, Artificial Sequence
  Synthetic Construct
KKAAAVLLPVLLAAP

SEQ ID NO: 35 - protein, 15 aa, Artificial Sequence
  Synthetic Construct
KKAAAVLLPVLLAAP

SEQ ID NO: 36 - protein, 15 aa, Artificial Sequence
  Synthetic Construct
KKAAAVLLPVLLAAP

SEQ ID NO: 37 - protein, 15 aa, Artificial Sequence
  Synthetic Construct
KKAAAVLLPVLLAAP

SEQ ID NO: 38 - protein, 15 aa, Artificial Sequence
  Synthetic Construct
KKAAAVLLPVLLAAP

SEQ ID NO: 39 - protein, 15 aa, Artificial Sequence
  Synthetic Construct
KKAAAVLLPVLLAAP

SEQ ID NO: 40 - protein, 15 aa, Artificial Sequence
  Synthetic Construct
KKAAAVLLPVLLAAS

SEQ ID NO: 41 - protein, 6 aa, Artificial Sequence
  Synthetic Construct
GRGESP

SEQ ID NO: 42 - protein, 6 aa, Artificial Sequence
  Synthetic Construct
GRGDSP

SEQ ID NO: 43 - protein, 6 aa, Artificial Sequence
  Synthetic Construct
GRGDNP

SEQ ID NO: 44 - protein, 8 aa, Artificial Sequence
  Synthetic Construct
RRRRRRRH

SEQ ID NO: 45 - protein, 19 aa, Artificial Sequence
  Synthetic Construct
KHKHKHKHKGKHKHKHKHK

(19) United States
(12) Patent Application Publication
(10) Pub. No.: US 2005/0025821 A1
(43) Pub. Date: Feb. 3, 2005

(54) Lipid-comprising drug delivery complexes and methods for their production

(76) Inventors: Pierrot Harvie, Vancouver (CA); Ralph W. Paul, Seattle, WA (US)

Correspondence Address: MORRISON & FOERSTER LLP, 755 PAGE MILL RD, PALO ALTO, CA, 94304-1018, US

(21) Appl. No.: 10/850,873

(22) Filed: May 20, 2004

Related U.S. Application Data
(63) Continuation of application No. 10/136,187, filed on Apr. 30, 2002.
(60) Provisional application No. 60/287,786, filed on Apr. 30, 2001.

(51) Int. Cl. A61K 48/00; A61K 9/127; C12N 15/88

(52) U.S. Cl. 424/450; 435/458; 514/44

(57) ABSTRACT

Novel stable, concentrated, biologically active and ready-to-use lipid-comprising drug delivery complexes and methods for their production are described. The. complexes of the invention comprise a drug, at least one lipid species, optionally at least one polycation, and at least one targeting factor. The at least one lipid species may comprise a pegylated lipid. The complexes of the invention may provoke lower levels of inflammatory cytokines such as tumor necrosis factor-α (TNF- α). The method described herein provides for the large scale production of lipid-comprising drug delivery systems useful for gene therapy and other applications.

### CROSS-REFERENCE TO RELATED APPLICATIONS

This application claims the benefit of U.S. Provisional Application Ser. No. 60/287,786, filed Apr. 30, 2001, the disclosure of which is incorporated herein by reference in its entirety.

### STATEMENT OF RIGHTS TO INVENTIONS MADE UNDER FEDERALLY SPONSORED RESEARCH

Not applicable.

### TECHNICAL FIELD

The present invention relates to lipids and their use as vehicles for the transfer. of nucleic acids into cells. More specifically, this invention relates to lipid-comprising drug delivery complexes which are stable, biologically active, and capable of being concentrated, and to methods for their production. The complexes of the invention may reduce levels of inflammatory cytokines such as tumor necrosis factor-α (TNF-α).

### BACKGROUND ART

The development of new forms of therapeutics which use macromolecules such as proteins or nucleic acids as therapeutic agents has created a need to develop new and effective means of delivering such macromolecules to their appropriate cellular targets. Therapeutics based on either the use of specific polypeptide growth factors or specific genes to replace or supplement absent or defective genes are examples of therapeutics which may require such new delivery systems. Clinical application of such therapies depends not only on the efficacy of new delivery systems but also on their safety and on the ease with which the technologies underlying these systems can be adapted for large scale pharmaceutical production, storage, and distribution of the therapeutic formulations. Gene therapy has become an increasingly important mode of treating various genetic disorders. The potential for providing effective treatments, and even cures, has stimulated an intense effort to apply this technology to diseases for which there have been no effective treatments. Recent progress in this area has indicated that gene therapy may have a significant impact not only on the treatment of single gene disorders, but also on other more complex diseases such as cancer, and on immune modulation and treatment of infectious diseases. However, a-significant obstacle in the attainment of efficient gene therapy has been the difficulty of designing new and effective means of delivering therapeutic nucleic acids to cell targets. Thus, an ideal vehicle for the delivery of exogenous genes into cells and tissues should be highly efficient in nucleic acid delivery, safe to use, easy to produce in large quantity and have sufficient stability to be practicable as a pharmaceutical. It would further be advantageous to localize drug delivery to a specific cellular target site.

Non-viral vehicles, which are represented mainly by cationic liposome formulations, are one type of vehicle which have, for the following reasons, been considered for use in gene therapy. First, the plasmid DNA required for liposome-mediated gene therapy can be widely and routinely prepared on a large scale and is simpler and may carry less risk than the use of viral vectors, such as retroviruses. Second, liposome-mediated gene delivery, unlike retroviral-mediated gene delivery, can deliver either single or double stranded RNA or DNA. Thus, DNA, RNA, or an oligonucleotide can be introduced directly into the cell. Further, unlike retroviral-mediated gene delivery, there is no limitation on the size of nucleic acid which can be delivered by liposomes. Moreover, cationic liposomes are for the most part non-toxic, non-immunogenic and can therefore be used repeatedly in vivo as evidenced by the successful in vivo delivery of genes to catheterized blood vessels (Nabel, E. G., et al. (1990) Science, 249:1285-1288), lung epithelial cells (Brigham, K. L., et al. (1989) Am. J Respir. Cell Mol. Biol., 195-200, Stribling, R., et al. (1992) Proc. Natl. Acad. Sci. U.S.A., 89:11277-11281), and other systemic uses (Zhu, N., et al. (1993) Science, 261:209-211, Philip, R., et al. (1993) Science, 261:209-211; Nabel, G. et al (1994) Hum. Gene Ther., 5:57-77) of cationic liposomes.

There are also examples of non-viral gene therapy formulations which do not utilize liposome formulations. See, for example, Kircheis et al. (2001) Gene Therapy Molec. Biol. 6:159-167; Rungsardthong et al. (2001) Brit. Pharm. Conf. Abstract Book:78. A successful ex vivo gene therapy treatment for severe combined immunodeficiency syndrome was reported (Hacein-Bey-Abina et al. (2002) NEJM 346(16):1185-1193.

A variety of cationic liposome formulations are known in the art, including the commercially available cationic liposome reagent DOTMA/DOPE (N-1,-(2, 3-dioleoyloxy) propyl-N,N,N-trimethyl ammonium chloride/dioleoyl phosphatidylethanolamine), (Felgner, P. L. et al. (1987) Proc. Natl. Acad. Sci. U.S.A., 84: 7413-7417), and a cationic liposome formulation designated DC-Chol/DOPE (3 βN-(N′,N′-dimethylaminoethane)-carbamoyl cholesterol/dioleoyl phosphatidylethanolamine) (Gao, X., and Huang, L. (1991) Biochem. Biophys. Res. Commun., 179:280-285), which was shown to be relatively non-toxic and more efficient than DOTMA/DOPE. Following extensive in vivo studies (Plautz, G. E., et al. (1993) Proc. Natl. Acad. Sci. U.S.A., 90:4645-4649, Stewart, M. J., et al. (1992) Hum. Gene Ther., 3:267-275) in which DC-Chol/DOPE was demonstrated to be both safe and efficacious as a nucleic acid delivery system, this formulation was approved by the U.S. Food and Drug Administration (FDA) and the U.K. Medicines Control Agency (MCA), and has been used in two separate gene therapy clinical trials (Nabel, G. J., et al. (1993) Proc. Natl. Acad. Sci. U.S.A., 90:11307-11311, Caplen, N. J., et al. (1995) Nature Medicine, 1:39-46). However, cationic liposomes comprising lipids such as DOTAP/cholesterol are known to provoke production of inflammatory cytokines, such as TNF-α (Whitmore, M., et al. Gene Therapy 1999 November; 6(11):1867-75).

Anionic liposomes have been well characterized in the past 3 decades as drug delivery systems (Lasic, D. D. (1998) Trends in Biotechnology 16:307-321), and may have longer circulation lifetimes than cationic liposomes. However, anionic liposomes have no electrostatic interaction with DNA, and have lower cell binding capacity compared to cationic liposomes. These factors have limited their progress in terms of non viral gene delivery systems (Legendre, J. Y. & Szoka, F. C., Jr. (1992) Pharm Res 9:1235-42). However, recent studies using pre-compacted DNA surrounded by anionic lipid have shown promising results in term of transfection capacity (Hagstrom, J. E., Sebestyen, M. G., Budker, V., Ludtke, J. J., Fritz, J. D. & Wolff, J. A. (1996) Biochimica et Biophysica Acta—Biomembranes 1284:47-55; Ibáñez, M., Gariglio, P., Chávez, P. & Baeza, I. (1996) Biochem. Cell Biol. 74:633-643; Lee, R. J. & Huang, L. (1996) Journal of Biological Chemistry 271:8481-8487; Shangguan, T., Cabral-Lilly, D., Pudrandare, U., Godin, N., Ahl, P., Janoff, A. & Meers, P. (2000) Gene Therapy 7:769-783; Mastrobattista et al.; (2001) Cancer Gene Therapy 8(6):405-413).

U.S. Pat. Nos. 5,795,587 and 6,008,202 disclose nucleic acid/lipid/polycation drug delivery complexes and their use as vehicles for the transfer of nucleic acids or other macromolecules into cells. Liposome formulations are also described in U.S. Pat. Nos. 5,753,262, 6,056,973, 6,147,204, 6,011,020, 5,013,556, and 5,976,567 and in WO 93/05162, WO 97/11682, and WO 98/00110. Formulations incorporating cationic and/or neutral lipids are also described in U.S. Pat. Nos. 5,939,401, 6,071,533, 5,948,767 and 5,059,591.

Complexes exhibiting a positively charged surface have a greater binding affinity to cell surfaces than complexes having a neutral or negatively charged surface. However, they also interact with other serum components in vivo, reducing circulation lifetimes. The incorporation of hydrophilic polymers such as polyethylene glycol, or other surface structures that provide a steric barrier to serum protein binding, significantly increase liposome circulation lifetimes (Harasym, T. O., et al. (1998) Advanced Drug Delivery Reviews 32:99-118.)

The use of polyethylene glycol (PEG)-modified lipids is well established for liposome encapsulated drugs, and its ability to enhance delivery of anti-cancer drugs to tumor sites has been proven (Lasic, D. D., Trends in Biotechnology, (1998) 16(7):307-321). Further, inclusion of PEG in liposomes can decrease the size of the liposome particles and increase liposome stability (Harvie et al. (2000) J. Phar Sci. 89(5):652-663). Pegylated lipids are also known to control surface properties of lipid-based gene transfer systems. However, pegylated lipid incorporation into lipid-DNA complexes causes a concentration dependent reduction of in vitro transfection activity, a result that can be partially attributed to a reduction in particle binding to cells (Harvie, P., et al. (2000) J. Phar Sci. 89(5):652-663).

Peptides or proteins have been attached to liposome formulations for the purpose of targeting the liposomes to particular cell type(s). A recent review examined methods of protein conjugation onto liposomes and the effects of surface bound proteins on the liposomes' biological behavior (Harasym, T. O., et al. (1998) Advanced Drug Delivery Reviews 32:99-118.) The presence of a conjugated protein can significantly alter the attributes of targeted liposomes. Specifically, protein conjugation can result in dramatic increases in liposome size, enhanced immunogenicity, and increased plasma elimination. Peptides, which are smaller than proteins, can cause less increase in particle size and immunogenicity than proteins. Targeted anionic lipid carriers have been generated using DOPE-PEG-folate as a ligand and in vitro transfection activity enhancement was observed (Lee, R. J. & Huang, L. (1996) Journal of Biological Chemistry 271:8481-8487; and U.S. Pat. No. 5,908,777).

LHRH receptor is known to be expressed in high percentage in breast, endometrial, ovarian, and prostate cancer cells (Schally, A. V. and A. Nagy, (1999) Eur J Endocrinol. 141(1):1-14). The arginine-glycine-aspartic acid (RGD) motif is known to interact with (αVβ3 and (αVβ5 integrin receptors, which are often up-regulated in solid tumor blood vessels, and in tumor cells themselves. RGD motifs were previously shown to be efficient in enhancing drug delivery and anti-cancer activity in vivo (Arap, W., R. Pasqualini, and E. Ruoslahti, (1998) Science 279(5349):377-80). The RGD motif has also been covalently associated with poly-L-Lysine and its activity in association with cationic liposomes in vitro has been previously demonstrated (Colin, M., et al., (2000) Gene Ther. 7(2):139-52; Harbottle, R. P., et al., (1998) Hum Gene Ther. 9(7):037-47).

Cell membrane-translocating peptides (MTLP) interact directly with and penetrate the lipids of the cell membrane lipid bilayer (Fong et al. (1994) Drug Development Research 33:64). The central hydrophobic h-region of the signal sequence of Kaposi's fibroblast growth factor, AAVLLPVLLAAP (SEQ ID NO:1), is considered to be a membrane translocating peptide. This peptide has been used as a carrier to deliver various short peptides (<25 mer) through the lipid bilayer into living cells in order to study intracellular protein functions and intracellular processes (Lin et al. (1996) J. Biol. Chem. 271:5305; Liu, et al. (1996) Proc. Natl. Acad. Sci USA 93:11819; Rojas et al. (1997) Biochem. Biophys. Res Commun. 234:675).

A number of synthetic polymers are known which penetrate lipid membranes. These polymers can be likened to MTLPs which facilitate entry into, or exit from, a compartment through translocation through a membrane. However, the polymers are synthetic, rather than a naturally occuring species. A number of these synthetic polymers have been investigated for their ability to disrupt endosomal membranes (being membrane-disruptive at endosomal pH) while being non-disruptive towards cellular membranes (being non-membrane disruptive at neutral pH) and their use has been suggested in drug delivery systems (Stayton et al. (2000) J. Controll. Release 65:203-220; WO 99/34831) and tested in certain cationic lipid formulations (Cheung et al. (2001) Bioconj. Chem. 12:906-910). These polymers have been found to be capable of inducing red blood cell hemolysis in vitro (Lackey et al. (1999) Bioconj. Chem. 10:401-405; Murthy et al (1999) J. Controll. Release 61:137-143; Mourad et al. (2001) Macromolecules 34:2400-2401; Seki et al. (1984) Macromolecules 17:1692-1698) and have additionally been incorporated into cationic liposome formulations for use in the delivery of nucleic acid to cells (Stayton et al. (2001) Molec. Therapy vol 3 May pg S194 Poster Abstract ASGT “Ph-Sensitive Polymer Additives for Enhancing Lipoplex Transfections”; Stayton et al. (2000) Molec. Therapy vol 1 May pg S243 poster Abstract ASGT 2000 “A Nonviral Liposomal Complex Designed to Overcome the Multiple Barriers to Gene Transfer”).

There is a continued need for stable, biologically active, lipid-comprising drug delivery complexes which are capable of being formulated at high concentration. There is also a need for stable, biologically active, lipid-comprising drug delivery complexes which can achieve higher concentration levels of nucleic acid. There is further a need for stable lipid-comprising drug delivery complexes with enhanced transfection activity and specificity. There is also a need for stable lipid-comprising drug delivery complexes which provoke lower levels of inflammatory cytokine production, and which result in a reduced inflammation response when administered in vivo.

### DISCLOSURE OF THE INVENTION

Provided are lipid complexes for the delivery of biologically active nucleic acid to particular cells. The lipid complexes are formulated to deliver nucleic acid to cells in a form which is biologically active and which may be delivered to the particular cells in vitro, ex vivo or in vivo and particularly formulations which may be delivered intravenously for use in vivo. The lipid complexes are formulated such that they protect nucleic acids from degradation by serum components such that the nucleic acid retains its biologic activity; are appropriately sized particularly when in vivo, such that they are not immediately cleared from circulation by the RES system or other organs known in the art to be first pass circulatory clearance organs; and deliver an effective therapeutic or diagnostic amount of biologically active nucleic acid into particular cells. These properties may be assayed by measuring the level of transfection of the lipid complexes in vitro or in vivo, measuring the mean diameter of the cells after incubation in serum and by determining the amount of complement opsonization by the lipid complexes. Preferred are lipid complexes which are also of low toxicity and high target cell specificity.

Lipid complexes exhibiting these properties may be generated using the components and methods as described herein to generate particular formulations of lipids and compacted nucleic acid. As described herein, particular combinations of these components result in stable complexes which can deliver an effective amount of biologically active nucleic acid to a particular cell or tissue type for use in the treatment or diagnosis of a variety of diseases, conditions or syndromes.

In the embodiments described herein, the lipid/compacted DNA complexes of the invention are characterized in that they have the properties described herein, or properties equivalent to those described herein and further, can be formulated reproducibly so as to exhibit these properties.

The drug/lipid/targeting factor complexes of this invention, which optionally comprise a polycation, are generally stable, capable of being produced at relatively high concentration, and retain biological activity over time in storage. These complexes may further reduce levels of inflammatory cytokine (for example, TNF-α) production, and may result in a reduced inflammation response when administered in vivo, as compared to, for example, LPD formulations which do not comprise a targeting factor. When formulated with nucleic acids, these complexes may achieve high nucleic acid concentration levels, may demonstrate enhanced transfection activity and specificity, and may also enhance targeted delivery to target cells and tissues and enhanced intracellular uptake at such target cells and tissues. When the drug to be delivered is a nucleic acid, the complexes may increase intracellular expression levels of the delivered gene, resulting in a therapeutic and/or prophylactic and/or diagnostic effect. These complexes may further be adjusted to allow for optimized circulation times and tissue targeting capabilities, depending on the target tissue and drug load. Such complexes are of utility in the delivery of nucleic acids, proteins and other macromolecules to cells and tissues. The delivery of nucleic acids to cells and tissues is useful for therapeutic uses, prophylactic uses, and for diagnostic purposes.

Accordingly, in a particular embodiment is provided a lipid-nucleic acid complex comprising a compacted nucleic acid, a polycation, a targeting factor, and a lipid, wherein:

the targeting factor increases cellular bioavailability of the nucleic acid by a means other than interaction with a specific outer cell surface membrane receptor; the complex does not comprise a protamine or a salt thereof; and the mean diameter of the complex is greater than about 100 nm and less than 400 nm.

In certain examples of the embodiments decribed herein, the targeting factor is a membrane-disruptive polymer.

In other examples, the mean diameter of the complex is about 300 nm or less. In certain other emboidments, the mean diameter of the complex is about 200 nm or less.

In particular examples of the complexes described herein, the complex ftrther comprises a shielding moiety.

In certain embodiments, the shielding moiety increases the circulatory half-life of the complex, reduces binding of serum components to the complex, or reduces complement opsonization of the complex.

In particular examples, the shielding moiety comprises polyethylene glycol (PEG). In other examples, the shielding moiety is PEG. In still other examples of the complexes, the shielding moiety comprises a pegylated lipid.

In certain embodiments, the polycation is a synthetic polycation, a polycationic polypeptide or salt thereof. In particular examples, the polycation is a synthetic polycation. In certain complexes, the synthetic polycation is selected from the group consisting of polycationic methacryloxy polymers, polycationic methacrylate polymers and polycationic poly(alkenylimines).

In certain examples of the complexes, the polycationic methacrylate polymer is comprised of dimethylamino methacrylate. In other examples, the synthetic polycation is selected from the group consisting of polyethyleneimine (PEI), poly(2-methacryloxyethyltrimethyl ammonium bromide) (PMOETMAB), and a co-polymer of dimethylamino methacrylate and methacrylic ester.

In particular embodiments as described herein, the targeting factor is a membrane-disruptive synthetic polymer.

In particular embodiments as described herein, the targeting factor functions to increase cellular bioavailability by increasing transcription of the nucleic acid of the complex, by increasing uptake of the nucleic acid into the cell, by increasing uptake into a cellular compartment, by increasing exit of the nucleic acid from a cellular compartment, or by increasing transport of nucleic acid across a cell membrane.

In particular embodiments of the complexes, the targeting factor is a membrane translocating peptide (MTLP). In some embodiments, the membrane translocating peptide is selected from the group consisting of H₂N-KKAAAVLLPVLLAAP-COOH (Elan094) (SEQ ID NO:2), H₂N-KKKAAAVLLPVLLAAP (ZElan094) (SEQ ID NO:3), H₂N-kkkaavllpvllaap (ZElan207) (SEQ ID NO:4), and H₂N-KKKAAAVLLPVLLAAPREDL (ZElan094R) (SEQ ID NO:5).

In certain other examples, the targeting factor comprises a nuclear localization sequence. In certain complexes the nuclear localization sequence is SV 40 NLS.

In particular examples of the complexes described herein, the complex fuirther comprises a co-lipid.

In certain examples of the complexes described herein the targeting factor is conjugated to a PEG moiety.

In particular examples of the complexes described herein, the lipid is a cationic lipid.

In particular complexes, the cationic lipid is 1,2-bis(oleoyloxy)-3-trimethylammoniopropane (DOTAP). In certain embodiments, the cationic lipid is DOTAP.

In particular examples of the complexes, the co-lipid is selected from the group consisting of cholesterol, diphytanoyl phosphatidylethanolamine (DPHPE), dioleoyl phosphatidylethanolamine (DOPE), dioleoyl phosphatidylcholine (DOPC), dilauryl phosphatidylethanolamine (DLPE), 1,2-distearoyl-sn-glycero-3-phosphatidylethanolamine (DSPE), and dimyristoyl phosphatidylethanolamine (DPME).

In another embodimerit of the complexes described herein is provided a lipid-nucleic acid complex comprising a compacted nucleic acid and at least one lipid species that is fusogenic, wherein: the complex has an aqueous core; and the mean diameter of the complex is greater than about 100 nm and less than 400 nm.

In yet another embodiment is provided a lipid-nucleic acid complex comprising a compacted nucleic acid, a polycation, a targeting factor and at least one lipid species, wherein: the at least one lipid species is an anionic lipid; the complex has an aqueous core; the complex comprises at least one fusogenic moiety; the mean diameter of the complex is greater than about 100 nm and less than 400 nm; and, wherein the complex does not comprise protamine or a salt thereof.

In particular examples of the complex, the mean diameter of the complex is greater than about 100 nm and less than 200 nm. In certain embodiments of the complexes described herein, the mean diameter of the complex is determined by incubation in 50% serum in buffer for about 12 hour.

In certain examples the complex has reduced binding to complement C3A and C5A.

In particular embodiments of the complexes described herein, the fusogenic lipid is a cone forming lipid. In certain examples of the complex, the cone forming lipid is dioleoyl phosphatidylethanolamine (DOPE), 1,2-dioleoyl-sn-glycero-3-[phospho-L-serine] (DOPS), or N,N dioleyl-N,N-dimethyl-1,6-hexanediammonium chloride (TODMAC6).

In other embodiments, the fusogenic lipid is pH sensitive.

In certain embodiments, the lipid is anionic at physiological pH, and fusogenicity is increased at about pH 5.5 to about pH 4.5 relative to physiological pH. In particular examples, at about pH 4.5 the lipid is neutral or cationic. For complexes comprising anionic lipids, including fusogenic anionic lipids, a polycation is required for successful formulation. The complexes described above may optionally comprise a targeting factor, either specific or non-specific which may, or may not, be conjugated to any other component of the complex.

In certain examples, the lipid is cholesteryl hemisuccinate (CHEMS) or 1,2-dioleoyl-sn-glycero-3-[phosphoethanolamine-N-dodecanoyl] NC₁₂-DOPE).

In certain examples of the complexes described herein, the lipid is neutral or cationic.

In particular examples of the complexes where the complex contains a fusogenic moiety, including a fusogenic lipid, the polycation is selected from the group consisting of synthetic polycationic, polycationic polypeptides, and salts thereof. In certain, wherein the polycation is a synthetic polycation. In particular examples, the synthetic polycation is selected from the group consisting of polycationic methacryloxypolymers, polycationic methacrylate polymers and polycationic poly(alkenylimines). In other examples, the synthetic polycationic methacrylate polymer is a polymer comprising dimethylamino methacrylate. In particular examples, the synthetic polycation selected from the group consisting of polyethyleneimine (PEI), poly(2-methacryloxyethyltrimethyl ammonium bromide) (PMOETMAB), and a co-polymer of dimethylamino methacrylate and methacrylic ester.

In particular examples of the complexes as described herein, the complex further comprises at least one co-lipid.

In particular examples, the complex comprises 1,2-distearoyl-sn-glycero-3-phosphotidylethanolamine (DSPE).

In still other examples, the complex further comprises at least one targeting factor that increases cellular bioavailability of the nucleic acid. In particular examples of the complexes, the presence of the targeting factor results in an increase in transcription of the nucleic acid, an increase in the uptake of nucleic acid into the cell, an increase in the uptake of nucleic acid into a cellular compartment, an increase in an exit of the nucleic acid from a cellular compartment, or an increase in transport of the nucleic acid across a membrane.

In certain examples, the targeting factor is selected from the group consisting of folate, insulin, an Arg-Gly-Asp (RGD) peptide, luteinizing hormone releasing hormone (LHRH), a membrane translocating peptide (MTLP) and a compound comprising a nuclear localization sequence. In particular examples, the targeting factor is selected from the group consisting of galactose-H₂N-KKAAAVLLPVLLAAP-COOH (Elan094) (SEQ ID NO:6), galactose-H₂N-KKKAAAVLLPVLLAAP (ZElan094) (SEQ ID NO:7), galactose-H₂N-kkkaavllpvllaap (ZElan207) (SEQ ID NO:8), and galactose-H₂N-KKKAAAVLLPVLLAAPREDL (ZElan094R) (SEQ ID NO:9).

In certain examples where the lipid is fusogenic, the lipid undergoes a structural change between physiologic pH and pH about 4.5 resulting in increased fuiogenicity.

In certain examples of the complexes described herein, the complex is shielded.

In particular examples where the complex is shielded, the complex fuirther comprises a compound containing polyethylene glycol moieties. In some examples, the compound is a pegylated lipid.

In another aspect is provided a method for preparing a lipid-nucleic acid complex comprising a compacted nucleic acid and at least one lipid species that is fusogenic, comprising:
a) mixing an aqueous micelle mixture comprising a lipid and at least one lipophilic surfactant with a nucleic acid mixture comprising a nucleic acid, wherein the lipid has or assumes fusogenic characteristics, and wherein at least one of the mixtures contains a component that causes the nucleic acid to compact; and
b) after the mixing removing the lipophilic surfactant from mixture resulting from step a).

In certain embodiments of the method described above, the method further includes at least one targeting agent in at least one of the mixtures of step a).

In certain embodiments are provided lipid-nucleic acid complexes prepared by the methods described above.

Also provided are complexes as described herein as prepared by the methods described above.

In another aspect is provided a method of delivering a nucleic acid to a cell comprising contacting the cell with a complex as described herein.

A further embodiment of the above method includes where the delivery is in vivo to an individual.

Yet another embodiment of the above methods includes where the delivery is intravenous.

In particular embodiments of the above-described methods, the individual is a human.

Certain embodiments further provide use of the complexes as described herein in the manufacture of a medicament for the treatment or diagnosis of a disease, condition, or syndrome.

### BRIEF DESCRIPTION OF THE DRAWINGS

FIGS. 1A-1D show the effect of targeting factor-pegylated lipid conjugate incorporation on size distribution (mean diameter) and polydispersity of lipid-protamine-DNA (LPD) and dialyzed lipid-protamine-DNA (DLPD) complexes.

FIGS. 2A-2D show in vitro luciferase expression and fold enhancement in MDA-MB-231 and LL/2 cells after transfection with LPDs containing different concentrations of DSPE-PEG_{5K}-LHRH or DSPE-PEG_{5K}-RGD.

FIGS. 3A-3D show in vitro luciferase expression and fold enhancement in MDA-MB-231 and LL/2 cells after transfection with DLPDs containing different concentrations of DSPE-PEG_{5K}-LHRH or DSPE-PEG_{5K}-RGD.

FIGS. 4A and 4B show the effect of serum on LPD mean diameter and particle polydispersity.

FIGS. 5A and 5B show the effect of serum on LPD formulation transfection activity and fold enhancement in MDA-MB-231 cells.

FIG. 6A shows in vitro luciferase expression in MDA-MB-231 cells after transfection with LPDs containing DSPE-PEG_{5K}-LHRH in competition assays.

FIG. 6B shows in vitro luciferase expression in MDA-MB-231 cells after transfection with DSPE-PEG_{5K}-LHRH in competition assays.

FIG. 7 shows in vitro luciferase expression in MDA-MB-231 cells after transfection with DSPE-PEG_{5K}-RGD in competition assays.

FIG. 8 shows serum TNF-α levels 2 hours after intravenous injection of formulations containing 50 μg DNA.

FIG. 9 is a chart showing the recoveries of ZElan207 from control solutions (white bars) and from mouse serum (black bars) at time points 10, 30, 60, and 120 mm.

FIG. 10 is a chart showing the recoveries of ZElan094 from control solutions (white bars) and from mouse serum (black bars) at time points 10, 30, 60, and 120 min.

FIG. 11 is a dose titration study showing the transfection levels of HEPG2 cells with DC-chol:DOPE LPDs containing increasing concentrations of the ZElan094 MTS peptide.

FIG. 12 shows transfection of ASGPR bearing liver cells (HepG2 cells) with DC-chol:DOPE LPD complexes containing various adsorbed MTLP-galactose targeting ligands.

FIG. 13 shows transfection of ASGPR non-bearing liver cells (HepSKI cells) with DC-chol:DOPE LPD complexes containing various adsorbed MTLP-galactose targeting ligands.

FIG. 14 is a dose titration study showing the transfection of ASGPR bearing liver cells (HepG2 cells) with DC-chol:DOPE LPD complexes containing increasing concentrations of Elan094-Gal (SEQ ID NO:6).

FIG. 15 shows Luciferase expression in tumours following in vivo administration of LPDs containing Elan219 (DOPE-Elan094) (SEQ ID NO:12) by direct intratumoral injection to BalbC mice engrafted with MDA-MB-231 breast tumors.

FIG. 16 shows in vitro luciferase expression of anionic DLPD formulations in MDA-MB-231 cells.

FIGS. 17A and 17B show the effect of serum on anionic DLPD formulation mean diameter and particle polydispersity.

FIG. 18 shows the effect of serum on anionic DLPD formulation transfection activity in MDA-MB-231 cells.

FIG. 19 shows the effect of serum on anionic DLPD formulation transfection activity in MDA-MB-231 cells.

FIGS. 20A and 20B show transfection activity of anionic DLPD formulations in CHO-K1 cells.

FIG. 21 shows transfection activity of anionic DLPD and targeted anionic DLPD in MDA-MB-231 cells.

FIG. 22 shows the effect of serum on DLPD mean diameter (A) and particle polydispersity (B) prior to transfection assay following addition of 2 and 5% lipid ligand.

FIG. 23 shows the effect of serum on transfection activity in MDA-MB-231 cells for targeted LPD following addition of 2 or 5 mol % lipid ligand. RLU/mg luciferase expression in (A) or fold enhancement (B) over base PEG formulation.

FIG. 24 shows effect of serum on the serum effect on LPD size (A) and (B) on transfection activity in MBA-MD-231 cells for targeted LPD following addition of 10 free DSPE-PEG and 5% lipid ligand.

FIG. 25 shows anionic DLPD transfection activity in CHO-K1 cells.

FIG. 26 shows the effect of DNA concentration on DLPD mean diameter (A) and particle polydispersity (B).

FIG. 27 shows transfection activity in Skov3-ip1 cells for anionic DLPD at different DNA concentrations.

FIG. 28 shows anionic DLPD and targeted anionic DLPD transfection activity in KB cells. (A) luciferase expression, (B) fold enhancement over CHEMS:DOPE base formulation.

FIG. 29 shows a comparison of cationic LPD vs. anionic DLPD effect on in vitro cell proliferation in an MTS cell toxicity assay.

FIG. 30 shows a DSPE-PEG_{5K}-Folate titration in CHEMS:DOPE anionic DLPD formulation.

FIG. 31 shows luciferase expression and fold enhancement of expression of luciferase in SKOV3-ip1 cells following incorporation of different cationic polymer-condensed DNA into anionic DLPDs.

FIG. 32 shows luciferase expression in KB cell following incorporation of different cationic polymer-condensed DNA into anionic DLPDs.

FIG. 33 shows the effect of various polymer-condensed DNA complexes on transfection activity in KB cells.

FIG. 34 shows the effect of various polymer-condensed DNA incorporation into CHEMS:DOPE anionic LPD transfection activity in KB cells.

FIG. 35 shows the effect of various polymer-condensed DNA incorporation into CHEMS:DOPE: 0.5% DSPE-PEG_{5K} anionic LPD transfection activity in KB cells.

FIG. 36 shows the effect of various polymer-condensed DNA incorporation into CHEMS:DOPE: 0.5% DSPE-PEG_{5K} anionic LPD transfection activity in KB cells.

FIG. 37 shows the effect of various polymer-condensed DNA incorporation into NC₁₂-DOPE:DOPE: anionic DLPD transfection activity in KB cells.

FIG. 38 shows the effect of various polymer-condensed DNA incorporation into NC₁₂-DOPE:DOPE: 0.5%;DSPE-PEG_{5K} anionic LPD transfection activity in KB cells.

FIG. 39 shows the effect of various polymer condensed DNA incorporation into NC₁₂-DOPE:DOPE: 0.5%;DSPE-PEG_{5K}-Folate anionic LPD transfection activity in KB cells.

FIG. 40 shows the fold enhancement for anionic DLPDs where the DNA was compacted with PEI over conventional anionic LPD prepared with protamine-compacted DNA. Luciferase data from FIGS. 34-39.

FIG. 41 shows the anionic LPD PEG-folate fold enhancement over LPD-PEG following transfection in KB cells.

FIGS. 42A and 42B show the effect of PPAA incorporation into LPD on KB cells in vitro transfection, cells were transfected with 0.1 μg DNA/well. C and D show fold enhancement of PPAA incorporation into LPD formulation on transfection enhancement in KB cells from A and B.

FIG. 43 shows the zeta potential (A) and mean diameter (B) of LPD with and without PPAA throughout a titration of pH.

FIG. 44 shows the effect of PPAA incorporation into LPD, LPD-PEG and LDP-PEG-folate formulation and the effect on KB cells in vitro transfection

FIG. 45 shows the effect of DSPE-PEG_{5K}-Folate addition to LPD with or without PPAA on in vitro cell proliferation.

FIG. 46 shows the effect of DSPE-PEG_{5K}-Folate addition to LPD containing or not containg PPAA on in vitro cell proliferation.

FIG. 47 shows the effect of PPAA addition into LPD formulations containing extra DSPE-PEG_{2K} on transfection activity in KB cells.

FIGS. 48A-48E show the effect of PPAA/DNA ratio on transfection activity in KB cells, with both 2% (B and C) and 10% (D and E) PEG incorporation.

FIG. 49 shows the effect of chloroqine on transfection activity in KB cells.

FIGS. 50A and 50B show the effect of bafilomycin on transfection activity in MDA-MB-231 cells, in LPDs without (A) and with (B) PPAA.

FIGS. 51A-51C show the effect of bafilomycin on transfection activity in KB cells, in LPDs with and without PPAA.

FIG. 52 shows day 70 mean diameter of MDA-MB-231 in vivo tumor growth following administration of LPD-folate HSV TK1 formulations.

### BEST MODES FOR CARRYING OUT THE INVENTION

Provided are lipid complexes for the delivery of biologically active nucleic acid to particular cells or tissues. The lipid complexes are formulated to deliver nucleic acid to cells in a form which is biologically active and which may be delivered to particular cells in vitro, ex vivo or in vivo and particularly formulations which may be delivered intravenously for use in vivo. The lipid complexes are formulated such that they protect nucleic acid from degradation by species present in serum in vivo or in vitro such that the nucleic acid may be successfully transfected into cells; are of appropriate mean diameter, particularly when in vivo, that they are not cleared from circulation prior to achieving a therapeutic or diagnostic effect; and deliver an effective amount of biologically active nucleic acid into particular cells. These properties may be assayed by measuring the level of transfection of the lipid complexes in vitro or in vivo, measuring the mean diameter of the cells after incubation in serum and by determining the amount of complement opsonization by the lipid complexes. Preferred are lipid complexes that are also of low toxicity and high target cell specificity.

Lipid complexes exhibiting these properties may be generated using the components and methods as described herein to produce particular formulations of lipids and compacted nucleic acid which may further include one or more of the following components: co-lipids, shielding moieties, fusogenic moieties and specific or non-specific targeting factors, as well as a polycation to achieve nucleic acid compaction. As described herein, particular combinations of these components result in stable complexes which can deliver an effective amount of biologically active nucleic acid to a desired cell or tissue type for use in the treatment or diagnosis of a variety of diseases, conditions or syndromes.

Characterization of Lipid Complexes

A lipid complex that will deliver a therapeutically or diagnostically effective amount of biologically active nucleic acid to a cell will be characterized, by both in vitro and in vivo methods, by a number of properties which are indicative of successful in vivo or ex vivo delivery of the particular nucleic acid to the particular cell. The properties which are indicative of successful delivery of nucleic acid to cells include: the mean diameter of the complex, both before and after incubation in serum; the transfection efficiency of the nucleic acid in vivo and/or in vitro; protection of the nucleic acid from degradation by serum species; and the level of complement opsonization.

As used herein, and as well-understood in the art, a “therapeutically effective amount” of a nucleic acid delivered to a cell is an amount such that beneficial or desired results, including clinical results, are obtained. For the purposes of this invention, beneficial or desired clinical results can include one or more, but are not limited to, alleviation or amelioration of one or more symptoms, diminishment of the extent of a condition, stabilization of the (i.e., not worsening) condition, prevention of spread of disease, delay or slowing of disease progression, amelioration or palliation of the disease state or condition from which an individual suffers, and remission (whether partial or total), whether detectable or undetectable.

The term a “diagnostically effective amount” of nucleic acid is used herein to describe a level of nucleic acid which is expressed in a particular cell such that the presence of the nucleic acid may be detected by conventional techniques in the art in the particular cell, tissue or, for example, tumor in which the nucleic acid is expressed. For example, expression of nucleic acid only in tumor tissue permits the diagnosis of conditions associated with the tumor, or permits the identification of the tumor location.

Measurement of the properties listed above make it possible to identify lipid complexes which will have diagnostic or therapeutic utility. For example, a mean diameter for a complex of less than 400 nm prior to incubation in serum is important for several reasons. Generally, particles of diameter greater than 400 nm will usually have a reduced circulatory half life compared to similar smaller sized (e.g., charge, targeting factor, shielding moiety) complexes. A number of factors effect the circulatory half-life of complexes, including mean particle diameter or size and complement opsonization. Larger particles or particles that fix complement (complement opsonization) are cleared more quickly by the RES system and are also more likely to be removed by first pass clearance organs (also referred to as first pass trafficking organs), such as the lungs or liver, upon intravenous administration. Additionally, some mechanisms of cellular uptake cannot proceed, or are less efficient, with larger particles, thus reducing the amount or rate of delivery of nucleic acid to the nucleus. Additionally, if the base lipid complex formulation (lipid/DNA/optional polycation) is greater than 400 nm, then the complex is less suitable for the incorporation of additional moieties, such as targeting factors and/or shielding moieties which may be conjugated to a lipid component of the complex or may be associated with the outside of the lipid complex while not being conjugated to a lipid. Both shielding moieties and targeting factors may increase the effective mean diameter of the lipid complex in buffer or in serum.

The mean diameter of the lipid complex after incubation in serum is indicative of the amount of species present in the serum that have bound to or are associated with, the complex, and thus is indicative of the species present in vivo that may interact, bind to, or associate with the complex. One result of such interactions, (where “interaction”, and its congnates, are used herein to be inclusive of the terms “bind” and “associate with”) is an increase in the mean diameter. For the reasons discussed above, a mean diameter of greater than 400 nm is contraindicated for formulations intended for in vivo or ex vivo administration. The non-specific interaction of the complex with these species results in increased particle size, which may lead to shortened circulatory half-life or aggregation of the particles, and may as well reduce cellular bioavailability of the nucleic acid by reducing the rate or amount of nucleic acid taken up by the cells. Additionally, as pointed out in the Background of Invention, smaller particles tend to show greater size stability than larger particles.

The term “cellular bioavailability” as used herein refers to the availability of the lipid complex in the prescribed compartment of a particular cell with the nucleic acid in a biologically active form, that is, in a form which can be biologically functional.

Species present in serum which may interact with the complexes include for example, serum proteins (e.g., albumin, serum complement), hormones, vitamins, co-factors and others. If the complex interacts with one or more of these species, then the size of the complex may increase after incubation in serum compared to the particle size measured in buffer without prior incubation in serum. Measurement of the size of the complex after incubation in serum may be accomplished using techniques known in the art, for example, as described above, and those described in the Examples.

The complexes described herein may be incubated in mouse, human, horse, rabbit or other serum formulations used in the art. The solution will typically comprise approximately 50% serum with the balance of the solution comprising buffer, for example HEPES or other buffers as described herein and known in the art to be suitable for particular liposome formulations. Solutions may also comprise at least 30%, at least 40%, at least 50%, at least 60%, or at least 70% serum. The complexes are typically incubated in serum for approximately 1 hour. Incubation times may be for at least 30 minutes, at least 45 minutes, at least 1 hour, at least 2 hours, at least 3 hours, at least 6 hours, at least 8 hours, or at least 12 hours. Incubation times may also be approximately 1 hour, approximately 2 hours, approximately 6 hours, approximately 8 hours or approximately 12 hours.

The diameter of the complexes produced by the methods of the present invention may be, for example, about 20 nm to about 500 nm, about 150 nm to about 200 nm, less than about 400 nm, less than about 350 nm, less than about 300 nm, less than about 250 nm, less than about 200 nm, less than about 150 nm, less than about 100 nm. In certain preferred embodiments, the mean diameter of the complex is from about 350 nm to about 50 nm, from about 300 nm to about 100 nm, from about 200 nm to about 100 nm.

Furthermore, smaller particles may be more suitable for use as nucleic acid delivery vehicles. Particle diameters can be controlled by adjusting the nucleic acid/lipid/polycation/targeting factor ratios in the complex, or by size exclusion methods, such as, for example, by passing the complexes through filters. The desired particle diameter may further depend on the cell or tissue type to be targeted. For example, particle diameters of approximately 100-200 nm are particularly preferred for targeting tumor cells, although it is to be understood that other sizes may also be suitable. For targeting lymph nodes, particle diameters of approximately 100 nm are particularly preferred, although it is to be understood that other sizes may also be suitable. For targeting liver cells, smaller particles of about 20 nm are particularly preferred, although it is to be understood that other sizes may also be suitable.

The mean diameter of the complexes may be measured by methods known to those of ordinary skill in the art, including for example, electron microscopy, gel filtration chromatography or by means of quasi-elastic light scattering using, for example, a Coulter N4SD particle size analyzer, as described in the Examples.

As stated above, smaller complexes tend to be more stable over time. The stability of the complexes of the present invention is measured by specific assays to determine the physical stability and biological activity of the complexes over time in storage. The physical stability of the complexes is measured by determining the diameter and charge of the complexes by methods known to those of ordinary skill in the art, including for example, electron microscopy, gel filtration chromatography or by means of quasi-elastic light scattering using, for example, a Coulter N4SD particle size analyzer, or by measuring zeta-potential with a Malvern zeta sizer, as described in the Examples. The physical stability of the complex is “substantially unchanged” over storage when the diameter of the stored complexes is not increased by more than 100%, preferably by not more than 50%, and most preferably by not more than 30%, over the diameter of the complexes as determined at the time the complexes were prepared.

Assays utilized in determining the biological activity of the complexes vary depending on what drug is contained in the complexes. For example, if the drug is nucleic acid encoding a gene product, the biological activity can be determined by treating cells in vitro under transfection conditions utilized by those of ordinary skill in the art for the transfection of cells with admixtures of DNA and liposome complexes. For example, the transfection activity of complexes comprising nucleic acids may be tested using complexes comprising a reporter gene, where reporter genes include, but are not limited to, the chloramphenicol acetyl transferase gene, the luciferase gene, the β-galactosidase gene, the human growth hormone gene, the alkaline phosphatase gene, the red fluorescent protein gene, and the green fluorescent protein gene. Cells which may be transfected by the complexes includes those cells which may be transfected by admixture DNA/liposome complexes. The activity of the stored complexes is then compared to the transfection activity of complexes prepared by admixture. If the drug is an antisense deoxyribonucleic acid then biologic activity may be determined by inhibition of expresssion of the endogenous gene complementary to the drug.

For effectiveness as a nucleic acid delivery complex, the lipid complex must also be characterized with regard to additional properties including the protection that the complex provides to the nucleic acid from degradation by species in serum. This protection from degradation is also an aspect of the “shielding” of the complex which may be provided by shielding moieties, for example a compound comprising PEG. When the drug is a nucleic acid, the nucleic acid is vulnerable to degradation by, for example, nucleases, including RNAases or DNAases, or other species present in serum. As described herein, the lipid complex itself provides protection for the nucleic acid or other drug from degradation by components of serum or other biological fluids, including nucleases. Additionally, as described herein, the incorporation of shielding moieties can also help protect or “shield” nucleic acids from degradation. The sensitivity of nucleic acids to degradation, or the amount of degradation of a nucleic acid may be measured by techniques known to those of skill in the art, including measurement of transfection activity as described above, for example by Pico Green® staining, ethidium bromide staining and gel electrophoresis.

For the nucleic acid to be therapeutically or diagnostically effective, a sufficient amount of intact or biologically active nucleic acid (e.g., for deoxyribonucleic acid in a condition to be accurately transcribed) must be delivered to the cells. Thus, a certain amount of nucleic acid must not be degraded by nucleases or other species present in the serum such that the activity of the nucleic acid is adequate for a therapeutic or diagnostic amount of nucleic acid to be expressed. Accordingly, in certain embodiments, less than 5%, less than 10%, less than 20% or less than 30% of the nucleic acid present in the lipid complex has been degraded. The exact amount of nucleic acid which must be delivered for effective use of the complexes, depends upon the particular use intended and cell type to which the nucleic acid delivered. It is preferrable that at least 50%, at least 60%, at least 70%, at least 80% or at least 90% of the nucleic acid be biologically active.

A further property for characterizing the lipid complexes is the measurement of transfection activity (which may also be referred to as transfection efficiency). Transfection activity may be measured after incubation in serum to determine the possible in vivo effects of serum.components on the transfection activity of the complex and predict in vivo effects on activity, or may be measured without prior incubation in serum. Methods for measuring transfection activity are known in the art and described herein, particularly in the Examples. Incubation in serum prior to measurement of transfection activity may be for the duration and formulations of serum as described above.

Typical levels of in vitro transfection activity for between 0.1-10 μg of DNA total per 5×10⁴ cells are 1×10⁴ to 1×10⁶ RLU/mg protein, when measuring, for example transfection activity of the luciferase gene.

In preferred embodiments, the transfection activity of the lipid complex should be such that the desired therapuetic or diagnostic result is achieved, as described above. For example, when the diagnostic nucleic acid delivered to a cell encodes the green fluorescent protein, the protein must be expressed in the cell at levels which can be detected above the background level of fluorescence. A skilled practioner should be able to determine appropriate levels for determining whether transfection activity is sufficient to result in the desired effect for the purposes of diagnosis or treatment.

A further property which characterizes the complex is complement opsonization. Standard complement opsonization assays known in the art, see for example Ahl et al. (1997) Biochemica et Biophysica Acta 1329:370-382. Exemplary assays include the complement fixation, or as otherwise well known in the art, the complement opsonization assay. In the complement opsonization assay, the lipid complex is incubated with a predetermined amount of serum (e.g., rabbit, human, or guinea pig). Red blood cells (e.g., sheep, rabbit, human) that have been treated with antibodies that react specifically to the species of red blood cell (e.g., if sheep red blood cells are used the secondary antibody would be a rabbit-anti-sheep red blood cell antibody) are then incubated with the lipid complex treated with serum. The level of predetermined serum referred to earlier is based on the level of serum necessary to lyse approximately 95% of the secondary antibody treated red blood cells when preincubated only with buffer. If the lipid complexes opsonize/bind/ or interact with serum complement proteins the complement components become limiting and when incubated with secondary antibody coated red blood cells the degree of lysis of the red blood cells (e.g., hemolysis) is reduced compared to the buffer control. The degree of hemolysis can be measured by a number of methods known in the art including spectrophotometrically. By varying the dilution of serum a standard curve can be generated using control buffer or control lipid formulations. The dilution of serum which results in 50% hemolysis is referred to as the CH50 and can be used to compare lipid formulations. Generally the lipid formulations of the invention have a CH50 that is between 2 and 1000-fold of a control buffer.

The level of complement opsonization in serum is indicative of the extent of interactions, particularly non-specific interactions, which the complex will have with species present in the serum in vivo. As described earlier, non-specific interactions with species present in serum may reduce the circulatory half-life and/or cellular bioavailability of the nucleic acid to be delivered to the cell. Shielding moieties may be incorporated into the lipid complex to reduce the number or intensity of interactions with species in the cell and thus increase the circulatory half-life and/or cellular bioavailability of the nucleic acid.

As described above, typically, a complex should be characterized with respect to nucleic acid protection from degradation, mean diameter after and/or prior to incubation in serum, the level of complement opsonization and transfection activity. Complexes may also be tested for cell toxicity as described herein and shown in the Examples. Toxicity can be expressed as a survival percentage. In certain embodiments, a minimum of 50% survival is preferred. In particular embodiments, survival of 60-80% is preferred.

As described above, the circulatory half-life of complexes is effected by a number of factors, including mean diameter, shielding, targeting factors, etc. Thus, complexes may be further characterized by measurement of their circulatory half-life. Generally, a longer circulatory half-life is preferable. That is, an increase in circulatory half-life of a complex upon incorporation of a shielding moiety such as PEG will typically result in more nucleic acid delivery to the particular cell. However, it is also possible that, for certain formulations, the complex is so well shielded that it is never bioavailable to (e.g., able to be taken up by) cells. The measurement of the circulatory half-life may be able to distinguish between this case and one in which the complex is aggregating under certain conditions.

Methods for measuring circulatory half-life are known in the art and include radiotracer deposition, HPLC, or PCR. The length of the preferred circulatory half-life of complexes will vary depending on a variety of factors. Such factors include, the condition to be treated or diagnosed, the severity of the condition, the cell type or cell types targeted, the location of the cell types targeted, the method of delivery of the complex, the frequency of delivery of the complex, the amount of drug delivered, and the toxicity of the drug being delivered, and, for in vivo or ex vivo delivery, the sex, weight, age and general health of the individual to whom the complex is being administered. A skilled practioner should be able to account for these factors when determining the type of complex to be used and the amount and frequency of delivery of the complex.

In certain embodiments, preferred properties for lipid complex size include a mean diameter of less than 400 nm, transfection efficiency of at least 70% in serum compared to transfection not in serum, nucleic acid protection of at least 50%, and a reduction in the ability to fix complement of at least 50%.

In certain embodiments, the complex will have the following characteristics: reduced complement opsonization have a mean diameter after incubation in 50% serum for 1 hour of less than 400 nm, less than 300 nm, less than 200 nm, less than 150 nm, or less than 100 nm; have transfection activity of 1% to 100% of that in the absence of serum, preferably at least 30%, at least 40%, at least 50%, at least 60%, at least 70% of that in serum.

The complexes formed by the methods described herein may also be characterized in relation to their toxicity, circulatory half-life and stability over time, as described herein. For a complex to be suitable for the uses as described herein, particularly in vivo diagnostic or treatment uses, the complexes should also be suitable for preparation as “one vial” formulations. “One vial” formulations, in addition to being effective for the desired use and of low toxicity and therapeutic or diagnostic efficacy, as are all formulations for use in vivo, should additionally be stable over time when all components of the complex have been formulated together. As described above, stability refers to both physical characteristics (e.g. mean diameter) and biological activity (e.g. transfection level).

The term “reproducible complexes” and its congnates, are used to describe complexes which routinely have the properties of complexes prepared by the methods described herein, and as described in the previous section. For example, complexes are characterized by the assay methods and measurements as described here, including size after incubation in serum, transfection levels, and complement opsonization. The complexes may also have properties which are equivalent to those described in the previous section, but are obtained by different assay methods. For example, where the properties of the are determined by means other than measurement of mean diameter after incubation in serum or by complement opsonization, or by measurement of transfection levels in cells other than those described herein (e.g. cells other than KB, LL2 or MDA-231 cells) and/or using different reporter genes or probes to determine transfection levels. A skilled practioner would be able to compare such characteristics and determine such equivalence. In the embodiments described herein, the lipid/nucleic acid complexes of the invention are characterized in that they have the properties described above, or properties equivalent to those described above and further, can be formulated reproducibly so as to exhibit these characteristics.

Additional methods suitable for testing drug delivery complexes of the invention may be found in U.S. Pat. Nos. 5,795,587 and 6,008,202, which are hereby incorporated by reference in their entirety.

Therapeutic formulations using the complexes of the invention preferably comprise the complexes in a physiologically compatible buffer such as, for example, phosphate buffered saline, isotonic saline, or low ionic strength buffer such as 5% dextrose or 10% sucrose in H₂O (pH 7.4-7.6) or in HEPES (pH 7-8, a more preferred pH being 6.8-7.4).

Drug Delivery Complexes

Provided by certain embodiments of the present invention are complexes of at least one fusogenic lipid and compacted nucleic acid with the above-described properties, or equivalent properties, which comprise at least one anionic or pH sensitive fusogenic lipid. Also provided by particular embodiments of the present invention are complexes of non-fusogenic anionic lipids and compacted nucleic acid with the above-described properties, or equivalent properties, which further comprise at least one fusogenic moiety. In certain examples, the fusogenic moiety may be a fusogenic lipid. In other examples, the targeting factor or shielding factor may comprise a fusogenic moiety as described herein. For complexes comprising anionic lipids, including fusogenic anionic lipids, a polycation is required to compact the nucleic acid. The complexes described above may optionally comprise a targeting factor, either specific or non-specific, which may, or may not, be conjugated to any other component of the complex. The complexes described may also optionally comprise a shielding moiety which may or may not be conjugated to any other component of the complex. The complexes may also comprise one or more co-lipids.

In another embodiment is provided a complex having the properties of, or properties equivalent to, those described above, comprising lipid, compacted nucleic acid and a targeting factor which increases cellular bioavailability by a means other than targeting of a specific cell surface receptor, as measured by an increase in gene expression. The complex may optionally comprise a polycation, and/or a shielding factor and/or fusogenic moiety(s) and/or one or more co-lipids

Each of the complexes described herein may further comprise a shielding moiety. Examples of shielding moieties include compounds comprising polyethylene glycol and other compounds which reduce the interaction or binding of the complex to species present in vivo or in vitro, such as serum complement protein, co-factors, hormones or vitamins.

In particular embodiments where the lipid species comprises a pegylated lipid, the total content of pegylated lipid, as a percentage of total lipid content, will be in the range of 0% to approximately 20%. In other embodiments the range of pegylated lipid will be approximately 0-10%, approximately 0-6%, approximately 0-5%, approximately 0-4% or approximately 0-3%. In certain embodiments, the total content of pegylated lipid will be approximately 2.5%, approximately 4%, approximately 5%, approximately 10%, approximately 15% or approximately 20%. A complex may contain a both pegylated and non-pegylated lipid of a particular type, for example, pegylated and non-pegylated DSPE. In certain embodiments, the total pegylated lipid content is no more than approximately 10%.

In certain embodiments, degradation of nucleic acid can be measured by techniques well known in the art, for example, Pico Green® staining, ethidium bromide staining or gel electrophoresis. Techniques for measuring the amount of complement fixed by a particular complex are described herein and well known in the art. See, for example Ahl et al. (1997) Biochemica et Biophysica Acta 1329:370-381.

In certain embodiments of the complex described herein, at least one co-lipid may be non-fusogenic, in other embodiments at least one co-lipid may be fusogenic. In particular embodiments at least one co-lipid may be a neutral phospholipid.

The drug delivery complexes as described herein may be formulated with any of the lipids; targeting factors; polycations; shielding moieties; drugs, in particular nucleic acids; described herein, unless indicated otherwise. Additionally, the drug delivery complexes-described herein may be made by and used with the methods herein described according to the guidelines set out herein.

Certain embodiments further provide use of the complexes as described herein in the manufacture of a medicament for the treatment or diagnosis of a disease, condition, or syndrome.

By “drug” as used throughout the specification and claims is meant any molecular entity, which is either monomeric or oligomeric, and which, when complexed with the lipid(s), optional polycation, and targeting factor, is being administered to an individual for the purpose of providing a therapeutic or prophylactic effect to the recipient, or which is administered for diagnostic purposes. Thus, macromolecules having an overall net negative charge or regions of negativity would be expected to be capable of forming the delivery complexes of this invention. Macromolecules which are particularly suitable for use with the complexes of this invention are, for example, DNA, RNA, oligonucleotides or negatively charged proteins. However, macromolecules having a positive charge (e.g., large cationic proteins) would also be expected to be capable of forming the complexes of this invention by sequentially complexing the cationic macromolecule with anionic molecule or polymer and then with cationic lipid, or by incorporating the cationic macromolecule into complexes comprising anionic polymer or lipid. In preferred embodiments, the drug is a nucleic acid, and the term nucleic acid and drug will be used interchangeably from this point on.

“Polyethylene glycol” and “PEG” refer to compounds of the general formula H(OCH₂CH₂)ₙOH, wherein n may be any integer greater than 1. Preferred PEG formulations have an average molecular weight of about 750-20,000. As used herein, “PEG” and “polyethylene glycol” are meant to encompass PEG compositions which may optionally include one or more functional groups (such as, e.g., methoxy, biotin, succinyl, nickel or conjugating PEG to another moiety, such as a lipid or a targeting factor.

“Pegylated lipid” is used herein to indicate a lipid which is conjugated to a polyethylene glycol (PEG) moiety.

“Targeting factor-pegylated lipid conjugate” is used herein to indicate a targeting factor which has been conjugated to a pegylated lipid. The targeting factor may be conjugated, for example, to the PEG moiety of the pegylated lipid.

“Targeting factor-lipid conjugate” is used herein to indicate a targeting factor which has been conjugated to a lipid.

“Targeting factor”, as used herein, indicates a synthetic or naturally occuring moiety which increases cellular (for example, intracellular) bioavailability of the drug. The targeting factor may effect increased cellular bioavailability at the desired location(s) through specific and/or non-specific interactions with a cell membrane, such as an outer cell surface membrane, nuclear membrane or endosomal membrane. The targeting factor may act specifically, for example, by preferentially binding a certain type(s) of cells (e.g., cancer cells) over other types of cells, the targeting factor binds to or interacts with the targeted cell type with at least 1.5×, at least 2×, at least 5×, at least 10×, at least 100×, at least 200× greater affinity than other cell types. The targeting factor may also act by increasing cellular uptake of the drug, for example, by facilitating drug transport across the cellular membrane (see, e.g., MTLP peptides), (e.g., outer cell surface membrane, nuclear membrane or endosomal membrane) thereby producing a therapeutic and/or prophylactic and/or diagnostic level of drug in the cell. In other embodiments the targeting factor increases the rate or amount of drug entry into, or exit from, a cellular compartment. The targeting factor may also increase cellular bioavailability through increasing transcription of nucleic acid. The targeting factor may also be multifunctional, comprising both specific targeting elements and non-specific elements which increase drug uptake at the target cells or sites following targeting particular cell types. A non-limiting example of a multifunctional targeting factor is galactose-Elan094 as described in detail in the Examples infra. Examples of non-specific elements include targeting factors which increase cellular bioavailability by a means other than a specific outer cell surface membrane receptor, such as, for example, membrane-disrupting synthetic polymers, including pH sensitive membrane-disrupting synthetic polymers. More than one targeting factor may be incorporated into a complex to enhance either specific or non-specific targeting.

As used herein, the term “membrane-disruptive synthetic polymer” or “membrane-disrupting synthetic polymer” refers to synthetic polymers, such as poly(alkylacrylic acid) polymers which do not disrupt cellular membranes under typical physiological conditions (e.g. of pH, temperature or light conditions) but when the conditions are altered, do disrupt cellular membranes. For example, pH sensitive membrane-disrupting synthetic polymers do not disrupt cellular membranes at physiological pH (e.g. approx. pH 7 to approx. pH 8.5) but do disrupt cellular membranes at a different pH, for example, endosomal pH (e.g. approx. pH 4.5 to approx. pH 6). A pH-sensitive endosomal membrane-disruptive synthetic polymer would refer to a polymer as described above which disrupts endosomal membranes at endosomal pH, but would leave cell-surface or nuclear membranes intact.

An “RGD motif” indicates a peptide which comprises an arginine-glycine-aspartic acid (RGD) sequence.

“DSPE-PEG_{5K}-RGD” is used herein to indicate DSPE-PEG_{5K}-succinyl-ACDCRGDCFCG-COOH (SEQ ID) NO:10).

“DSPE-PEG_{5K}-LHRH” is used herein to indicate pyrGLU-HWSY_{D}K(εNH-succinyl-PEG_{5K}-DSPE)LRPG-COOH (SEQ ID NO:11).

“MTLP” is used herein to indicate a membrane translocating peptide, i.e., a peptide which facilitates translocation of the lipid/drug complex and/or the drug across a cellular membrane.

“MTLP-lipid” is used herein to indicate a lipid which is conjugated to a MTLP sequence.

“DOPE-094” and “Elan 219” are used interchangeably herein to indicate DOPE-succinyl-KKAAAVLLPVLLAAP (SEQ ID NO:12).

“Elan094” is used herein to indicate the peptide sequence KKAAAVLLPVLLAAP (SEQ ID NO:13).

The terms “polypeptide”, “oligopeptide”, “peptide” and “protein” are used interchangeably herein to refer to polymers of amino acids of any length. The polymer may be linear or branched, it may comprise modified amino acids, it may contain one or more non-peptide bonds, and it may be assembled into a complex of more than one polypeptide chain. The terms also encompass an amino acid polymer that has been modified naturally or by intervention; for example, by disulfide bond formation, glycosylation, lipidation, acetylation, phosphorylation, prenylation, myristolyation, palmitolyation, or any other manipulation or modification, such as conjugation with a labeling component. Also included within the definition are, for example, polypeptides containing one or more analogs of an amino acid (including, for example, unnatural amino acids, etc.), non-peptide bond, as well as other modifications known in the art. It further encompasses polymers made from L-amino acids and/or D-amino acids.

The terms “polynucleotide”, “oligonucleotide”, and “nucleic acid” are used interchangeably herein to refer to polymers of nucleotides of any length. The terms also include analogues and derivatives of oligonucleotides known in the art.

“Cationic complex”, as used herein, is meant to include a drug/lipid/targeting factor complex, which optionally comprises polycation, having a net positive charge and/or a positively charged surface. It is meant to include cationic liposomes, micelles, colloidal solutions, mixed micelles, and more amorphous lipid structures. The net charge of a complex may be measured by the migration of the complex in an electric field by methods known to those in the art such as by measuring zeta potential (Martin, A., Swarick, J., and Cammarata, A., Physical Pharmacy & Physical Chemical Principles in the Pharmaceutical Sciences, 3rd ed. Lea and Febiger, Philadelphia, 1983).

“Anionic complex”, as used herein, is meant to include a drug/lipid/polycation/targeting factor complex having a net negative charge and/or a negatively charged surface. It is meant to include anionic liposomes, micelles, colloidal solutions, mixed micelles, and more amorphous lipid structures.

As used herein, the term “anionic lipid” refers to a lipid which is negative at physiological pH, that is between approximately pH 7 and approximately 8.5.

Similarly, a “neutral lipid” is a lipid which is neutral or charge-balanced at physiological pH, and a “cationic lipid” is a lipid which is positively charged at physiological pH.

Lipids which are described as “pH sensitive” lipids may also be classed as “anionic”, “cationic” or “neutral” depending on their charge at physiological pH. For example, DOPE may be referred to as a neutral lipid because it is neutral at approximately pH 7, however it is a pH sensitive lipid which is anionic at pH approximately 9.

The term “fusogenic” may be used to describe either lipids or other components of the complexes described herein. The term “fusogenic moiety” refers to both fusogenic lipids and other fusogenic components of the complex unless noted otherwise or indicated by context. As used herein the term “fusogenic” refers to a moiety which enhances or enables the translocation of the complexes (or drugs) described herein across a cellular membrane. The membrane may be either an outer cell surface membrane, endosomal membrane or a nuclear membrane. The fusogenic moiety may increase the transport of the complex, or components of the complex, including for example nucleic acid, across a cell surface membrane into the interior of a cell, or increase the entry into, or exit from, a cellular compartment. Such compartments could be, for example, endosomes or the nucleus. Examples of complex components which may be fusogenic include, for example, lipids, targeting factors, or shielding moieties. In certain embodiments the fusogenic moiety may be for example a targeting factor such as a membrane-disruptive synthetic polymer, or for example, a targeting factor comprising a membrane translocating sequence (e.g. MTLP).

The term “fusogenic lipid” may be used to refer to lipids which undergo a change in structure or charge at endosomal pH, when compared to their charge or structure at physiological pH, which results in the lipid becoming more fusogenic. These fusogenic lipids may be anionic lipids, neutral lipids or pH sensitive lipids which are characterized in that when the pH is changed from approximately pH 7 to approximately pH 4.5, the lipid undergoes a change in charge or structure such that it becomes more fusogenic. The change in charge or structure may also occur at pH's approximately 4.5 to approximately 6. The change in charge or structure may, in some embodiments, be linked to entry into, for example an endosome, and as such the pH may range from that of early to late endosomes (e.g. approximately pH 4.5, 5, 5.5 or 6). In certain embodiments the complex comprises at least one fusogenic lipid, such as 1,2-dioleoyl phosphatidylethanolamine (DOPE), 1,2-dioleoyl-sn-glycero-3-[phosphoethanolamine-N-dodecanoyl] (NC₁₂-DOPE), cholesteryl hemisuccinate (CHEMS), 1,2-dioleoyl-sn-glycero-3-[phospho-L-serine] (DOPS). In certain embodiments, when the pH is lowered to approximately pH 4.5, the fusogenic anionic lipid undergoes a change in charge to become neutral or cationic. In other embodiments, the fusogenic pH sensitive lipid may undergo a change in charge upon a lowering of pH to approximately 4.5 such that a neutral or anionic lipid becomes cationic or neutral. In other embodiments, when the pH is lowered to pH approximately 4.5 the fusogenic lipid undergoes a change in structure such that it assumes a hexagonal or cone-forming structure. Additional fusogenic lipids of this type are known in the art and may be used in the formulations, complexes and methods described herein. Some examples of these “fusogenic” lipids change structure to adopt a hexagonal structure, while other examples of these lipids undergo a change in charge from being negatively charged anionic lipids to neutral lipids, or, from neutral lipids to positively charged, cationic lipids. These fusogenic lipids may also include those referred to as “cone-forming” lipids in the art. The term “fusogenic lipid” may also be used to refer to lipids that exhibits molecular shape properties of cone formation such that the lipid framework comprises a small cross sectional head group and a larger acyl chain cross-sectional area. Without wishing to be bound by theory these lipids are thought to induce a nonbilayer hexagonal H_{II}, phase

The change in charge of a lipid or combination of lipids may be determined by measuring zeta potential, as described above and in the Examples. The structure of a lipid or combination of lipids under different pH conditions may be determined by methods known to those in the art and as described in the Examples. Such methods include for example, electron microscopy, particluarly negative stain electron microscopy (see, for example, Lee & Huang (1996) supra), and others, including, but not limited to atomic force microscopy, cryoelectron microscopy, freeze fracture microscopy, ³¹P NMR and lipid mixing experiments.

As used herein, the term “compacted nucleic acid”, and its congnates, refer to a nucleic acid which has been “compacted” or “condensed”. Examples of “compacting” or “condensing” agents include polycations, such as synthetic polycations (e.g. polycationic methacryloxy polymers, polycationic methacrylate polymers and polycationic poly(alkenylimines), such as PEI, polymers comprising dimethylamino methacrylate, such as co-polymers of dimethylamino methacrylate and methacrylic ester (e.g Eugadrit® 100, Eugadrit® EPO), and poly(2-methacryloxyethyltrimethyl ammonium bromide (PMOETMAB)); polycationic polypeptides (e.g. histones, protamines, spermidine, polyarginine, polylysine, etc.); polycationic polypeptide salts. The terms “condensed” and “compacted” and their congnates and combinations such as “compaction agent” or “condensation agent”, etc. may be used interchangeably unless otherwise noted herein. Preferred are polycations other than protamine and salts thereof.

The term “synthetic polycation” may be used to described polycations which are capable of compacting nucleic acid and which are suitable for use with lipids and the formation of liposomes. Examples of synthetic polycations include poly(alkenylimines) (e.g. polyethylene imine (PEI)), polycationic methacrylate polymers (e.g. polymers comprising dimethylamino methacrylate and co-polymers of dimethylamino methacrylate and methacrylic ester, for example Eudragit® polycations, Eudragit® E100, Eudragit® EPO), and polycationic methacryloxy polymers (e.g. poly(2-methacryloxyethyltrimethyl ammonium bromide (PMOETMAB)). The term synthetic polycation is not intended to include polycationic polypeptides and their salts, such as protamines, histones, poly-L-lysine and the like.

The term “micelle” or its congnates can be used to describe a lipid monolayer, which is distinguished from a liposome which is a lipid bilayer.

Targeting Factors

The targeting factor may comprise, for example, modified lipids, peptide, protein, polycations, synthetic polymers, synthetic compounds, receptor ligands, small molecules, vitamins, hormones, metals, carbohydrates, membrane-disruptive synthetic polymers, membrane-disruptive polymers or endosomal membrane-disruptive synthetic polymers, or nucleic acids which function to direct the complex to a particular tissue or cell type, or which facilitate drug transport across the cellular membrane, including, but not limited to an outer cell surface membrane, nuclear membrane or endosomal membranes. In other embodiments the targeting factor increases the rate or amount of drug entry into, or exit from, a cellular compartment. Targeting factors may also increase transcription within the nucleus. Potential targets include, but are not limited to, liver cells, blood cells, kidney cells, prostate cells, lung epithelial cells, lung endothelial cells, fat cells, epithelial cells, endothelial, fibroblast cells and tumor cells. In a preferred embodiment, the target is a tumor cell.

Examples of suitable targeting factors include, but are not limited to, asialoglycoprotein, insulin, low density lipoprotein (LDL), growth factors, galactose, adhesion molecules, lectin, nucleic acids, folate, MTLPs, membrane-disruptive synthetic polymers, membrane-disruptive polymers, endosomal membrane-disruptive synthetic polymers, poly(alkylacrylic acids) and monoclonal and polyclonal antibodies directed against cell surface molecules. In a preferred embodiment, the targeting factor is luteinizing hormone-releasing hormone (LHRH). In another preferred embodiment, the targeting factor comprises an adhesion molecule. In another embodiment the targeting factor is a pH sensitive membrane-disruptive synthetic polymer. Non-limiting examples of a pH sensitive membrane-disruptive synthetic polymers are poly(alkylacrylic acids). In certain embodiments the polycrylic acid may be poly(propyl acrylic acid) (PPAA), poly(ethyl acrylic acid) (PEAA). In other embodiments, the poly(alkylacrylic acid) is PPAA. Other membrane-disrupting synthetic polymers are known in the art and are described in, for example, Lackey et al. (1999) Bioconj. Chem. 10:401-405; Murthy et al. (1999) J. Controll. Release 61:137-143; Stayton et al. (2000) J. Controll. Release 65:203-220; and WO 99/34831. (Cheung et al. (2001) Bioconj. Chem. 12:906-910), Lackey et al. (1999) supra and Murthy et al. (1999) supra also describe the preparation of pH sensitive membrane-disruptive synthetic polymers.

A non-limiting example of an adhesion molecule is a peptide which comprises an arginine-glycine-aspartic acid (RGD) motif. A nonlimiting example of a suitable RGD motif peptide is H₂N-ACDCRGDCFCG-COOH (RGD4C) (SEQ ID NO:14). A nonlimiting example of a MTLP is H₂N-KKAAAVLLPVLLAAP-COOH (Elan094) (SEQ ID NO:2). Other suitable examples of MTLP-comprising targeting factors include: H₂N-KKAAAVLLPVLLAAP-COOH (Elan094) (SEQ ID NO:2), H₂N-KKKAAAVLLPVLLAAP (ZElan094) (SEQ ID NO:3), H₂N-kkkaavllpvllaap (ZElan207) (SEQ ID NO:4), H₂N-KKKAAAVLLPVLLAAPREDL (ZElan094R) (SEQ ID NO:5); H₂N-GLFGAIAGFIENGWEGMIDGWYG-COOH (Influenza HA-2 (INF6)) (SEQ ID NO:15); H₂N-GLFEALLELLESLWLLEA-COOH (JTS1) (SEQ ID NO:16); H₂N-HHHHHWYG-COOH (H₅WYG) (SEQ ID NO:17); H₂N-WEAALAEALAEALAEHLAEALAEALEALAA-COOH (GALA) (SEQ ID NO:18); H₂N-WEAKLAKALAKALAKHLAKALAKALKACEA-COOH (KALA) (SEQ ID NO:19); VP22 (HSV-1); H₂N-CPCILNRLVQFVKDRISVVQAL-COOH (Retrovirus intra-cellular domain(Mo-MuLv)) (SEQ ID NO:20); H₂N-RQIKIWFQNRRMKWKK-COOH (Homeobox domain penetration) (SEQ ID NO:21); H₂N-RQPKIWFPNRRKPWKK-COOH (Homeobox domain penetration) (SEQ ID NO:22); H₂N-PLSSIFSRIG-COOH (PreS2 domain) (SEQ ID NO:23); H₂N-RGGRLSYSRRRFSTSTGR-COOH (SynBI)) (SEQ ID NO:24); protein transduction domains (PTDs) (e.g. H₂N-YGRKKRRQRRR-COOH (TAT) (SEQ ID NO:25); H₂N-RQIKIWFQNRRMKWKK-COOH (Antp) (SEQ ID NO: 21); H₂N-RRRRRRR-COOH (Arg) (SEQ ID NO:26); and H₂N-HHHHHHHHH-COOH (His) (SEQ ID NO:27). In particular embodiments, the MTLP is H₂N-KKKAAAVLLPVLLAAP (ZElan094) (SEQ ID NO:3), H₂N-kkkaavllpvllaap (ZElan207) (SEQ ID NO:4), or H₂N-KKKAAAVLLPVLLAAPREDL (ZElan094R) (SEQ ID NO:5), where the lower case letters indicate D-amino acids. Additional targeting factors include targeting factor-comprising compounds selected from the group consisting of H₂N-K(dansyl)KKAAAVLLPVLLAAP (ZElan094) (SEQ ID NO:28), H₂N-k(dansyl)kkaavllpvllaap (ZEla207) (SEQ ID NO:29), H₂N-K(dansyl)-H₂N-KKKAAAVLLPVLLAAP (ZElan094) (SEQ ID NO:30), H₂N-kkkaavllpvllaap (ZElan207) (SEQ ID NO:4), H₂N-K-KKAAAVLLPVLLAAPREDL (ZElan094R) (SEQ ID NO:5), des-Pro-KKAAAVLLPVLLAAS-Galactose (Elan094G) (SEQ ID NO:31), S(Galactose)KKAAAVLLPVLLAAP (GElan094) (SEQ ID NO:32), Cholesteryl-succinyl-KKAAAVLLPVLLAAP (Elan218) (SEQ ID NO:33), DOPE-succinyl-KKAAAVLLPVLLAAP (Elan219) (SEQ ID NO:34), Cholesteryl-succinyl-kkaaavllpvllaap (All d-Elan218) (SEQ ID NO:35), DSPE-PEG_{5K}-succinyl-KKAAAVLLPVLLAAP (DSPE-PEG_{5K}-Elan218) (SEQ ID NO:36), DMPE-PEG_{5K}-succinyl-KKAAAVLLPVLLAAP (DMPE-PEG_{5K}-Elan218) (SEQ ID NO:37), DSPE-PEG_{5K}-succinyl-KKAAAVLLPVLLAAP (DSPE-PEG_{5K}-Elan219) (SEQ ID NO:38), and DMPE-PEG_{5K}-succinyl-KKAAAVLLPVLLAAP (DMPE-PEG_{5K}-Elan219) (SEQ ID NO:39), wherein the lower case letters representing amino acids indicate D-amino acids. Incorporation of the dansyl (dansylated chloride) and des-Pro tags in the Elan moieties listed above “tags” or labels the molecule such that the moieties containing these tags may be tracked/monitored experimentally. One skilled in the art would appreciate that this may or may not be incorporated in the formulation depending on in vivo use, either diagnostic or therapeutic. In one embodiment, the complex further comprises a pegylated lipid. In another embodiment, the complex further comprises DSPE-PEG_{5K}-LHRH (SEQ ID NO:11).

When the targeting factor is a peptide, L-amino acids and/or D-amino acids may be used. Generally, peptides composed of L-amino acids are less stable in serum than those made with D-amino acids, and thus may be preferred for complexes requiring a shorter circulation half life, or where optimal tissue uptake in the desired cells occurs in those tissues exposed to or in contact with the administered formulation over the relatively shorter lifetime of the peptide, for example, when targeting lung, liver, or heart cells. Alternatively, peptides composed of D-amino acids may be preferred when longer circulation half lives are required.

Membrane translocating peptides or targeting factor peptides may be synthesized using chemical methods (see, e.g., U.S. Pat. Nos. 4,244,946, 4,305,872 and 4,316,891; Merrifield et al. J. Am. Chem, Soc. 85:2149, 1964; Vale et al. Science 213:1394, 1981; Marki et al. J. Am. Chem. Soc. 103:3178, 1981); recombinant DNA methods (e.g, Maniatis, Molecular Cloning, a Laboratory Manual, 2d ed. Cold Spring Harbor Laboratory, Cold Spring Harbor N.Y., 1990) or other methods known to those skilled in the art.

Chemical methods include, but are not limited to, solid phase peptide synthesis. Briefly, solid phase peptide synthesis consists of coupling the carboxyl group of the C-terminal amino acid to a resin and successively adding N-alpha protected amino acids. The protecting groups may be any known in the art. Before an amino acid is added to the growing peptide chain, the protecting group of the previous amino acid is removed (Merrifield J. Am. Chem. Soc. 85:2149 1964; Vale et al. Science 213:1394, 1981; Marki et al. J. Am. Chem. Soc. 103:3178, 1981). The synthesized peptides are then purified by methods known in the art.

Preferably, solid phase peptide synthesis is done using an automated peptide synthesizer such as, but not limited to, an Applied Biosystems Inc. (ABI) model 431 A using the “Fastmoc” synthesis protocol supplied by ABI. This protocol uses 2-(1H-Benzotriazol-1-yl)-1,1,3,3-tetramethyluronium hexafluorophosphate (HBTU) as coupling agent (Knorr et al. Tet. Lett. 30:1927, 1989). Syntheses can be carried out on 0.25 mmol of commercially available 4-(2′,4′-dimethoxyphenyi-(9-fluorenyl-ethoxycarbonyl)-aminomethyl) phenoxy polystyrene resin (Rink H. Tet. Lett. 28:3787, 1987). Fmoc amino acids (1 mmol) are coupled according to the Fastmoc protocol. N-methylpyrrolidone (NMP) is used as solvent, with HBTU dissolved in N,N-dimethylformamide (DMF). The following side chain protected Fmoc amino acid derivatives are used: FmocArg(Pmc)OH; FmocAsn(Mbh)OH; FmocAsp(tBu)OH; FmocCys(Acm)OH: FmocGlu(tBu)OH; FmocGln(Mbh)OH; FmocHis(Tr)OH, FmocLys(Boc)OH; FmocSer-(tBu)OH; FmocThr(tBu)OH; FmocTyr(tBu)OH. (Abbreviations: Acm:acetamidomethyl; Boc:tert-butoxycarbonyl; tBu:tert-butyl; Fmoc:9-fluorenylmethoxy-carbonyl; Mbh;4,4′-dimethoxybenzhydryl; Pmc:2,2,5,7,8-pentarnethylchro-man-6-sulfonyl; Tr:5 trityl).

Deprotection of the Fmoc group is effected using approximately 20% piperidine in NMP. At the end of each synthesis, the amount of peptide is assayed by ultraviolet spectroscopy. A sample of dry peptide resin (about 3-10 mg) is weighed, then 20% piperidine in DMA (10 ml) is added. After 30 min sonication, the UV (ultraviolet) absorbance of the dibenzofulvene-piperidine adduct (formed by cleavage of the N-terminal Fmoc group) is recorded at 301 nm. Peptide substitution (in mmol/g) is calculated according to the equation:

$$Substitution = \frac{A \times v \times 1000}{7800 \times w}$$

where A is the absorbance at 301 nm, v the ml of 20% piperidine in DMA, 7800 the extinction coefficient (mol/dm³/cm) of the dibenzofulvene-piperidine adduct, and w the mg of peptide-resin sample. The N-terminal Fmoc group is cleaved using 20% piperidine in DMA, then acetylated using acetic anhydride and pyridine in DMA. The peptide resin is thoroughly washed with DMA, CH₂C₁₂ and diethyl ether.

Methods used for cleavage and deprotection (King et al. Int. J. Peptide Protein Res. 36:255. 1990) include, but are not limited to, treating the air-dried peptide resin with ethylmethyl-sulfide (EtSMe), ethanedithiol (EDT) and thioanisole (PhSMe) for approximately 20 min and adding 95% aqueous trifluoracetic acid (TFA). Approximately 50 ml of these reagents are used per gram of peptide-resin in a ratio of TFA:EtSMe:EDT:PhSme (10:0.5:0.5:0.5). The mixture is stirred for 3 h at RT under an N₂ atmosphere, filtered and washed with TFA (2×3 ml). The combined filtrate is evaporated in vacuo and anhydrous diethyl ether is added to the yellow/orange residue. The resulting white precipitate is isolated by filtration. Purification of the synthesized peptides is done by standard methods including, but not limited to, ion exchange, affinity, sizing column and high performance liquid chromatography, centrifugation or differential solubility.

The targeting factor may be conjugated, for example, to the PEG moiety of a pegylated lipid. See, e.g., Harasym, T. O., et al. (1998) Advanced Drug Delivery Reviews 32:99-118 and references described therein, which describes peptide finctional groups suitable for linking to functional groups on a PEG moiety or as described infra. Other examples for synthesis of peptide targeting factors to finctionalized PEG moieties include:

Amine-specific PEGylation mPEG-ALD

mPEG-Propionaldehyde (mPEG-ALD) Synthesis

PEGs bearing aldehyde groups undergo reductive amination reactions with primary amines in the presence of sodium cyanoborohydride. Unlike other electrophilically activated groups, the aldehyde reacts only with amines. Although aldehyde is much less reactive than the NHS esters, this reaction takes place under mild conditions (pH 6-9.5, 6-24 hours) and has been shown to be useful for attaching PEG to surfaces (Harris, J. M. et al (1984) J. Polym. Sci. Polym. Chem. Ed. 22:341) and proteins (U.S. Pat. No. 5,824,784; Wirth, P. et al. (1991) Bioorg. Chem. 19:133). At lower pH, selective reaction at the N-terminus becomes possible. The stability of the attachment (a secondary amine is formed upon reduction) is important for such applications as preparation of affinity supports and immobilized enzymes. Proteins modified in this fashion retain amine groups and associated charge in solution, which can be important for maintaining protein conformation and activity. These conjugates can be conveniently characterized by quantitation of lysine in their hydrolysis products by amino acid analysis. mPEG-ALD has also been used to form acetal linkages with hydroxyl groups of polyvinyl alcohol (Llanos, G. R. & Sefton, J. V. (1991) Macromol. 24:6065). The propionaldehyde derivative offered here has the advantage of being much more stable in basic media than the acetaldehyde derivative (Llanos, G. R. & Sefton, J. V. (1991) Macromol. 24:6065; Harris, J. M., et al. (1991) in “Water-Soluble Polymers,” S. W. Shalaby, C. L. McCormick, and G. B. Butler, Eds., ACS, Washington, D.C., Chapter 27). The mPEG-ALDs are very popular for N-terminal PEGylation of proteins and two mPEG-ALDs, 20,000 and 30,000 Da, are being used in Phase III and Phase II clinical trials with two different proteins, respectively.
Amine-specific PEGylation mPEG-BTC 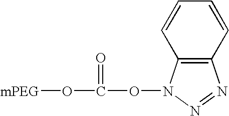
mPEG-Benzotriazole Carbonate

The benzotriazole carbonate derivative of mPEG (mPEG-BTC) is an exceptional alternative to the succinimidyl carbonate (mPEG-SC). While not as reactive as some of the NHS active esters, mPEG-BTC is an efficient modifier of peptide and protein amino groups, producing a stable urethane (carbamate) linkage. MPEG-BTC is sufficiently reactive to produce extensively modified PEG-proteins under mild conditions within short periods of time. The MPEG-BTC is an intermediate for several cGMP syntheses and is expected to begin cGMP synthesis as a final product in 2001. 5,000 and 20,000 Da have been the most commonly used molecular weights.
Aminespecific PEGylation mPEG-SPA and mPEG-SBA 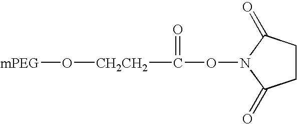
mPEG-Succinimidyl Propionate (mPEG-SPA) 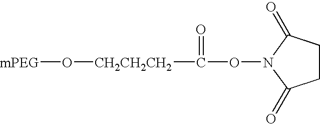
mPEG-Succinimidyl Butanoate (mPEG-SBA)

The NHS esters of PEG carboxylic acids are the most popular derivatives for coupling PEG to proteins. Reaction between lysine and terminal amines and the active esters produces a stable amide linkage (Olson, K. et al, (1997) J. M. Harris & S. Zalipsky Eds., Poly(ethylene glycol), Chemistry & Biological Applications, pp 170-181, ACS, Washington, D.C.; U.S. Pat. No. 5,672,662.) The NHS esters of PEG carboxylic acids have been used in several human applications: mPEG-SPA 5,000 has been used for attachment to a protein antagonist, which has successfully completed clinical trials and an NDA has been filed (the MPEG-SPA 5,000 process is validated and suitable for commercial use).

Alternatively, the targeting factor may be conjugated to another moiety, such as a lipid, hydrophobic anchor or polymer. Non-limiting examples include Cholesteryl-succinyl-KKAAAVLLPVLLAAP (Elan218) (SEQ ID NO:33), DOPE-succinyl-KKAAAVLLPVLLAAP (Elan219) (SEQ ID NO:34), and Cholesteryl-succinyl-kkaaavllpvllaap (All d-Elan218) (SEQ ID NO:35). These targeting factor-lipid conjugates may be included in the complexes, or may be further conjugated to a pegylated lipid for inclusion in the complexes. See, e.g., Harasym, T. O., et al. (1998) Advanced Drug Delivery Reviews 32:99-118 for a review of methods of conjugating targeting factors to lipids. Specific examples of conjugating targeting factors (such as MTLPs) to lipids may be found in the Examples. The targeting factor may also be linked to the moiety by a suitable linker, such as, for example, carbon spacers, cleavable linkers which may be cleaved enzymatically by enzymes present at cell surfaces (e.g., metalloproteases), or which may be cleaved by change in pH or temperature, amide-amide linkers, amide-disulfide linkers, carbamate-disulfide linkers, glycolamidic ester linkers, ester-amide linkers, ester-disulfide linkers, hydrazone linkers, and amide-thioester linkers.

In certain embodiments, the targeting factor is not linked to any other component of the complex.

The targeting factors of the invention may also be multifuinctional, comprising both specific targeting elements and non-specific elements which increase drug uptake at the target cells or sites following targeting particular cell types.

Examples of “specific” targeting factors include ligands (e.g. natural or synthetic nucleic acids, proteins, peptides, small molecules, etc., such as, but not limited to asialoglycoprotein, insulin, low density lipoprotein (LDL), growth factors, galactose, lectin, folate, and monoclonal and polyclonal antibodies directed against cell surface molecules etc., as disclosed herein which may or may not be modified as known in the art) of cell surface receptors of any type described herein. Such targeting factors may also be referred to as cell surface membrane receptor associated targeting factors, or, targeting factors which are mediated by an outer cell surface membrane receptor, or targeting factors which increase cellular bioavailability by the targeting of a specific outer cell surface membrane receptor. Examples of targeting factors which target “specific” outer cell surface receptors include, but are not limited to, hormones, antibodies, vitamins, etc. These molecules may also be said to bind to “classic” outer cell surface membrane receptors.

Examples of “non-specific” targeting elements include targeting factors which increase the cellular bioavailability of a drug, particularly a nucleic acid, by a means other than the targeting of a specific outer cell surface membrane receptor. Included are targeting factors which increase the transcription of nucleic-acid in the nucleus, increase the uptake of nucleic acid into a cell, increase the uptake of nucleic acid into a cellular compartment, increase the exit of nucleic acid from a cellular compartment, or increase transport of the nucleic acid across a membrane (e.g. cell surface, nuclear or endosomal membrane). Non-limiting examples of non-specific targeting factors (targeting factors which increase the cellular bioavailability of a drug, particularly a nucleic acid, by a means other than the targeting of a outer cell surface membrane receptor) include membrane-disruptive synthetic polymers, including pH sensitive membrane-disruptive synthetic polymers (e.g., poly(alkylacrylic acids; PPAA, PEAA); MTLPs (e.g., H₂N-KKAAAVLLPVLLAAP-COOH (Elan094) (SEQ ID NO:2), H₂N-KKKAAAVLLPVLLAAP (ZElan094) (SEQ ID NO:3), H₂N-kkkaavllpvllaap (ZElan207) (SEQ ID NO:4), H₂N-KKKAAAVLLPVLLAAPREDL (ZElan094R) (SEQ ID NO:5); H₂N-GLFGAIAGFEENGWEGMIDGWYG-COOH (Influenza HA-2 (INF6)) (SEQ ID NO:15); H₂N-GLFEALLELLESLWLLEA-COOH (JTS1) (SEQ ID NO:16); H₂N-HHHHHWYG-COOH (H₅WYG) (SEQ ID NO:17); H₂N-WEAALAEALAEALAEHLAEALAEALEALAA-COOH (GALA) (SEQ ID NO:18); H₂N-WEAKLAKALAKALAKHLAKALAKALKACEA-COOH (KALA) (SEQ ID NO:19); VP22 (HSV-1); H₂N-CPCILNRLVQFVKDRISVVQAL-COOH (Retrovirus intra-cellular domain(Mo-MuLv)) (SEQ ID NO:20); H₂N-RQIKDWFQNRRMKWKK-COOH (Homeobox domain penetration ) (SEQ ID NO:21); H₂N-RQPKIWFPNRRKPWKK-COOH (Homeobox domain penetration) (SEQ ID NO:22); H₂N-PLSSIFSRIG-COOH (PreS2 domain) (SEQ ID NO:23); H₂N-RGGRLSYSRRRFSTSTGR-COOH (SynB1)) (SEQ ID NO:24); protein transduction domains (PTDs) (e.g., H₂N-YGRKKRRQRRR-COOH (TAT) (SEQ ID NO:25); H₂N-RQJKIWFQNRRMKWKK-COOH (Antp) (SEQ ID NO:21); H₂N-RRRRRRR-COOH (Arg) (SEQ ID NO:26); and H₂N-HHHHHHHHH-COOH (His) (SEQ ID NO:27). Such targeting factors may also be referred to as non-outer cell surface receptor mediated (or associated) targeting factors. Non-specific targeting factors may be conjugated to another component of the complex (e.g., lipid, pegylated lipid, including co-lipids or pegylated co-lipids), or may be present without being conjugated to any other component of the complex.

An “increase” or “enhancement” in cellular bioavailability for deoxyribonucleic acids can be measured by an increase in gene expression of a nucleic acid, techniques for which are well known to those of skill in the art. An “increase” related to the transcription of, uptake into, or exit from a given cellular compartment, or transport across a membrane as described in the preceding paragraph, refers to an increase in the rate as well as an increase in the total amount of nucleic acid transcribed, taken up, released or transported. Techniques for measuring the increase in cellular availability of a drug which is not a nucleic acid are also known in the art. Such techniques include the labeling of the drug with a probe, such as a fluorescent or radioactive probe and measuring the amount of probe within the target cells or cellular compartment.

A single targeting factor may encompass one or more types of specific and/or non-specific targeting activities or two separate targeting factors may be conjugated together to form a multifuinctional targeting factor. The complexes described herein may also include a single non-specific targeting factor which may or may not be conjugated to another component of the complex, for example a lipid or pegylated lipid species. Additionally, where a complex comprises more than one targeting factor, the individual targeting factors may or may not be conjugated to each other. When a complex comprises more than one targeting factor, the individual targeting factors may independently be specific or non-specific, and may independently be conjugated to lipid or pegylated lipid, or not conjugated to a lipid or pegylated lipid. The targeting factors, both specific and non-specific, should result in increased cellular bioavailability in the particular cell type targeted.

Examples of particular targeting factors or ligands for particular cell types are well known in the art. For example, Kichler, et al., ((2000) Journal of Liposome Research 10:443-460) and Arap et al. (Nature Med. (2002) 8(2):121-127) each describe particular motifs that may be used to target particular organs or cell types. For example, Arap et al., supra, describe peptide sequences which may be incorporated into targeting factors for the targeting of specific organs, tissues, or cell types, for example, bone marrow, muscle, prostate, fat and skin. Kichler, et al, supra describe the use of a variety of ligands to target cells such as hepatocytes, macrophages, breast/pancreatic cancer cells, ovarian cancer cells, lung epithelial cells, T-lymphocytes, lung endothelial cells, alveolar macrophages, neurons and fibroblasts. These and other ligand sequences known in the art may be used as targeting factors in the complexes described herein, where selection of a given sequence for use as a targeting factor will depend upon the specific organ, cell or tissue type being targeted and the disease, condition or syndrome being treated or diagnosed.

Nonlimiting examples of multifunctional targeting factors include KKAAAVLLPVLLAAS-Galactose (Elan094G) (SEQ ID NO:40), and S(Galactose)-KKAAAVLLPVLLAAP (GElan094) (SEQ ID NO:32). Alternatively, more than 1 targeting factor may be included in a complex to produce a complex with multifunctional targeting activity.

In one embodiment, the targeting factor(s) may be present in the complex at a concentration of, for example, about 0.01 μM to about 50 mM, for example, about 0.01 μM to about 1 mM, for example, about 0.01 μM to about 500 μM, for example, about 5 μM to about 200 μM, for example, about 10 μM, for example, about 100 μM.

Pegylated Lipids

The PEG moiety may be selected from the group consisting of, for example, 750-20,000 molecular weight PEG, preferably 1000-10,000 molecular weight PEG, more preferably 2K-5K molecular weight PEG. In one embodiment, the complex may comprise more than one type of PEG moiety (for example, PEG molecular weight 5K and PEG molecular weight 2K). The PEG moiety may further comprise a suitable functional group, such as, for example, methoxy, N-hydroxyl succinimide (NHS), carbodiimide, etc., for ease of conjugating PEG to the lipid or to the targeting factor. See, e.g., Table 2 of Harasym, T. O., et al. (1998) Advanced Drug Delivery Reviews 32:99-118 for examples of suitable functional groups. Functionalized PEG moieties may be purchased from, for example, Shearwater Polymer Inc. (Huntsville, Ala.) and Avanti Polar Lipid Inc. (Alabaster, Ala.). In a preferred embodiment, the PEG moiety is N-[methoxy(polyethylene glycol)-5K] (PEG_{5K}). Other types of hydrophilic polymers may be substituted for the PEG moiety, including, for example, poloxamer and poloxamine. See, e.g., Feldman, L. J., et al. (1997) Gene Therapy 4(3):189-198; Lemieux, P., et al. (2000) Gene Therapy 7(11):986-91; Moghimi, M., et al. (2000) Trends In Biotechnology 18:412-420; Torchilin, V. P. (1998) Journal of Microencapsulation 15(1): 1-19; Claesson, P. M., et al. (1996) Colloids & Surfaces A-Physicochemical & Engineering Aspects 112(2):-3, 131-139.

The PEG moiety may be conjugated to any suitable lipid, such as, for example, the lipids described herein to form the “pegylated lipid”. Preferably, the PEG moiety is covalently attached to the lipid. Preferred lipids include dioleoylphosphatidyl-ethanolamine (DOPE), cholesterol, and ceramides. Lipids comprising a polar end (such as, e.g., phosphatidylethanolamines, including DOPE, DPPE and DSPE), which may be utilized for conjugating to PEG, are preferred for ease of synthesis of pegylated lipids. See, for example, Table 2 and references described therein in (Harasym, T. O., el al. (1998) Advanced Drug Delivery Reviews 32:99-118) for non-limiting examples of suitable functionalized lipids. In a preferred embodiment, the lipid is 1,2-distearoyl-sn-glycero-3-phosphotidylethanolamine (DSPE) or dimyristoyl phophatidylethanolamine (DMPE). In a preferred embodiment, the pegylated lipid comprises 1,2-distearoyl-sn-glycero-3-phosphatidylethanolamine-N-[methoxy(polyethylene glycol)-5K] (DSPE-PEG_{5K}) or dimyristoyl phosphatidylethanolamine-N-[methoxy(polyethylene glycol)-5K] (DSPE-PEG_{5K}).

The PEG moiety may be conjugated to the lipid by methods known in the art. See, for example, Woodle, M. C. (1998) Adv. Drug Delivery Reviews 32:139-152 and references cited therein; Haselgruber, T., et al. (1995) Bioconjug Chem 6:242-248; Shahinian, S., et al. (1995) Biochim Biophys Acta 1239:157-167; Zalipsky, S.; et al. (1994) FEBS Lett. 353:71-74; Zalipsky, S.; et al. (1997) Bioconjug Chem. 8(2):111-118; Zalipsky, S.; et al. (1995) Bioconjug Chem. 6( ): 705-708; Hansen, C. B.; et al. (1995) Biochim Biophys Acta. 1239(2):133-44; Allen, T. M.; et al. (1995) Biochim Biophys Acta 1237(2):99-108; Zalipsky, S. (1995) Bioconjug Chem 6(2): 150-65; Zalipsky, S. (1993) Bioconjug Chem 4(4): 296-9; and Zalipsky, S. (1995) in Stealth Liposomes. (Eds: Lasic, D., et al.) CRC Press, Boca Raton, Fla., p. 93-102. Pegylated lipids are also available commercially from, for example, Shearwater Polymer Inc. (Huntsville, Ala.).

It is to be understood that compounds other than lipids, such as, for example, peptides, hydrophobic anchors or polymers, carbohydrates, metals or other ions may be used for conjugating with PEG to form the complexes of the invention, provided the compounds anchor PEG to the lipid complex, and allow PEG to be displayed on the surface of the lipid complex.

While not wishing to be bound by theory, the charge shielding effect provided by PEG may enhance the circulatory half-life of the complexes. Shielding may also increase the resistance (decrease the sensitivity) of nucleic acid to degradation, for example by nucleases or other species present in vitro or in vivo (e.g., hyuralonic acid, poly(Asp)) and/or decrease or prevent interactions between individual complex particles or interactions with other species present in vitro or in vivo that may lead to increased complex particle size or aggregation of complex particles. Accordingly, in a preferred embodiment, the. complex comprises a neutral surface. In another preferred embodiments, the complex is charge shielded.

In certain embodiments, the complex is shielded to increase the circulatory half-life of the complex or shielded to increase the resistance of nucleic acid to degradation, for example degradation by nucleases.

As used herein, the term “shielding”, and its congnates such as “shielded”, refers to the ability of “shielding moieties” to reduce the non-specific interaction of the complexes described herein with serum complement or with other species present in serum in vitro or in vivo. Shielding moieties may decrease the complex interaction with or binding to these species through one or more mechanisms, including, for example, non-specific steric or non-specific electronic interactions. Examples of such interactions include non-specific electrostatic interactions, charge interactions, Van der Waal's interactions, steric-hindrance and the like. For a moiety to act as a shielding moiety, the mechanism or mechanisms by which it may reduce interaction with, association with or binding to serum complement or other species does not have to be identified. Methods for determing whether a complex binds serum species, and therefore methods for determing whether a moiety can act as a shielding moiety, are known in the art and as described herein, particularly in the Examples. For example, the measurement of complex size after incubation in serum or the complement opsonization assay.

Other moieties which will act as shielding moieties may be identified by their ability to block binding of serum complement, or the serum complement pathway. For example, the C3A or C5 proteins of the complement pathway. If a moiety is not recognized by (e.g., does not bind) at least one of the components of serum complement or the serum complement pathway, then the moiety should act as a shielding moiety. In particular examples, if a moiety does not bind to or interact with at least one of the C3A or C5 proteins, then the moiety will should not be bound by or interact with serum complement. Methods for determining whether a moiety will bind to or interact with serum complement (e.g., proteins C3A or C5) will be known to those of skill in the art. Methods and techniques standard in the art can be used to measure such binding or interaction. See for example, Ahl et al. (1997) Biochemica et Biophysica Acta 1329:370-382.

Incorporation on the surface of the complexes described herein of a moiety which does not bind, associate with, or interact with serum complement or other serum species results in the shielding of the complex. In other words, the components (e.g., lipids) of the complex that would be recognized by or would interact with components of serum are instead shielded from the serum components (e.g., serum proteins, for example, albumin, serum complement, hormones, vitamins, co-factors and others) and therefore are not accessible to serum components and thus are not bound by, associate with, or do not interact with these components, incuding serum complement. The complex therefore can be described as “shielded”. A moiety capable of providing shielding can be termed a “shielding moiety”.

Shielding, as described above, may also be measured by the level of complement opsonization, as described herein. In particular embodiments, the shielding moiety will reduce complement opsonization by approximately 30%, approximately 40%, approximately 50%, approximately 60%, approximately 65%, approximately 70%, approximately 75%, or approximately 80%. In other embodiments, the shielding moiety will reduce complement opsonization by at least 40%, at least 50%, at least 55%, at least 60%.

It should be noted, that “shielding moieties” may be multifunctional. For example, a shielding moiety may also function as, for example, a targeting factor. A shielding moiety may also be referred to as multifunctional with respect to the mechanism(s) by which it shields the complex. While not wishing to be limited by proposed mechanism or theory, one example of such a multifunctional shielding moiety is the pH sensitive endosomal membrane-disruptive synthetic polymers, such as PPAA or PEAA. Certain poly(alkylacrylic acids) have been shown to disrupt endosomal membranes while leaving the-outer cell surface membrane intact (Stayton et al. (2000) J. Controll. Release 65:203-220; Murthy et al. (1999) J. Controll. Release 61:137-143; WO 99/34831) thus, as described above, increasing cellular bioavailability and functioning as a targeting factor. However, as shown in the Examples, PPAA reduces binding of serum complement to complexes in which it is incorporated, thus, as described above, functioning as a shielding moiety.

As will be understood by those of skill in the art, it is important that incorporation of a shielding moiety does not eliminate the complex's ability to be delivered to cells. Therefore, in some embodiments, complexes incorporating a shielding moiety will further comprise a targeting factor. For example, a complex may comprise a cell surface receptor ligand (e.g., folate, an RGD peptide, an LHRH peptide, etc.) which may, for example be conjugated to a lipid or pegylated lipid and optionally also incorporate PPAA. In certain embodiments, the lipid-targeting factor conjugate is DSPE-PEG_{5K}-RGD (SEQ ID NO:10) or DSPE-PEG_{5K}-Folate.

In other embodiments, the amount or ratio of shielding moiety incorporated in a complex formulation is limited, so as not to eliminate-the complex's delivery to cells. Thus in particular examples, the complexes comprise less than about 15%, less than about 12%, less than about 10%,. less than about 8%, less than about 7%, less than about 5%, less than about 4%, less than about 3%, or less than about 2% shielding moiety. In particular embodiments, the amount of shielding moiety is about 10%, about 8%, about 5% or about 2%. A complex may also incorporate more than one shielding moiety. In certain embodiments the amount of shielding moiety is at least 2% or at least 5% or at least 8% or at least 10%.

In certain embodiments, the shielding moiety may be conjugated to another component of the complex, for example a lipid or pegylated lipid. In certain examples, the shielding moiety may be conjugated to a co-lipid or pegylated co-lipid. In other embodiments, the shielding moiety is not conjugated to any other component of the complex.

In particular embodiments, the complex is shielded by incorporation of compounds comprising polyethylene glycol moieties (PEG) or by the incorporation of synthetic polymers. In particular examples of the complexes described herein, the shielded complex may comprise one or more synthetic polymers, including for example, membrane disruptive synthetic polymers, pH sensitive membrane-disruptive synthetic polymers, pH sensitive endosomal membrane-disruptive synthetic polymers, or poly(alkylacrylic acid) polymers. Particular examples of membrane disruptive polymers include the poly(alkylacrylic acid) polymer poly(ethyl acrylic acid) (PEAA) and poly(propyl acrylic acid) (PPAA). Other suitable synthetic polymers, particularly poly(alkylacrylic acid) polymers, are described in the art and will be known to those of skill in the art. Thus, in certain embodiments, the shielding moiety is a pH sensitive membrane-disruptive synthetic polymer, pH sensitive endosomal membrane-disruptive synthetic polymer, or poly(alkylacrylic acid) polymer. In other embodiments the shielding moiety may be PPAA. In other embodiments, the shielding moiety is a compound comprising polyethylene glycol moieties.

It is also possible that shielding the complexes may reduce the toxicity of the complexes.

The pegylated lipid and/or targeting factor-pegylated lipid conjugate and/or targeting factor-lipid conjugate may comprise, for example, from about 0.01 to about 30 mol percent of the total lipids, more preferably, from about 1 to about 30 mol percent of the total lipids. The pegylated lipid and/or targeting factor-pegylated lipid conjugate and/or targeting factor-lipid conjugate may comprise, for example, from about 1 to about 20 mol percent, from about 1 to about 10 mol percent of the total lipids, from about 2 to about 5 mol percent, about 1 mol percent, about 2 mol percent, about 3 mol percent, about 4 mol percent, about 5 mol percent, about 10 mol percent, about 15 mol percent, about 20 mol percent of the total lipids. The complex may comprise a pegylated lipid without conjugated targeting factor as well as a targeting factor-pegylated lipid conjugate. The complex may also comprise a targeting factor-pegylated lipid conjugate and a targeting factor-lipid conjugate. The complex may comprise more than one targeting factor-pegylated lipid conjugate or targeting factor-lipid conjugate. The PEG moiety may be the same or different when more than one pegylated lipid is present in the complex. In one non-limiting example, the targeting factor-pegylated lipid conjugate may comprise PEG molecular weight 5K, and the pegylated lipid without conjugated targeting factor may comprise PEG molecular weight 750-2K. The complex may also comprise a pegylated lipid and a targeting factor conjugated to a lipid. In one embodiment, the complex comprises a targeting factor-pegylated lipid conjugate and a targeting factor-lipid conjugate. Alternatively, in other embodiments, the complex comprises a targeting factor that is not conjugated to lipid or pegylated lipid, and comprises a pegylated lipid.

Drug

The drug may be, for example, a nucleic acid or a protein. In a preferred embodiment, the drug is a nucleic acid. The nucleic acid may be, for example, DNA or RNA. The nucleic acid may be single-stranded or double-stranded, and may be linear or closed circular. In a preferred embodiment, the drug is a nucleic acid sequence encoding a gene product having therapeutic utility. In another preferred embodiment, the drug is a nucleic acid sequence encoding a gene product having prophylactic utility. In another preferred embodiment, the drug is a nucleic acid sequence encoding a-gene product having diagnostic utility. In another preferred embodiment, the drug is a nucleic acid sequence encoding an antisense mRNA to another target mRNA which is expressed in the target cells or diseased tissue or diseased cells. In another preferred embodiment, the nucleic acid comprises an EIA gene. Other preferred genes include, for example, genes which encode tumor suppressor proteins, anti-angiogenic proteins, inhibitory proteins, suicide protein, reporter protein, an antisense RNA directed to a target mRNAs (e.g., thymidine kinase), cytokines (e.g., β-interferon), other immune modulators, antigens, adjuvants, or peptide fragments thereof, and genes which encode antigens. In another preferred embodiment, the drug may comprise more than one gene, coding for two or more different proteins.

Examples of complexes with diagnostic utility include those comprising genes which express proteins, including reporter proteins, which are detectable, either qualitatively or quantitatively, by methods known in the art. Such methods may include in vitro, in vivo, or ex vivo techniques. Exemplary reporter proteins (and genes encoding them) include, but are not limited to, the chloramphenicol acetyl transferase gene, the luciferase gene, the β-galactosidase gene, the human growth hormone gene, the alkaline phosphatase gene, the red fluorescent protein gene, and the green fluorescent protein gene. Examples of detection of reporter genes, such as the luciferase gene, are described in the Examples.

It is understood that in the present invention, preferred nucleic acid sequences are those capable of directing protein expression. Such sequences may be inserted by routine methodology into plasmid expression vectors known to those of skill in the art prior to mixing with lipids and/or polycation and/or targeting factor to form the lipid-comprising drug delivery complexes of the present invention. It is understood that where the nucleic acid of interest is contained in plasmid expression vectors, the amount of nucleic acid recited herein refers to the plasmid containing the nucleic acid of interest.

Polycations

Inclusion of polycations in the complexes of the invention may allow for higher concentrations of complexes to be formulated, and may also decrease particle size. Further, condensing the nucleic acids with polycation may help to protect the nucleic acid from degradation and aid in delivery of nucleic acid to its site of action. Accordingly, the complex preferably comprises a polycation. When formulating a cationic complex, the addition of polycation is optional, although the addition of polycation is preferred. When formulating an anionic complex, the addition of polycation is essential. It is to be understood that, generally, when forming anionic complexes comprising a nucleic acid, a greater amount of polycation will be necessary to neutralize the negative charge from the nucleic acid than when forming cationic complexes. Preferably, when forming anionic complexes, an excess charge ratio of at least about 0.8:1 polycation:nucleic acid, at least about 1:1 polycation:nucleic acid, at least about 2:1 polycation:nucleic acid, at least about 4:1 polycation:nucleic acid, at least about 6:1 polycation:nucleic acid, at least about 12:1 polycation:nucleic acid, at least about 20:1 polycation:nucleic acid, at least about 30:1 polycation:nucleic acid will be formed.

When a polycation is to be mixed with nucleic acid and lipids, the polycation may be selected from organic polycations having a molecular weight of between about 300 and about 200,000. These polycations also preferably have a valence of between about 3 and about 1000 at pH 7.0. The polycations may be natural or synthetic amino acids, peptides, proteins, polyamines, carbohydrates and any synthetic cationic polymers. Nonlimiting examples of polycations include polyarginine, polyornithine, protamines and polylysine, polybrene (hexadimethrine bromide), histone, cationic dendrimer, polyhistidine, spermine, spermidine and synthetic polypeptides derived from SV40 large T antigen which has excess positive charges and represents a nuclear localization signal, synthetic polycations, and inorganic cations such as, for example, Ca⁺⁺ ions. In one embodiment, the polycation is poly-L-lysine (PLL). In preferred embodiments, the polycation is not protamine or a protamine salt.

In certain embodiments the polycation is a synthetic polycation such as, but not limited to polycationic poly(alkenylimines) (e.g., polyethyleneimine), polycationic methacrylate polymers (e.g., polymers comprising dimethylamino methacrylate or co-polymers of dimethylamino methacrylate and methacrylic ester), or polycationic methacryloxy polymers (e.g., poly(2-methacryloxyethyltrimethyl ammonium bromide) (PMOETMAB)). In certain embodiments, the polycationic methacrylate polymer is a polymer comprising dimethylamino methacrylate. In particular embodiments the synthetic polycation is selected from the group consisting of polyethyleneimine (PEI), poly(2-methacryloxyethyltrimethyl ammonium bromide (PMOETMAB), and co-polymers of dimethylamino methacrylate and methacrylic ester. In certain embodiments, the co-polymer of dimethylamino methacrylate and methacrylic ester contains approximately 25% dimethyl aminoethyl polymer and the balance methacryclic ester. In certain embodiments the synthetic polymer is selected from the group consisting of PEI and PMOETMAB.

In another more preferred embodiment, the polycation is a polycationic polypeptide having an amino acid composition in which arginine residues comprise at least 30% of the amino acid residues of the polypeptide and lysine residues comprise less than 5% of the amino acid residues of the polypeptide. In addition, preferably histidine, lysine and arginine together make up from about 45% to about 85% of the amino acid residues of the polypeptide and serine, threonine and glycine make up from about 10% to about 25% of the amino acid residues of the polypeptide. More preferably, arginine residues constitute from about 65% to about 75% of the amino acid residues of the polypeptide and lysine residues constitute from about 0 to about 3% of the amino acid residues of the polypeptide.

In addition to the above recited percentages.of arginine and lysine residues, the polycationic polypeptides of the invention may also contain from about 20% to about 30% hydrophobic residues, more preferably, about 25% hydrophobic residues. The polycationic polypeptide to be used in producing drug/lipid/polycation/targeting factor complexes may be up to 500 amino acids in length, preferably about 20 to about 100 amino acids in length; more preferably, from about 25 to about 50 amino acids in length, and most preferably from about 25 to about 35 amino acids in length.

In one embodiment, the arginine residues present in the polycationic polypeptide are found in clusters of 3-8 contiguous arginine residues and more preferably in clusters of 4-6 contiguous arginine residues.

In another embodiment, the polycationic polypeptide is about 25 to about 35 amino acids in length and about 65 to about 70% of its residues are arginine residues and 0 to 3% of its residues are lysine residues.

The polycationic polypeptides to be used in formulating the complexes of the invention may be provided as naturally occurring proteins, particularly certain protamines having a high arginine to lysine ratio as discussed above, as a chemically synthesized polypeptide, as a recombinant polypeptide expressed from a nucleic acid sequence which encodes the polypeptide, or as a salt of any of the above polypeptides where such salts include, but are not limited to, phosphate, chloride and sulfate salts. See, for example, U.S. Pat. Nos. 6,008,202 and 5,795,587.

In one embodiment, a drug such as DNA could be complexed with an excess of polycation such that a net positively charged complex is produced. This complex, by nature of its positive charge, could favorably interact with negatively charged lipid(s) to form a DNA/ lipid/polycation/targeting factor complex.

Lipids

Suitable cationic lipid species include, but are not limited to: 3β[⁴N-(¹N,⁸N-diguanidino spermidine)-carbamoyl] cholesterol (BGSC); 3β[N,N-diguanidinoethyl-aminoethane)-carbamoyl] cholesterol (BGTC); N,N′,N²,N³ Tetra-methyltetrapalmitylspermine (cellfectin); N-t-butyl-N′-tetradecyl-3-tetradecyl-aminopropion-amidine (CLONfectin); dimethyldioctadecyl ammonium bromide (DDAB); 1,2-dimyristyloxypropyl-3-dimethyl-hydroxy ethyl ammonium bromide (DMRIE); 2,3-dioleoyloxy-N-[2(sperminecarboxamido)ethyl]-N,N-dimethyl-1-propanaminium trifluorocetate) (DOSPA); 1,3-dioleoyloxy-2-(6-carboxyspermyl)-propyl amide (DOSPER); 4-(2,3-bis-palmitoyloxy-propyl)-1-methyl-1H-imidazole (DPIM) N,N,N′,N′-tetramethyl-N,N′-bis(2-hydroxyethyl)-2,3 dioleoyloxy-1,4-butanediammonium iodide) (Tfx-50); 1,2 bis(oleoyloxy)-3-(trimethylammonio) propane (DOTAP); N-1-(2,3-dioleoyloxy) propyl-N, N, N-trimethyl ammonium chloride (DOTMA) or other N-(N, N-1-dialkoxy)-alkyl-N, N, N-trisubstituted ammonium surfactants; 1,2 dioleoyl-3-(4′-trimethylammonio) butanol-sn-glycerol (DOBT) or cholesteryl (4′ trimethylammonia) butanoate (ChOTB) where the trimethylammonium group is connected via a butanol spacer arm to either the double chain (for DOTB) or cholesteryl group (for ChOTB); DORI (DL-1,2-dioleoyl-3-dimethylaminopropyl-β-hydroxyethylammonium) or DORIE (DL-1,2-O-dioleoyl-3-dimethylaminopropyl-β-hydroxyethylammonium) (DORIE) or analogs thereof as disclosed in WO 93/03709; 1,2-dioleoyl-3-succinyl-sn-glycerol choline ester (DOSC); cholesteryl hemisuccinate ester (ChOSC); lipopolyamines such as dioctadecylamidoglycylspermnine (DOGS) and dipalmitoyl phosphatidylethanol amine (DPPE) or the cationic lipids disclosed in U.S. Pat. No. 5,283,185, cholesteryl-3β-carboxyl-amido-ethylenetrimethyl-ammonium iodide, 1-dimethylamino-3-trimethylammonio-DL-2-propyl-cholesteryl carboxylate iodide, cholesteryl-3β-carboxyamidoethyleneamine, cholesteryl-3β-oxysuccinamido-ethylenetrimethylammonium iodide, 1-dimethylamino-3-trimethylammonio-DL-2-propyl-cholesteryl-3β-oxysuccinate iodide, 2-(2-trimethylammonio)-ethylmethylamino ethyl-cholesteryl-3β-oxysuccinate iodide, 3β-N-(N′, N′-dimethylaminoethane) carbamoyl cholesterol (DC-chol), and 3β-N-(polyethyleneimine)-carbamoylcholesterol.

Examples of preferred cationic lipids include N-t-butyl-N′-tetradecyl-3-tetradecyl-aminopropion-amidine (CLONfectin), 2,3-dioleoyloxy-N-[2(sperminecarboxamido)ethyl]-N,N-dimethyl-1-propanaminium trifluoroacetate (DOSPA), 1,2-bis(oleoyloxy)-3-(trimethylammonio)propane (DOTAP), N-[1-(2,3, dioleoyloxy)propyl]-N,N,N-trimethylammonium chloride) (DOTMA), cholesteryl-3β-carboxyamidoethylenetri-methylammonium iodide, 1-dimethylamino-3-trimethylammonio-DL-2-propyl-cholesteryl carboxylate iodide, cholesteryl-3β-carboxyamidoethyleneamine, cholesteryl-3β-oxysuccin-amidoethylenetrimethyl-ammonium iodide, 1-dimethylamino-3-trimethylammonio-DL-2-propyl-cholesteryl-3β-oxysuccinate iodide, 2-(2-trimethylammonio)ethylmethylamino ethyl-cholesteryl-3β-oxysuccinateiodide, 3βN-(N′, N′dimethyl-aminoethane)-carbamoyl-cholesterol (DC-chol), and 3βN-(polyethyleneimine)-carbamoyl cholesterol.

In particular complex formulations, the cationic lipid is DOTAP. In certain other complex formulations, the cationic lipid is DOTAP and the complex further comprises one or more co-lipids. In particular examples the co-lipid is neutral or pH sensitive. In some embodiments the co-lipid may be pegylated. hi certain embodiments the co-lipid is at least one lipid selected such as cholesterol, 1,2-distearoyl-sn-glycero-3-phosphotidylethanolanine (DSPE), dimyristoyl phosphotidylethanolamine (DMPE), dilauryl phophatidylethanolamine (DLPE), dimyristoyl phosphotidylethanolamine (DMPE), diphytanoyl phosphatidylethanolamine (DPHPE), dipalmitoyl phosphatidylethanolamine (DPPE), and 1,2-dioleoyl phosphatidylethanolamine (DOPE). Co-lipids may in some formulations be a neutral lipid, such as CHOL, DSPE or DMPE. Certain DOTAP formulations may contain more than one co-lipid, for example CHOL or DSPE, and DSPE may or may not be pegylated. The co-lipid may be bound to a membrane-disruptive synthetic polymer, such as, membrane-disruptive polymers, endosomal membrane-disruptive synthetic polymers, or poly(alkylacrylic acids) (e.g., PPAA or PEAA).

In certain examples, where the complex comprises at least one lipid which is a cationic lipid and the targeting factor is a membrane-disruptive synthetic polycation (e.g., PPAA, PEAA), the complex will further comprise at least one additional lipid (co-lipid or helper lipid). Examples of suitable co-lipids include additional cationic lipids or neutral lipids as described herein. In particular examples the co-lipid is neutral or pH sensitive. In certain embodiments the co-lipid is at least one lipid such as cholesterol, 1,2-distearoyl-sn-glycero-3-phosphotidylethanolamine (DSPE), dilauryl phophatidylethanolamine (DLPE), dimyristoyl phosphotidylethanolamine (DMPE), 1,2-dioleoyl phosphatidylethanolamine (DOPE), diphytanoyl phosphatidylethanolamine (DPHPE), dipalmitoyl phosphatidylethanolamine (DPPE). The co-lipid may be pegylated or non-pegylated and may be conjugated to a targeting factor or may be a pegylated-targeting factor lipid conjugate. In particular examples, the co-lipid is DSPE.

In some cases, the preferred lipid or combination of lipids used will depend on the route of administration of the complexes. For example, DOTAP/cholesterol is relatively more stable in serum, and thus is a preferred lipid combination when the liposome is to be injected intravenously. DC-Chol/DOPE is relatively less stable in serum than DOTAP/cholesterol, and thus may be preferred for intratumoral or intraperitoneal injection, where faster drug release rate is preferred. For intravenous delivery, complexes comprising DSPE-PEG may be preferred, while for intratumoral delivery, complexes comprising DMPE-PEG may be preferred.

Those of skill in the art would readily understand that liposomes containing more than one cationic lipid species may also be used to produce the complexes of the present invention. For example, liposomes comprising two cationic lipid species, lysyl-phosphatidylethanolamine and β-alanyl cholesterol ester have been disclosed (Brunette, E. et al. (1992) Nucl. Acids Res., 20:1151).

Anionic lipids which may be used to form the complexes of the invention include, but are not limited to, cholesteryl hemisuccinate (CHEMS), N-glutaryl phosphatidylethanolamine (NGPE), phosphatidylglycerol, phosphatidylinosityl, cardiolipin, 1,2-dioleoyl-sn-glycero-3-[phospho-L-serine] (DOPS), 1,2-dioleoyl-sn-glycero-3-[phospho-rac-1-glycerol] (DOPG), 1,2-dioleoyl-sn-glycero-3-[phosphoethanolamine-N-dodecanoyl (NC₁₂-DOPE), and phosphatidic acid or a similar phospholipid analog, for example, 1,2-diacyl-SN-glycero-3-phosphate derivatives; phosphatidylglycerol and 1,2-diacyl-SN-glycero-3-[phospho-RAC-(1-glycerol)] derivative; phosphatidylserine and all 1,2-diacyl-SN-glycero-3-[phospho-L-serine] derivatives. Additional anionic lipids of use in the complexes described herein include, cardiolipin, tetraoleoyl-cardiolipin and derivatives thereof; and, 1,2-Dioleoyl-sn-glycero-3-succinate and derivatives. In a preferred embodiment, the anionic lipid is CHEMS. In another preferred embodiment, the anionic lipid is DOPS. In another preferred embodiment, the anionic lipid is DOPG.

Neutral lipids which may be used to form the complexes of the invention include, but are not limited to, lyso lipids of which lysophosphatidylcholine (1-oleoyl lysophosphatidylcholine) is an example, cholesterol, or neutral phospholipids including 1,2-dioleoyl phosphatidylethanolamine (DOPE), 1,2-distearoyl-sn-glycero-3-phosphotidylethanolamine (DSPE), dioleoyl phosphatidylcholine (DOPC), dilauryl phophatidylethanolamine (DLPE) or dimyristoyl phophatidylethanolamine (DMPE) as well as various lipophylic surfactants, containing polyethylene glycol moieties, of which Tween-80 is one example. Preferred neutral lipids include, for example, cholesterol, DOPE, DSPE, DMPE, DPHPE and DLPE.

At least one of the lipids may be a frsogenic lipid. In certain embodiments, the fusogenic lipid is an anionic lipid or a pH sensitive lipid which is characterized in that when the pH is changed from approximately pH 7 to approximately pH 4.5 the lipid undergoes a change in charge or structure such that it becomes more fusogenic. The pH at which the fusogenic change in structure or charge occurs includes the range of endosomal pH's, including both late and early endosome pH, for example approximately 4.5 to 6. Such lipids are well known to those of skill in the art. In certain embodiments the lipid is at least one of 1,2-dioleoyl phosphatidylethanolamine (DOPE), 1,2-dioleoyl-sn-glycero-3-[phosphoethanolamine-N-dodecanoyl (NC₁₂-DOPE), cholesteryl hemisuccinate (CHEMS), and 1,2-dioleoyl-sn-glycero-3-[phospho-L-serine] (DOPS). Additional fusogenic anionic lipids include 1,2-diacyl-SN-glycero-3-phosphate derivatives; phosphatidylglycerol and 1,2-diacyl-SN-glycero-3-[phospho-RAC-(1-glycerol)] derivative; phosphatidylserine and all 1,2-diacyl-SN-glycero-3-[phospho-L-serine] derivatives. Additional anionic lipids of use in the complexes described herein include, cardiolipin, tetraoleoyl-cardiolipin and derivatives thereof; and, 1,2-Dioleoyl-sn-glycero-3-succinate and derivatives. In certain embodiments, when the pH is lowered to approximately pH 4.5, the fusogenic anionic lipid undergoes a change in charge to become neutral or cationic. In other embodiments, the fusogenic pH sensitive lipid may undergo a change in charge upon a lowering of pH to approximately 4.5 such that a neutral or anionic lipid becomes cationic or neutral. In other embodiments, when the pH is lowered to pH approximately 4.5 the lipid undergoes a change in structure such that it assumes a hexagonal structure.

The fusogenic lipid may also be described as a lipid which undergoes a change in charge or structure upon a change in pH from physiological pH (e.g., approximately pH 7 to approximately pH 8.5) to endosomal pH (e.g., approximately pH 4.5 to approximately pH 6.5) such that it becomes more fusogenic.

The fusogenic lipid may also be described as a lipid that exhibits molecular shape properties of cone formation such that the lipid framework comprises a small cross sectional head group and a larger acyl chain cross-sectional area. Without wishing to be bound by theory these lipids are thought to induce a nonbilayer hexagonal H_{II} phase (Gaucheron, J. et al. “Synthesis and Properties of Novel Tetraalkyl Cationic Lipids” published on the internet for Bioconj. Chem. Apr. 5, 2002 (http://pubs3.acs.org/acs/joumals/toc.page?incoden=bcches&indecade=&involume=0&inissue=0)). These fusogenic lipids are often termed “cone forming” lipids in the art. Cone forming lipids may also be cationic.

A pH senstive lipid which is anionic at physiological pH may also be classed as an anionic lipid. Similarly a pH senstive lipid which is neutral at physiological pH may also be classed as a neutral lipid.

In particular embodiments where the complex is anionic (net negative charge), the lipid species comprises at least one anionic or neutral lipid, including pH sensitive lipids, which undergoes a change in charge or structure upon a change from physiological to endosomal pH as described above. In certain embodiments the fusogenic lipid is DOPE, DOPS, CHEMS or NC₁₂-DOPE.

While certain embodiments of the complexes as described herein comprise an anionic fusogenic lipid (e.g., NC₁₂-DOPE), in other embodiments, the complex may comprise an anionic lipid that is not fusogenic (e.g., DOPG). In certain embodiments of complexes comprising non-fusogenic anionic lipids, the complex should further comprise a component which is fusogenic (a fusogenic moiety). Examples of such fusogenic moieties include certain targeting factors. As one of skill in the art would recognize, a targeting factor that increases transport of the drug, particularly a nucleic acid, across a cellular membrane can be considered a fusogenic component, other fusogenic factors which may be included are MTLPs. For example, the synthetic polymers such as the membrane-disrupting synthetic polymers described herein may increase the fusogenic capacity of a complex. Accordingly, in particular embodiments, where the lipid is a non-fusogenic lipid, the complex will further comprise a poly(alkylacrylic acid). In certain embodiments the complex may further comprises a pH sensitive endosomal membrane-disruptive synthetic polymer.

Additional combinations of lipids and the other components of the complex (e.g., polycation, targeting factor, shielding factor) will be apparent to one of skill in the art and such combinations can be tailored to the intended use of the particular complex. For example, taking into account whether the complex is to be used in vitro, in vivo, or ex vivo, or, the particular cell type to be targeted, or whether the complex is intended to function as a diagnostic or therapeutic complex.

The complexes of the invention may have a net positive, neutral or negative charge. In a preferred embodiment, the complex has a net positive charge. In another preferred embodiment, the complex has a net negative charge.

In the cationic liposomes utilized to produce the drug/lipid/targeting factor complexes of this invention, which may optionally comprise polycation, the cationic lipid is present in the liposome at from about 0.1 to about 100 mole % of total liposomal lipid, preferably from about 7 to about 70 mole % and most preferably about 20 to about 50 mole %. The neutral lipid, when included in the liposome, may be present at a concentration of from about 0 to about 99.9 mole % of the total liposomal lipid, preferably from about 30 to about 90 mole %, and most preferably from 50 to 70 mole %. The negatively charged lipid, when included in the cationic complex, may be present at a concentration ranging from about 0.1 mole % to about 100 mole % of the total liposomal lipid, preferably from about 7 mole % to about 70 mole %, and most preferably about 20 to about 50 mole %. The liposomes may contain, for example, a cationic and a neutral lipid such as DOTAP and cholesterol or DC-Chol and DOPE. Molar ratios of DOTAP: cholesterol may be, for example, between about 1:1 to about 5:3, for example about 5:4.

It is further contemplated that for the cationic complexes utilized to form complexes of the invention, the ratio of lipids may be varied to include a majority of cationic lipids in combination with cholesterol or with mixtures of lyso or neutral lipids. When the cationic lipid of choice is to be combined with another lipid, preferred lipids include cholesterol, DOPE, and DLPE. In one embodiment, the cationic complex does not comprise a lipid which is negatively charged at a pH of about 6.0-8.0.

In the anionic liposome, micelles, or mixed micelles utilized to produce the complexes of this invention, the anionic lipid is present in the liposome, micelles, or mixed micelles at from about 0.1 to about 100 mole % of total lipid, preferably from about 20 to about 75 mole % and most preferably about 30 to about 65 mole %. The neutral lipid, when included in the liposome, micelles, or mixed micelles, may be present at a concentration of from about 0 to about 99.9 mole % of the total lipid, preferably from about 25 to about 80 mole %, and most preferably from 35 to 70 mole %. The positively charged lipid, when included in the anionic complex, may be present at a concentration ranging from about 0.1 mole % to about 10 mole % of the total lipid. The liposome, micelles, or mixed micelles may contain, for example, an anionic and a neutral lipid, such as, for example, CHEMS or DOPS as the anionic lipid and DOPE, cholesterol, or DLPE as the neutral lipid. Molar ratios for CHEMS:DOPE or DLPE may be, for example, about 1:10 to about 10:1, for example about 2:8 to about 4:6, for example about 3:7. Molar ratios for DOPS:cholesterol may be, for example, about 25:75 to about 85:15, for example about 50:50 to about 60:40, for example about 55:45. It is to be understood that the ratios of various lipids and the ratios of lipids to polycation to drug may vary in order to achieve the preferred charge, particle size, transfection activity, etc. In one embodiment, the anionic complex does not comprise a lipid which is positively charged at a pH of about 6.0-8.0.

Examples of lipid:polycation:nucleic acid ratios for the cationic complexes may include, for example about 12 nmol:0.1 μg:1 μg to about 12 nmol:10 μg:1 μg, for example about 12 nmol:1 μg:1 μg, for example about 12 nmol:0.97 μg:1 μg, for example about 12 nmol:0.9 μg:1 μg, for example about 12 nmol:0.6 μg:1 μg.

Examples of lipid:polycation:nucleic acid ratios for the anionic complexes may include, for example about 50 nmol:2 μg: 1 μg to about 70 nmol:2 μg:1 μg, for example about 53 nmol:2 μg:1 μ, for example about 65 nmol:2 μg:1 μg.

The nucleic acid/lipid/targeting factor complexes of the present invention, which optionally contain polycation, produce particles of varying diameters upon formulation. As pointed out in the Background of Invention smaller particles tend to show greater size stability than larger particles. Furthermore, smaller particles may be more suitable for use as nucleic acid delivery vehicles. Particle diameters can be controlled by adjusting the nucleic acid/lipid/polycation/targeting factor ratios in the complex, or by size exclusion methods, such as, for example, by passing the complexes through filters. The desired particle diameter may further depend on the cell or tissue type to be targeted. For example, particle diameters of approximately 100-200 nm are particularly preferred for targeting tumor cells, although it is to be understood that other sizes may also be suitable. For targeting lymph nodes, particle diameters of approximately 100 nm are particularly preferred, although it is to be understood that other sizes may also be suitable. For targeting liver cells, smaller particles of about 20 nm are particularly preferred, although it is to be understood that other sizes may also be suitable. The diameter of the complexes produced by the methods of the present invention may be, for example, about 20 nm to about 500 nm, about 150 nm to about 200 nm, less than about 400 nm, less than about 350 nm, less than about 300 nm, less than about 250 nm, less than about 200 nm, less than about 150 nm, less than about 100 nm.

The complexes formed by the methods of the present invention are preferably stable for, for example, up to about six months, up to about one year, at least about one year when, for example, stored at 4° C. The complexes may be stored in, for example, 10% sucrose, 5% dextrose, or other suitable buffers such as HEPES upon collection from the sucrose gradient or they may be lyophilized and then reconstituted in an isotonic solution prior to use. In a preferred embodiment, the complexes are stored in solution. A preferred buffer for storing anionic complexes is HEPES pH 7.2. It is to be further understood that the charge of the complexes of this invention may be affected not only by the lipid composition of the complex but also by the pH of the solution in which the complexes are formed. For example, increasing pH (more basic) will gradually neutralize the positive charge of the tertiary amine of the cationic lipid DC-Chol. The preferred pH range may be, for example, about pH 1 to about pH 14, about pH 2 to to about pH 9. In one embodiment, for cationic complexes, the pH is preferably about pH 7. For anionic complexes, a preferred pH range is about pH 6.8-7.4, for example about pH 7.2. Those of skill in the art would further understand that the preferred pH range will depend on the lipid composition of the complexes, and that the preferred pH is selected so as to limit instability of the lipids and/or other components of the complexes.

Binding Model

The method of producing these complexes is based on a binding model between two oppositely charged polymers (e.g., negatively charged nucleic acid and positively charged lipids) in which the formation of large unstable aggregates is avoided by neutralizing the negative charge of the drug via the use of an excess amount of positive charge in the form of cationic liposomes or polycation or cationic liposomes and polycation.

To produce drug/lipid/targeting factor or drug/lipid/polycation/targeting factor complexes with a net positive charge, the positive charge excess of lipid to drug or of lipid and polycation to drug may be up to about a 30-fold positive charge excess in the complex of total lipids to drug or of lipid and polycation to drug, preferably about a 2 to 10-fold charge excess and most preferably about a 2 to 6-fold charge excess. Cationic complexes preferably have a zeta potential of about 20 to about 50 mV. Complexes which possess a positive charge on their surface may have similar preferred ranges of surface charge excess to drug. For example, to produce a nucleic acid/lipid complex having a positive charge excess of lipid to nucleic acid, mole amounts of cationic liposomal lipid to be mixed with 1 μg of nucleic acid to produce a nucleic acid/lipid complex which has positive charge excess of lipid to nucleic acid at pH 6.0-8.0 may range from about 0.1 nmol to about 200 nmol of lipid, preferably about 5 nmol to about 100 nmol lipid, depending on the positive charge content of the cationic liposome. Of course, if the drug were a protein, the amount of lipid to be mixed with 1 μg of negatively charged protein would be at least 10-fold less than the amount of lipid to be mixed with 1 μg of DNA as shown above since proteins are less charge dense than nucleic acids. Those of ordinary skill in the art would readily understand that depending upon the positive charge content of the cationic liposomes, different mole amounts of different cationic liposomes would have to be mixed with an equivalent amount of drug to produce a positive charge excess of lipid to drug.

When a drug/lipid/polycation/targeting factor complex having a net positive charge and/or a positively charged surface is to be produced, the inclusion of the polycation reduces the amount of lipid which must be mixed with drug to the extent that the positive charge from the lipid may be less than the negative charge from the drug. This reduction in the amount of lipid reduces the toxicity of the polycation-containing formulations. Mole amounts of cationic liposomes to be used in formulating nucleic acid/lipid/polycation/targeting factor complexes may range from about 0.1 nmol to about 200 nmol lipid per 1 μg nucleic acid, more preferably from about 1 to about 25 nmoles lipid per 1 μg nucleic acid depending on the positive charge content of the cationic liposomes. Mole amounts of anionic liposomes to be used in formulating nucleic acid/lipid/polycation/targeting factor complexes may range from about 0.1 nmol to about 150 nmol lipid per 1 μg nucleic acid, more preferably from about 50 to about 150 nmoles lipid per 1 μg nucleic acid depending on the negative charge content of the anionic liposomes. It is to be generally understood that in producing the nucleic acid/lipid/targeting factor and nucleic acid/lipid/polycation/targeting factor complexes of the present invention, the mole amount of liposomes required to produce these complexes will increase as the concentration of nucleic acid mixed with the liposomes is increased. It will be further understood that the amounts of lipid, nucleic acid, and polycation may be varied depending on the charge and concentration of the targeting factor.

Those of ordinary skill in the art would readily understand that when the complexes of the present invention are purified, the positive charge excess of cationic liposomes to drug or of cationic liposomes and polycation to drug immediately prior to mixing will be greater than the positive charge excess in the purified complexes since the purification step may result in the removal of excess free lipids and/or free polycation and/or free targeting factor. Similar effects may be observed for anionic complexes.

In order to illustrate how the charges attributed to cationic lipid, drug and polycation may be determined at pH 6.0-8.0 the following example is provided. Assuming the drug to be delivered is DNA, one determines the negative charge of the DNA to be delivered by dividing the amount of DNA to be mixed, or the amount of DNA in the complex, by 330, the molecular weight of a single nucleotide where one nucleotide equals one negative charge. Thus, the negative charge for 1 μg of DNA is 3.3 nmols.

For 10 nmol of DC-Chol/DOPE (2:3) liposomes one calculates the effective charge of the lipid by multiplying the amount of total liposomal lipid (10 nmol) by 0.4 (40% of the total liposomal lipid is the cationic lipid DC-Chol) to yield 4 nmol DC-Chol lipid in the liposomes. Since at pH 6-8, one molecule of DC-Chol has one positive charge, the effective positive charge of liposomal lipid at the time of mixing, or in the complex, is 4.0 nmol. Of course, those of skill in the art would readily understand that other cationic lipids may have a lesser or greater amount of positive charge per molecule of cationic lipid at pH 6-8.0 than DC-Chol.

Assuming the polycation to be mixed to form the complex is a bromine salt of poly-L-lysine (PLL), the positive charge of PLL at the time of mixing is obtained by dividing the amount of PLL to be mixed by 207, the molecular weight of one lysyl residue where one lysyl residue equals one positive charge. Thus, the positive charge for 1 μg of PLL is approximately 5.0 nmols. To calculate the positive charge contributed by lysyl residues in a formed complex, the amount of lysine present in the complex is divided by the molecular weight of one lysyl residue taking into account the weight of a counterion, if present.

It is further to be understood by those skilled in the art that the net charge of the complex may be determined by measuring the amount of DNA, lipid, targeting factor, and when present, polycation in the complex by the use of an appropriate analytical technique such as the use of radioisotopic labelling of each component or by elemental analysis. Once the amounts of each component (DNA, lipid, targeting factor and when present, polycation) in a complex at a given pH are known, one could then calculate the approximate net charge of that complex at the given pH taking into account the pK's of the components which may be known or determined analytically.

Alternatively, complexes with a net negative charge or negatively charged surface may be produced by mixing polycation to nucleic acid at at least a 0.8 fold positive charge excess (i.e., resulting in a polycation/nucleic acid complex with a negative charge). Preferably, the polycation to nucleic acid is mixed at least a 1-fold (i.e., resulting in a polycation/nucleic acid complex with a neutral charge), at least a 2-fold, at least a 4-fold, at least a 6-fold, at least a 12-fold, at least a 20-fold, at least a 30-fold positive charge excess. Anionic liposome, micelles, or mixed micelles may subsequently be mixed with the polycation/nucleic acid to yield at least 1-fold negative charge excess (i.e., resulting in a lipid/polycationlnucleic acid complex with a neutral charge), preferably, at least a 2-fold, at least a 5-fold, at least a 10-fold negative charge excess. More preferably, the lipid to polycation/nucleic acid is mixed at about a 3-fold to about a 7-fold positive charge excess, even more preferably at about a 4-fold or about a 6-fold positive charge excess. It is to be understood that these ranges may be adjusted according to the concentration and charge of the targeting factor in the complex. The anionic complexes formed preferably have a zeta potential of about −20 to about −50 mV. Complexes which possess a negative charge on their surface may have similar preferred ranges of surface charge excess to drug. Those of ordinary skill in the art would readily understand that depending upon the negative charge content of the anionic lipids, different mole amounts of different anionic lipids would have to be mixed with an equivalent amount of drug/polycation/targeting factor to produce a negative charge excess.

Methods of Making the Drug Delivery Complexes

A method for producing the complexes described herein is provided, the method comprising combining drug, lipid, optionally a polycation, and targeting factor to form a complex.

The complexes may be produced, for example, by slowly adding nucleic acid to the solution of liposome/polycation/targeting factor and mixing, wherein the mixing is allowed to proceed second after addition of DNA. The liposome/polycation/targeting factor mix may be added into a single chamber from a first inlet at the same time the nucleic acid is added to the chamber through a second inlet. The components are then simultaneously mixed by mechanical means in a common chamber. A preferred method of making the complexes comprises first mixing the nucleic acid with the polycation and then adding the lipid/targeting factor suspension. Another preferred method of making the complexes comprises first mixing the nucleic acid with the polycation, then adding the lipid suspension and subsequently adding the targeting factor suspension. The methods described herein may be altered to accomodate those formulations where a targeting factor is not present, or where a polycation is not present. Similarly, the methods may also accommodate where more than one targeting factor or lipid or co-lipid is present, or where shielding factors are included.

In particular embodiments of the methods described above, the nucleic acid and polycation are mixed and then an aqueous micellar mixture comprising at least one lipid and at least one lipophilic surfactant is mixed with the compacted nucleic acid/polycation mixture. The resulting mixture is then treated to remove the lipophilic surfactant, resulting in liposomes. In particular variations of this method, the lipophilic surfactant is removed by dialysis. Methods for dialyzing lipid mixtures are well known in the art.

In a particular embodiment is provided a method for preparing a lipid-nucleic acid complex comprising a compacted nucleic acid and at least one lipid species that is fusogenic, comprising:
a) mixing an aqueous micelle mixture comprising a lipid and at least one lipophilic surfactant with a nucleic acid mixture comprising a nucleic acid, wherein the lipid has or assumes fusogenic characteristics, and wherein at least one of the mixtures contains a component that causes the nucleic acid to compact; and
b) after the mixing removing the lipophilic surfactant from mixture resulting from step a).

In certain embodiments of the method described above, the method further includes at least one targeting agent in at least one of the mixtures of step a).

The above-described method, or “micelle-lipophilic surfactant method” may also be performed with or without including the polycation if the lipid species are cationic. If the lipid species are anionic at physiological pH, inclusion of a polycation is required and use of the micelle-lipophilic surfactant method is crucial for the production of reproducible complexes. This method does however, as shown by the results in the Examples generated using the micelle-lipophilic surfactant method, generate reproducible complexes for complexes comprising pH sensitive, fusogenic and cationic lipids as well.

In certain embodiments of the micelle-lipophilic surfactant method, the lipophilic surfactant is N-Octyl-B-D-glucopyranoside (OGP). In other embodiments the lipophilic surfactant may be, but not limited to non-ionic detergents (e.g., OPG, Triton® X-100, Tween 20, Tween 40, Tween 80, NP-40 and others known in the art). The range of removal of the lipophilic surfactant is at least 90%, at least 92%, at least 95%.

The above method may also be altered, as will be known by those of skill in the art to include the incorporation of shielding moieties. The shielding moieties may be included in either the micelle or DNA mixture.

Methods for producing the liposomes and mixed micelles to be used in the production of the lipid-comprising drug delivery complexes of the present invention are known to those of ordinary skill in the art. A review of methodologies of liposome preparation may be found in Liposome Technology (CFC Press NY 1984); Liposomes by Ostro (Marcel Dekker, 1987); Methods Biochem Anal. 33:337-462 (1988) and U.S. Pat. No. 5,283,185. Such methods include freeze-thaw extrusion and sonication. Both unilamellar liposomes (less than about 200 nm in average diameter) and multilamellar liposomes (greater than about 300 nm in average diameter) may be used as starting components to produce the complexes of this invention.

The invention further relates to a method for producing these complexes where the method may optionally include the step of purifying these formulations from excess individual components. For the production of the complexes of this invention, inclusion of the purification step is a preferred embodiment.

Where purification of the complexes from excess free DNA, free lipids, free targeting factor and/or free polycation is desired, purification may be accomplished by centrifugation through a sucrose density gradient or other media which is suitable to form a density gradient. However, it is understood that other methods of purification such as chromatography, filtration, phase partition, precipitation or absorption may also be utilized. Purification methods include, for example, purification via centrifugation through a sucrose density gradient is utilized, or purification through a size exclusion column (e.g., a Sepharose CL4B column (Sigma, St Lous, Mo.)). The sucrose gradient may range from about 0% sucrose to about 60% sucrose, preferably from about 5% sucrose to about 30% sucrose. The buffer in which the sucrose gradient is made can be any aqueous buffer suitable for storage of the fraction containing the complexes and preferably, a buffer suitable for administration of the complex to cells and tissues, such as those described supra. Preferred buffers include 5% dextrose or pH 6.8-7.4 HEPES.

Additional methods suitable for making drug delivery complexes of the invention may be found in, for example, U.S. Pat. Nos. 5,554,382; 5,776,486, 5,795,587 and 6,008,202; European Patent No. 703,778; Castor, T. P.: “Supercritical Fluid Liposome Formulations,” presented at a symposium entitled “Liposomes and Vesicles: Fundamentals and Applications,” at AIChE Annual Meeting, San Francisco, Calif., Nov. 13-18, 1994; Chu, L. and Castor, T. P.: “Solubility of Phospholipids in Supercritical Fluids,” presented at the 12th Symposium on Thermophysical Properties, Boulder, Colo., Jun. 19-24, 1994; and in Castor, T. P.: “An Improved Liposome Manufacturing Process,” NSF Phase I SBIR Report on Grant No. ISTI-8961217, December, 1990; which are hereby incorporated by reference in their entirety.

Methods for Administering the Drug Delivery Complexes

The complexes of the invention may be used to deliver drug to cells by contacting the cells with the complex. In a preferred embodiment, the complexes of the present invention may be used in vivo as vectors in gene therapy. Alternatively, the cells may be contacted with the complex in vitro, or ex vivo. The complexes may be used to treat, diagnose or prevent a disease, condition or syndrome, non-limiting examples of which include cancer, bacterial infections, viral infections (e.g., with DNA vaccines), parasitic infections, immune deficiencies, gene defects (e.g., by administering Factor VIf or Factor Xa), and gene deficiencies (e.g., inherited genetic diseases).

The cells may be contacted with the complex in vivo, the method comprising administering the complex to an animal or human in an amount effective to deliver the drug into the cells of the animal or the human. The amount of drug administered to the individual will depend on the type of individual, the condition being treated, the particular drug used, the condition of the patient, etc. For example, when administering nucleic acid, the concentration of nucleic acid may range from, for example, about 1 μg/ml to about 5 mg/ml, about 150 μg/ml to about 500 μg/ml, about 200 μg/ml, about 150 μg/ml, about 125 μg/ml, at least about 150 μg/ml, at least about 200 μg/ml. The total amount of nucleic acid administered to a mouse in one dose maybe, for example, about 20 μg to about 300 μg, for example approximately 100 μg. The total amount of nucleic acid administered to a human in one dose maybe, for example, about 1.32 mg to about 19.8 mg, for example approximately 6.6 mg.

The cells may also be contacted with the complex ex vivo, using cells recovered from an animal or human. The method comprises adding the complex to the cells ex vivo in an amount effective to deliver the drug into the cells. The amount of drug administered to the cells ex vivo will depend on the type of cells, the amount of cells, the condition being treated, the particular drug used, the condition of the patient to which the cells will be re-administered to, etc. For example, when administering nucleic acid, the concentration of nucleic acid may range from, for example, about 1 μg/ml to about 5 mg/ml, about 150 μg/ml to about 500 μg/ml, about 200 μg/ml, about 150 1 μg/ml, about 125 μg/ml, at least about 150 μg/ml, at least about 200 μg/ml. The total amount of nucleic acid administered in one dose to a mouse may be, for example, about 20 μg to about 300 μg, for example approximately 100 μg. The total amount of nucleic acid administered to human cells in one dose maybe, for example, about 1.32 mg to about 19.8 mg, for example approximately 6.6 mg.

The complex may be administered, for example, orally, subcutaneously, nasally, intratumorally, intravenously, intratracheally, intraperitoneally, intracranially, intraepidemally, intramuscularly, or by injection into the spinal fluid. In a preferred embodiment, the complex is administered intravenously, for example, into the portal vein. The complex may be administered as, for example, an aerosol, liquid solution, dry powder or gel.

When administered in vivo, the complexes preferably induce lower levels of inflammatory cytokines (such as, for example, TNFα). The complexes may further provoke reduced inflammation responses. When the complexes of the invention are administered to mice intravenously, serum levels 2 hours after injection are preferably less than about 2000 picograms TNFα/ml serum per μg DNA delivered, more preferably less than about 1000, more preferably less than about 500, more preferably less than about 200, more preferably less than about 100, more preferably less than about 50, still more preferably less than about 20 picograms TNFα/ml serum per μg DNA delivered.

All articles, patent applications or patents referenced herein are hereby incorporated by reference in their entirety.

The following examples illustrate various aspects of the invention but are intended in no way to limit the scope thereof.

### EXAMPLES

Targeting Factor-Pegylated Lipid Conjuizates

The luteinizing hormone-releasing hormone (LHRH) and an 11 amino acid peptide containing one arginine-glycine-aspartic acid (RGD) motif were selected as ligand models for testing of targeting factor-pegylated lipid conjugate comprising complexes. The ligand choices were based on their specificity for their receptor and their small size. The ligands were conjugated to a DSPE-PEG_{5K} lipid anchor (Shearwater Polymer, Inc., Huntsville, Ala.) and were incorporated into lipid:protamine sulfate:DNA (LPD) and dialyzed lipid:protamine sulfate:DNA (DLPD) formulations at different concentrations ranging from 1 to 20 mol percent of lipid concentration, as described infra.

DSPE-PEG_{5K}-succinyl-ACDCRGDCFCG-COOH (DSPE-PEG_{5K}-RGD) (SEQ ID NO:10) and pyrGLU-HWSY_{D}K(εNH-succinyl-PEG_{5K}-DSPE)LRPG-COOH (DSPE-PEG_{5K}-LHRH) (SEQ ID NO:11) were obtained from Integrated Biomolecules (Tucson, Ariz.).

Synthesis of Membrane Translocating Peptides

The following membrane translocating peptides were synthesised by Anaspec (San Jose, Calif.) using a fmoc chemical synthesis method. Suitable finoc chemical syntheses are described supra. Majiscule letters denote L-amino acids, and miniscule letters denote D-amino acids.

| | | |
|---|---|---|
| ZElan094: | H₂N-KKKAAAVLLPVLLAAP | (SEQ ID NO:3) |
| ZElan207: | H₂N-kkkaavllpvllaap | (SEQ ID NO:4) |
| ZElan094R: | H₂N-KKKAAAVLLPVLLAAPREDL | (SEQ ID NO:5) |

Synthesis of Membrane Translocating Peptide-Galactose Conjugates

The following two membrane translocating peptide-galactose conjugates were synthesized by Integrated Biomolecules Corporation (Tucson, Ariz.) as described below:

| | | |
|---|---|---|
| Elan094G: | des-Pro-KKAAAVLLPVLLAAS-Galactose | (Formula weight: 1660) |
| | (SEQ ID NO:31) | |
| Gelan094: | S(Galactose)KKAAAVLLPVLLAAP | (Formula weight: 1757) |
| | (SEQ ID NO:32) | |

The Elan094-galactose (Elan094G) (SEQ ID NO:31) conjugate is synthesized by solid phase Fmoc chemistry, using a super-acid labile Wang-type resin. The Fmoc-serine(tetra-acetyl-galactose) is coupled using DMAP and DlPCDl. Uncoupled sites are capped with acetic anhydride. Subsequent chain elongation is carried out by normal cycles of Fmoc amino acid coupling. Double coupling is conducted where coupling efficiency was observed below 97%. Protection of side groups for lysine residues utilized Dde which can be orthogonally cleaved without use of high acidic conditions—viz., hydrazine hydrate. The same condition simultaneously removes the acetate protection from the carbohydrate moiety. Resin coupled deprotected peptide is washed copiously with dimethyl formamide and methanol to remove all deprotection contaminants. Final cleavage of the peptide is conducted with 2% TFA in dichloromethane, immediately being neutralized in piperidine. Product is isolated through solvent evaporation, prior to being purified by HPLC on a C 18 solid support.

The Galactose-Elan094 (GalElan094) (SEQ ID NO:31) conjugate is synthesized by solid phase Fmoc chemistry, using a Proline pre-loaded super-acid labile Wang-type resin. The next immediate two amino acids (Ala-Ala) are coupled as a protected dipeptide unit (Fmoc-Ala-Ala) to prevent elimination of the proline through diketopiperizine formation. Subsequent chain elongation is carried out by normal cycles of Fmoc amino acid coupling. Double coupling is conducted where coupling efficiency is observed below 97%. The Fmoc-serine(tetra-acetyl-galactose) is coupled using PyBOP with only a slight excess over the theoretical peptide substitution. Protection of side groups for lysine residues utilized Dde, which could be orthogonally cleaved without use of high acidic conditions—viz., hydrazine hydrate. The same condition simultaneously removes the acetate protection from the carbohydrate moiety. Deprotected peptide is washed copiously with dimethylformamide and methanol to remove all deprotection contaminants. Final cleavage of the peptide is conducted with 2% TFA in dichloromethane, immediately being neutralized in piperidine. Product is isolated through solvent evaporation, prior to being purified by HPLC on a C18 solid support.

Synthesis of Membrane Translocating Sequence-lipid Coniugates

The following three lipid-MTLP conjugates were synthesised by Integrated Biomolecules Corporation as described below:

| | | |
|---|---|---|
| Elan218: | Cholosteryl-succinyl-KKAAAVLLPVLLAAP | Formula weight: 1943.5. |
| | (SEQ ID NO:33) | |
| Elan219: | DOPE-succinyl-KKAAAVLLPVLLAAP | Formula weight: 2299.4 |
| | (SEQ ID NO:34) | |
| All d-Elan218: | Cholesteryl-succinyl-kkaaavllpvllaap | Formula weight: 1943.5 |
| | (SEQ ID NO:35) | |

The Elan218 conjugate is synthesized by solid phase F-moc chemistry, using a Proline pre-loaded super-acid labile Wang-type resin. The next immediate two amino acids (Ala-Ala) were coupled as a protected dipeptide unit (Fmoc-Ala-Ala) to prevent elimination of the proline through diketopiperizine formation. Subsequent chain elongation is by normal cycles of Fmoc amino acid coupling. Double coupling is conducted where coupling efficiency was observed below 97%. Cholesteryl-(C3)-hemisuccinate was coupled using PyBOP with only a slight excess over the theoretical peptide substitution. Protection of side groups for lysine residues utilized Dde which can be orthogonally cleaved without use of high acidic conditions—viz, hydrazine hydrate. Deprotected peptide is washed copiously with distilled water (DW) and methanol to remove all deprotection contaminants. Final cleavage of the peptide is conducted with 2% TFA in dichloromethane, immediately being neutralized in piperidine. Product is isolated through solvent evaporation and resuspension in methanol, prior to being purified by BPLC on a C4 solid support.

The Elan219 (SEQ ID NO:34) conjugate is synthesized by solid phase F-moc chemistry, using a Proline pre-loaded super-acid labile Wang-type resin. The next immediate two amino acids (Ala-Ala) are coupled as a protected dipeptide unit (Fmoc-Ala-Ala) to prevent elimination of the proline through diketopiperizine formation. Subsequent chain elongation is by normal cycles of Fmoc amino acid coupling. Double coupling conducted where coupling efficiency was observed below 97%. DOPE-succinate is coupled using PyBOP with only a slight excess over the theoretical peptide substitution. Protection of side groups for lysine residues utilized Dde which could be orthogonally cleaved without use of high acidic conditions—viz, hydrazine hydrate. Deprotected peptide is washed copiously with DMF and methanol to remove all deprotection contaminants. Final cleavage of the peptide is conducted with 2% TFA in dichloromethane, immediately being neutralized in piperidine. Product is isolated through solvent evaporation and resuspension in methanol, prior to being purified by HPLC on a C4 solid support.

All _{D-}Elan218 (SEQ ID NO:35) conjugate: this all D-amino acid conjugate is synthesized by solid phase F-moc chemistry, using a Wang-type resin. The Fmoc-D-Proline is coupled using DMAP and DIPCDI. Uncoupled sites are capped with acetic anhydride. The next immediate two amino acids (_{D}Ala-_{D}Ala) are coupled as a protected dipeptide unit (Fmoc-_{D}Ala-_{D}Ala) to prevent elimination of the proline through diketopiperizine formation. Fmoc-_{D}Ala-_{D}Ala is synthesized by solution phase chemistry and purified and characterized before incorporation into the solid phase synthetic system. Coupling of the protected dipeptide unit (Fmoc-_{D}Ala-_{D}Ala) is performed with PyBOP with only a slight excess over the theoretical peptide substitution. Subsequent chain elongation is by normal cycles of Fmoc amino acid coupling. Double coupling conducted where coupling efficiency is observed below 97%. Final cleavage of the peptide is conducted with 95% TFA with 2.5% TIS and 2.5% H₂O. Product is isolated through solvent evaporation, crude purification by precipitation with tertiary butyl ether. Final purification performed by HPLC on a C 18 solid support.

### METHODS AND MATERIALS FOR CATIONIC COMPLEXES

Preparation of Cationic Liposome Complexes

The liposomes were prepared as follows: 6000 nmol of 1,2-dioleoyl-3-trimethylammonium-propane (DOTAP) (Avanti Polar Lipid Inc., Alabaster, Ala.) at 20 mg/ml in chloroform and 6000 nmol of cholesterol (Avanti Polar Lipid Inc., Alabaster, Ala.), also prepared at 20 mg/ml in chloroform (J. T. Baker, Phillipsburg N.J.), were mixed in a borosilicate tube and dried for 1 hr under nitrogen at 4 liters per minute (LPM) using a N-EVAP™ (Organomation, Berlin, Mass.). The resulting lipid films were hydrated in 2 ml of 5% USP dextrose (Abbott Laboratories, North Chicago, Ill.) to achieve a final lipid concentration of 6 mM. The multilamellar vesicles (MLVs) generated were sonicated for 30 sec using a sonicating bath (Laboratory Supplies Co, Inc, Hicksville, N.Y.). The resulting lipid vesicles were sized and typically had a diameter from 300-500 nm, as determined in unimodal mode using a Coulter Sizer N4Plus (Beckman Coulter, Miami, Fla.).

For the liposomes comprising 10 mol % pegylated lipid, 6000 nmol DOTAP, 4800 nmol cholesterol and 1200 nmol of 1,2-distearoyl-sn-glycero-3-phosphotidylethanolamine-N-[methoxy(polyethylene glycol)-5K] (DSPE-PEG_{5K}) (Shearwater Polymer, Inc., Huntsville, Ala.) were prepared as described above. Typically, targeted liposomes comprising 10 mol % targeting factor-pegylated lipid conjugate or targeting factor-lipid conjugate were prepared using 6000 nmol DOTAP, 4800 nmol cholesterol and 1200 nmol of either DSPE-PEG_{5K}-succinyl-ACDCRGDCFCG-COOH (DSPE-PEG_{5K}-RGD) (SEQ ID NO:10), pyrGLU-HWSY_{D}K(εNH-succinyl-PEG_{5K}-DSPE)LRPG-COOH (DSPE-PEG_{5K}-LHRH) (SEQ ID NO:11), cholesteryl-succinyl-KKAAAVLLPVLLAAP (SEQ ID NO:33), DOPE-succinyl-KKAAAVLLPVLLAA-P (SEQ ID NO:34), or cholesteryl-succinyl-kkaaavllpvllaap (SEQ ID NO:35). The mol % of targeting factor-pegylated lipid conjugate or targeting factor-lipid conjugate was varied from 0-20%, with the corresponding mol % of cholesterol ranging from 50-30%. The targeting factor-pegylated lipid conjugates were synthesized by Integrated Biomolecule Corporation (Tucson, Ariz.). Briefly, for targeted liposomes, DOTAP, cholesterol and DSPE-PEG_{5K}-RGD (SEQ ID NO:10) or DSPE-PEG_{5K}-LHRH (SEQ ID NO:11) and/or MTLP-lipid were solubilized in chloroform at 20 mg/ml and were evaporated under nitrogen as described above. The lipid film was then re-solubilized in methanol:dichloromethane 1:1 (methanol from VWR, West Chester, Pa. and dichloromethane from EM Science, Gibbstown, N.J.), re-evaporated under nitrogen prior to hydration in 5% dextrose USP, and subsequently processed as described above for the DOTAP:cholesterol liposomes.

Preparation of DNA-Protamine Complexes

The plasmid pCMVinLUC, containing the fire fly luciferase gene under the control of the CMV promoter, was constructed as follows: the luciferase gene from the pGL3-basic vector (Promega, Madison, Wis., Genebank accession #U47295), was excised as a 1982 basepair Sma I to Sal I restriction fragment. Similarly, a 3482 bp Sal I to Sma I restriction fragment of pUCCMVb (Clontech, Palo Alto, Calif., GeneBank accession # U02451) containing the vector backbone and the CMV promoter/SV-40 intron sequence was excised and the two Sma I to Sal I fragments were ligated together. The resulting 5464 bp plasmid, pCMVinLUC, was isolated by standard molecular techniques (Sambrook et al., 1989) and purified by Althea Technologies (San Diego, Calif.).

The plasmid DNA pCMVinLUC was mixed with protamine sulfate USP (Elkins-Sinn, Cherry Hill, N.J.) at a mass ratio of 1:1, 1:0.9 or 1:0.97 as indicated using an Orion Sage™ syringe pump mixing device (VWR, West Chester, Pa.) at a flow rate of 25 ml/min using 20 ml syringes. Briefly, DNA and protamine sulfate were separately diluted to 2× final concentration in milli-Q water pH 7. Equal volumes of each solution were loaded into syringes and mixed into a glass reservoir via a T-fitting using an Orion syringe pump (VWR, West Chester, Pa.) at a flow rate of 25 ml/min. Optionally, the solution was inverted 2× and excess protamine was removed using 15 ml Slide-A-Lyzero® dialysis cassettes, 10,000 MWCO (Pierce, Rockford, Ill.), dialysed against 5% dextrose pH 7. The DNA/protamine complex was stored up to 2 weeks at 4° C. prior to use, and the final DNA concentration was determined via Pico Green® assay (P-7589 Pico Green® dsDNA Quantitation Kit from Molecular Probes, Eugene, Oreg.).

Preparation of Lipid-Protamine-DNA (LPD) Formulations

LPDs were prepared as described by Li, S., et al., Gene Therapy, 1998. 5(7): p. 930-937. Lipid:protamine:DNA ratios of 12 nmol:0.9 μg:1 μg, 12 nmol:0.97 μg:1 μg, and 12 nmol:1 μg:1 μg were used for these experiments, as indicated. As a typical example, 150 μl of liposomes at 6mM were mixed with 197 μl of 5% dextrose USP followed by the addition of 153 μl of pre-compacted DNA at 490 μg/ml. This operation was performed under moderate vortex agitation (speed #3), and the resulting LPD solution contained 150 μg DNA/ml. The LPDs were sized with a N4Plus Coulter Sizer using unimodal mode, and typically had a mean diameter from 150-250 nm. The surface zeta potential was determined using a Malvern zeta sizer (Malvern Instrument Inc, Sacramento, Calif.). Typically, LPD formulations showed an average zeta-potential of 25-45+/−5.0 mVolts with and without pegylated lipid or targeting factor-pegylated lipid conjugates.

Preparation of Dialyzed Lipid-Protamine-DNA (DLPD)

DLPDs were generated by a modified version of the method previously described by Harvie, P., F. M. Wong, and M. B. Bally, Biophys J, 1998. 75(2): p. 1040-51. Briefly, 900 nmol of total lipids were dried under nitrogen as described above, and re-suspended in 200 mM of N-Octyl-B-D-glucopyranoside (OGP Sigma, St. Louis Mo.) to form a micellar solution. 350 μl of protamine pre-compacted DNA at 0.9:1 protamine: DNA ratio were added to the lipid micelle solution under mild vortex agitation (speed 3). Particles formed spontaneously, i.e. the solution became cloudy. The mixture was then dialyzed against 5% dextrose for 48 h at 4° C. using 500 μl Slide-A-Lyzero dialysis cassettes (MW cut-off 10,000) (Pierce, Rockford, Ill.). The dextrose solution was replaced twice a day. Particle sizes were assessed as described above and typically were in the 150-300 nm range.

LHRH Receptor and Integrin Receptor Expression by Flow Cytometry

Tissue culture media were obtained from Biowhittaker (Walkersville, Md.).

MDA-MB-231 cells (a human breast carcinoma cell line), LL/2 (Lewis lung carcinoma), NCI-H69 (a small cell carcinoma cell line), and Skov3-ip1 (an ovarian adenocarcinoma cell line) (all from ATCC, Manassas, Va.) were grown at 37° C. with 5% or 10% CO₂ in DMEM 10% FBS containing penicillin/streptomycin antibiotic in a 150 cm³ flask. Cells were detached using 5 ml of EDTA 0.5M or 5 ml of trypsin-EDTA (0.05% trypsin 0.53 mM EDTA.4Na) and 1×10⁶ cells were subsequently distributed into a 14 ml Falcon FACS tube, washed once with PBS containing 1% FBS (FACS buffer), and suspended in 100 μl FACS buffer on ice. Cells were incubated for 1 h on ice with 5 μl of the appropriate antibody, either anti-αVβ3 FITC-conjugated or anti-αVβ5 FITC-conjugated (Chemicon International, Temecula, Calif.), or the antibody from the anti-human LHRH receptor clone A9E4 (Biogenesis, Kingston, N.H.). 10 μl of the isotype match antibody anti-CD8 IgG₁K (Pharmingen, San Diego, Calif.) was used as a control. After incubation with the appropriate antibody, the cells were washed 3 times with PBS 0.095M and re-suspended in 1.0 ml of 2% paraformaldehyde solution. 10,000 cells were run on a BD FACScan (Becton Dickson, San Jose, Calif.) and the data were analyzed using FACSNet™ (Becton Dickson, San Jose, Calif.).

LHRH Receptor And Integrin Receptor Expression On Target Cells Isolated From Nude Mice Bearing SO MDA-MB-231

Female nude Balb/C mice were injected subcutaneously with 1×10⁷ cells in the right flank. Tumors were harvested 8 weeks after cell injection when the average tumor volume reached 1 cm³. Cells were re-suspended in F12-DMEM (Biowhittaker, Walkersville, Md.) using a glass pestle Dounce homogenizer. 1×10⁶ cells were subsequently distributed in a Falcon FACS tube, washed once with PBS containing 1% FBS (FACS buffer), and were suspended in 100 μl FACS buffer on ice. Cells were incubated for 1 h on ice with 5 μl of the appropriate antibody or the appropriate isotype match as above, washed 3 times with FACS buffer, and re-suspended in 0.5 ml of 2% paraformaldehyde. 10,000 cells were analyzed on a BD FACScan as above.

Di-I Labeled LPD Binding to Cells

LPDs were labeled with 1,1′-dioctadecyl-3,3,3′,3′-tetramethylindo-carbocyanine perchlorate (DiI) (Molecular Probes, Eugene, Oreg.). Di-I is a non-exchangeable, non-metabolizable fluorescent lipid tracer (Claassen, E., J Immunol Methods, 1992. 147(2): p. 231-40). Typically, 6.67 μl of LPDs (containing 1 μg DNA) were diluted in 93 μl 5% dextrose USP and 1 μl of a DiI stock solution (at 50 μg/ml in methanol) was added to the LPD solution. After addition of DiI, LPDs were incubated at room temperature for 30 min prior to use. 100 μl of fluorescent LPDs were incubated with 1×10⁶ cells at 37° C. for 1 h, followed by 3 washes in PBS 0.095M, and re-suspended in 1.0 ml of 2% paraformaldehyde solution. 10,000 cells per sample were analyzed on a BD FACScan as described above.

In vitro Transfection

16-24 hr prior to transfection, 5×10⁴ cells (MDA-MB-231, LL/2 or HepG-2 (ATCC, Manassas, Va.)) in 500 μl/well of appropriate media (ATCC catalog describes appropriate media for each cell type) containing 10% FBS were seeded in a 48 well plate (Costar, Coming, N.Y.) and incubated overnight at 37° C. in 5% or 10% CO₂. The following day, the media was removed and replaced with 500 μl of fresh serum-free media. Transfections were performed using 1 μg DNA/well (typically 6.67 μl from the LPD stock solution containing DNA at 150 μg/ml) and cells were incubated for 4 h at 37° C. in 5% or 10% CO₂ Six replicates per LPD or DLPD formulation were tested. After transfection, luciferase activity was assayed as described (Promega Luciferase Assay Kit, Cat. No. E1501, Madison, Wis.). Briefly, the cell culture media was removed and replaced by fresh media containing 10% FBS, and the cells were incubated at 37° C. in 5% or 10% CO₂ for a further 48 h. Each well was washed with 1 ml PBS 0.095 M pH7.4 (Biowhittaker, Walkersville, Md.) and suspended in 200 μl of 1× luciferase reporter buffer (RLB) (Promega, Madison, Wis.). The cells were then subject to 3 cycles of freeze/thaw at −70° C./37° C., respectively. Cells were harvested and spun for 10 min at 14,000 rpm. 20 μl of the supernatant was assayed for luciferase activity by addition of 100 μl of luciferase substrate (Promega, Madison, Wis.), followed by a 10 sec incubation time at room temperature before luminescence relative units were measured using a Berthold (Aliquippa, Pa. autolumat B953 luminometer. The total protein concentration per sample was determined using a commercial kit of Coomassie plus dye (Pierce, Rockford, Ill.).

In vitro Transfection Assessment After Serum Incubation

Transfections were performed as described above except that 100 μl of LPDs were incubated for 1 h at 37° C. in 100 μl 5% dextrose USP or in 100 μl of 50% mouse serum (Cederlane, Homby, ON, Canada) prior to transfection in serum free media. Mean diameter of the LPD formulations following serum incubation was performed using the Coulter Sizer as described above.

Competition Assays

Three competitions assays were performed using MDA-MB-231 cells in order to assess specificity of transfection-mediated-DSPE-PEG_{5K}-LHRM (SEQ ID NO:10) or DSPE-PEG_{5K}-RGD (SEQ ID NO:11). Transfections were performed as described above except that the culture media was supplemented with free LHRH or RGD peptides, or antibody against the LHRH or the integrin receptor as competition agents. 10 mol % pegylated lipid or targeting factor-pegylated lipid conjugate was used for each experiment.

The first competition assay was performed using 1 μg DNA/well. This DNA concentration required a high concentration of free LHRH to achieve a 100 fold excess of free LHRH which was toxic for the cells. Similarly, with the RGD formulation, this DNA concentration required a high concentration of free RGD in order to achieve 100 fold excess of free RGD (3.25 μl of dansyl-labeled RGD ((Integrated Biomolecule Corporation, Tucson, Ariz.) from a solution at 5 mg/ml in H₂O) which was toxic for the cells.

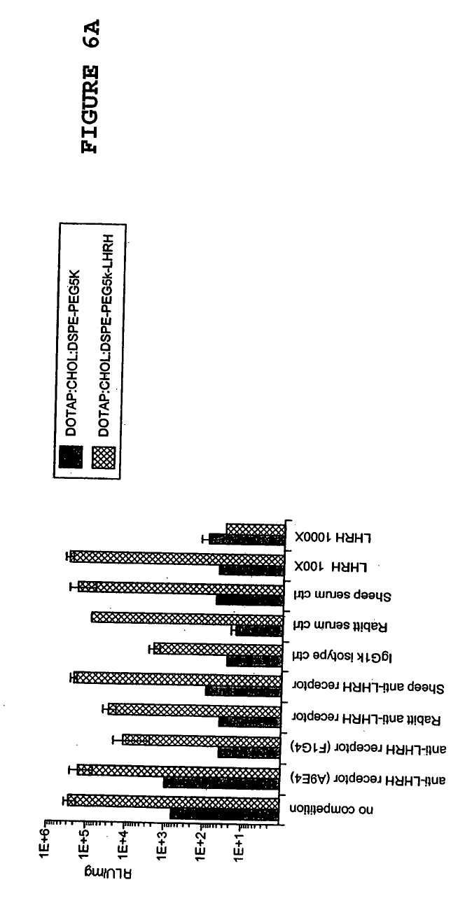

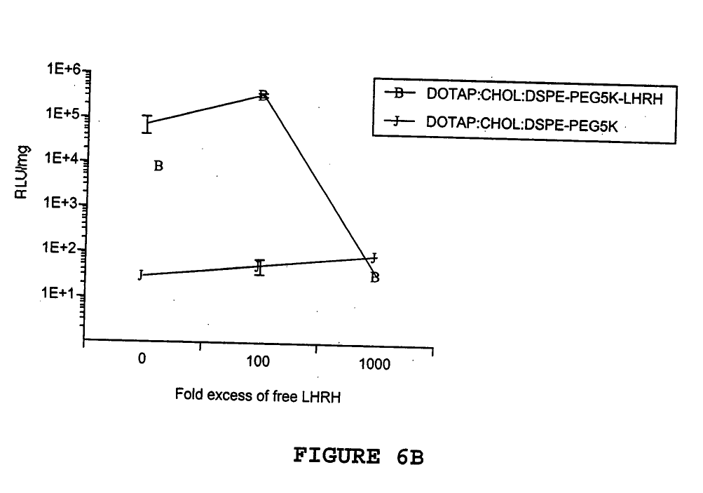

In order to decrease LHRH associated toxicity, a second competition assay was performed using 0.1 μg DNA/well (FIGS. 6A and 6B). 0.1 μg DNA/well corresponds to 862.62 ng DSPE-PEG_{5K}-LHRH (SEQ ID NO:10) or 168.6 ng of LHRH/well using a 12 nmol:1 μg lipid:DNA ratio and 10 mol % DSPE-PEG_{5K}-LHRH (SEQ ID NO:10) in the total lipids in the formulation. Transfections were performed as described above. The second competition assay was performed using 17.5 μg/well (5 μl) of anti-LHRH receptor F1G4, and 15 μg/well (50 μl) of anti-LHRH receptor A9E4 from Biogenesis (Kingston, N.H.). The appropriate isotype match antibody (17.5 μg/well), anti-CD3 IgG_{1K} (Pharmingen, San Diego, Calif.), was used as a control. Additionally, a 100-fold and 1000 fold excess of LHRH was tested as a competition agent: the media was supplemented with 6.5 μl or 65 μl of a stock solution of 2.5 mg/ml of [D-Trp]-LHRH from Sigma (St. Louis, Mo.). Moreover, in this particular experiment, 2 anti-LHRH receptor polyclonal serums were tested: 5 μl/well of a rabbit serum anti-LHRH receptor (Biogenesis, Kingston, N.H.) and 50 μl/well sheep serum anti-LHRH receptor (Biogenesis, Kingston, N.H.). The corresponding volumes of control serum were used as an isotype control. Control serums were purchased from Sigma (St. Louis, Mo.).

LPDs were also directly incubated with an anti-LHRH antibody or an isotype control (Anti-IgG) antibody before addition of LPDs to cells in order to block the LHRH molecule on the LPD surface (data not shown). Briefly, 6.67 μl of 1/10 diluted DSPE-PEG_{5K}-LHRH LPDs at 150 μg DNA/ml were mixed with 50 μl of mouse anti-LHRH antibody or control (Dako Corporation, Carpinteria, Calif.) and incubated for 1 h at room temperature prior to transfection as described above.

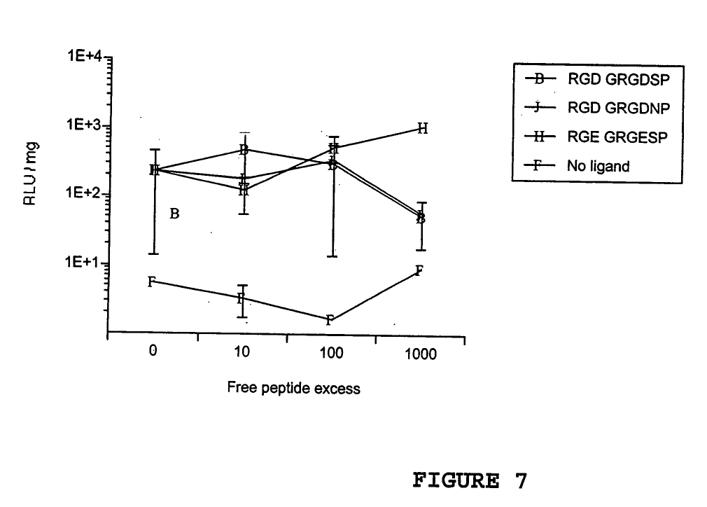

Transfections were also performed using 0.1 μg DNA/well in order to decrease RGD induced cell detachment from the culture plate, and transfection mediated DSPE-PEG_{5K}-RGD (SEQ ID NO:11) was blocked with 3 different RGD peptides: GRGESP (SEQ ID NO:41), GRGDSP (SEQ ID NO:42) and GRGDNP (SEQ ID NO:43) (Gibco Life Science, Gettysburg, Md.) were used as competitive agents (FIG. 7). Transfections were performed as described above except that the media was supplemented with 10, 100 or 1000-fold excess of peptide, corresponding to 6.5 μl (from stock solution diluted 1/10 in H₂O), 6.5 μl or 65 μl of peptide stock solution at 2.5 mg/ml.

### METHODS AND MATERIALS FOR ANIONIC COMPLEXES

Preparation Of Anionic Mixed Micelle Complexes

Mixed micelles were prepared as follows: 2191.5 nmol of 1,2-dioleoyl-sn-glycero-3-(phospho-L-serine) (DOPS) or 1,2-dioleoyl-sn-glycero-3-(phospho-rac-1-glycerol) (DOPG) (Avanti Polar Lipid Inc., Alabaster, Ala.), both at 20 mg/ml in chloroform (J. T. Baker, Phillipsburg, N.J.), were mixed with 1793.05 nmol of cholesterol (Avanti Polar Lipid Inc., Alabaster, Ala.), also prepared at 20 mg/ml in chloroform. Lipids were mixed a borosilicate tube and dried for 1 hr under nitrogen at 4 LPM using a N-EVAP™ (Organomation, Berlin, Mass.). The resulting lipid films were hydrated in 0.15 ml of 200 mM N-Octyl-B-D-glucopyranoside (OGP) (Sigma, St Louis, Mo.), to achieve a final lipid concentration of 26.56 mM. Micellar solutions generated were sonicated for 30 sec using a sonicating bath (Laboratory Supplies Co., Inc., Hicksville, N.Y.).

For the 10 mol % pegylated lipid formulations, 2191.5 nmol of DOPS or DOPG and 1394.59 nmol cholesterol and 398.45 nmol of 1,2-disteraoyl-sn-glycero-3-phosphotidylethanolamine-N-[methoxy(polyethylene glycol)-5000](DSPE-PEG_{5K}) (Shearwater Polymer Inc., Huntsville, Ala.) were prepared as described above. Typically, for 10 mol % targeting factor-pegylated lipid conjugate, targeted DLPD were prepared using 2191.5 nmol of DOPS or DOPG, 1394.59 nmol cholesterol and either 398.45 nmol DSPE-PEG_{5K}-succinyl-ACDCRGDCFCG-COOH (DSPE-PEG_{5K}-RGD) (SEQ ID NO: 10) or 398.45 nmol of pyrGLU-HWSY_{D}K(εNH-succinyl-PEG_{5K}-DSPE)LRPG-COOH (DSPE-PEG_{5K}-LHRH) (SEQ ID NO:11). The conjugated lipids were synthesized by Integrated Biomolecule Corporation (Tucson, Ariz.). Briefly, for targeted liposomes, anionic lipid, cholesterol and DSPE-PEG_{5K}-LHRH (SEQ ID NO:11) or DSPE-PEG_{5K}-RGD (SEQ ID NO:10) were solubilized in chloroform at 20 mg/ml and were evaporated under nitrogen as described above. The lipid film was then re-solubilized in methanol:dichloromethane 1:1 (methanol from VWR, West Chester, Pa. and dichloromethane from EM science, Gibbstown, N.J.), re-evaporated under nitrogen prior to hydration in 0.15 ml 200 mM OGP, and subsequently processed as described above.

pH sensitive micelles were prepared as described above for DOPS/cholesterol mixed micelles using 1461.0 nmole CHEMS (Sigma, St Louis, Mo.). (either cholesteryl hemisuccinate tris salt at 20 mg/ml in 200 mM OGP or cholesteryl hemisuccinate morpholine salt at 20 mg/ml in chloroform) and 3409.0 nmole DOPE (Avanti Polar Lipid Inc., Alabaster, Ala.) to achieve a final lipid concentration of 32.47 mM.

Preparation Of DNA-Protamine Complexes

DNA-protamine complexes were prepared as described above for cationic complexes, except the plasmid DNA pCMVinLUC was mixed with protamine sulfate USP (Elkins-Sinn, Cherry Hill, N.J.) at a 2:1 mass ratio.

Preparation Of Dialyzed Lipid-Protamine-DNA (DLPD) Formulations

Lipid:protamine:DNA ratios were prepared to give a 6:1 negative charge excess ratio. This corresponded to a ratio of approximately 53 nmol lipid: 2 μg protamine: 1 μg DNA for the DOPG/cholesterol and DOPS/cholesterol formulations. For complexes without pegylated lipid, lipid ratios were 5.5:4.5 DOPG:chol or 5.5:4.5 DOPS:chol. For complexes containing pegylated lipid, the amount of cholesterol was reduced accordingly. For example, for a complexes containing pegylated lipid at 10 mol % of total lipids, the lipid ratios were 5.5:3.5:1 DOPG or DOPS:chol:pegylated lipid.

Typically, 0.15 ml of mixed micelles prepared as describe above were mixed with 350 μl of DNA pre-compacted with protamine at a 2:1 protamine:DNA ratio under mild vortex agitation (speed 3). Particles formed spontaneously, i.e. solution became cloudy. The mixture was then dialyzed again milli-Q water for 48 h at 4° C. using 500 μl Slide-A-Lyzer dialysis cassettes (MW cut-off 10,000) (Pierce, Rockford, Ill.). The milli-Q water was replaced twice a day, and in the last dialysis step the milli-Q water was replaced with 5% dextrose USP. Particle sizes were assessed as described above and typically were in the 100-200 nm range as determined using a N4Plus coulter (Beckman Coulter, Miami, Fla.) sizer in unimodal mode. The surface zeta-potential was determined using a Malvern zeta sizer (Malvern Instrument Inc, Sacramento, Calif.). Typically, these DLPD formulations showed a zeta-potential of −35.0 to −45.0+/−5.0 mVolts with and without pegylated lipid.

Preparation Of pH Sensitive DLPD Formulations

Lipid:protamine:DNA ratios were prepared to give a 4:1 negative charge excess ratio. This corresponded to a ratio of approximately 65 nmol lipid: 2 μg protamine: 1 μg DNA for the CHEMS/DOPE formulation. For complexes without pegylated lipid, lipid ratios were 3:7 CHEMS:DOPE. For complexes containing pegylated lipid, the amount of DOPE was reduced accordingly. For example, for a complexes containing pegylated lipid at 10 mol % of total lipids, the lipid ratios were 3:6:1 CHEMS:DOPE:pegylated lipid.

Typically, 4870 nmol of total lipids (e.g., 1461 nmol CHEMS and 3409 nmol DOPE) were dried under nitrogen as described above and re-suspended in 150 μl of 200 mM of N-Octyl-B-D-glucopyranoside (OGP) (Sigma, St Louis, Mo.) to form a micellar solution. 350 μl of protamine pre-compacted DNA at a 0.9:1 protamine:DNA ratio were added to the lipid micelle solution under mild vortex agitation (speed 3). Particles formed spontaneously, i.e. solution became cloudy. The mixture was then dialyzed in a 500 μl Slide-A-Lyzer® dialysis cassette (MW cut-off 10,000) (Pierce, Rockford, Ill.) against 5% dextrose pH 7.4 for 48 h at 4° C. Particles sizes were assessed as described above, and typically were in a 200-300 nm range as determined using a N4Plus Coulter Sizer in unimodal mode. The surface zeta potential was determined using a Malvern zeta sizer (Malvern Instrument Inc, Sacramento, Calif.). Typically, these anionic DLPD formulations had a zeta-potential of −35.0 to −40.0+/−5.0 mVolts with or without pegylated lipid.

Preparation Of Control Cationic LPD Formulations

Cationic LPD formulations comprising DOTAP:Chol were prepared as described supra, except the lipid:protamine:DNA ratio was 12 nmol:2 μg: 1 μg.

Di-I Labeled DLPD Binding to Cells

DLPDs were labeled with 1,1′-dioctadecyl-3,3,3′,3′-tetramethylindo-carbocyanine perchlorate (DiI) (Molecular Probes, Eugene, Oreg.). Typically, 13.3 μl of DLPDs (containing 1 μg DNA) were diluted in 86.7 μl 5% dextrose USP and 1 μl of a DiI stock solution (at 50 μg /ml in methanol) was added to the DLPD solution. After addition of DiI, DLPDs were incubated at room temperature for 30 min prior to use. 100 μl of fluorescent DLPDs were incubated with 1×10⁶ cells at 37° C. for 1 h, followed by 3 washes in PBS 0.095 M, and re-suspended in 1.0 ml of 2% paraformaldehyde solution. 10,000 cells were analyzed on a BD FACScan (Becton Dickinson, San Jose, Calif.) and data were analyzed using FACSNet™ (Becton Dickinson, San Jose, Calif.).

In vitro Transfection

16 hr prior to transfection, 5×10⁴ cells (CHO-K1 cells (an ovarian cancer cell line isolated from adult Chinese hamsters), MDA-MB-231 cells (a human breast carcinoma cell line), KB cells, or HepG-2 (a hepatocellular carcinoma cell line) (all cell lines from ATCC, Manassas, Va.)) in 500 μl/well of appropriate media containing 10% FBS were seeded in 48 well plate (Costar, Coming, N.Y.) and incubated overnight at 37° C. in 5% or 10% CO₂. The following day, the media was removed and replaced with 500 μl of fresh serum-free media. Transfections were performed using 1 μg DNA/well (typically 13.3 μl from the DLPD stock solution (at 75 μg DNA/ml) and cells were incubated for 4 h at 37° C. in 5% or 10% CO₂. Six replicates per DLPD formulation were tested. After transfection, luciferase activity was assayed as described infra. Briefly, the transfection cell culture media was replaced by fresh media containing 10% FBS, and the cells were incubated at 37° C. in 5% or 10% CO₂ for a further 48 h. Each well was washed with 1 ml PBS 0.095 M pH7.4 (Biowhittaker, Walkersville, Md.) and suspended in 200 μl of 1× luciferase reporter buffer (RLB) (Promega, Madison, Wis.). The cells were then subjected to 3 cycles of freeze/thaw at −70° C. and 37° C., respectively. Cells were harvested and spun down for 10 min at 14,000 rpm. 20 μl of the supernatant was assayed for luciferase activity by addition of 100 μl of luciferease substrate (Promega, Madison, Wis.) followed by a 10 sec incubation time at room temperature before relative luminescence units were measured using a Berthold (Aliquippa, Pa. autolumat B953 luminometer. The total protein concentration per sample was determined using a commercial kit of Coomassie plus dye (Pierce, Rockford, Ill.).

In vitro Transfection Assessment After Serum Incubation

Transfections were performed as described above except that 100 μl of DLPDs were incubated for 1 h at 37° C. in 100 μl 5% dextrose USP or in 100 μl of 50% mouse serum (Cederlane, Homby, ON, Canada) prior to transfection in serum free media. DLPD mean diameter and zeta-potential measurement following serum incubation were performed as described above.

### EXAMPLE 1

Physical Properties of LPD and DLPD

LPD and DLPD formulations were sized and the zeta-potential was measured in 5% dextrose USP at pH 5.0. The mean particle size and population size distributions (represented by the polydispersity value) for formulations containing 10 mol % of pegylated lipid as a percent of total lipids are shown in Table 1. Typically, zeta-potentials range from 35 to 45 mV for a conventional LPD composed of 12 nmol lipid (DOTAP:CHOL 1:1 mol ratio): 0.9 μg protamine: 1.0 μg DNA. Incorporation of DSPE-PEG_{5K} yielded similar results. DSPE-PEG_{5K}-LHRH (SEQ ID NO: 11) incorporation only modified slightly the LPD surface potential. However, when DSPE-PEG_{5K}-RGD (SEQ ID NO:10) was used a 15 mV decrease in the zeta-potential was observed. Data shown in Table 1 are from a single experiment. Sizing data were repeated for every experiment presented (data not shown).

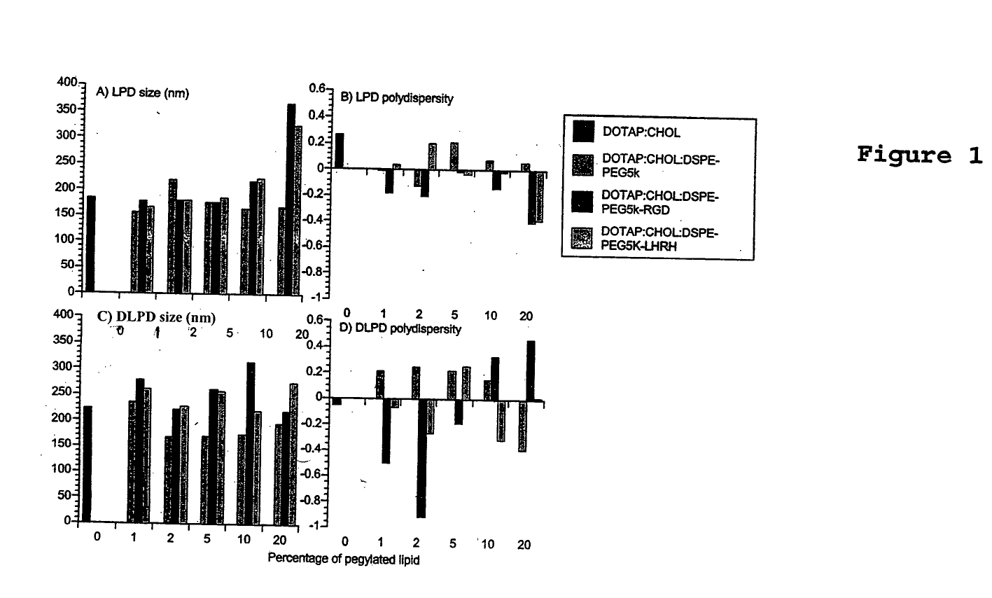

Data shown in FIG. 1 are from different runs of the same experiment shown in Table 1.

FIGS. 1A and 1B show that incorporation of targeting factor-pegylated lipid conjugate (DSPE-PEG_{5K}-LHRH (SEQ ID NO:11) or DSPE-PEG_{5K}-RGD (SEQ ID NO:10)) in LPD formulations up to 10 mol % of total lipids did not significantly affect the particle size. However, 20 mol % targeting factor-pegylated lipid conjugate incorporation in LPD resulted in a significant size increase over DSPE-PEG_{5K} pegylated formulation. DLPD formulations did not demonstrate the same size increase effect up to 20 mol % targeting factor-pegylated lipid conjugate incorporation as shown in FIGS. 1C and 1D. Both LPD and DLPD formulations are within the desired size range (<350 nm).

The effect of 10 mol % DSPE-PEG_{5K} addition to LPD formulations on the achievable DNA concentration is shown in Table 2.

The complexes comprised a ratio of 12 nmol lipid: 1 μg protamine: 1 μg DNA. LPD formulations which did not contain PEG were only able to achieve a maximum concentration of 150 μg DNA/ml (data not shown). The above formulations were able to generate formulations containing DNA at at least 200 μg DNA/ml.

### EXAMPLE 2

LHRH Receptor and αVβ Integrin Receptor Expression In Different Cells

MDA-MB-231, Skov3-ip1 (SKOV3-ip1), LL/2 and NCI-H69 (H-69) cells were investigated for their expression of the LHRH and integrin receptors by FACS analysis. As shown in Table 3, MDA-MB-231 and SKOV3-IP1 cells express both αVβ3 and αVβ5 integrin receptors. Interestingly, αVβ3 integrin receptor expression is higher in MDA-MB-231 than in SKOV3-IP1 cells, which express higher levels of αVβ5 integrin receptor. LHRH receptor expression level is lower than the expression of the integrin receptors in MDA-MB-231, although in MDA-MB-231* and MDA-MB-231** anti-αvβ5 receptors were expressed at slightly lower levels than LHRH receptor. SKOV3-IP1 showed 89.4% of the cell population expressing LHRH. H-69 and LL/2 did not show LHRH receptor expression using the human anti-human LHRH receptor. Data shown in Table 3 are from a single, typical experiment. (n=1 experiment per cell line).

### EXAMPLE 3

LPD Cell Binding Using DiI As a Lipid Marker

LPDs (lipid:protamine:DNA ratio of 12 nmol:1 μg: 1 μg) were labeled as described above with DiI, a fluorescent lipid tracer, and LPD binding to cells was assessed by flow cytometry. Results shown in Table 4 showed a higher percentage of cells bound to conventional LPDs compared with the DSPE-PEG_{5K} pegylated LPD formulation. These results support the concept that pegylated lipids decrease the electrostatic LPD-cell binding as compared with conventional LPDs. Addition of DSPE-PEG_{5K}-LHRH (SEQ ID NO:11) in the LPD formulation significantly restored the LPD-cell binding. DSPE-PEG_{5K}-RGD (SEQ ID NO:10) in the LPD formulation partially restored LPD-binding over DSPE-PEG_{5K} LPD, but showed lower cell binding when compared to DSPE-PEG_{5K}-LHRH (SEQ ID NO:11). The same trend is observed for mean fluorescence intensity as shown in Table 5.

### EXAMPLE 4

In vitro Luciferase Expression

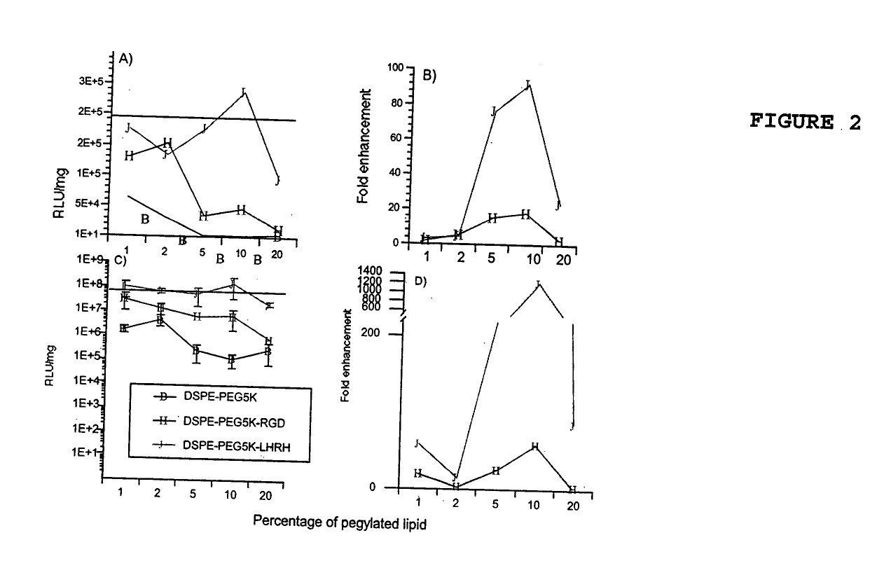

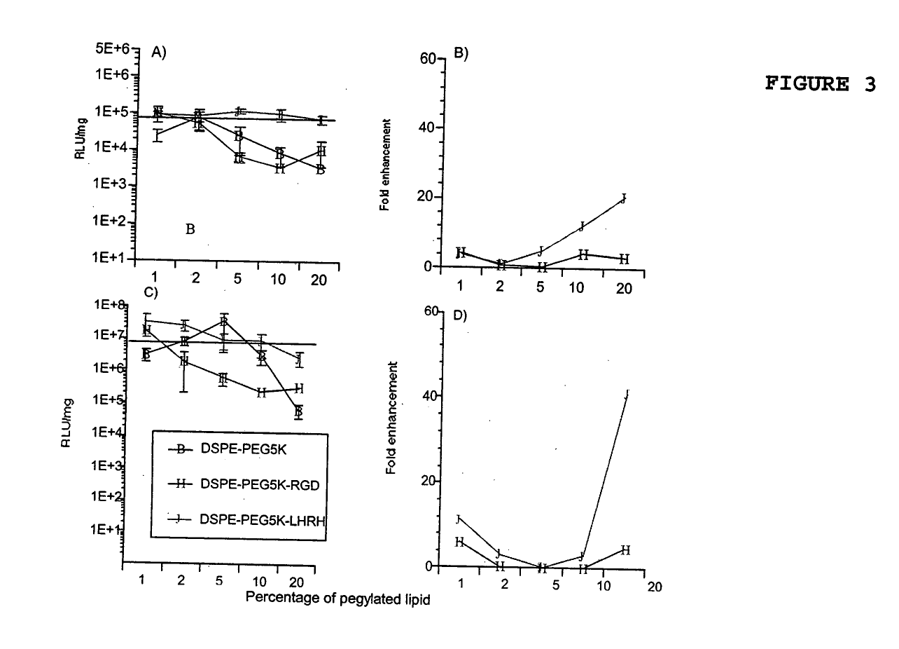

LPD (FIG. 2) and DLPD (FIG. 3) transfection activity and fold enhancement over DSPE-PEG_{5K} in 2 different cell lines (MDA-231, LL2) are shown. LPD and DLPD formulations contained 12 nmol lipid: 1 μg protamine: 1 μg DNA, and the pegylated lipid was incorporated at 1-20 mol % of total lipids. N=6 independent transfections per formulation group. The solid line in FIGS. 2A, 2C, 3A and 3C show luciferase expression in DOTAP/cholesterol LPDs and DPLDs which do not contain pegylated lipid. A dose-effect up to 10 mol % targeting factor-pegylated lipid conjugate in LPD formulations is seen for transfection of MDA-MB-231 cells (FIGS. 2A and 2B). A much smaller effect is observed for DLPD formulations in MDA-MB-231 cells up to 20 mol% targeting factor-pegylated lipid conjugate (FIGS. 3A and 3B). The LL/2 transfection experiment was repeated because fast growing cells in the first experiment resulted in over-confluent cell density (only the second experiment is shown in FIGS. 2 and 3). In the second LL/2 experiment, to avoid an over-confluent plate, transfections were stopped after 24 h, and the results showed a dose effect up to 10 mol% for targeted LPD formulations (FIGS. 2B and 2D). A smaller dose effect was observed for the DLPD formulation (FIGS. 3B and 3D). No effects were observed in the HEPG-2 cell lines for either LPD or DLPD formulations (data not shown). However, the HEPG-2 cell line is known to express low or no levels of LHRH and integrin receptors.

### EXAMPLE 5

LPD Stability in Mouse Serum

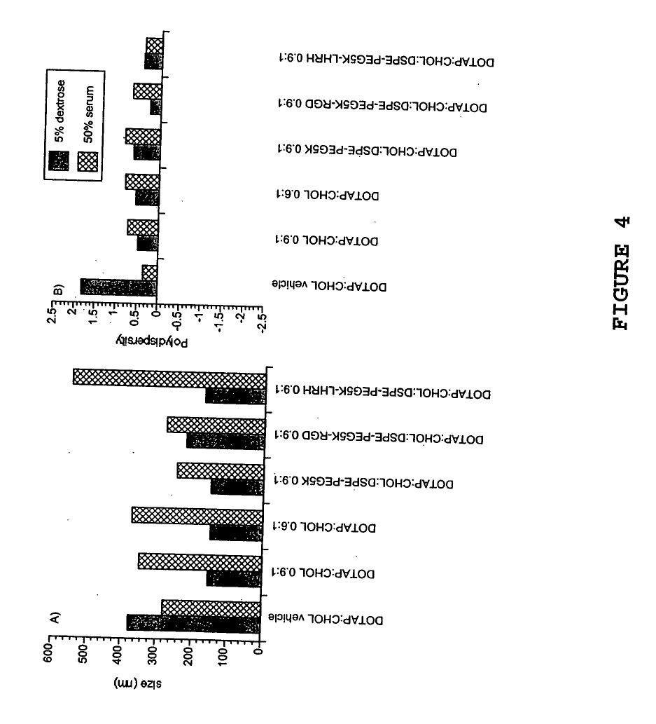

As a model for LPD stability in vivo, LPDs were pre-incubated for 1 h at 37° C. in mouse serum prior to transfection in serum-free media. In this experiment, cells were harvested 24 hours post transfection. The particle size and transfection abilities on MDA-MB-231 cells were evaluated. The DOTAP:CHOL vehicle contains no protamine/DNA. The ratios shown indicate the ratio of protamine to DNA (e.g., DOTAP:CHOL 0.9:1 indicates a protamine:DNA ratio of 0.9:1 in the formulation). All formulations contained 12 nmol lipid: 0.9 or 0.6 μg protamine: 1 μg DNA, and the pegylated lipids were included at 10 mol % of total lipids. N=6 independent transfections per formulation. DSPE-PEG_{5K} addition at 10 mol % in an LPD formulation prevented serum-mediated size increase (FIG. 4A). DSPE-PEG_{5K}-LHRH (SEQ ID NO:11) addition to LPD formulation resulted in a size enlargement after serum incubation, comparable to formulation without DSPE-PEG_{5K}. However, the particle sizes may be adjusted by adjusting the ratios of lipids, and of lipids to protamine to DNA. The formulation comprising DSPE-PEG_{5K}-RGD (SEQ ID NO:10) is less susceptible to serum than the LHRH formulation.

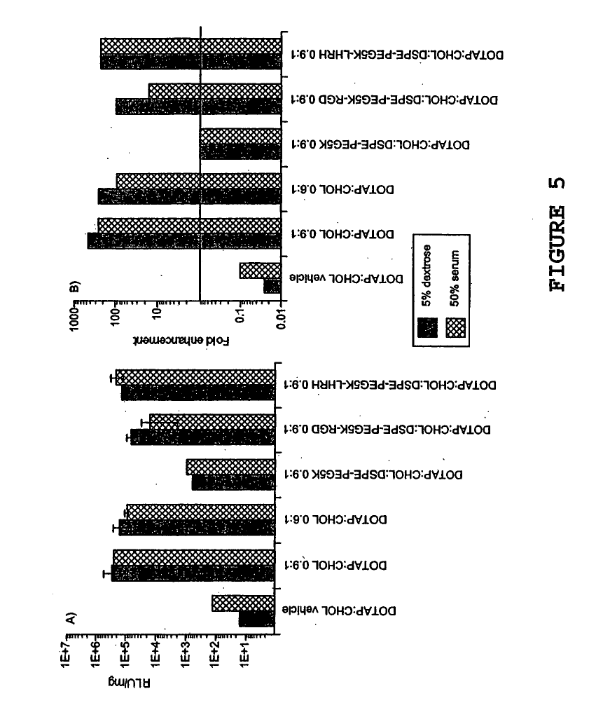

As expected, the DSPE-PEG_{5K} addition in LPD formulation at 10 mol % resulted in a 2 log decrease in terms of transfection activity compared to the basic DOTAP:CHOL LPD formulation activity (FIG. 5). However, DSPE-PEG_{5K}-LHRH (SEQ ID NO:11) addition to LPD formulation restored the transfection level to comparable to that observed in 5% dextrose for a conventional LPD (DOTAP:Chol) formulation. DSPE-PEG_{5K}-RGD (SEQ ID NO:10) addition showed a slightly lower transfection level restoration. Within assay variability (+1 SD), transfection potency of the LHRH bearing formulations remained stable with a slight decrease seen in the RGD bearing formulations.

### EXAMPLE 6

Competition Assays

In order to assess LHRH and RGD specificity for DSPE-PEG_{5K}-LHRH (SEQ ID NO:11) and DSPE-PEG_{5K}-RGD (SEQ ID NO:10) mediated transfection, we carried out competition assays as described supra. N=3 independent transfections per formulation group. All formulations contained 12 nmol lipid: 0.9 μg protamine: 1 μg DNA, and the pegylated lipids were included at 10 mol % of total lipids.

The results demonstrated a 1000-fold competition effect using 1000 fold excess of LHRH (FIG. 6A). However, no antibody specific to LHRH or the LHRH receptor was able to block ligand-mediated transfection. The data from the 100 and 1000-fold LHRH excess are presented again in FIG. 6B, which shows a clear inhibition of transfection activity using a 1000-fold excess of LHRH. FIG. 7 shows inhibition of transfection activity of DSPE-PEG_{5K}-RGD (SEQ ID NO:10) comprising complexes by 1000-fold excess of free RGD peptides (GRGDSP (SEQ ID NO:42) and GRGDNP (SEQ ID NO:43)). As expected, a 1000-fold excess of a free RGE peptide (GRGESP (SEQ ID NO:41)) did not inhibit transfection activity.

### EXAMPLE 7

LPD Cell-Binding Competition Assays

To correlate transfection activity decrease with a reduction of LPD-binding to cells, a competition assay was performed using Di-I labeled LPDs. The competition assay was performed using 10 mol % DSPE-PEG_{5K}-LHRH LPDs, and the competition agent was a 1000-fold excess of LHRH. We were not able to demonstrate a significant decrease in terms of percentage of LPD binding to SKOV3-IP1 cells or in terms of mean fluorescence intensity under conditions in which we clearly demonstrated inhibition of transfection activity in MDA-MB-231 cells (data not shown). However, as shown in Table 3, SKOV3-IP1 cells have a much higher density of receptors than MDA-MB-231 cells, and thus a 1000-fold excess of LHRH would not necessarily inhibit LHRH- mediated binding as much as in MDA-MB-231 cells.

### EXAMPLE 8

TNFα Response to LPD Formulations

Different LPD formulations were injected intravenously into mice into order to determine the level of TNF-α response to the formulations. Two different pCMV-In-Luc luciferase DNA preparations (Lot No. CP991216A containing 83 EU/mg and Lot No. CP000907A containing 0.17 EU/mg (Althea Technologies, San Diego, Calif.)) containing different levels of endotoxin were used to determine whether endotoxin level affected TNFα response.

All LPD formulations were prepared as described supra, except that protamine was added to the cationic liposomes, followed by DNA addition. All formulations containing DNA had an endotoxin level of 4.15 EU/mg, except the low endotoxin (low EU) formulation group which contained an endotoxin level of 0.0085 EU/mg. LPDs had a lipid:protamine:DNA ratio of 12 nmol:0.6 μg:1 μg, with the lipids comprising DOTAP:cholesterol at a 1:1 molar ratio. Where pegylated lipids or DOPE-094 (Elan 219) (SEQ ID NO:12) were added to the lipids, the amount of cholesterol was reduced accordingly. For example, for an LPD comprising 10 mol % DSPE-PEG_{5K}, the lipid ratios were 5:4:1 DOTAP:cholesterol:DSPE-PEG_{5K}. LPD formulations were sized as described supra, and are presented in Table 6.

Naïve female C57B/6 mice were injected intravenously with 333 μl (containing 50 μg pCMVinLUC DNA) of the formulations (N=3 mice per formulation). After 2 hours, mice were anesthetized and blood samples obtained via cardiac stick. The blood samples were centrifuged to obtain serum (the serum can be stored at −70° C.), the red blood cells were discarded, and an ELISA assay for TNFα was performed on the serum using a commercially available kit (Cat. No. MTA00 Mouse TNFα Immunoassay) from R&D Systems (Minneapolis, Minn.).

Briefly, the ELISA assay was performed as follows: All reagents were brought to room temperature, and reagents and standard dilutions were prepared. 50 μL of Assay Diluent was added to each well. 50 μL of standard, serum control or serum sample were added to each well. The solutions were mixed by gently tapping the plate. frame for 1 minute, and the plate was then covered with the adhesive strip provided and subsequently incubated for 2 hours at room temperature. Each well was aspirated and washed 5 times with 400 μL of Wash Buffer. 100 μL of Mouse TNF-α Conjugate was added to each well. The plate was covered with a new adhesive strip and incubated for another 2 hours at room temperature. The aspiration wash step was repeated as above. 100 μL of Substrate Solution was added to each well, and the samples were incubated for 30 minutes at room temperature. 100 μL of Stop Solution was added to each well, and the plate was gently tapped to ensure thorough mixing. Optical density was read at 450 nm (correction wavelength set at 540 nm or 570 nm).

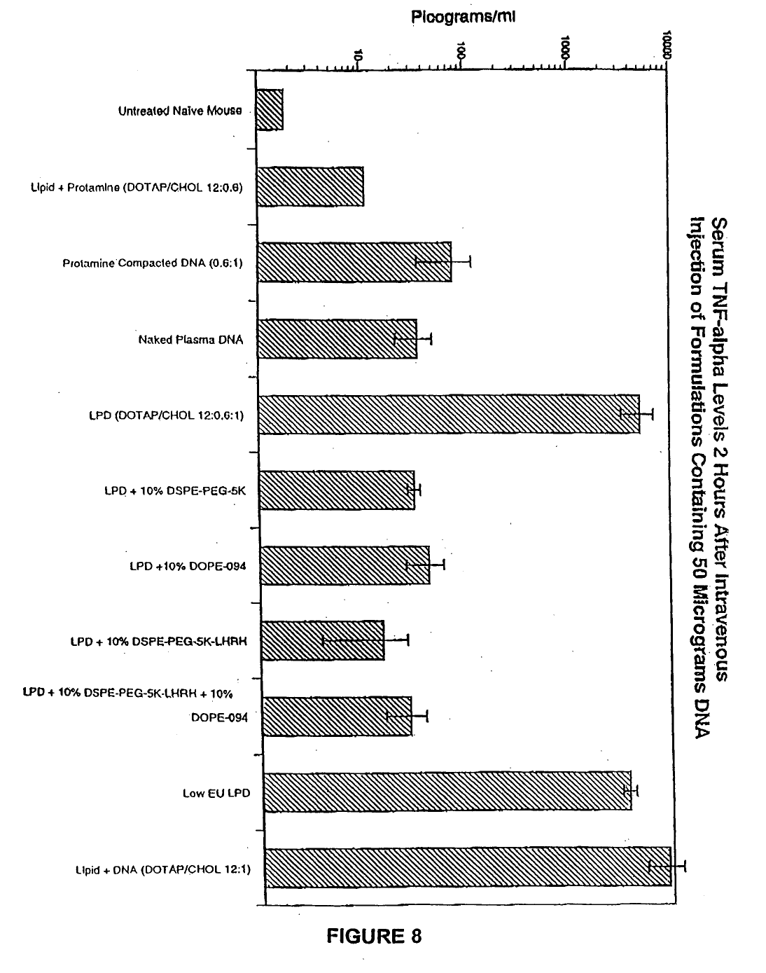

As shown in FIG. 8, injection of a standard DOTAP/CHOL LPD formulation provoked a strong TNF-α response, and a liposome formulation without protamine compaction resulted in an even higher TNF-α response than the LPD. The individual components (naked plasmid DNA, lipid+protamine, and protamine compacted DNA) of the DOTAP/CHOL LPD formulation did not provoke a significant TNF-α response. The LPD formulation with a lower endotoxin level (“low EU LPD”) resulted in a similar TNF-α response than a LPD with a higher endotoxin level, indicating that the TNF-α response was due to the LPD formulation, and not endotoxin levels. The formulations comprising 10% DSPE-PEG_{5K}, 10% DSPE-PEG_{5K}-LHRH (SEQ ID NO:11), and/or 10% DOPE-094 (Elan 219) (SEQ ID NO:12) significantly reduced the TNF-α response.

### EXAMPLE 9

Analysis of MTLP Peptide Stability in Serum over Time

The MTLP peptide solutions were prepared at 1 mg/mL and 20 μL aliquots placed in tubes. To one set of tubes an equal volume of mouse serum was added and to a second set 0.9% saline solution was added as negative a control. The tubes were incubated at 37° C. A control and a sample tube were removed at various timepoints (10, 30, 60 and 120 min) and any reaction quenched using 70:30 acetonitrile:water (160 μL). Each quenched sample was then analysed by HPLC-UV at 220 nm with a C18 column (5 μm, 300Å, 250×4.6 mm id) for 45 minutes. Mobile phase A was 10:90 acetonitrile: 0.1% trifluoroacetic acid in water and mobile phase B was 0.1% trifluoroacetic acid in acetonitrile.

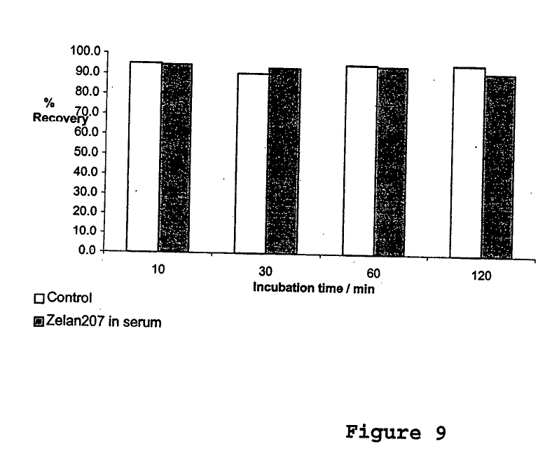

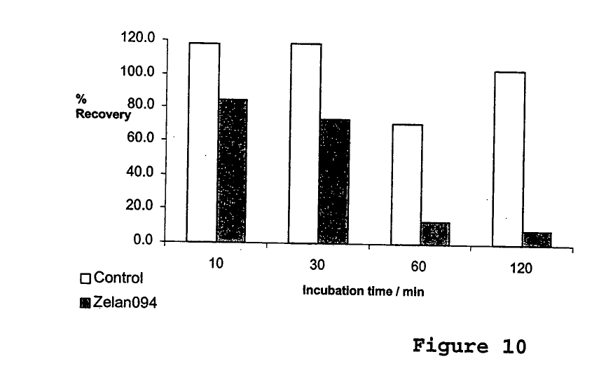

FIG. 9 shows that ZElan207 (D form) is stable in mouse serum up to 2 h. ZElan094 (L Form) degrades in mouse serum over 2 hr, with degradation starting after 10 min (FIG. 10), although up to 60% of the ligand is still detectable at 30 minutes following incubation. Thus, ZElan207 would be suitably stable for use in vivo via intravenous administration, especially where more prolonged circulation half-life is required. ZElan094 may be suitable in vivo via intravenous administration where a shorter serum half-life is required or where optimal tissue uptake occurs in those tissues exposed to or in contact with the administered formulation within 30 minutes of in vivo intravenous administration, such as the liver.

### EXAMPLE 10

Use of MTLP-galactose Conjugates Absorbed To LPDs in Order To Deliver Gene Complexes To Hepatocytes in vitro

For in vitro cell testing of the galactosylated peptides, the following cell lines were used: HepG2 (ATCC, Manassas, Va.): Hepatocarcinoma cell line expressing the asialyglycoprotein (ASGP) receptor. Hep-SK1 (ATCC, Manassas, Va.): Liver adenocarcinoma cell line that does not express the ASGP receptor. These two cell lines were tested for the presence of the ASGP receptor by westen blotting with an anti-asialoglycoprotein receptor antibody from Calbiochem (Nottingham, UK). This confirmed the presence of the receptor in HepG2 cells and the absence of the receptor from Hep-SK1 cells (data not shown).

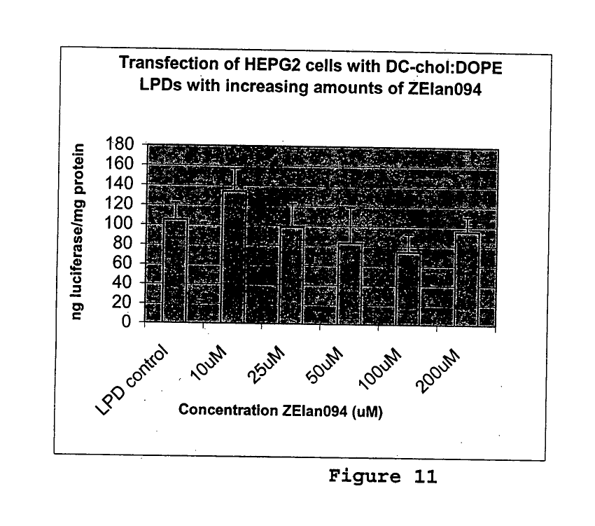

LPDs were made as described supra, wherein the lipids comprised DC-Chol/DOPE and optionally, targeting factor. Dose titration studies with ZElan094 indicated that 10 μM MTLP peptide gave optimal in vitro cell transfection, an example of which is shown in FIG. 11.

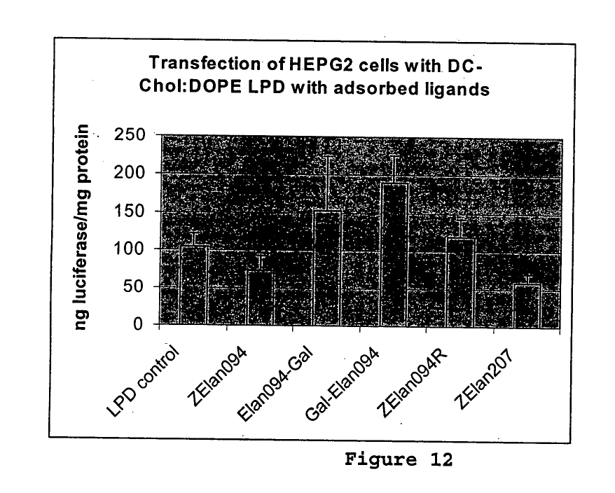

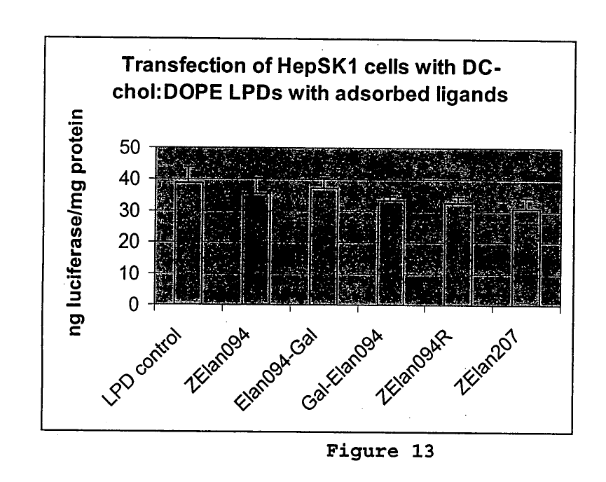

Galactosylated ligands (2mg/ml stock in dH₂O) were diluted to a working dilution of 1 mM, from which ligand was added to the LPD formulations to give a final concentration of 100 μM, and the formulations were incubated for 30 minutes at room temperature. Cells were washed 3 times with PBS and 250 μl of the final LPD formulation containing 1 μg DNA and 100 μM of MTLP-galactose conjugate in OptiMEM was added to each well of cells (n=6). The formulations were incubated with the cells for 4 h, at which point the formulations were aspirated and fresh serum containing complete medium was added to the cells. Cells were harvested 48 h post transfection and were assayed for total cell protein expression and luciferase reporter gene expression. The galactosylated-Elan094 (SEQ ID NO:6) ligands, Elan094 (SEQ ID NO:2) ligands, and Zelan207 (SEQ ID NO:4) ligand were screened using DC-Chol:DOPE LPDs with the results from a typical experiment outlined in FIGS. 12 and 13 using 100 uM of galactosylated-Elan094 (SEQ ID NO:6) ligands in the LPD formulation. Increased transfection was observed for Elan094G, GElan094 and the ligand ZElan094R (which contains an endosomal escape peptide sequence attached to the C-terminus of ZElan094) in HepG2 cells (FIG. 12) while no increase in transfection was observed in the non-ASGPR containing cells, Hep-SK 1 (FIG. 13). Thus, multifunctional targeting factors such as galactose-Elan094 (SEQ ID NO:6) can increase transfection activity over a single fimctional targeting factor.

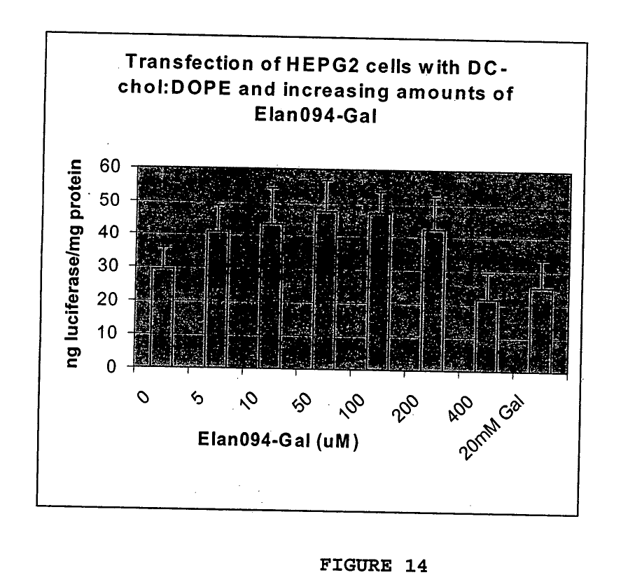

A dose titration study of Elan094G, used in conjunction with DC-chol:DOPE LPDs, indicated that there was a dose response (FIG. 14), with optimal transfection obtained at 50 uM (with the 10 uM and 100 uM doses were very similar). The addition of 20 mM free galactose did compete with 100 uM LPD-GElan094, and reduced transfection to background levels, indicating free galactose competition with GElan094.

### EXAMPLE 11

Dose Titration of Lipid-MTLP into LPDs and in vitro Transfection

LPD formulations were produced as described supra with the addition of Elan218 or Elan219. In vitro cell transfections were carried out as described supra in a number of cell types. Targeting factor was included as either targeting factor-lipid conjugates or as targeting factor-lipid conjugates conjugated to a pegylated lipid. The optimal percentage of Elan218 or Elan219 depended both on the LPD base formulation and on the cell type. The results for two different cell types, a human liver cell line (HepSK1) and a human breast cell line (MDA-MB-231), are shown in Tables 7 and 8. The optimal concentration for the Elan218 conjugate with the HepSK1 cells and the MD-MBA-231 cells in the DOTAP:Chol:DMPE-PEG:Elan218 LPDs is 10 mol % (Tables 7 and 8, respectively). The optimal concentration for the Elan219 conjugate with the MDA-MB-231 cells in the DOTAP:CHOL:DMPE-PEG:Elan219 LPDs is 5 mol % .

### EXAMPLE 12

Use of LPDs for Gene Delivery in vivo

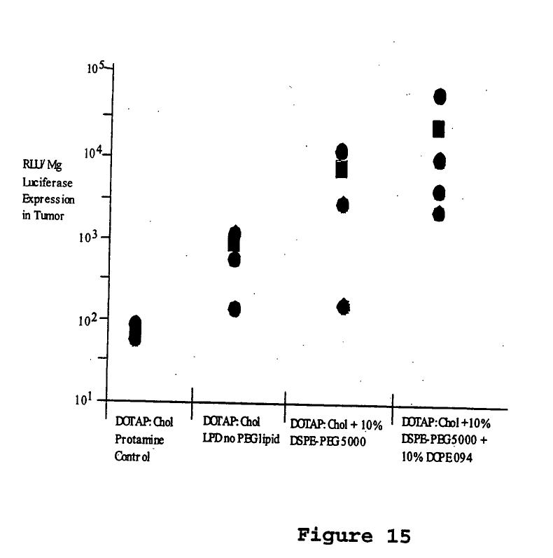

LPD formulations were made as described. Ba1bC nude mice were engrafted with 4×10⁶ MDA-MB-231 human breast cells 6 weeks prior to intratumoral injection of LPDs containing 50 ug of luciferase DNA. “DOTAP:CHOL:protamine control” is a lipid/protamine formulation without DNA. FIG. 15 shows tumour expression of the luciferase reporter gene 16 h following administration. The animals treated with the LPD formulation containing 10% Elan219 showed a higher level of luciferase expression in the tumor cells than the other formulations.

### EXAMPLE 13

Physical Properties Of Anionic DLPD

Anionic DLPD formulations were sized and the zeta-potential was measured in 5% dextrose USP. The mean particle size and population size distributions (represented by the polydispersity value) are shown in Table 9. Typically, the zeta-potential range from −15 to −50 mV for anionic DLPD composed either of DOPS:CHOL or DOPG:CHOL (55:45 lipid mol ratio) in 5% dextrose at either pH 4.5 or 7.5. For the pH sensitive formulation (CHEMS/DOPE) a zeta potential shift from −42.0 mVolt at pH 7.5 to +26.0 mVolt at pH 4.5 clearly indicated the pH sensitive effect. Control cationic LPDs were prepared at a 6 nmol:2 μg:1 μg lipid:protamine:DNA ratio. Data shown in Table 9 are from a single experiment and are representative of data observed for 5 additional experiments where these formulations were generated. SD for the zeta potential were calculated based on five readings from the same sample.

DSPE-PEG_{5K} addition did not increase particle size. The above formulations comprising 10 mol % targeting factor-pegylated lipid conjugate had large particle sizes. However, the particle size may be adjusted by adjusting the ratios of lipids as well as the ratios of total lipid to polycation to nucleic acid.

### EXAMPLE 14

Maximum DNA Concentration

A DNA dose titration was performed in order to determine the maximal DNA concentration achievable within a DOPS:CHOL or DOPG:CHOL anionic formulation with or without 10 mol % DPSE-PEGSK₅. Results shown in Table 10 show that it is possible to generate a DOPS:CHOL DLPD up to a DNA concentration of about 125 μg DNA/ml and at least up to 150 μg DNA/ml with 10 mol % DPSE-PEG_{5K}. DOPG/CHOL formulations demonstrated a DNA concentration of at least about 150 μg DNA/ml, and about 125 μg DNA/ml with 10 mol % DPSE-PEG_{5K}. Further experiments presented infra for the anionic complexes herein have been prepared using DLPDs prepared at 75 μg DNA/ml.

### EXAMPLE15

Anionic DLPD Transfection Activity

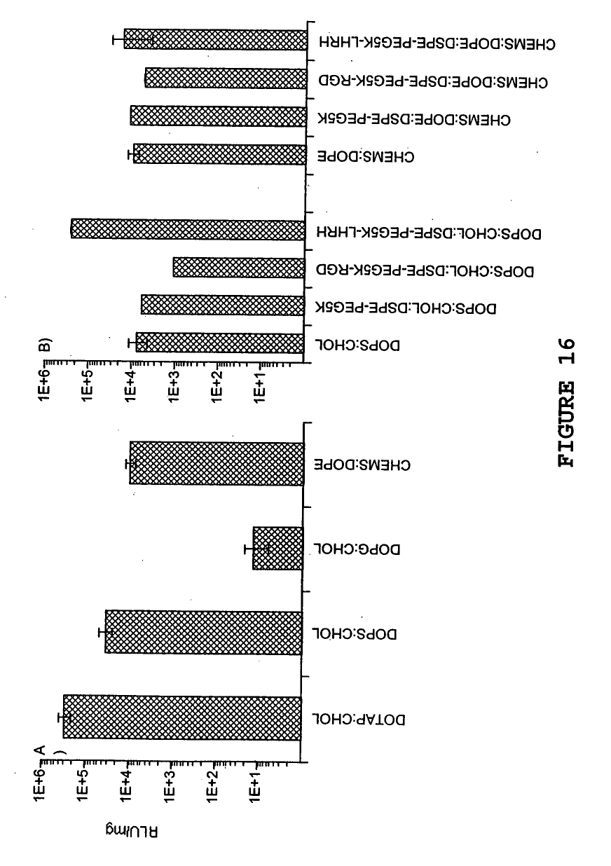

As shown in FIG. 16, anionic DLPDs and targeted anionic DLPDs are transfection competent in MDA-MB-231 cells at a level comparable to conventional cationic LPDs comprising DOTAP:CHOL as lipid.

The use of DOPS or CHEMS as the anionic lipids generated formulations with greater transfection activity compared to DOPG (FIG. 16A). Addition of DSPE-PEG_{5K}-LHRH (SEQ ID NO:11) to DOPS:Chol DLPDs demonstrated more than 1 log transfection enhancement in MDA-MB-231 cells over base formulations with or without DSPE-PEG_{5K}. For CHEMS formulation only a slight transfection enhancement was observed in presence of DSPE-PEG_{5K}-LHRH (SEQ ID NO:11) (FIG. 16B). However, no transfection enhancement with DSPE-PEG_{5K}-RGD (SEQ ID NO:10) ligand was observed either in DOPS or CHEMS formulations, although the formulations still showed high transfection levels.

Cell binding to DiI-labeled DLPDs were investigated by FACS analysis (Table 11). Data shown in Table 11 are from a single experiment. Anionic DLPD were shown to have lower cell binding compared to DOTAP:CHOL LPDs although equivalent levels of transfection activity were demonstrated. These data indicated that anionic DLPD are more potent in term of transfection capacity compared to cationic LPD.

### EXAMPLE 16

Effect of Serum Addition to Anionic DLPD on Particle Size

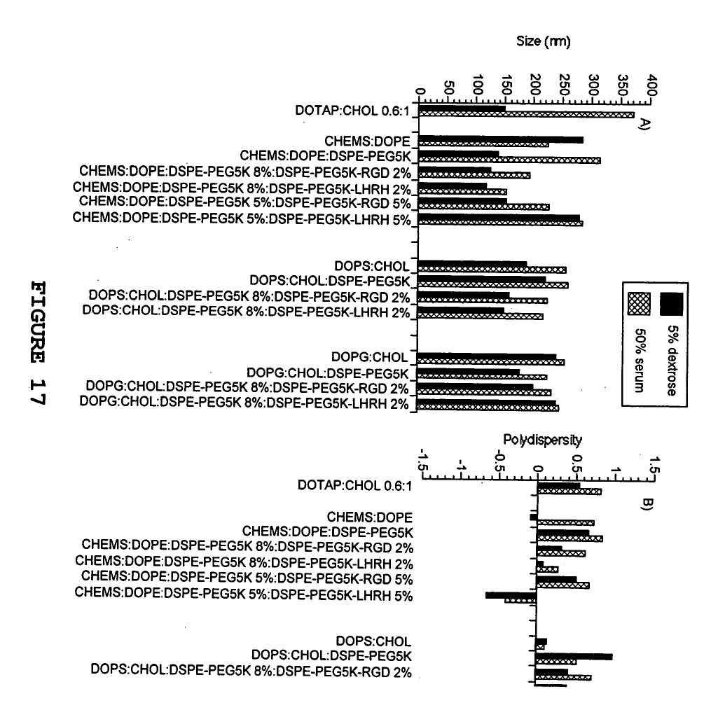

As a model for DLPD stability in vivo, DLPDs were pre-incubated for 1 h at 37° C. in mouse serum prior to performing transfection in serum free media. Anionic DLPD were not as affected by serum in terms of particle size increase compared to DOTAP:CHOL cationic LPDs. 5 mol % targeting factor-pegylated lipid conjugate addition to DOPS:CHOL or DOPG:CHOL DLPD resulted in particle size increase, with or without serum incubation (not shown). However, by using 2 mol % ligand and 8% of free extra DSPE-PEG_{5K} we were able to avoid serum mediated DLPD size increase (FIG. 17). The formulations shown had particle sizes within the accepted range.

### EXAMPLE 17

Effect of Serum Addition on DLPD Transfection Activitv

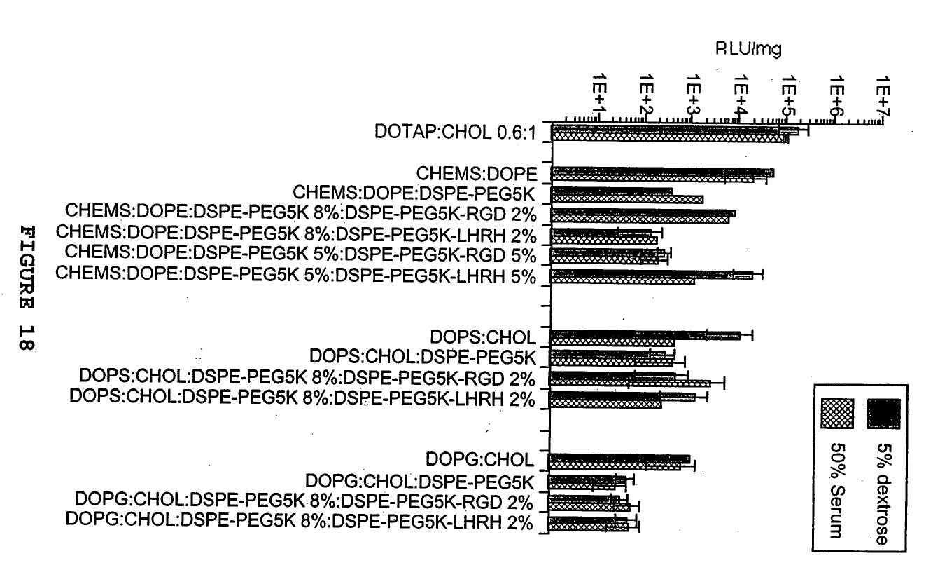

To access serum effects on DLPD in vivo transfection activity, DLPDs were pre-incubated for 1 h at 37° C. in mouse serum prior to performing transfections in serum free media. As observed for cationic LPDs, serum incubation reduces transfection activity, although, in this particular experiment, the decrease in transfection activity after serum incubation was smaller than usually observed (FIG. 18). The DOPS:CHOL formulation was shown to be more susceptible to serum-mediated transfection decrease compared to the other anionic formulations evaluated. This effect did not seem to be related to a change in DLPD particles size. Moreover, as expected, DSPE-PEG_{5K} addition to DLPD formulations at 10 mol % resulted in a 1 to 2 log decrease of transfection activity, which is comparable to that observed with DOTAP:CHOL:DSPE-PEG_{5K}. Interestingly, 5% mol DSPE-PEG_{5K}-LHRH (SEQ ID NO:11) addition to DLPD formulations even in presence of 5% extra free DSPE-PEG_{5K} enhanced transfection level by 2 log, an effect comparable to the one observed supra for targeted cationic LPD (data for DOPG:CHOL and DOPS:CHOL not shown).

### EXAMPLE 18

Effect of Serum Addition on Particle Size and Transfection Activitv in Additional Anionic Formulations

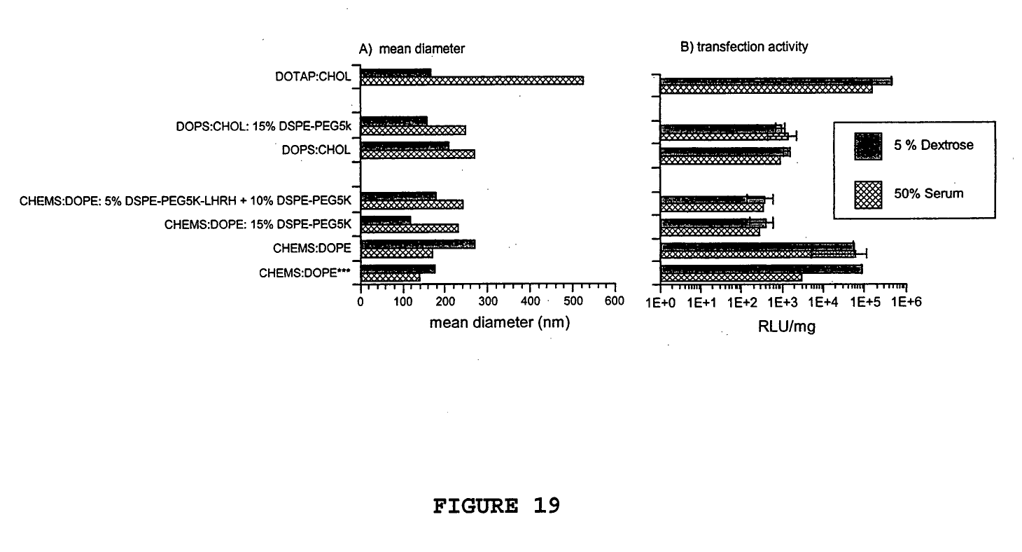

The effect of serum addition on LPD size and transfection activity in MBA-MD-231 cells is shown in FIG. 11A and 11B. Anionic DLPD formulations were less susceptible to serum mediated particle aggregation compared to cationic LPDs. For the DOPS:CHOL formulation, addition of 10% extra pegylated lipid was not able to prevent the serum mediated DLPD size increase associated with 5% DSPE-PEG_{5K}-LHRH (SEQ ID NO:11) presence (not shown). Anionic DLPD transfection activity following serum incubation seem to be less susceptible to serum. However, for pH sensitive formulations, although protection against size increase was observed, the transfection activity was less than that for the CHEMS:DOPE formulation. Two different salts of CHEMS were tested in this experiment as described supra, and both demonstrated the same transfection activity in dextrose (CHEMS:DOPE*** indicates morpholine salt). However, the morpholine salt appeared more sensitive to the serum effect as transfection decreased following serum incubation (FIG. 19B).

### EXAMPLE 19

Anionic Transfection in CHO-K1 Cells

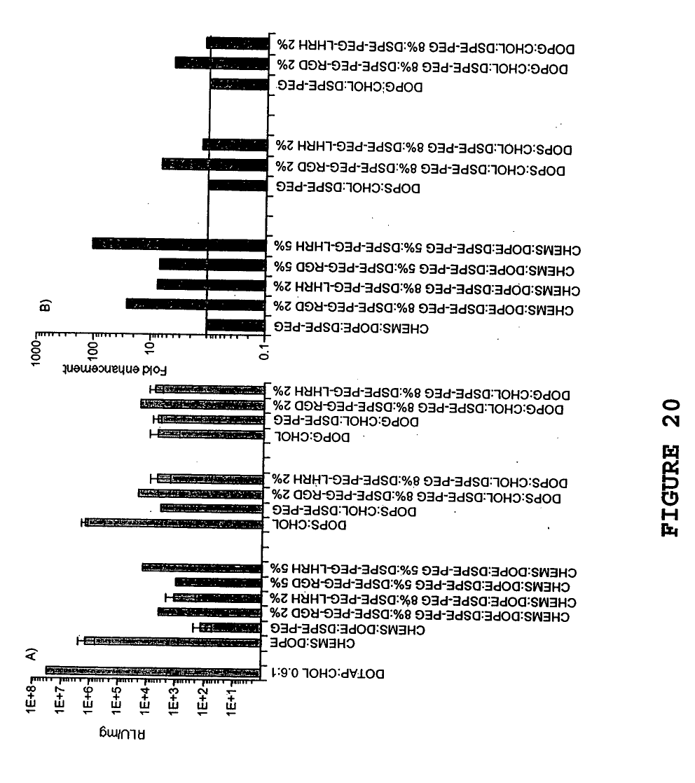

Transfection experiments were performed as above in CHO-K1 cells. As observed in FIGS. 20A and 20B, similar to results shown supra for MDA-MB-231 cells, DSPE-PEG_{5K} addition at 10 mol % to anionic CHEMS:DOPE and DOPS:CHOL DLPD formulations resulted in a 2 log decrease in transfection activity. This effect is comparable to that observed with DOTAP:CHOL:DSPE-PEG_{5K}. However, 5% mol DSPE-PEG_{5K}-LHRH (SEQ ID NO:11) addition to DLPD formulations even in the presence of 5% extra free DSPE-PEG_{5K} enhanced transfection level by 2 log for all formulations (DOPS:CHOL and DOPG:CHOL data not shown). This effect is comparable to that observed supra with cationic LPDs. However, all formulations shown demonstrate transfection activity in CHO-K1 cells.

### EXAMPLE 20

In vivo Transfection of Tumors by DNA/Lipid/Protarnine/Targeting Factor Complexes

Nude mice between 6 and 12 weeks of age are inoculated intraperitoneally with 2×10⁶ SKOV3-IP1 human ovarian carcinoma cells in a total injection volume of 0.5 mls of PBS. After 6-7 weeks of tumor cell engraftment, animals are injected with different formulations of pCMV-luc plasmid DNA and DNA/lipid/protamine/targeting factor in a total volume of 1.0 ml (5% Dextrose final, isotonic solution). Animals are sacrificed 16 hours post formulation injection. Tumor nodules are removed and lysed. Luciferase protein concentrations are determined according to the Luciferase Assay described above.

Alternatively, the animals are injected with LPD formulations comprising a gene with therapeutic utility, such as a plasmid containing the E1A gene (Althea, San Diego, Calif.), and tumor size and animal survival rates are monitored and compared with control animals to determine the therapeutic effectiveness of the lipid complex.

### EXAMPLE 21

In vitro Characterization of Additional Anionic Dialysed DNA/Lipid/Protamine Complexes

Anionic Dialysed DNA/Lipid/Protamine Complexes (DLPDs) for the formulations as listed in Table 12 were prepared and analyzed as described above according to the formulas described below.

Mixed micelles composed of anionic lipid (DOPS or DOPG) and cholesterol in a 55:45 molar ratio or CHEMS:DOPE at 7:3 molar ratio (pH sensitive formulation) were prepared in 200 mM N-Octyl-B-D-glucopyranoside (OGP). Following lipid solubilization in OGP, protamine:DNA complexes were added to mixed micelle solutions and dialyzed for 48 h against Milli-Q water with 3 solution changes. In the final solution change water was replaced by 20 mM HEPES pH 7.2.

The physical properties of anionic DLPDs were evaluated as described above, with formulations sized and zeta-potential measured in 5% dextrose USP. The mean particle size and population size distributions (represented by the polydispersity value) are shown in Table 12. Typically, zeta-potential ranges from −15 to −50 mV for anionic DLPD composed either of DOPS:CHOL or DOPG:CHOL (55:45 lipid mol ratio) either in 5% dextrose at pH 4.5 or 7.5. Interestingly, for the pH sensitive formulation a zeta potential shift from −42.0 mVolt at pH 7.5 to +26.0 mVolt at pH 4.5 clearly indicated the pH sensitive effect. Data shown in Table 12 are from a single experiment and are representative of data observed for 5 additional experiments where these formulations were generated. SD for the zeta potential were calculated based on five readings from the same sample.

To determine the maximum achievable concentration of DNA, a DNA dose titration was performed for DOPS:CHOL or DOPG:CHOL anionic formulations with or without 10 mol % DPSE-PEG_{5K}. Results are shown in Table 13 and show that it is possible to generate DOPS:CHOL DLPD up to a DNA concentration of 125 μg DNA/ml and up to 150 μg DNA/ml with the PEG formulation

FACS analysis representing the percentage of cell binding for anionic DLPD Di-I labeled after 1 h incubation with MDA-MB-231 cells are shown in Table 14.

### EXAMPLE 22

In vitro Transfection of Anionic Dialysed DNA/Lipid/Protamine Complexes

Anionic DLPD were prepared as discussed above. 5×10⁴ MDA-MB-231 cells were plated 24 hours prior to transfection with formulations delivering 1 μgDNA/well in a 48 well plate. Cells were transfected for 4 hrs. in serum free media and harvested 48 hours post transfection for luciferase expression. N=6 per formulation group. DOTAP formulation was generated using the general formula: 12 nanomoles lipid (DOTAP:CHOL); 2 μg Protamine:1 μg DNA. For the anionic DLPD 53 nanomoles lipid 2 μg Protamine: 1 μg DNA for anionic DLPD, X represent DSPE-PEG_{5K} and DSPE-PEG_{5K}-RGD/LHRH incorporated at 10 mole % were used.

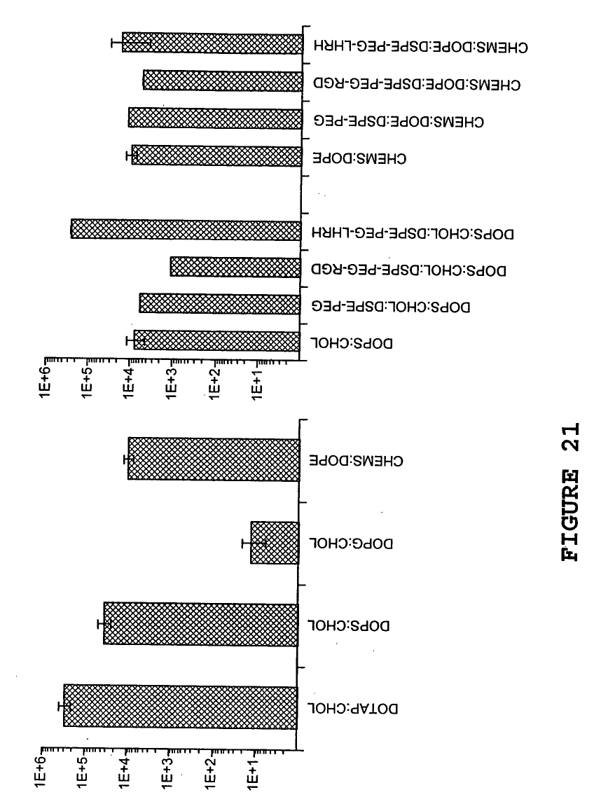

As shown in FIG. 21, anionic DLPD and targeted anionic DLPD were transfection competent in MDA-MB-231 cells at a level comparable to conventional cationic LPD (DOTAP formulation). Bars represent RLU/mg luciferase expression following MBA-MD-231 cells transfection with anionic DLPD. Moreover, the use of DOPS or CHEMS as the anionic lipids generated formulations with greater transfection activity compared to DOPG. Addition of DSPE-PEG_{5K}-LHRH (SEQ ID NO:11) to DOPS:Chol DLPDs demonstrated more than 1 log transfection enhancement in MDA-MB-231 cells over base formulations with or without DSPE-PEG_{5K}. However, no transfection enhancement with DSPE-PEG_{5K}-RGD (SEQ ID NO:10) ligand was observed either in DOPS or CHEMS formulations. For CHEMS formulation only a slight transfection enhancement was observed in presence of DSPE-PEG_{5K}-LHRH (SEQ ID NO:11).

### EXAMPLE 23

Effect of 50% Mouse Serum on Anionic Dialyzed DNA/Lipid/Protamine Complexes

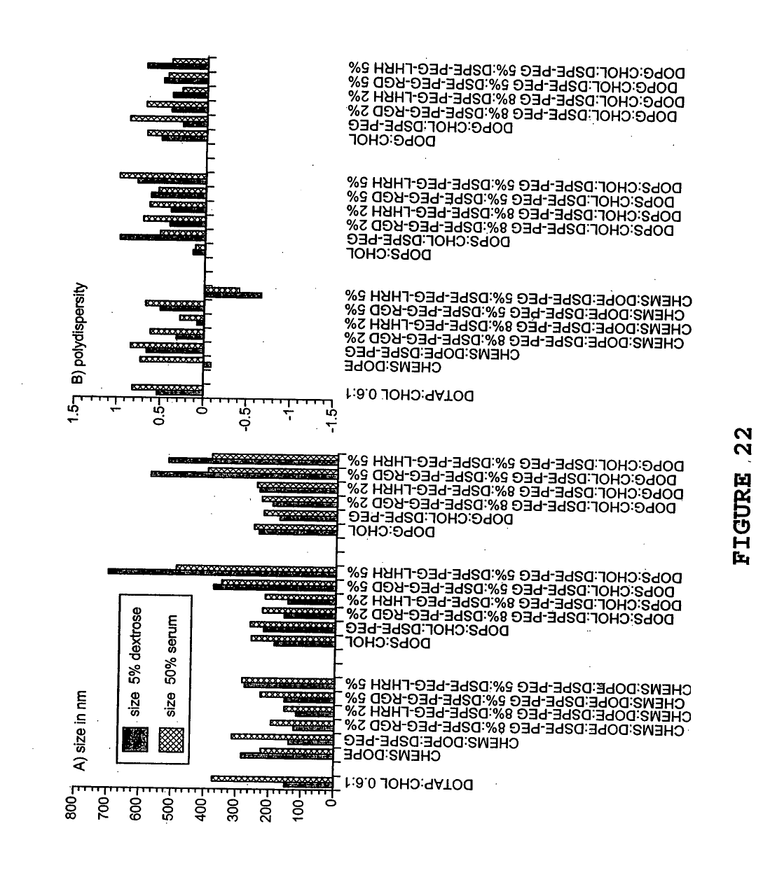

As a model for DLPD stability in vivo, DLPDs were pre-incubated for 1 h at 37° C. in 50% mouse serum prior to performing transfection in serum free media. Results are shown in FIG. 22, where bars represent the mean diameter in nm or the polydispersity for DLPD following DLPD incubation at 37° C. in 5% dextrose (solid bars) or in 50% serum (dash bars). Anionic DLPD were not affected by serum in terms of particle size increase compared to DOTAP:CHOL cationic LPDs which were susceptible to size increase after serum incubation. 5 mol % ligand addition to DOPS:CHOL or DOPG:CHOL DLPD resulted in particles size increase, with or without serum incubation. However, by using 2 mol % ligand and 8% of free extra PEG serum-mediated DLPD size increase was avoided.

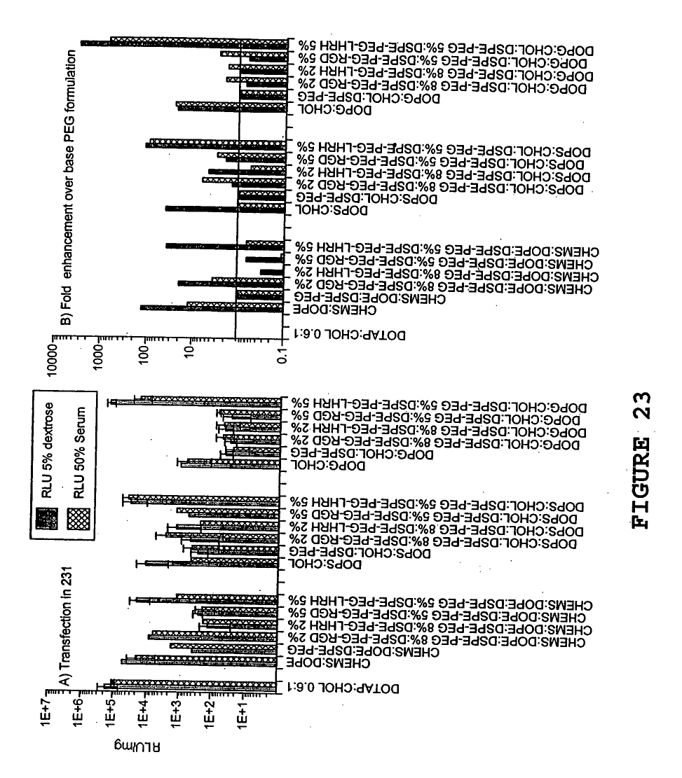

As a model for in vivo transfection activity, the transfection activity of DLPD following incubation in mouse serum was determined. DLPDs were pre-incubated for 1 hr at 37° C. in 50% mouse serum prior to performing transfections in serum free media and results are shown in FIG. 23. Bars represent RLU/mg luciferase expression (A) or fold enhancement over base PEG formulation (B) for DLPD following DLPD incubation at 37° C. in 5% dextrose (solid bars) or in 50% serum (dash bars). 5×10⁴ MDA-MB-231 cells were plated 24 hours prior to transfection with formulations delivering 1 μgDNA/well in a 48 well plate. Cells were transfected for 4 hrs. in serum free media and harvested 48 hours post transfection for luciferase expression. N=6 independent transfections per formulation group. DOPS:CHOL and DOPG:CHOL formulations were generated using the general formula: 53 nanomoles lipid (DOPS/G:CHOL:X);2 μg Protamine:1 μg DNA. pH sensitive formulations were generated using the general formula: 64.9 nanomoles lipid (CHEMS:DOPE:X);2 μg Protamine:1 μg DNA. DSPE-PEG_{5K}-RGD/LHRH were incorporated at 2 or 5 mole % and completed at 10 mol % using non conjugate DSPE-PEG_{5K}.

As observed for cationic LPDs, serum incubation reduces transfection activity, although, in this particular experiment decrease in transfection activity after serum incubation was smaller than usually observed. DOPS:CHOL were shown to be more susceptible to transfection decrease after serum incubation compared to other anionic formulations evaluated. This effect did not seem to be related to a change in DLPD particles size. DSPE-PEG_{5K} addition to DLPD formulations at 2 or 5 mol % results in a 2 log decrease of transfection activity comparable to previous observations with DOTAP:CHOL:DSPE-PEG_{5K}. 5% mol DSPE-PEG_{5K}-LHRH (SEQ ID NO:11) addition to DLPD formulations, even in presence of 5% extra free DSPE-PEG_{5K}, enhanced transfection level by 2 log, an effect comparable to the one observed for targeted cationic LPD.

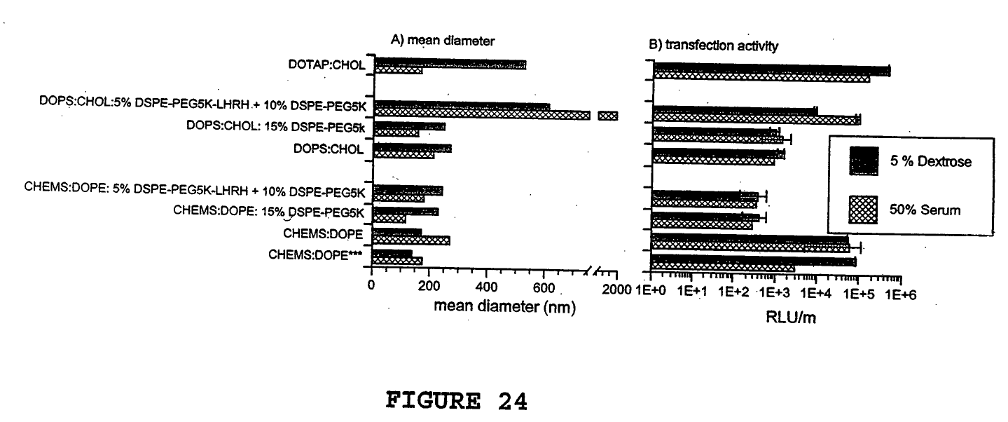

FIG. 24A represents the serum effect on LPD size and FIG. 24B on transfection activity in MBA-MD-231 cells. Bars represent particles mean diameter in A) or RLU/mg luciferase expression in B) following DLPD incubation at 37° C. in 5% dextrose (solid bars) or in 50% serum (dash bars).

5×10⁴ MDA-MB-231 cells were plated 24 hours prior to transfection with formulations delivering 1 μgDNA/well in a 48 well plate. Cells were transfected for 4 hrs. in serum free media and harvested 48 hours post transfection for luciferase expression. N=6 independent transfections per formulation group. DOPS:CHOL and DOPG:CHOL formulations were generated using the general formula: 53 nanomoles lipid (DOPS/G:CHOL:X);2 μg Protamine: 1 μg DNA. pH sensitive formulation were generated using the general formula: 64.9 nanomoles lipid (CHEMS:DOPE:X);2 μg Protamine:1 μg DNA. DSPE-PEG_{5K}-RGD/LHRH were incorporated at 5 mole % and 10 mol % of DSPE-PEG_{5K} were used. *** represents CHEMS morpholine salt.

As previously observed, anionic DLPD were less susceptible to serum mediated particle aggregation compared to cationic LPD. For DOPS:CHOL formulations an addition of 10% extra PEG was not able to prevent serum-mediated DLPD size increase associated with DSPE-PEG_{5K}-LHRH (SEQ ID NO:11) presence. Anionic DLPD transfection activity following serum incubation seems to be less susceptible to serum, contrary to results reported previously in FIG. 23. However, for pH sensitive formulations, although protection against size increase was observed, no targeting effect was generated. Two different salts of CHEMS were tested in this experiment, and both appear to have the same transfection activity in dextrose whereas that the morpholine salt appears more sensitive to serum effect as transfection decreased following serum incubation.

### EXAMPLE 24

Anionic DLPD Transfection Activity in CHO-K1 Cells

5×10⁴ MDA-MB-231 cells were plated 24 hours prior to transfection with formulations delivering 1 μgDNA/well in a 48 well plate. Cells were transfected for 4 hrs. in serum free media and harvested 48 hours post transfection for luciferase expression. N=6 independent transfections per formulation group. DOPS:CHOL and DOPG:CHOL formulations were generated using the general formula: 53 nanomoles lipid (DOPS/G:CHOL:X); 2 μg Protamine: 1 μg DNA. pH sensitive formulations were generated using the general formula: 64.9 nanomoles lipid (CHEMS:DOPE:X); 2 μg Protamine:1 μg DNA. DSPE-PEG_{5K}-RGD/LHRH were incorporated at 2 or 5 mole % and completed at 10 mol % using non-conjugated DSPE-PEG_{5K}.

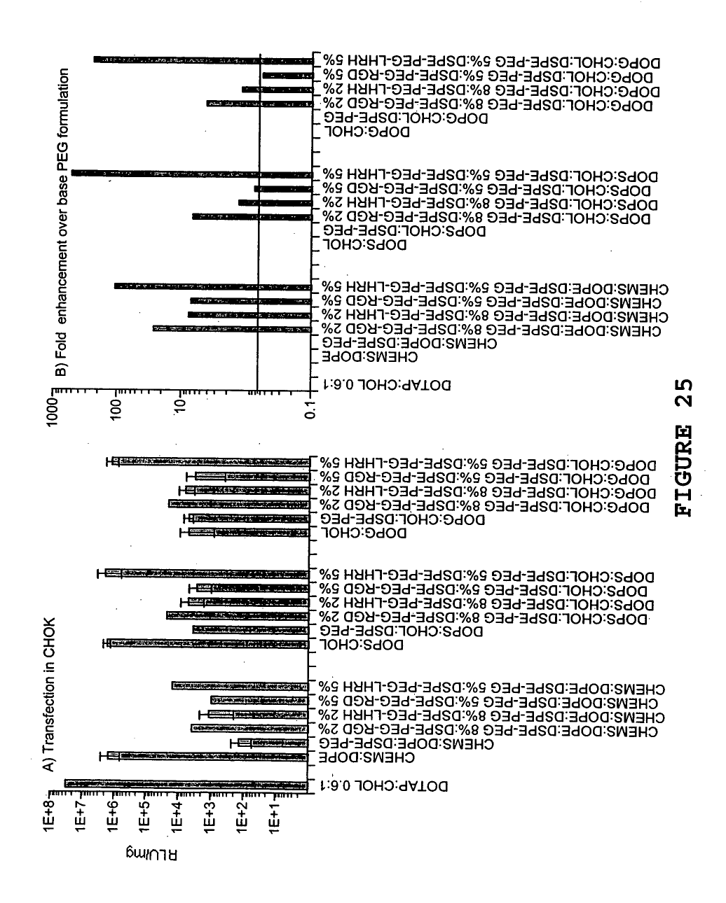

As observed in FIG. 23 and 24B) for MDA-MB-231, 2 or 5 mol % DSPE-PEG_{5K} addition to anionic DLPD formulation results in a 2 log decrease in transfection activity in CHOK1 cells, as shown in FIG. 25 (bars represent RLU/mg luciferase expression in A) or fold enhancement B) over base formulation for DLPD following DLPD incubation at 37° C. in 5% dextrose). This effect is comparable to previous observation with DOTAP:CHOL:DSPE-PEG_{5K}. However, 5% mol DSPE-PEG_{5K}-LHRH (SEQ ID NO:11) addition to DLPD formulations even in the presence of 5% extra free DSPE-PEG_{5K} enhanced transfection level by 2 log. This effect is comparable to previous observations with cationic LPDs. The DSPE-PEG_{5K}-LHRH-mediated transfection enhancement appears to only be present in DOPS or DOPG formulations, this effect was moderated for the pH sensitive formulations.

### EXAMPLE 25

Effect of Anionic DLPD DNA Concentration on Particle Size and Transfection Activity

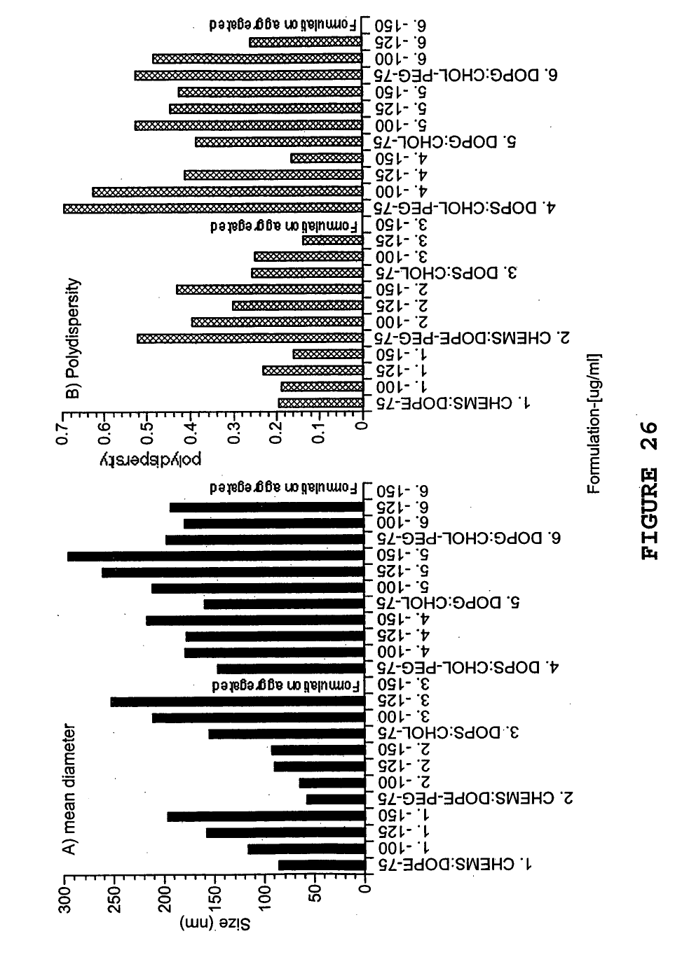

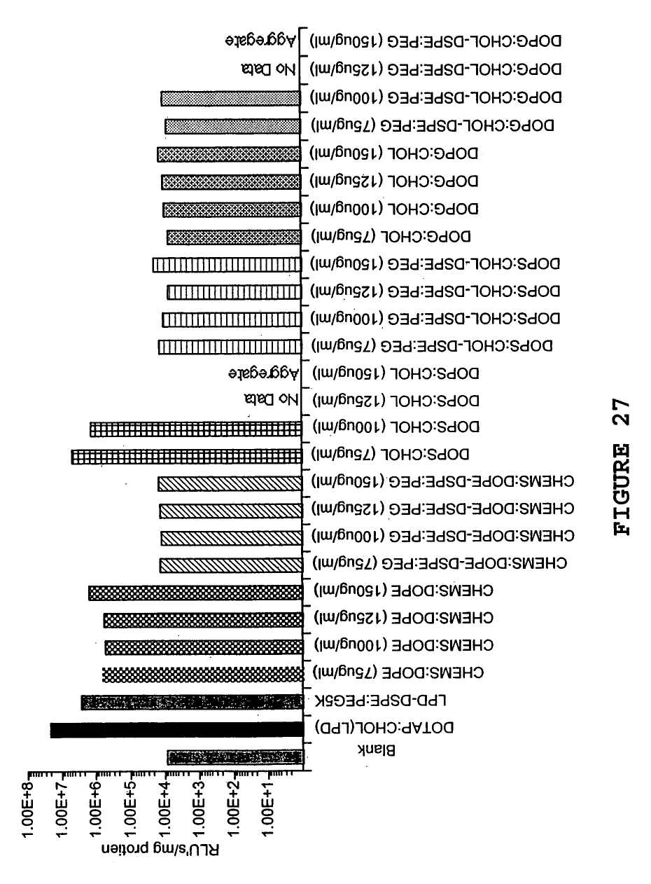

The maximum DNA concentration achievable in the anionic DLPDs prepared as described above was examined as described below. DLPDs were prepared at DNA concentrations ranging from 75, 100, 125, to 150 μg DNA/ml. The data is shown in Table 13 and FIG. 26, where solid bars represent the mean diameter in nm and dash bars represents polydispersity for anionic DLPDs. For CHEMS:DOPE formulations it was possible to prepare small particles (<200 nm) at concentrations up to and including 150 μg DNA/ml. Concentrations greater than 150 μg aggregated. However for DOPS:CHOL and DOPG:CHOL formulation aggregation was observed at 150 μg DNA/ml. DSPE-PEG_{5K} addition at 10 mol % in these formulation appears to reduce DLPD size. The use of 10 mol % DSPE-PEG_{5K} allowed the preparation of DOPS:CHOL and DOPG:CHOL formulations with mean particle diameter smaller than 200 nm at 125 μg/ml.

To determine if different DNA concentrations effect DLPD activity, in vitro transfection was realized in Skov3-ip1 cells. 5×10⁴ Skov3-ip1 cells were plated 24 hours prior to transfection with formulations delivering 1 μgDNA/well in a 48 well plate. Cells were transfected for 4 hrs. in serum free media and harvested 48 hours post transfection for luciferase expression. N=6 independent transfections per formulation group. DOPS:CHOL and DOPG:CHOL formulations were generated using the general formula: 53 nanomoles lipid (DOPS/G:CHOL:X);2 μg Protamine: 1 μg DNA. pH sensitive formulations were generated using the general formula: 64.9 nanomoles lipid (CHEMS:DOPE:X); 2 μg Protamine:1 μg DNA. DSPE-PEG_{5K} was incorporated at 10 mole in the formulation. No significant differences in transfection activity between formulations was observed (see FIG. 27, where bars represent RLU/mg luciferase expression for DLPD).

As expected, DSPE-PEG_{5K} addition to DLPD formulations 10 mol % results in a 2 log decrease of transfection activity comparable to previous observations compared to base formulation without DSPE-PEG_{5K}. DOPG formulations were not functional in term of transfection activity as observed previously in MDA-MB-231 cells.

As exemplified by Examples 21-25, anionic dialyzed lipid-protamine-DNA (DLPD) formulations were generated and were characterized in vitro and in vivo. DOPS (1,2-dioleoyl-sn-glycero-3-[phospho-L-serine]), DOPG (1,2-dioleoyl-sn-glycero-3-[phospho-rac-1-glycerol]) and CHEMS (cholesteryl hemisuccinate) were selected as anionic lipids to interact with positively charged protamine sulfate-DNA complexes prepared at a 2:1 (μg protamine:ug DNA) ratio. Mixed micelles composed of anionic lipid (DOPS or DOPG) and cholesterol in a 55:45 molar ratio or CHEMS:DOPE at 7:3 molar ratio (pH sensitive formulation) were prepared in 200 mM N-Octyl-B-D-glucopyranoside (OGP). Following lipid solubilization in OGP, protamine:DNA complexes were added to mixed micelle solutions and dialyzed for 48 h against Milli-Q water with 3 solution changes. In the final solution change water was replaced by 20 mM HEPES, pH 7.2. Mixed micelles appear to be a better lipid structure to start with in terms of preventing particle aggregation compared to the same lipid in a liposome configuration. A 6:1 charge ratio of anionic lipid to excess of protamine sulfate cationic charge from protamine:DNA 2:1 was demonstrated to be the most efficient in order to generate anionic DLPD composed of DOPS:CHOL or DOPG:CHOL. (i.e. for 1 μg DNA at a 2:1 μg protamine: μg DNA we have 3.03 nmol negative charge interacting with 8.2 nmol protamine sulfate. This resulting complex has 5.17 nmol excess of positive charge. To make anionic LPDs composed of DOPS:CHOL or DOPG:CHOL, a 6 fold excess of anionic charge from the anionic lipid is needed. As the anionic lipids used have 1 nmol positive charge per nmol lipid 31.02 nmol of DOPS or DOPG per ug DNA was needed. However, for CHEMS:DOPE formulations, a 4:1 charge ratio of anionic lipid to excess protamine sulfate was sufficient to generate anionic DLPDs (e.g., for 1 μg DNA used 20.68 nmol CHEMS at pH 7.2 was used). DPLD particle sizes typically ranged from 200-300 nm mean diameter and their zeta-potentials were from −35 to −50 mVolt.

Anionic DLPDs transfection characteristics are comparable to cationic LPDs in vitro in different cell lines (CHO-K1, MBA-MD-231), despite the observation, that DLPD cell binding was lower compared to cationic LPDs. Decreased binding of DLPDs to cells compared to LPDs was demonstrated by flow cytometry using DiI fluorescent-labeled DLPD. Addition of 5 mol % of lipid-conjugated ligands such as DSPE-PEG_{5K}-lutenizing hormone releasing hormone (DSPE-PEG_{5K}-LHRH (SEQ ID NO:11)) or DSPE-PEG_{5K}-RGD (SEQ ID NO:10) (an 11 amino acid peptide containing one RGD motif covalently attached to DSPE-PEG_{5K} lipid anchor) into anionic DLPD formulations generated particles with larger diameters (0.5 to 1 μm). In these formulations the lipid targeting ligands enhanced transfection activity by 2 log over base formulation. Subsequent addition of extra DSPE-PEG_{5K} without targeting ligands (up to 10 mol % total DSPE-PEG_{5K}) into DLPD formulations did reduce the particle size of the targeting ligand-containing DPLD formulations. However, addition of 10 mol % DSPE-PEG_{5K} into anionic DLPD formulations makes possible DNA concentrations of up to 125 ug DNA/ml. The mean diameter of such formulations is under 200 nm.

In Examples 21-25 generation of anionic DLPDs with the same transfection activity in vitro as cationic LPDs is clearly demonstrated. Anionic DLPDs were shown to have lower cell binding compared to DOTAP:CHOL LPDs, although equivalent levels of transfection activity were demonstrated. These data suggest that anionic DLPDs are more potent in terms of transfection capacity compared to cationic LPDs. Incorporation of DSPE-PEG_{5K}-LHRH (SEQ ID NO:11), in contrast to DSPE-PEG_{5K}-RGD (SEQ ID NO:10), into DLPDs resulted in an increase in transfection activity over DSPE-PEG DLPD formulations. However, addition of 10 mol % DSPE-PEG_{5K} promotes the preparation of prepared those formulation at 150 μg DNA/ml.

### EXAMPLE 26

In vitro Characterization of Anionic Dialysed DNA/Lipid/Polycation Complexes Incorporating NC₁₂-DOPE and DSPE-PEG_{5K}-Folate

Anionic dialyzed DNA/lipid/polycation complexes incorporating NC₁₂-DOPE (Shangguan et al. (1998) Biochim Biophys Acta 1368:171-83) and DSPE-PEG_{5K}-Folate were prepared using the techniques described above for anionic DLPDs and detailed as below, and formulated with the amounts of NC₁₂-DOPE and DSPE-PEG_{5K}-Folate as described below.

Typically, 2922 nmol of NC₁₂-DOPE (1,2-dioleoyl-sn-glycero-3-[phosphoethanolamine-N-dodecanoyl]) (Avanti Polar lipid Inc, Alabaster Ala., product # 790384 lot#181PE-N120-15) were mixed with 2890 nmol of DOPE (Avanti Polar lipid Inc, Alabaster Ala., product #850725) in chloroform (J. T. Baker, Phillipsburg N.J., cat# 9180). The lipid mixture in a borosilicate tube was dried for 1 hr under nitrogen at 4 LPM using a N-EVAP™ Organomation (Berlin, Mass.). The resulting lipid films which were formed were hydrated in 0.15 ml of 200 mM of N-Octyl-B-D-glucopyranoside (OGP, Sigma, St-Louis Mo., Cat no. 090001), achieving a final lipid concentration of 3.874 mM. The micellar solutions generated were sonicated for 30 sec using a sonicating bath (Laboratory supplies Co Inc., Hicksville, N.Y., serial #11463). pH sensitive micelles were prepared as described above using 1461.0 nmol cholesteryl hemisuccinate morpholine salt (CHEMS) (Sigma, St-Louis Mo., Cat no. C-5763) and 3409.0 nmol DOPE achieving a final lipid concentration of 3.246 mM.

For pegylated formulations, micelles were prepared as described in detail above and summarized briefly below. Typically, targeted DLPDs were prepared using 2922 nmol of NC₁₂-DOPE, 2893 nmol DOPE, and 22.2 nmol 1,2-disteraoyl-sn-glycero-3-phosphoethanolamine-N-[methoxy(polyethylene glycol)-5000] (DSPE-PEG_{5K}) (Avanti Polar lipid Inc, Alabaster Al, product #880220) or, using 22.2 nmol of DSPE-PEG_{5K}-Folate Lot No.10107601. DSPE-PEG_{5K}-Folate was synthesized by Northern Lipid Incorporated (Vancouver, BC). The lipid mixture was evaporated under nitrogen prior to lipid filmhydration with 0.15 ml lipids in 200 mM OGP and processed as described above.

To evaluate the possibility of generating NC₁₂-DOPE formulations with anionic DLPD a titration of lipid ratio (anionic lipid vs. cationic charge of compacted DNA) was performed in parallel to the effect of the helper lipid in the formulation. Charge ratios anionic lipid: protamine compacted-DNA 4:1, 6:1 and 8:1 were evaluated, where the formulations were composed of NC₁₂-DOPE:CHOL (1:1 mol ratio), NC₁₂-DOPE:DOPE (1:1 mol ratio) and NC₁₂-DOPE:DOPC (7:3 mol ratio). The formulations were characterized in regards to their mean particle size and population size distributions (represented by the polydispersity value). Data are shown in Table 15 where, NC₁₂-DOPE base DLPD formulations were prepared at 38.9:2:1, 58.9:2:1 and 77.9:2:1 ratio (nmol of anionic lipid: μg protamine: μg DNA). The final DNA concentration was 75 μg/ml. The numeric ratio following the formulation represents the charge ratio (ratio anionic lipid negative charge to the protamine compacted DNA positive charge). NC₁₂-DOPE:CHOL and NC₁₂-DOPE:DOPE were prepared at 1:1 lipid mol ratio. NC₁₂-DOPE:DOPC were prepared at 7:3 mol ratio. Typically, DLPD mean diameters were between 100-150 nm. Polydispersity values were typically under 0.5

### EXAMPLE 27

Transfection Activitv of Anionic DNA/Lipid/Polycation Complexes Containing NC₁₂-DOPE

The transfection activity of the NC₁₂-DOPE-containing LPDs in KB cells were determined as described above. 5×10⁴ cells were plated 24 hours prior to transfection with formulations delivering 1 μgDNA/well in a 48 well plate. Cells were transfected for 4 h in serum free media and harvested 48 hours post transfection for luciferase expression. N=6 per formulation group. NC₁₂-DOPE:X anionic DLPDs with ratios of nanomoles of anionic lipid: μg Protamine: μg DNA 38.9:2:1, 58.9:2:1 and 77.9:2:1 were prepared. In the lipid formula, X represent DOPC, DOPE or CHOL. For the CHEMS:DOPE formulation only 129 nanomoles of total lipid 2 μg Protamine:1 μg DNA was used. For 1 μg DNA at a ratio of 2:1 μg protamine: μg DNA 3.03 nmol of charge interact with 8.2 nmol protamine sulfate. This resulting complex has 5.1 7 nmol excess of positive charge prior to anionic lipid addition.

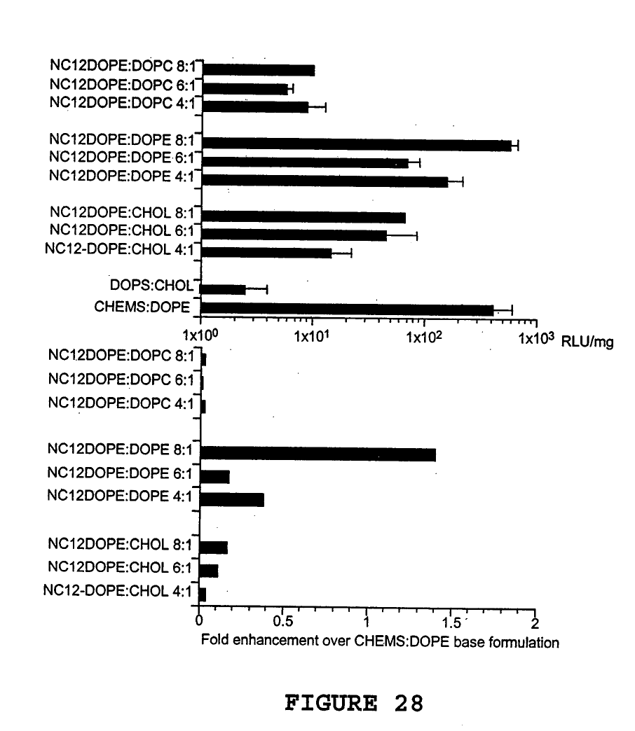

As shown in FIG. 28, anionic DLPDs were transfection competent in KB cells. Cholesterol and DOPE seem to be more effective helper lipids (co-lipids) than DOPC to use in combination with NC₁₂-DOPE.

Anionic DLPDs composed of NC₁₂-DOPE:X were evaluated at three different charge ratios, 4, 6 and 8-fold excess of anionic lipid negative charge to protamine-compacted DNA positive charge. As these anionic lipids have 1 nmol of negative charge per nmol lipid, 8 nmol of negative charge was needed to get optimal transfection, indicating that 77.92 nmol of NC₁₂-DOPE per μg of protamine-compacted DNA is optimal to make anionic DLPD with acceptable transfection activity.

### EXAMPLE 28

NC₁₂-DOPE-Containing Anionic DLPD Cell Toxicity

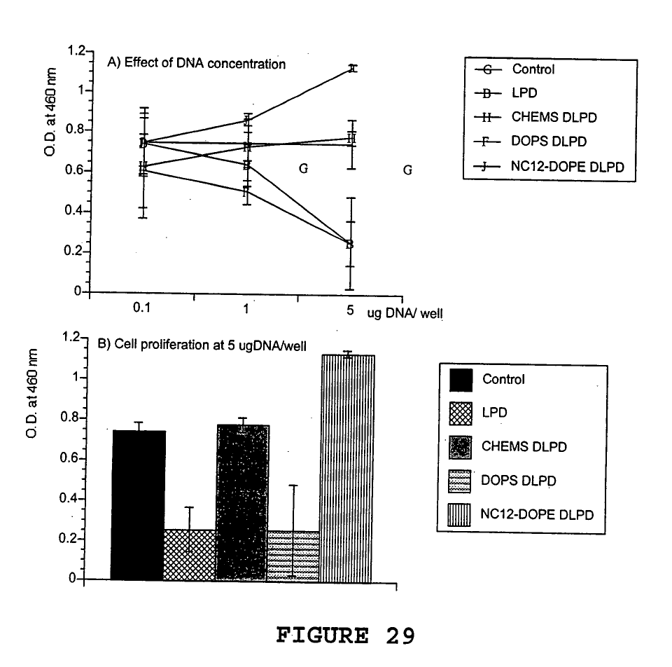

In vitro anionic LPD formulation cytotoxicity was determined using an MTS assay with cationic LPD or anionic DLPD at 3 different DNA doses per well. MTS assays are described in detail above in the methods section and summarized below. In FIG. 29, columns represent optical density (OD) read out at 490 nm. 5×10³ KB cells were plated 24 hours prior to incubation with formulations delivering 0.1 μg, 1 μg or 5 μg DNA/well in a 96 well plate. Cells were incubated with LPD or DLPD formulations for 4 h at 37° C. in serum free media. Following incubation, the cell culture media was removed and 100 μl of fresh cell culture media containing 20 μl of MTS reagent were added to cell and incubated for another 2 h at 37° C. prior to OD reading. vN=4 independent wells per formulation group.

Results show clear in vitro cell toxicity at a DNA concentration of 5 μg DNA/well for the DOTAP:CHOL cationic LPD formulation. The CHEMS:DOPE and NC₁₂-DOPE:DOPE formulations were not cytotoxic at all the DNA concentration evaluated. DOPS:CHOL has shown some level of cytotoxicity at 5 μg DNA/well.

### EXAMPLE 29

In vitro Titration of DSPE-PEG_{5K}-Folate in CHEMS:DOPE Formulation

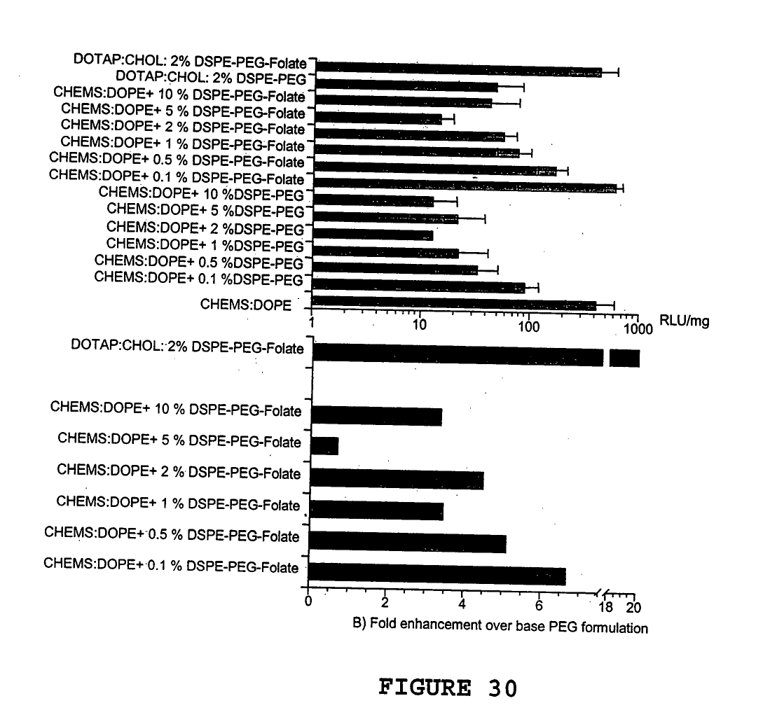

CHEMS:DOPE anionic DLPDs containing different concentrations of DSPE-PEG_{5K}-Folate were evaluated in terms of transfection activity in KB cells. This cell line was selected for its high level of folate receptor expression. A slight ligand dose-effect was observed from 0.1 to 10 mol % lipid-conjugated-ligand, with a maximum transfection enhancement observed with formulations containing 0.1 and 0.5mol % of DSPE-PEG_{5K}-Folate, as shown in FIG. 30.

In FIG. 30, columns represent RLU/mg luciferase expression (A) or fold transfection enhancement over base PEG formulation (B) in KB cells. 5×10⁴ cells were plated 24 hours prior to transfection with formulations delivering 0.1 μg DNA/well in a 48 well plate. Cells were transfected for 4 h in serum free media and harvested 48 hours post transfection for luciferase expression. N=6 independent transfection per formulation group. All formulations were generated using the general formula: 129 nanomoles total lipid (CHEMS:DOPE:X); 2 μg Protamine: 1 μg DNA. DSPE-PEG_{5K} and DSPE-PEG_{5K}-Folate were incorporated into formulations at concentrations ranging from 0.1 to 10 mole %.

### EXAMPLE 30

Preparation of Compacted DNA Using Different DNA Condensing Agents

The ability of a variety of DNA-condensing agents to compact DNA was investigated. For the data shown in Table 16, condensed DNA was prepared at 2:1 and 3.5:1 weight ratio of polycation:DNA and at a DNA concentration of 0.143 μg DNA/ml. PEI, Eudgragit® EPO, Eudragit®, E100, and PMOETMAB, were supplied by Elan Pharmaceuticals (Dublin, IR). RRRRRRRH (SEQ ID NO:44) and KHKHKHKHKGKHKHKHKHK (SEQ ID NO:45) peptides were synthesized by Research Genetics (ResGen, an Invitrogen Corporation, Huntsville, Ala.), while spermidine was purchased from Sigma (St. Louis, Mo.). All cationic polymers were solubilized in H₂O USP and the pH was adjusted to 5.5, a pH comparable to the protamine sulfate USP solution. Plasmid DNA was mixed with these cationic polymers at a 2:1 ratio using an Orion Sage™ (VWR, West Chester, Pa.) syringe pump mixing device as described above for protamine sulfate. PEI, and Eudragit® E100 were solubilized in H₂O USP and the solutions were acidified with HCl to increase solubility. The final pH of these polymer solutions was at pH 3.0. The other polymers evaluated had a pH comparable to protamine sulfate USP (pH ˜5.5). Polymer DNA complexes were immediately sized and mixed with anionic lipid to generate DLPD.

These formulations were compared to the protamine-compacted DNA base formulation. As shown in Table 16, all cationic polymers evaluated were able to compact DNA into particles with mean diameter ranging from 76 nm to 300 nm, either when used at 2:1 or at 3.5:1 μg μg ratio. The exceptions were the complexes made with spermidine, where at either charge ratio large particles formed, and PEI, where at a 3.5:1 μg:μg ratio large particle formation was observed following polymer addition to DNA solutions.

### EXAMPLE 31

Preparation of Anionic DLPDs with Varied DNA Condensing Agents

A variety of DNA condensing agents (cationic polymers/polysynthetic polycations) were evaluated for their ability to increase CHEMS:DOPE anionic DLPD transfection ability by replacing the protamine DNA complex by other cationic polymer-condensed DNA complex. Anionic DLPDs were generated as described above with mean diameter ranging from 100-300 nm. DNA expected for formulations containing PEI or Eudragit® E100 which formulation of which have shown aggregation immediately following lipid addition to the compacted DNA complex.

10 mol % DSPE-PEG_{5K} addition to the CHEMS:DOPE formulations avoided aggregation for PEI formulations at both charge ratios evaluated. The same effect was observed for Eudragit® E100-containing formulations prepared at the 3.5:1 charge ratio. 10 mol % DSPE-PEG_{5K} was ineffective to avoid aggregation of the spermidine-containing anionic lipid formulation prepared at a 3.5:1 charge ratio. The results are shown in Table 17.

### EXAMPLE 32

Anionic DLPD Preparation Using Different DNA Condensation Agent, Effect on Transfection Activity in SKOV3-ip1 Cells

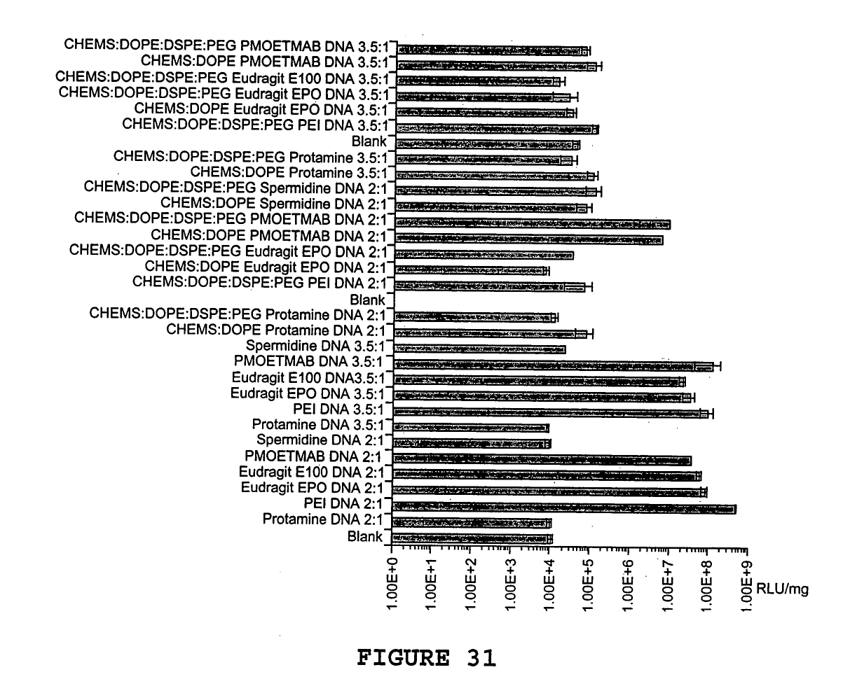

As shown in FIG. 31, most of the cationic polymers selected enhanced gene expression by 4-5 log over protamine-compacted DNA, except for the spermidine-compacted DNA complex. 5×10⁴ SKOV3-ip1 cells were plated 24 hours prior to transfection with formulations delivering 1 μgDNA/well in a 48 well plate. Cells were transfected for 4 h in serum free media and harvested 48 hours post transfection for luciferase expression. N=6 independent transfections per formulation group. CHEMS:DOPE pH sensitive formulations were generated using the general formula: 129 nanomoles total lipid 2 μg Protamine:1 μg DNA.

Addition of CHEMS:DOPE or CHEMS:DOPE:DSPE-PEG_{5K} lipid to the polymer-compacted DNA decreased gene expression by 3-4 log compared to polymer-only formulation. With the exception of the protamine-compacted DNA, where the addition of CHEMS :DOPE increased transfection by 3-fold over protamine-compacted DNA without lipid prepared at 2:1 charge ratio. Addition of 10 mol % DSPE-PEG_{5K} to CHEMS:DOPE protamine-comparted base formulation decreased the transfection activity by 3-fold.

The other polymers evaluated didn't show transfection enhancement over the protamine-compacted anionic formulation, except for PMOETMAB incorporated into CHEMS:DOPE formulations, with or without DSPE-PEG_{5K}. In this particular case a 3 log transfection increase over the CHEMS DOPE anionic formulation containing protamine-compacted DNA formulation was observed.

### EXAMPLE 33

Anionic DLPD Preparation Using Different DNA Condensation Agent, Effect on Transfection Activity in KB Cells

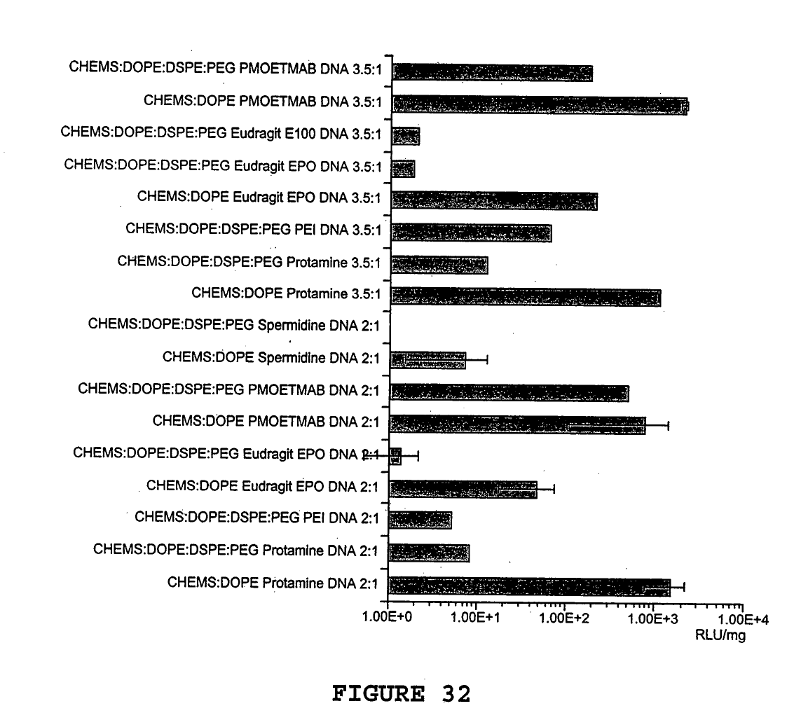

As shown in FIG. 32, addition of PMOETMAB to CHEMS:DOPE increased gene expression by 4 fold over the protamine DNA CHEMS:DOPE formulation when DNA was compacted at 3.5:1 μg:μg ratio. However, at a 2:1 charge ratio for all formulations containing cationic polymer-condensed DNA evaluated, no significant transfection enhancement over the protamine-compacted anionic DLPD formulations was observed.

Transfection was performed as described above and CHEMS:DOPE pH sensitive formulations were generated using the general formula: 129 nanomoles total lipid 2 μg Protamine: 1 μg DNA.

### EXAMPLE 34

Compaction of DNA With Different DNA Condensing Agents

In a second experiment the effectiveness of different DNA condensing agents was evaluated. Two cationic peptides, RRRRRRRH (SEQ ID NO:44) and KHKHKHKHKGKHKHKHKHK (SEQ ID NO:45), were evaluated. The peptide-compacted DNA particles were then compared to the protamine-compacted DNA particles prior to lipid addition.

PEI, Eudgragit® EPO, Eudragit® E100, PMOETMAB, RRRRRRRH (SEQ ID NO:44) and KHKHKHKHKGKHKHKHKHK (SEQ ID NO:45) peptide were solubilized in H₂O USP and pH was adjusted to 5.5, a pH comparable to the protamine sulfate USP. Plasmid DNA was mixed with these cationic polymers at a 2:1 ratio (μg;μg) at a DNA concentration of 0.143 mg/ml using an Orion Sage™ (VWR, West Chester, Pa.) syringe pump mixing device as described above for protamine sulfate. Polymer-DNA complexes were immediately sized and mixed with anionic lipid.

As shown in Table 18, all cationic polymers or DNA condensing agents evaluated were able to compact DNA into particles ranging from 25 nm to 121 nm mean diameter when used- at a 2:1 μg μg ratio.

The KHKHKHKGKHKHKHKHK (SEQ ID NO:45) peptide compacted DNA into very small particles with a mean diameter of 25 nm.

### EXAMPLE 35

NC₁₂-DOPE-Containing Lipid DNA Complex with Various Condensing Agents

NC₁₂-DOPE and CHEMS-based anionic DLPDs formulated with different DNA condensation agents were formulated as shown in Table 19. Formulations containing 0.5 mol % of DSPE-PEG_{5K} or DSPE-PEG_{5K}-Folate were prepared. Particle size characterization and population size distributions (represented by the polydispersity value) data are shown in Table 19. Typically, DLPD mean diameter was between 100-300 nm with most of the formulations showing polydispersity values under 0.5. All formulations generated show a negative zeta potential value ranging from −15 to −50 mVolt.

### EXAMPLE 36

Evaluation of Various Polvmer-Condensed DNA Complexes on Transfection Activity

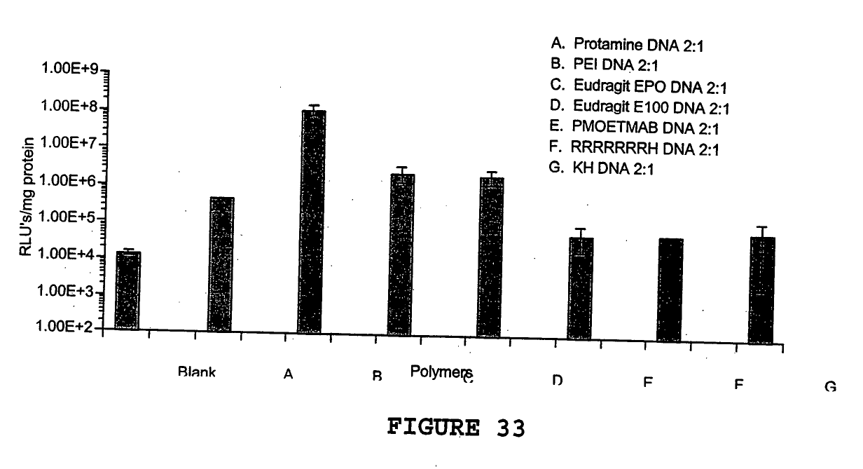

FIG. 33 shows the transfection activity in KB cells of different formulations of compacted DNA without lipid addition to the compacted DNA. In FIG. 33 bars represent RLU/mg luciferase expression following KB cells transfections. 5×10⁴ cells were plated 24 hours prior to transfection with polymer-compacted DNA formulations delivering 1 μg DNA/well in a 48 well plate. Cells were transfected for 4 h in serum free media and harvested 48 hours post transfection for luciferase expression. N=6 independent transfections per formulation group. KH represents the KHKHKHKHKGKHKHKHKHK (SEQ ID NO:45) peptide.

DNA compacted with PEI shows a 2 log transfection enhancement over the protamine sulfate compacted DNA. Eudragit® EPO or E100 compacted DNA shows a 1 log transfection enhancement over protamine sulfate compacted DNA. Others polymers evaluated didn't show transfection enhancement over protamine-compacted DNA. All polymer-DNA complexes were prepared with a 2:1 μg ;μg ratio.

### EXAMPLE 37

Anionic DLPD Preparation Using Different DNA Condensation Agents, Effect on Transfection Activity with CHEMS:DOPE Formulations

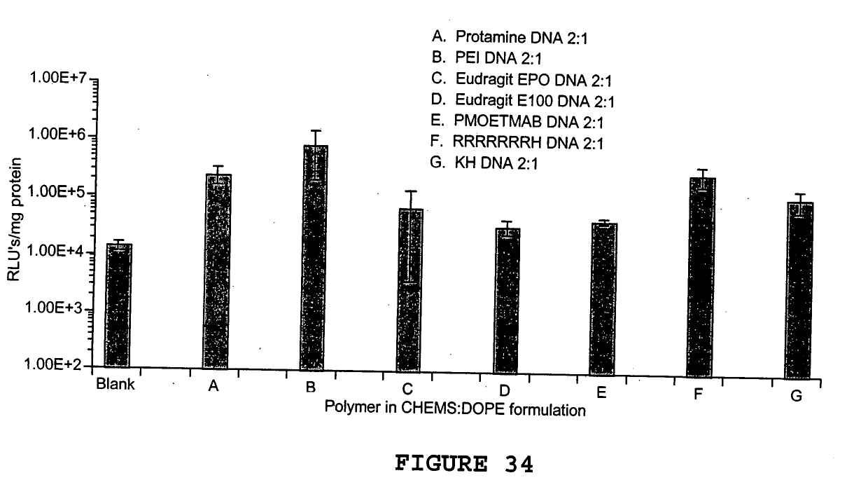

As shown in FIG. 34, addition of CHEMS:DOPE lipids to the polymer-compacted DNA decreases gene expression by 3-fold over the protamine-compacted DNA alone. In FIG. 34, bars represent RLU/mg luciferase expression following KB cell transfections.

5×10⁴ cells were plated 24 hours prior to transfection with formulations delivering 1 μgDNA/well in a 48 well plate. Cells were transfected for 4 h in serum free media and harvested 48 hours post transfection for luciferase expression. N=6 independent transfections per formulation group. CHEMS:DOPE pH sensitive formulations were generated using the general formula: 129 nanomoles lipid 2 μg cationic polymer: 1 μg DNA. KH represents the KHKHKHKHKGKHKHKHKHK (SEQ ID NO:45) peptide.

A 2 log decrease over the PEI-compacted DNA without lipid was observed compared to the PEI base anionic DLPD formulation. Eudragit® EPO, Eudragit® E100 and PMOETMAB base anionic DLPD formulations showed 1 log decreases over the corresponding polymer-compacted DNA alone without lipid addition. The use of the RRRRRRRH (SEQ ID NO:44) peptide showed a 2 fold-increase in transfection activity when formulated with CHEMS:DOPE compared to the peptide-compacted DNA alone. No change in the transfection activity was observed for the formulation containing the KHKHKHKHKGKHKHKHKHK (SEQ ID NO:45) peptide.

### EXAMPLE 38

Anionic DLPD Preparation Using Different DNA Condensation Agents, Effect on Transfection Activity with CHEMS:DOPE:DSPE-PEG_{5K} Formulations

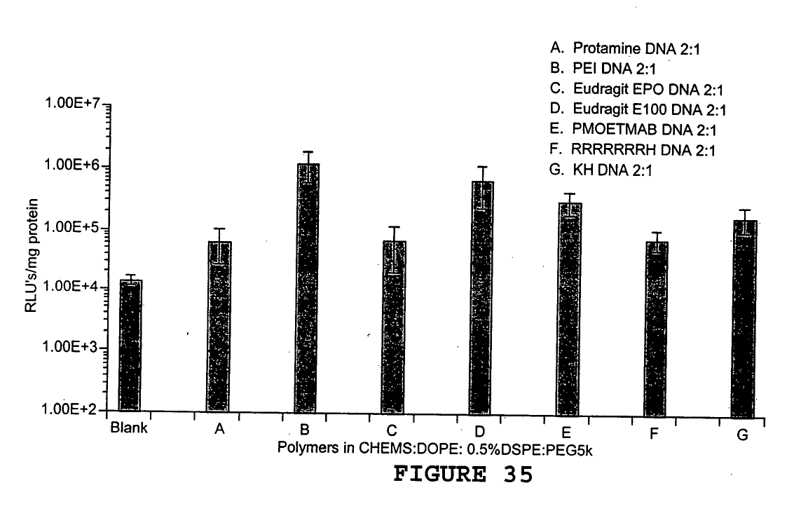

In FIG. 35, bars represent RLU/mg luciferase expression following KB cells transfections. 5×10⁴ KB cells were plated 24 hours prior to transfection with formulations delivering 1 μgDNA/well in a 48 well plate. Cells were transfected for 4 h in serum free media and harvested 48 hours post transfection for luciferase expression. N=6 independent transfections per formulation group.

CHEMS:DOPE: DSPE-PEG_{5K} pH sensitive formulations were generated using the general formula: 129 nanomoles lipid (CHEMS:DOPE:DSPE-PEG₅K); 2 μg cationic polymer:1 μg DNA. DSPE-PEG_{5K} was incorporated at 0.5 mol % into the DLPD formulation. KH represents the KHKHKHKHKGKHKHKHKHK (SEQ ID NO:45) peptide.

0.5 mol % pegylated lipid addition to CHEMS:DOPE anionic formulations containing protamine-compacted DNA resulted in a 5-fold decrease in transfection activity compared to the same formulations without PEG. However, when anionic DLPDs were formulated in presence of DSPE-PEG_{5K} and using PEI as a DNA-condensing agent the decrease in transfection activity was not observed. Eudragit® E100 or PMOETMAB were used to compact DNA, transfection enhancement for the PEG-bearing formulation over non-PEG-bearing formulation resulted in 1 log increase in transfection activity, approaching the absolute transfection level observed with PEI-containing anionic DLPDs.

### EXAMPLE 39

### Anionic DLPD Preparation Using Different DNA Condensation Agents, Effect on Transfection Activity with CHEMS:DOPE:DSPE-PEG_{5K}-Folate Formulations

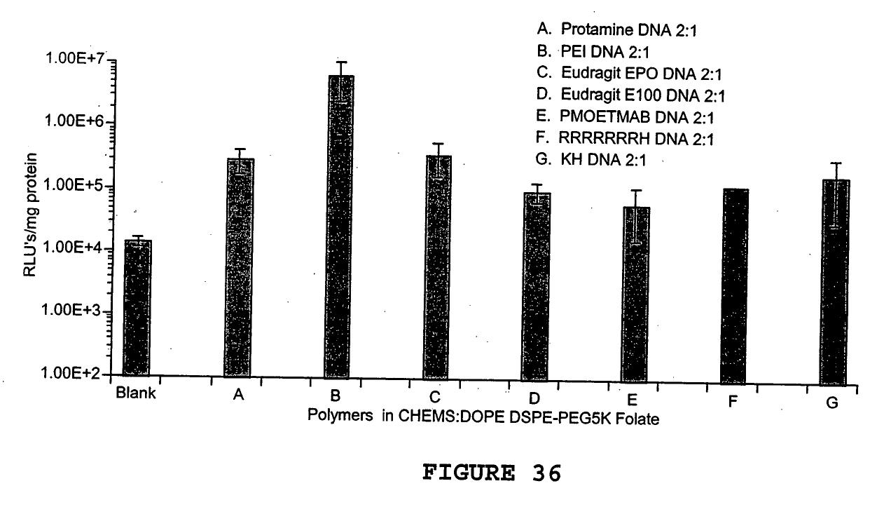

In FIG. 36, bars represent RLU/mg luciferase expression following KB cell transfections. Transfection of KB cells was as described in Example 38. CHEMS:DOPE: DSPE-PEG_{5K}-Folate pH sensitive formulations were generated using the general formula: 129 nanomoles lipid (CHEMS:DOPE:DSPE-PEG_{5K}-Folate);2 μg cationic polymer:1 μg DNA. DSPE-PEG_{5K}-Folate was incorporated at 0.5 mol % into the DLPD formulation. KH represents the KHKHKHKHKGKHKHKHKHK (SEQ ID NO:45) peptide.

0.5 mol % DSPE-PEG_{5K}-Folate lipid ligand addition into CHEMS:DOPE anionic formulations formulated with most of the polymer-compacted DNAs evaluated in this study, resulted in a 2 to 20 fold transfection enhancement over base formulations except for formulations containing DNA compacted with Eudragit® E100 or PMOETMAB. As previously observed in FIG. 34 and 35, when anionic DLPD were formulated using PEI as a DNA compaction agent transfection increased by 1 log over the same lipid formulation containing protamine-compacted DNA.

### EXAMPLE 40

### Anionic DLPD Preparation Using Different DNA Condensation Agent, Effect on Transfection Activity with NC₁₂-DOPE:DOPE Formulations

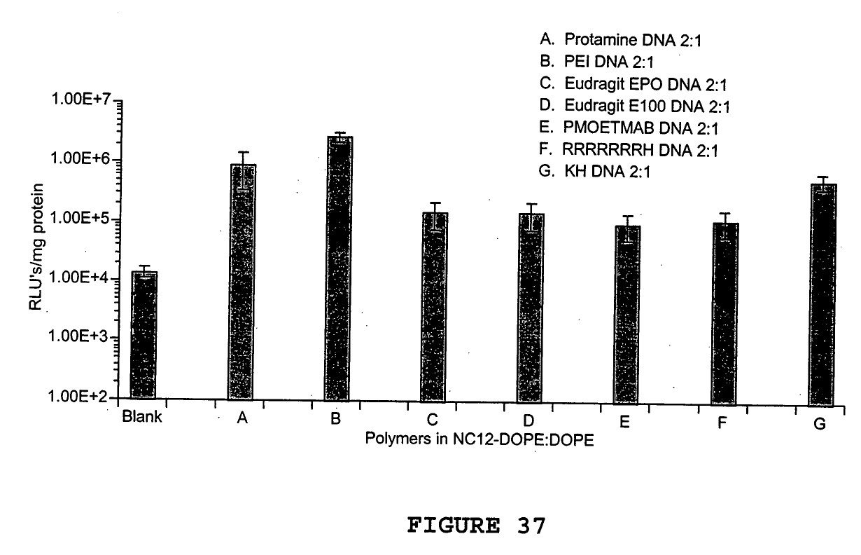

In FIG. 37, bars represent RLU/mg luciferase expression following KB cells transfections. KB cell transfection was performed as in the above Examples. NC₁₂-DOPE:DOPE formulations were generated using the general formula: 156 nanomoles lipid 2 μg cationic polymer:1 μg DNA. KH represents the KHKHKHKHKGKHKHKHKHK (SEQ ID NO:45) peptide.

As shown in FIG. 37, the use of NC₁₂-DOPE:DOPE anionic lipid formulation increase transfection activity with all the cationic polymers evaluated by 2 to 7 fold over the sane polymer condensed DNA formulated with CHEMS:DOPE anionic lipid, except for formulations containing Eudragit E100. As previously observed in FIGS. 33 to 36, PEI constantly gave the highest absolute transfection level.

### EXAMPLE 41

### Anionic DLPD Preparation Using Different DNA Condensation Agents, Effect on Transfection Activity with NC₁₂-DOPE:DOPE:DSPE-PEG_{5K} Formulations

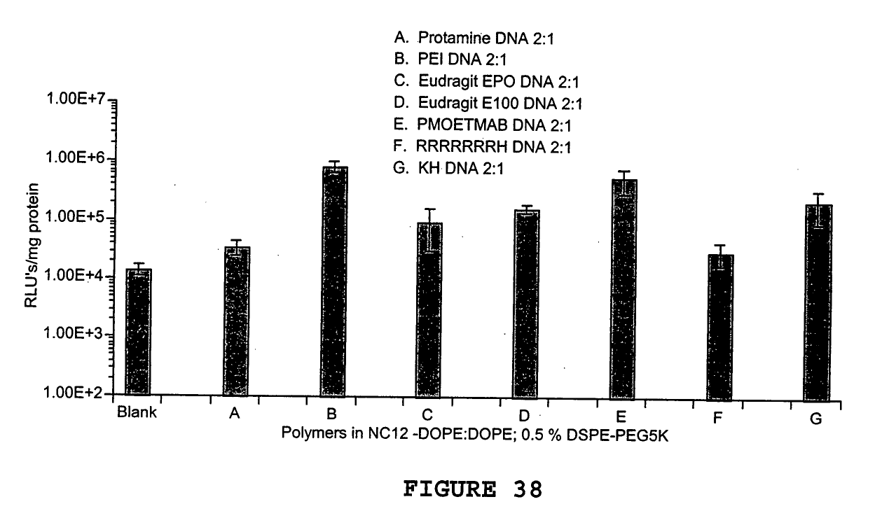

Transfection was performed as described above and results are presented in FIG. 38.

0.5 mol % pegylated lipid added to NC₁₂-DOPE:DOPE anionic formulations containing cationic polymer-compacted DNA results in a 2 to 12 fold decrease in transfection activity compared to the same formulation without DSPE-PEG_{5K} lipid addition. For formulations where DNA was compacted using PMOETMAB, where a 5-fold increase in transfection over the formulation without DSPE-PEG_{5K} lipid addition was observed. As previously observed in FIGS. 33-37, PEI constantly gave the highest absolute transfection level.

### EXAMPLE 42

Anionic DLPD Preparation Using Different DNA Condensation Agents, Effect on Transfection Activity with NC₁₂-DOPE:DOPE:DSPE-PEG_{5K}-Folate Formulation

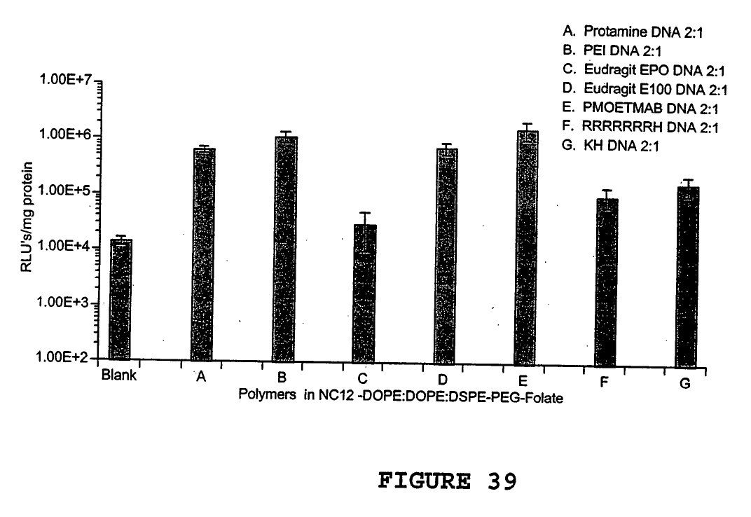

Transfection was performed as described above using the formulations prepared as described above. Results are shown in FIG. 39, in which bars represent RLU/mg luciferase expression following KB cell transfections.

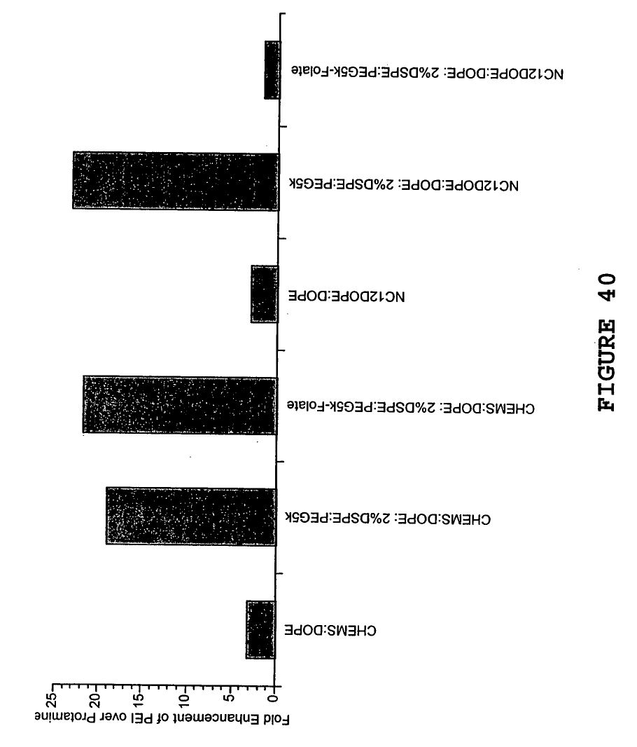

0.5 mol % DSPE-PEG_{5K}-Folate lipid ligand addition into NC₁₂-DOPE:DOPE anionic formulation formulated with different polycationic DNA compacting agents resulted in a 18 to 2-fold transfection enhancement over the corresponding base formulation containing DSPE-PEG_{5K}. Interestingly, the protamine-compacted DNA based formulation showed the highest folate effect (18 fold enhancement), although the PEI base formulation shows the highest absolute transfection level. Transfection activity data for PEI-containing formulations is shown in FIG. 40. On average PEI-containing formulations show 3 to 20-fold higher transfection levels compared to protamine-based anionic DLPD formulations. The effect was more pronounced for DSPE-PEG_{5K}-bearing formulations.

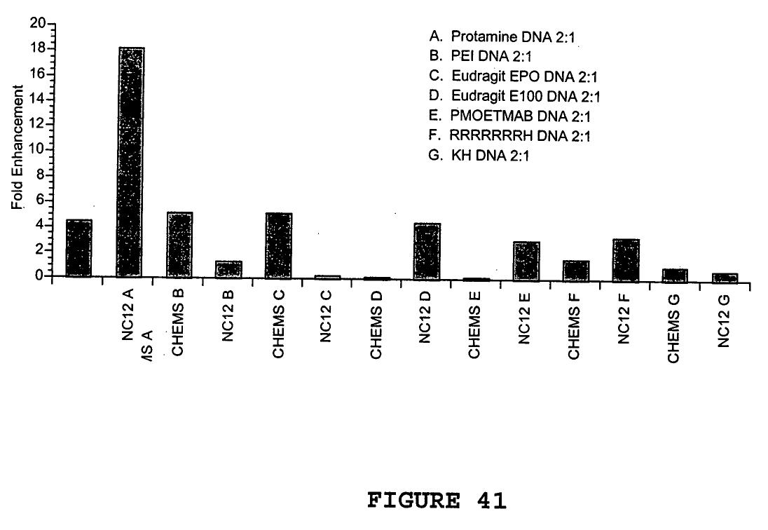

FIG. 41 illustrates the folate-mediated transfection enhancement in CHEMS:DOPE and NC₁₂-DOPE base formulations generated with different cationic polymer-condensed DNA. The incorporation of DSPE-PEG_{5K}-Folate into NC₁₂-DOPE:DOPE formulation shows 18-fold folate mediated transfection enhancement over when DNA was condensed with protamine sulfate.

### EXAMPLE 43

### Effect of pH on Anionic DLPD Zeta Potential

In order to evaluate the pH effect on anionic DLPD net charge, zeta potential measurements were obtained in 20 mM HEPES buffer at pH 7.2 and 4.2. As demonstrated in Table 20 CHEMS: DOPE anionic formulations showed a sensitivity to pH while formulations composed of NC₁₂-DOPE:DOPE did not show change in the zeta potential at pH 4.2.

DLDP mean diameter was measured using an unimodal mode at pH 7.2. CHEMS:DOPE pH sensitive formulations were generated using the general formula: 129 nanomoles lipid (CHEMS:DOPE)::2 μg cationic polymer: 1 μg DNA NC₁₂-DOPE:DOPE formulations were generated using the general formula: 156 nanomoles lipid (NC₁₂-DOPE:DOPE);2 μg cationic polymer:1 μg DNA. SD for the zeta potential have been calculated based on five readings from the same sample. n.d. equal not determined.

Examples 26-43 detail the incorporation of the anionic lipid N-dodecanoyl-DOPE (NC₁₂-DOPE) a fusogenic lipid into anionic dialyzed lipid-protamine-DNA (DLPD) formulations. The results were compared with the first generation of anionic DLPDs composed of CHEMS:DOPE. As previously described, anionic lipids NC₁₂-DOPE:DOPE in mixed micelle form interact with positively charged protamine sulfate-DNA complexes prepared at a 2:1 ratio (μg protamine:μg DNA) resulting in transfection-competent anionic lipid base particles.

NC₁₂-DOPE composed anionic particles have mean diameter values ranging from 100-300 nm and zeta-potentials ranging from −35 to −50 mVolt. NC₁₂-DOPE anionic DLPD transfection characteristics in KB cells were comparable to the CHEMS base formulation.

As determined in a MTS assay, anionic DLPDs appeared to be less cytotoxic in vitro compared to DOTAP:CHOL cationic LPDs, except for the anionic formulation composed of DOPS:CHOL.

Addition of 0.5 mol % of lipid-conjugated-ligands such DSPE-PEG_{5K}-Folate into anionic DLPD formulations generated particles of acceptable size 100-250 nm. More interestingly, these lipid conjugated targeting factors enhanced transfection activity by 4-6 fold over base formulations.

The incorporation into anionic DLPDs of different cationic polymers which are transfection competent by themselves were also investigated and compared to the protamine-compacted DNA base formulation. These new complexes of cationic polymers/DNA were incorporated into the NC₁₂-DOPE and the CHEMS:DOPE formulations prior to transfection evaluation in KB cells. PEI, as the DNA compacting agent added to anionic lipid generated anionic DLPD formulations showing higher transfection activity compared to protamine sulfate base formulations. PMOETAB-compacted DNA was also able to enhance anionic DLPD transfection. Eudragit® EPO and E100 incorporated into the anionic lipid formulations had lower transfection activity in KB cells compared to protamine sulfate-containing formulations.

In these examples, Examples 26-43, it has been clearly demonstrated that anionic DLPDs could be generated using NC₁₂-DOPE. The new DLPD formulations show comparable or better level of transfection than CHEMS:DOPE anionic DLPD formulations.

DSPE-PEG_{5K}-Folate is compatible with all NC₁₂-DOPE formulations tested and shows up to 18 fold transfection enhancement. 0.1 to 0.5 mol % DSPE-PEG_{5K}-Folate seems to be the optimal concentration of DSPE-PEG_{5K}-Folate for use in anionic DLPD formulations.

PEI containing anionic DLPDs seem to give higher transfection levels than protamine sulfate containing DLPDs.

### EXAMPLE 44

Incorporation of poly(propyl acrylic acid) PPAA into Cationic DNA/Lipid/Protamine Complexes

Liposome preparation: 6000 nmol of 1,2-dioleoyl-3-trimethylammonium-propane (DOTAP) (Avanti Polar lipid Inc, Alabaster Ala., product #890890 at 20 mg/ml in chloroform) and 6000 nmol of cholesterol (Avanti Polar lipid Inc, Alabaster Ala., product #700000), also prepared at 20 mg/ml in chloroform (J. T. Baker, Phillipsburg N.J., cat# 9180). The lipids were mixed in a borosilicate tube and dried for 1 hr under nitrogen at 4 LPM using a N-EVAP™ Organomation (Berlin, Mass.). The resulting lipid films were hydrated in 2 ml of 5% USP dextrose (Abbott, Laboratories, North Chicago, Ill. Cat# NDC-0074-7922-03 Lot 55-531-FW), to achieved a final lipid concentration of 6 mM. The multilamellar vesicles (MLVs) generated were sonicated for 30 sec using a sonicating bath (Laboratory Supplies Co INC, Hicksville, N.Y., serial #11463). The resulting lipid vesicles were sized and typically had a diameter from 300-500 nm, as determined in unimodal mode using a Coulter sizer N4Plus (Beckman Coulter, Miami, Fla., serial # AC52049). For the Pegylated liposome, 6000 nmol DOTAP, 4800 nmol cholesterol and 1200 nmol of 1,2-disteraoyl-sn-glycero-3-phosphoethanolamine-N-[methoxy(polyethylene glycol)-5K] (DSPE-PEG_{5K}) (Avanti Polar lipid Inc, Alabaster Ala., product #880220) were prepared as described above. Typically, targeted liposomes were prepared using 6000 nmol DOTAP, 4800 nmol cholesterol and 1200 nmol DSPE-PEG_{5K}-Folate Lot No.10107601. DSPE-PEG_{5K}-Folate was synthesized by Northern Lipid Incorporated (Vancouver, BC).

For targeted liposomes, DOTAP, Cholesterol lipid film and DSPE-PEG_{5K}-Folate were solubilized in chloroform at 20 mg/ml and were evaporated under nitrogen as described above. The targeted liposome complex was hydrated in HEPES 20 mM pH 7.2 and processed as described above for DOTAP:Cholesterol liposome.

Pre-compacted DNA-protamine complex preparation: was performed as described above in the methods.

Lipid-protamine-DNA (LPD) Preparation: LPDs were prepared as described by Li et al. (1998) Gene Therapy 5:930-937). Briefly, a lipid:protamine:DNA:ratio of 12 nmol total lipid: lug protamine:1 μg DNA were used for these experiments. Typically, 150 μl of liposomes at 6 mM were mixed with 326 μl of pre-compacted DNA at 230 μug/ml and 22.5 μl of 20 mM HEPES pH 7.2. For PPAA-containing LPD 22.5 μl of PPAA solution at 10 mg/ml in 20 mM HEPES was added to the compacted DNA instead of 22.5 μl of 20 mM HEPES as described above.

PPAA polymer was supplied by Dr. Allan Hoffman's laboratory (University of Washington, Seattle, Wash.). Synthesis and characterization of PPAA is described in Lackey et al. (1999) Bioconj. Chem. 10:401-405; Murthy et al. (1999) J. Controll. Release 61:137-143 and WO 99/34831. Final LPD preparations were sized and typically had a mean diameter from 150-200 nm with a N4Plus Coulter sizer using unimodal mode. The surface zeta potential was determined using a Malvern zeta sizer (Malvern Instrument Inc, Sacramento, Calif). Typically, LPD formulations have shown a positive zeta-potential with and without Pegylated lipid or lipid-conjugated-ligands and a negative zeta potential in presence of the PPAA in 20 mM HEPES pH 7.2.

In vitro transfection: 16 hr prior to transfection, 5×10⁴ cells, MDA-MB-231 or KB cell (ATTC, Manassas, Va., Cat no HTB-26 and cat no CCL-17) in 500 μl/well of appropriated media containing 10% FBS, were seeded in 48 well plate (Costar, Corning, N.Y., cat # 3548) and incubated over night at 37° C. in 10% CO₂. The following day, the medium was removed and replaced with 500 μl of fresh serum free media. Transfections were realized using 1 ug DNA/well (typically 6.67 ul from the LPD stock solution) and cells were incubated for 4 h at 37° C. in 10% CO₂. Six replicates per LPD formulation were tested.

After transfection, luciferase activity was assay as described above.

In vitro transfection assessment after serum incubation: Transfection was realized as described above except that 100 μl of LPDs were incubated for 1 h at 37° C. in 100 μl 20 mM HEPES pH7.2 or in 100 μl of 50% mouse serum (Cederlane Homby, ON, Cat No CL8000 Lot#1054) prior to transfection. The mean diameter of LPD formulation following serum incubation was performed using the Coulter sizer as described above.

Cell proliferation assay using MTS reagent: LPD cell toxicity was evaluated using cell titer 96 Aqueous non-radioactive cell proliferation assay from Promega (Promega Corporation, Madison, Wis. cat no G5421). Briefly, 5×10³ KB cells were plated 24 hours prior to incubation with formulations delivering 1 μg DNA/well or 5 μgDNA/well in a 96 well plate. Cells were incubated with LPD formulation for 4 h at 37° C. in serum free media. Following incubation the cell culture media was removed and 100 μl of fresh cell culture media containing 20 μl of MTS reagent was added to the cells and incubated for 2 h at 37° C. prior to taking the OD (optical density) reading.

Di-I labeled LPD binding to cells: LPDs were labeled with 1,1′-dioctadecyl-3,3,3′,3′-tetramethylindocarbocyanine perchlorate (DiI) (Molecular Probes, Eugene, OR, Cat no. D-282). Di-I is a non exchangeable, non metabolized fluorescent lipid tracer (Claassen (1992) J Immunol Methods 147:231-40).

Typically, 6.67 μl of LPDs (containing 1 ug DNA) were diluted in 93 μl of 20 mM HEPES pH 7.2 and 1 μl of a DiI stock solution of 500 μg/ml DiI in methanol was added to the LPD solution. LPDs were incubated at room temperature for 30 min prior to use. 100 μl of fluorescent LPDs were incubated with 1×10⁶ cells at 37° C. for 1 h and followed by 3 washes in PBS and re-suspended in 1.0 ml of 2% paraformaldehyde solution. 10,000 cells per sample were analyzed on a BD FACS as described above.

A dose titration of PPAA into LPD formulations was performed and formulations were sized and the zeta-potential was measured in 20 mM HEPES pH 7.2 and pH 4.2. The mean particle size and population size distributions (represented by the polydispersity value) were evaluated and results shown in Table 21. Typically, DOTAP:CHOL composed LPDs showed a mean diameter 150-300 nm. Addition of PPAA into formulation increased formulation sizes to the 400-500 nm range. LPD zeta-potential has been previously reported to ranges from +30 to +45 mV for formulation composed of DOTAP:CHOL in a 1:1 lipid mol ratio and a 12:1:1 nmol lipid: μg protamine: μg DNA. PPAA incorporation into such LPD formulations at a ratio of ≧3 μg PPAA/ug protamine compacted DNA changed the LPD surface potential to a negative value ranging from −35 to −20 mV. When zeta potential was measured in HEPES at pH 4.2 a positive zeta potential values was obtained. Data shown in Table 21 are from a single experiment.

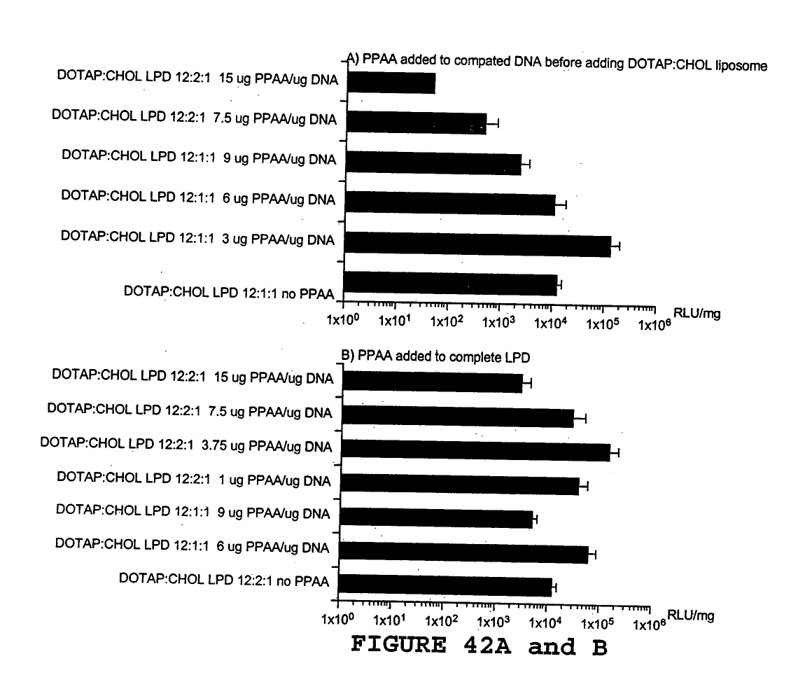

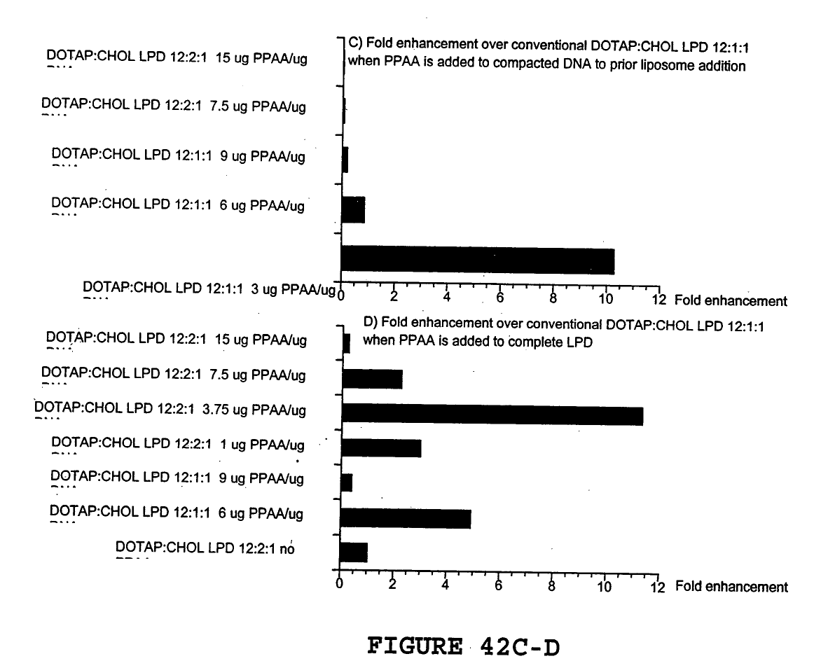

FIGS. 42A and B show that incorporation of PPAA into LPD formulation increases in vitro transfection in KB cells by 10 fold at a ratio of 3 μg PPAA/μg compacted protamine-DNA when PPAA was added directly to pre-compacted DNA. In comparaison, PPAA addition to complete LPD a ratio of 6 μg PPAA per μg DNAalso resulted in a 10-fold transfection enhancement at 12:1:1 (nmol lipid; μg Protamine: μg DNA.). However, when LPD were prepared at 12:2:1 ratio a 3.75 μg PPAA/μg DNA was required to obtained a 10-fold transfection enhancement over base LPD formulation. Columns in FIGS. 42A and B represent RLU/mg of luciferase expression following transfection in KB cells. FIGS. 42C and D represent the fold enhancement of the data presented in A and B compared to base PEG formulation in KB cells. 5×10⁴ cells were plated 24 hours prior to transfection with formulations delivering 0.1 μg DNA/well in a 48 well plate. Cells were transfected for 4 h in serum free media and harvested 48 hours post transfection for luciferase expression. N=6 independent transfections per formulation group. All formulations were generated using the general formula: 12 nanomoles lipid (DOTAP:CHOL); 1 μg Protamine: 1 μg DNA. Different ratio PPAA: μg DNA were incorporated into LPD formulation as described above.

### EXAMPLE 45

Protonation of poly(acrylic acid) PPAA in Cationic DNA/Lipid/Protamine Complexes

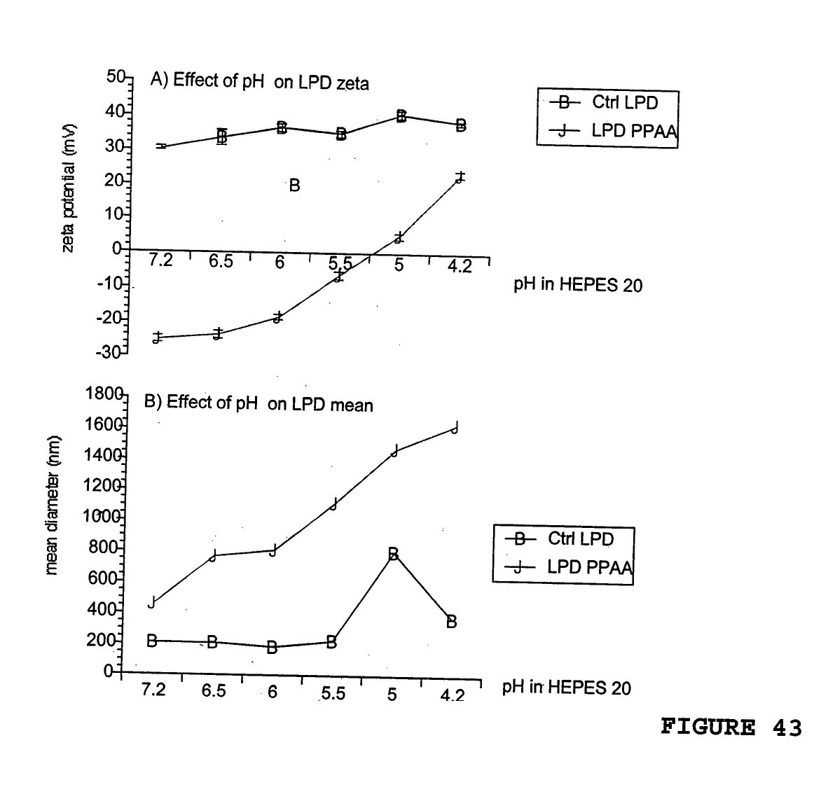

PPAA-containing cationic LPDs were prepared as described in Example 44. Protonation of the PPAA-containing LPDs was determined and concomitant changes in formulation zeta-potential was also monitored through a pH titration using 20 mM HEPES prepared at different pH values ranging from 7.2 to 4.2. Lines represent the zeta potential in FIG. 43A or the mean LPD diameter in FIG. 43B. All formulations were generated using the general formula: 12 nanomoles lipid (DOTAP:CHOL); 1 μg Protamine: 1 μg DNA; and 3 μg PPAA/ug of protamine compacted DNA. In this particular experiment PPAA was added directly to compacted DNA prior to liposome addition. Data indicated that LPD containing PPAA reverse charge at a pH lower than 5.5, while conventional LPD keep the same charge at all pH evaluated (FIG. 43A). This effect was also reflected on LPD mean diameter. Particles containing PPAA have shown a size increase proportional to a pH decrease while the mean diameter of conventional LPD without PPAA was not affected (FIG. 43B).

### EXAMPLE 46

Stability of poly(acrylic acid) PPAA-Containing Cationic DNA/Lipid/Protamine Complexes in Mouse Serum

As a model for LPD stability in vivo, LPDs without or with PPAA, added to pre-compacted DNA were pre-incubated for 1 h at 37° C. in mouse serum prior size measurement and transfection assessment. As shown in Table 22, DSPE-PEG_{5K} or DSPE-PEG_{5K}-Folate were added to LPD formulation at 2 and 10 mol % to increase particle stability in serum and promote formation of smaller size particles. As expected the DSPE-PEG_{5K} addition into LPD formulation resulted in LPD ranging from 150-300 nm in size whether or not they contained PPAA. PEG-bearing formulations without PPAA tend to have a mean particles size around 150 nm while in presence of PPAA the mean particles size tend to be in the 200-300 nm range. Table 22 shows the effect on particle mean diameter as measured in unimodal mode and zeta potential of the incorporation of PPAA into LPDs. LPD were prepared at 12:1:1 ratio (nmol lipid; μg Protamine: μg DNA). Final DNA concentration in LPD formulation was 150 μg DNA/ml.

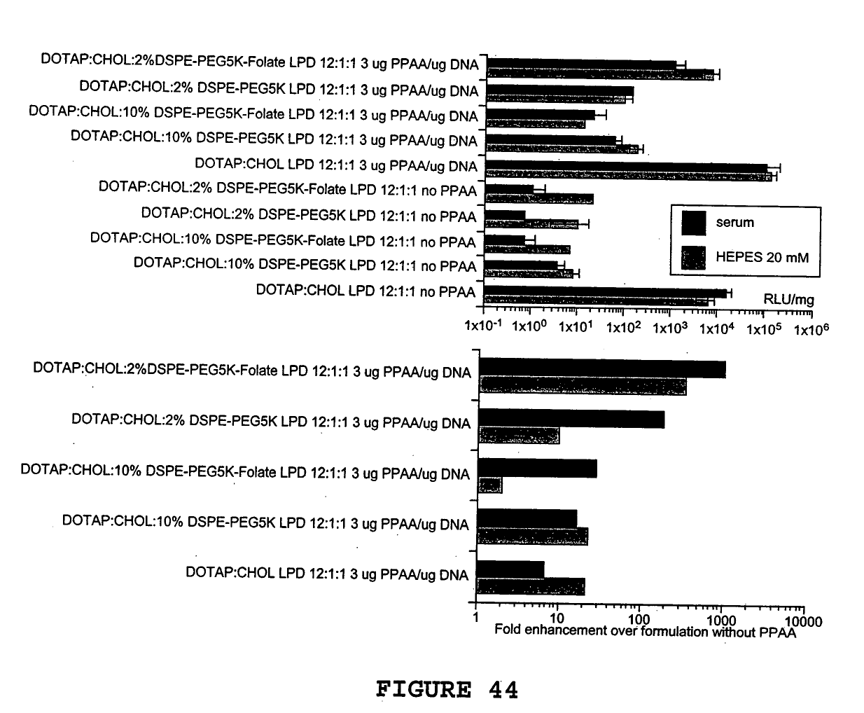

PPAA addition to LPD, LPD-PEG or LPD-PEG-Folate on in vitro transfection in KB cells was evaluated and results are shown in FIG. 44. For evaluation in mouse serum, LPD were incubated for 1 h at 37° C. in 50% HEPES 20 mM pH 7.2 or in 50% mouse serum prior cell transfection using 0.1 μg DNA/well. 5×10⁴ cells were plated 24 hours prior to transfection with formulations delivering 0.1 μgDNA/well in a 48 well plate. Cells were transfected for 4 h in serum free media and harvested 24 hours post-transfection for luciferase expression. N=6 independent transfection per formulation group. All formulations were generated using the general formula: 12 nanomoles lipid (DOTAP:CHOL:X); 1 μg Protamine:1 μg DNA. DSPE-PEG_{5K} and DSPE-PEG_{5K}-Folate were incorporated at a 2 and 10 mole % ratio. 3 μg PPAA/ug of protamine compacted DNA, was used in this particular experiment. PPAA was added directly to compacted DNA prior liposomes addition. LPD containing PPAA show 1 to 3 log transfection enhancement over the same formulation without PPAA. Moreover, this effect was similar or superior for the serum treated sample. The effect of PPAA incorporation into LPD, LPD-PEG and LDP-PEG-folate on KB cells in vitro transfection is shown in FIG. 44. Columns represent RLU/mg luciferase expression or fold transfection enhancement over base formulation without PPAACells were transfected with 0.1 μg DNA/well, prior transfection LPD were incubated for 1 h at 37° C. in 20 mM HEPES pH 7.2 or in 50% mouse serum.

### EXAMPLE 47

Effect of Addtion of DSPE-PEG_{5K}-Folate Addition to LPD with or without PPAA on in vitro Transfection in KB Cells

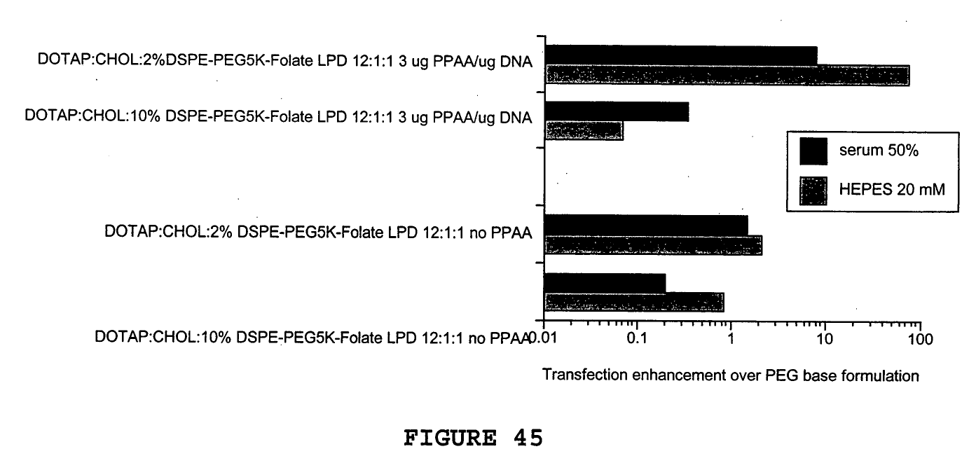

5×10⁴ KB cells were plated 24 hours prior to transfection with formulations delivering 1 μg DNA/well in a 48 well plate. Cells were transfected for 4 h in serum free media and harvested 24 hours post transfection for luciferase expression. N=6 independent transfection per formulation group. All formulations were generated using the general formula: 12 nanomoles lipid (DOTAP:CHOL:X); 1 μg Protamine: 1 μg DNA. DSPE-PEG_{5K} and DSPE-PEG_{5K}-Folate were incorporated at a 2 and 10 mole % ratio. FIG. 45 represents transfection fold enhancement over base PEG Formulation for LPD formulation containing 2 or 10 mol % DSPE-PEG_{5K}-Folate.

### EXAMPLE 48

Effect PPAA on Cell Toxicity

An MTS assay was performed to determine the effect of PPAA on cell toxicity. 5×10³ KB cells were plated 24 hours prior to incubation with formulations delivering 1 μgDNA/well or 5 μgDNA/well in a 96 well plate. Cells were incubated with LPD formulation for 4 h at 37° C. in serum free media, following incubation the cell culture media was remove and 100 μl of fresh cell culture media containing 20 μl of MTS reagent were added to cell and incubated 2 h at 37° C. prior to OD reading. N=6 independent transfection per formulation group. All formulations were generated using the general formula: 12 nanomoles lipid (DOTAP:CHOL:X); 1 μg Protamine: 1 μg DNA. DSPE-PEG_{5K} and DSPE-PEG_{5K}-Folate were incorporated at a 2 and 10 mole % ratio.

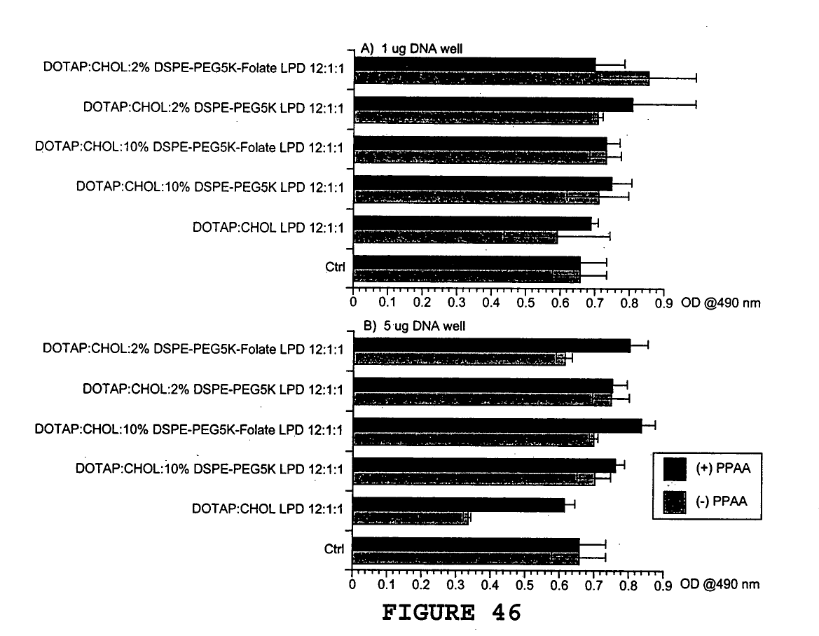

Results shown in FIG. 46 show that the addition of DSPE-PEG_{5K} or DSPE-PEG_{5K}-Folate into DOTAP:CHOL LPDs, prepared as described above, with or without PPAA, reduced LPD associated cell toxicity at a 5 μg DNA well concentration. Interestingly, PPAA without the PEG-containing lipids also reduced LPD associated cells toxicity in vitro. Columns represent optical density (OD) reading at 490 nm.

### EXAMPLE 49

Effect PPAA on Cell Binding in vitro

LPDs, prepared as described above, were labeled with DiI a fluorescent lipid tracer, and LPD binding to cells was accessed by flow cytometry. Results shown in Table 23 show a higher percentage of binding to cells by conventional LPDs compared to the pegylated LPD formulation. These results support the concept of using pegylated lipid to decrease the non-specific electrostatic LPD-cell binding. As expected, addition of DSPE-PEG_{5K}-Folate in an LPD formulation significantly restored the LPD-cell binding. However, the use of PPAA decreased LPD cell binding. Interestingly, in presence of PPAA the DSPE-PEG_{5K}-Folate-mediated cell binding over the PEG base formulation was superior, 1.2 fold for formulation without PPAA compared to 2.3 fold enhancement for PPAA-containing formulation. However, in terms of total mean fluorescence intensity the LPD binding to cells with PPAA-containing complexes was 2 to 5 time lower for all formulations evaluated.

### EXAMPLE 49

Effect of % Ratio of DSPE-PEG_{5K}-Folate on PPAA-Containing LPD Mean Diameter and Zeta Potential

It was shown above that a 2 mol % ratio of DSPE-PEG_{5K}-Folate was sufficient to show specific folate mediated transfection enhancement in vitro. However, in vivo in a murine tumor model, 2 mol % of DSPE-PEG was not sufficient to allow tumor gene expression following intravenous LPD administration. In order to increase LPD stability and have a LPD targeting effect at a lower ligand mol ratio, different percentages of DSPE-PEG_{2K} were incorporated into LPD formulation containing 2 mol % of DSPE-PEG_{5K}-Folate and results are shown in Table 24. No significant effect on particle size was observed for these DSPE-PEG_{2K} containing LPD.

The Zeta potential measurements show a shift from a negative zeta potential at pH 7.2 to a positive zeta potential at pH 4.2. Interestingly, the zeta potential shift was more pronounced for non- bearing PEG formulation compared to PEG bearing formulation. For all the formulation tested as expected for formulation without PPAA no shifts in the zeta potential as a function of the pH were observed.

LPD were prepared at 12:1:1 ratio (nmol lipid; μg Protamine: μg DNA). Final DNA concentration in LPD formulation was 150 μg DNA/ml. Results are summarized in Table 24.

In order to increase LPD stability and have a LPD targeting effect at a low ligand:mol ratio different percentage of DSPE-PEG_{2K} were incorporated into LPD formulation containing 2 mol % of DSPE-PEG_{5K}-Folate. Transfection activity for formulations with or without PPAA were investigated in KB cells. 5×10⁴ KB cells were plated 24 hours prior to transfection with formulations delivering 0.1 μg DNA/well in a 48 well plate. Cells were transfected for 4 h in serum free media and harvested 24 hours post transfection for luciferase expression. N=6 independent transfection per formulation group. All formulations were generated using the general formula: 12 nanomoles lipid (DOTAP:CHOL:X:Y); 1 μg Protamine:1 μg DNA. X and y represents DSPE-PEG_{2K} and DSPE-PEG_{5K}-Folate which were incorporated into LPD formulation at different mole % ratio as indicated above. 3 μg PPAA/ug of protamine compacted DNA, was used in this particular experiment, PPAA was added directly to compacted DNA prior liposomes addition.

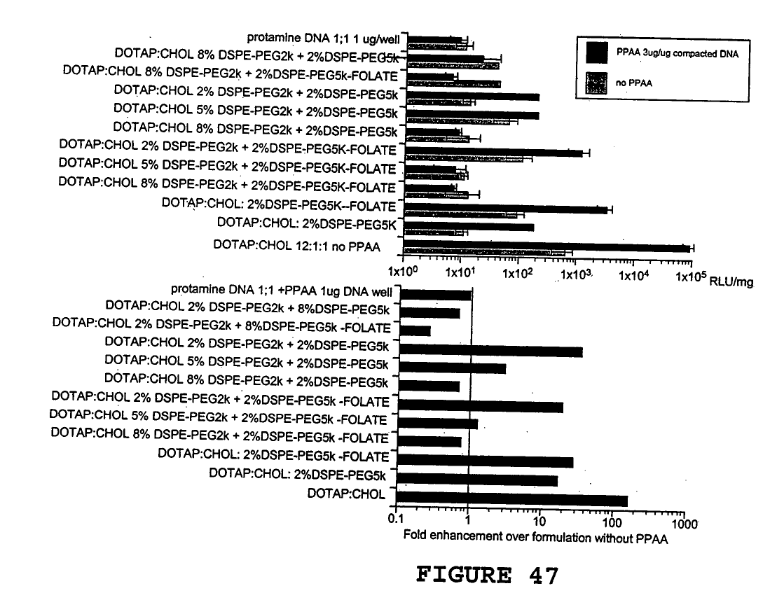

Addition of PPAA to LPD, LPD-PEG or LPD-PEG-Folate shows 1 to 2 log transfection enhancements over the same formulation without PPAA (FIG. 47). Columns represent RLU/mg luciferase expression in FIG. 47A) and transfection fold enhancement over base PEG Formulation in FIG. 47B). The effect of the addition of DSPE-PEG_{2K} to formulations containing DSPE-PEG_{5K} or DSPE-PEG_{5K}-Folate was also determined. DSPE-PEG_{2K} addition into LPD formulation should result in a better membrane surface shielding consequently reducing blood protein interaction with liposome membrane. As illustrated in FIG. 47, formulations containing 5 and 8 mol % of DSPE-PEG_{2K} and 2 mol % DSPE-PEG_{5K}-Folate failed to show PPAA transfection increase. However, for LPD formulation containing 2 mol % of DSPE-PEG_{2K} and 2 mol % DSPE-PEG_{5K}-Folate, PPAA addition increased transfection by 1 log over same formulation without PPAA.

### EXAMPLE 50

Effect of PPAA/DNA Ratio on PPAA-Containing LPD Mean Diameter, Zeta Potential and Transfection Activity

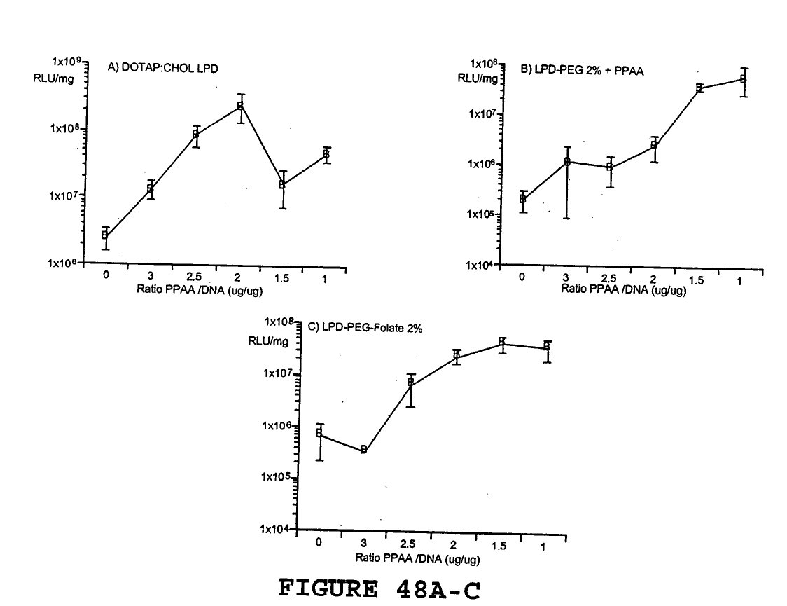

DOTAP:CHOL, DOTAP:CHOL-DSPE-PEG and DOTAP:CHOL:DSPE-PEG-Folate PPAA-containing LPDs were prepared as described above and transfection of KB cells was performed also as described above and as listed in Table 25. Formulations were prepared with both 2% (FIG. 48 A-C) and 10% (FIG. 48 D and E) PEG incorporation.

As shown in FIG. 48 A-C, decreasing the PPAA/DNA ration resulted in increased in vitro transfection activity. However, under 2.5 μg PPAA/ μg DNA led to mean diameter incease, despite incorporation of PEG in the formulation (Table 25).

### EXAMPLE 51

Effect of Lysomotrophic Agent on PPAA-Mediated Transfection in vitro

DOTAP:CHOL, DOTAP:CHOL-DSPE-PEG and DOTAP:CHOL:DSPE-PEG-Folate PPAA-containing LPDs were prepared as described above and transfection of KB cells with 0.1 μg/well DNA was performed also as described above. Lysomotrophic agent, either chloroquine (an anti-malarial drug, weak base protonated in endosome) or bafilomycin A (a specific ATPase inhibitor which reduces the amount of protons entering the endosome), capable of preventing endosomal acidification, were added in varying amounts to the transfection mixture half and hour prior to LPD addition to the cells.

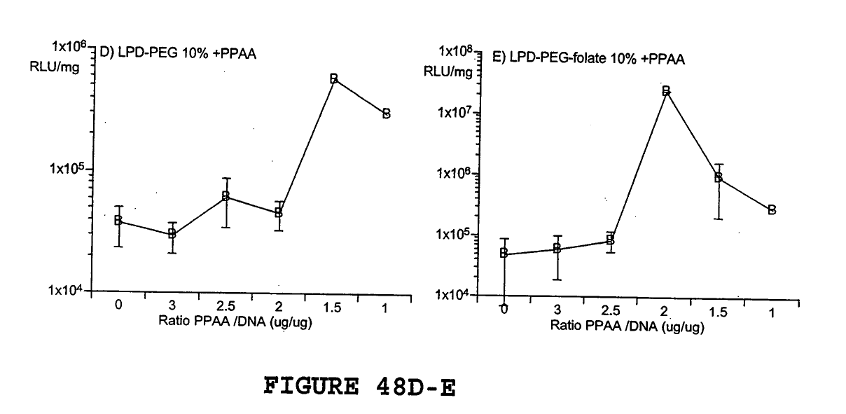

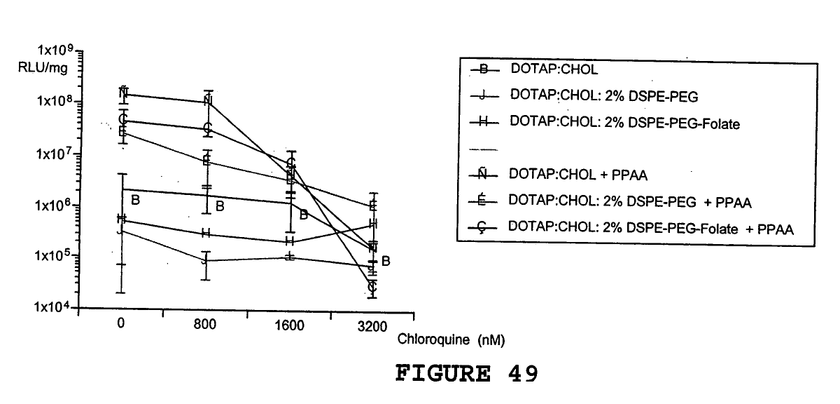

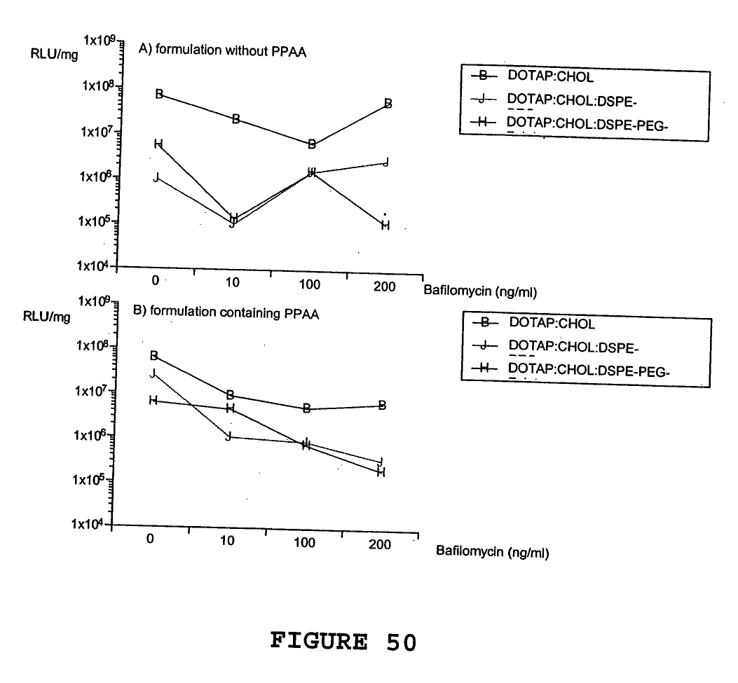

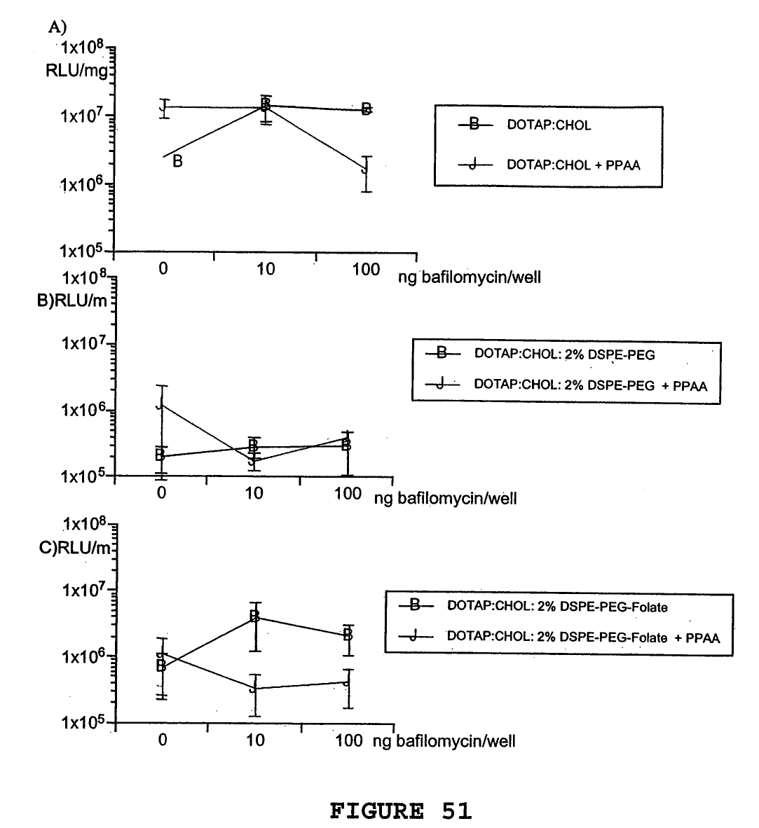

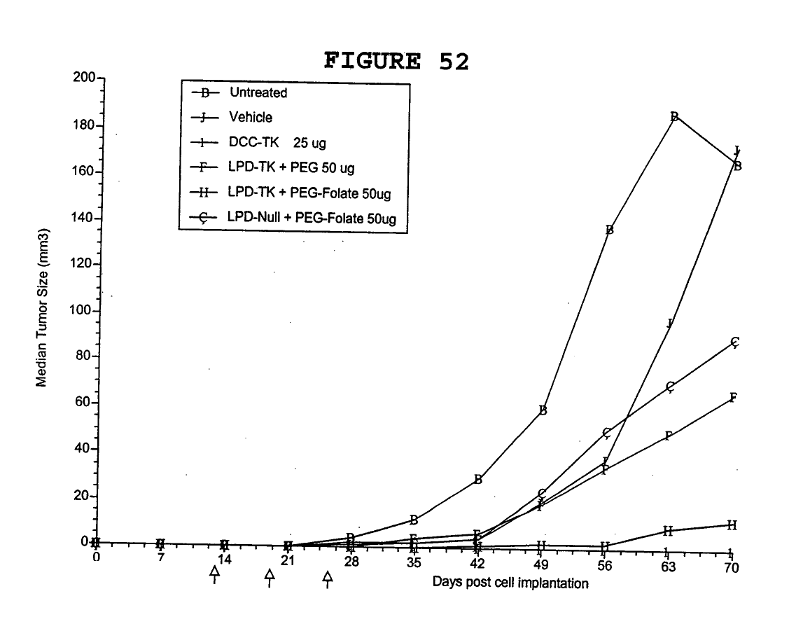

The results are shown in FIGS. 49-51. Chloroquine at 1600 nM blocked PPAA-mediated transfection, eliminating PPAA-mediated transfection enhancement. Bafilomycin A at 10 ng/well also appeard to block PPAA-mediated transfection. From these results it appears that PPAA-mediated transfection enhancement is dependent upon endosomal acidification.

### EXAMPLE 52

Effect of DSPE-PEG_{5K} and DSPE-PEG_{5K}-Folate Incorporation into LPD Formulations on in vitro Complement Activation

LPD formulations, shielded LPD formulations (LPD-PEG_{5K} incorporated at either 2 or 10 mole %) and targeted-shielded LPD formulations (LPD-PEG_{5K}-folate at either 2 or 10 mole %) containing the plasmid DNA pCMVinluc were prepared as described. A lipid:protamine: DNA: ratio of 12 nmol total lipid:1 μg protamine: 1 μg DNA was used for all experiments.

The Complement Opsonization was performed as described in Ahl et al. (Ahl et al., 1997, supra). Briefly, the LPD formulation, shielded LPD formulations (LPD-PEG_{5K} incorporated at either 2 or 10 mole %), targeted shielded LPD formulations (LPD-PEG_{5K}-folate at either 2 or 10 mole %), to be assayed were first incubated with freshly reconstituted lyophilized complement-positive human serum (Sigma, St. Louis, Mo.) for 30 minutes at 37° C. The final LPD lipid concentration was always 0.9 mM in these incubations. The human serum was diluted 6-fold using Dulbecco's phosphate-buffered saline (PBS) (Life Technologies, Gaithersburg, Md.). The complement level in the serum following this incubation with the LPD formulations was then determined according to standard clinical procedure (Stites and Rogers, 1991) using complement-dependent hemolysis of activated sheep erythrocytes (BioWhittaker, Inc., Walkersville, Md.). The serum dilution which gives 50% hemolysis, i.e. the CH50 value, is directly proportional to the serum complement levels. The percentage reduction in the serum CH50 value following the incubation with a LPD formulation indicates the level of LPD-induced complement activation and thus LPD complement opsonization. CH50 values were calculated by a linear fit to a log-log version of the von Krogh equation. All LPD-PEG_{5K} or LPD-PEG_{5K}-folate formulations were assayed along with PBS buffer and an unmodified LPD formulation as a positive control. Unmodified LPD formulations always had a high level of human complement opsonization. Unmodified LPD's typically reduced the serum CH50 level by at least 90% under our experimental conditions. The opsonization percentage of any LPD-PEG_{5K} or LPD-PEG_{5K}-folate formulation was calculated by the equation shown below using the CH50 values for the formulation and the controls.
Opsonization=100×(CH50_{PBS}−CH50_{LPD Formulation})/(CH50_{PBS}−CH50_{umodifed LPD})

n order to compare LPD formulations to shielded or targeted-shielded formuations, the reduction in the CH50 for the base LPD formulation was defined as 100% opsoniztion. The data presented in Table 26 demonstrate that incorporation of 2 mole % of PEG_{5K} in the DSPE-PEG_{5K} or DSPE-PEG_{5K}-Folate LPD formulations had little appreciable effect on opsonization of the complex. LPD formulations with 10 mol % DSPE-PEG_{5K} or DSPE-PEG_{5K}-Folate were found to significantly reduce the reduced the opsonization level to 60 and 77% of the unmodified LPD formulation.

In Examples 44-52, the incorporation of poly (acrylic acid) PPAA into LPD formulations is described and the resulting formulations characterized. PPAA is a pH sensitive polymer known for its membrane disruptive capacity at endosomal pH (Stayton et al. (2000) J Controll. Release 65:203-220). Addition of such a polymer is compatible with LPD formation at a specific ratio; ≧3 μg PPAA/ug DNA. This ratio seems to give optimal results in term of transfection enhancement (1 log over formulation without PPAA). Two methods of PPAA incorporation into LPD were investigated, PPAA was added either to the protamine-compacted DNA prior the liposome addition or directly to the final LPD preparation. Results indicated that the first approach where PPAA is added directly to compacted DNA was more appropriate for further study. Addition of PPAA into formulation increases LPD mean diameter from ˜200 nm for conventional LPD up to 400-800 nm for LPD formulation containing PPAA. However, addition of DSPE-PEG_{5K} or a lipid-conjugated-ligand such as DSPE-PEG_{5K}-Folate at a molar ratio ranging from 2 mol % to 10 mol % prevented PPAA-mediated LPD size increases. PPAA incorporation into LPDs enhanced in vitro transfection in KB cells by 10-100 fold. Moreover, addition of DSPE-PEG_{5K} or DSPE-PEG_{5K}-Folate into LPD formulations containing PPAA enhanced transfection up to 2-3 log over the DOTAP:CHOL:DSPE-PEG_{5K} or DOTAP:CHOL: DSPE-PEG_{5K}-Folate base formulation without PPAA. This transfection enhancement was conserved or improved even after a 1 h incubation at 37° C. in mouse serum prior to transfection assessment. Interestingly, transfection enhanced formulations containing PPAA have shown a shift in their zeta potential from a negative zeta potential at pH 7.2 to a positive zeta potential at pH 4.2 indicating the pH sensitive effect possibly associated with the pH sensitive polymer.

In Examples 44-52, the ability of PPAA to enhance transfection activity over the corresponding LPD base formulation is demonstrated. PPAA addition to protamine-compacted DNA at a 3:1 μg PPAA: μg DNA ratio enhanced LPD mediated KB cell transfection by 10-100 fold. Other ratios were also observed to increase transfection.

As indicated by the zeta potential measurement PPAA appears to have a pKa around 5.5. PPAA seems to reduce in vitro cytotoxicity associated with LPDs as demonstrated using a MTS assay. This result could be correlated with a lower particle binding as demonstrated by FACS analysis. Interestingly, addition of DSPE-PEG_{5K} or DSPE-PEG_{5K}-Folate seems to reduce PPAA-mediated LPD size increase. No serum-mediated aggregation has been observed for PEG LPD containing PPAA. LPDs containing PPAA demonstrated a zeta potential shift from negative at neutral pH to positive at acid pH, however, DSPE-PEG_{5K} addition to LPD-PPAA reduced this zeta potential shift.

### EXAMPLE 53

Intravenous and Intratumoral Injection of Tumor-Bearing Mice with Cationic LPDs Containing Nucleic Acid Encoding a a Therapeutic Gene: Non-Targeted and Targeted

Ninety-nine female Balb/c athymic nude mice were used in this study Mice were injected with 5×10⁶ MDA-MB-231 tumor cell subcutaneously in the right flank. Five days after tumor innoculation animals were treated with DCC or LPD formulations as described below:

| Groups: | #Injections | DNA μg |
|---|---|---|
| 1.) Untreated (no ganciclovir) | 0 | |
| 2.) Vehicle + Ganciclovir | 3x | 0 |
| 3.) DCC-TK (*Intratumoral) | 3x | 25 |
| 4.) LPD-TK | 3x | 25 |
| 5.) LPD-TK + 10% PEG | 3x | 100 |
| 6.) LPD-null + 10% PEG | 3x | 100 |
| 7.) DCC-TK (*Intratumoral) | 1x | 25 |
| 8.) LPD-TK | 1x | 25 |
| 9.) LPD-TK + 10% PEG | 1x | 100 |
| 10.) LPD-null + 10% PEG | 1x | 100 |

DC-Chol (DCC) liposomes were prepared as described in Yoo et al., (2001) Clinical Cancer Research 7:1237-1245 and LPDs were prepared as described above with DOTAP:CHOL at a 12:1:1 ratio of lipid:protamine:DNA with 25 (groups 3, 4, 7,8) or 100 μg (groups 5,6,9,10) of plasmid DNA. In groups 3-5 and 7-9 the plasmid DNA contained in the formulation was the pk2 CMV TK1 plasmid (Celltech, Santa Ana, Calif.) as the model therapeutic gene construct which represent the Herpes Simplex Type I thymidine kinase gene under the control of the CMV promoter. For groups 6 and 10 the plasmid was a control null plasmid p(e1a)K2 which represents a null deletion mutant of the E1A gene that will not code for a gene product. This plasmid serves as a DNA control. The mechanism of action of TK gene therapy is based on the introduction into the cell of the gene coding the HSV-1 TK enzyme. This TK is less discriminating of substrates than the mammalian TK and specifically converts a non-toxic pro-drug such as Ganciclovir (GCV) to its toxic metabolic the Ganciclovir triphosphate (GCV-TP). GCV-TP will be incorporated into cellular DNA, resulting in the formation of a replication dependent DNA double strand break and leading to cell growth arrest in S or G/2 M phase and apoptotic cell death (Tomicic et al., (2002) Oncogene 21:2141-2153).

Injections of control, DCC, or LPD formulations were performed once per week for groups 7-10 or 3 times per week for groups 1-6. LPD and Control formulations were injected intravenously (IV) and DCC formulations were injected intratumorally. Ganciclovir was injected intraperitoneally (IP) 2× daily for a total of 8 days at 100 mg/kg for Groups 2-6, or IP 2× daily for a total of 2 days at 100 mg/kg for (Groups 7-10) Group 1 was untreated and received no ganciclovir. Group 2 was the control group and received vehicle (DOTAP:CHOL PEG) and ganciclovir. All groups in the study were evaluated daily for survival and weekly for tumor growth as measured using calipers as performed in the art (individual animal tumor volumes were measured as well as means, medians and standard deviations for each group) and body weights. Tumor growth was determined by weekly caliper measurement of the tumor. The tumor volume (mm³) was calculated by multiplying the length, width, and the depth of each tumor and then dividing by 2 [(L×W×D)/2]. In accordance with good animal practices known in the art, animals were removed from the study once the tumor size reached 10% of total body weight or animals appeared moribund. Data presented in Table 27 represents the median tumor size at day 56 of study for each treated group.

Groups 1 and 2 representing untreated or vehicle control treated animals have a median tumor size of 1303 or 1250 respectively demonstrating progressive tumor growth.

Groups 3 and 7 animals were treated with direct intratumoral injection of the DCC formulation containing the thymidine kinase (TK) gene. Animals in these groups demonstrate therapeutic effectiveness of the TK gene and ganciclovir treatment as the median tumor size is reduced to 402 and 255 respectively. Although the DCC formulation demonstrates therapeutic benefit when injected intratumorally this formulation is not suitable for intravenous delivery as has been described in the art.

Groups 4 and 8 represent the non-PEGylated or unshielded LPD formulations containing the therapeutic TK gene injected intravenously and treated with ganciclovir. In these groups the median tumor size is to 1250 and 345, respectively.

Groups 5 and 9 represent LPD formulation that have been shielded with 10 mol % of PEG_{5K}. Animals in these groups have a median tumor size of 616 and 345 respectively at day 56 demonstrating therapeutic effectiveness of the TK gene and ganciclovir treatment. Shielding of the complex with PEG_{5K} has increased the therapuetic potential of the LPD formulation.

Groups 5-9 and 6-10 were formulated with 100 μg of the therapeutic gene construct (TK1) into the PEG shielded LPD compared to Groups 4 and 8 which contined only 25 μg of the therapeutic gene formulated into the base LPD formulation. It is important to note that base LPD formulations can not be formulated for in vivo use at higher DNA concentrations than 25 μg due to toxicity when administered.

Groups 6 and 10 represent PEG shielded LPD control formulation containing a null plasmid and have a median tumor size of 616 and 820 respectively.

### EXAMPLE 54

Intravenous and Intratumoral Injection of Tumor-Bearing Mice with LPDs Containing Nucleic Acid Encoding for Thvmidine Kinase: Folate-Targeted

One hundred and ten female Balb/c athymic nude mice were used in this study. Mice were injected with 5×10⁶ MDA-MB-231 tumor cells subcutaneously in the right flank. Seven days after tumor innoculation animals were treated with DCC or LPD formulations as described below:

| Group: | DNA μg |
|---|---|
| 1.) Untreated | 0 |
| 2.) Vehicle (Dotap/Chol + PEG) | 0 |
| 3.) DCC-TK (*Intratumoral) | 25 |
| 4.) LPD-TK | 25 |
| 5.) LPD-TK + 10% PEG | 25 |
| 6.) LPD-TK + 10% PEG | 50 |
| 7.) LPD-TK + 10% PEG | 100 |
| 8.) LPD-TK + 10% PEG-Folate | 50 |
| 9.) LPD-null + 10% PEG | 100 |
| 10) LPD-null + 10% PEG-Folate | 50 |

DC-Chol (DCC) liposomes were prepared as described in Yoo et al., (2001) Clinical Cancer Research 7:1237-1245, and LPDs were prepared as described above with DOTAP:CHOL, DOTAP:CHOL:DSPE:PEG, or DOTAP: CHOL:DSPE:PEG:FOLATE at a 12:1:1 ratio of lipid:protamine:DNA with 25 (groups 3,4), 50 (groups 6,8,10) or 100 μg (groups 7,9) of plasmid DNA. In groups 3-8 the plasmid DNA contained in the formulation was the pk2 CMV TK1 plasmid (Celltech) as the model therapeutic gene construct. For groups 9 and 10 the plasmid was a control null plasmid p(e1a)K2. Plasmid constructs and the theory of TK gene therapy is described above.

Injections of control, DCC, or LPD formulations were performed once per week for three weeks and were given intravenously for all groups except group 3 which received DCC injected intratumorally. Ganciclovir was administered intraperitoneally twice daily for two consecutive days beginning the day of the administration of the formulation for the three weeks the lipid formulations were administered at a dose of 100 mg/kg. Group 1 was untreated and received no ganciclovir. Group 2 was the control group and received vehicle (DOTAP:CHOL:PEG) and ganciclovir. All groups in the study were evaluated daily for survival and weekly for tumor growth as measured using calipers as performed in the art (individual aanimal tumor volumes were measured as well as means, medians and standard deviations for each group) and body weights. Tumor growth was determined by weekly caliper measurement of the tumor. The tumor volume (mm³) was calculated by multiplying the length, width, and the depth of each tumor and then dividing by 2 [(L×W×D)/2]. In accordance with good animal practices known in the art, animals were removed from the study once the tumor size reached 10% of total body weight or animals appeared moribund. Data presented in FIG. 52 for Groups 1-3, 6, 8, 10 represents the tumor growth curve up to day 70 of study for each treated group. The data presented for Groups 1-3, 6, 8, 10 compares the 50 μg DNA dose for shielded LPD (PEG LPD) and the targeted shielded LPD (PEG-Folate LPD).

As demonstrated in FIG. 52 and Table 28, the LPD-PEG-Folate formulation containing the TK plasmid reduced the tumor growth to a greater extent than the corresponding untargeted, LPD-PEG formulation, importantly the same LPD-PEG formulation containing a null plasmid had only a minimal effect on tumor growth. At day 70, tumor size data shows a median tumor size of 174 mm³ (range from 40-586 mm³) for the vehicle control group, compared to 37 mm³ (range from 0-239 mm³) for the LPD-PEG formulation and 18 mm³ (range from 0-199 mm3) for the LPD-PEG-Folate formulation, while the control plasmid formulated into LPD-PEG-Folate had a median tumor size of 142 mm³ (range from 0-1250 mm³). These data clearly suggested that this folate targeted LPD formulation is effective in reducing tumor growth in vivo at levels substantially better (8 fold improvement) than those for a non-ligand bearing formulation.

### EXAMPLE 55

### Transfection Efficiency of Targeted Anionic LPD Formulations in Tumor-Bearing Mice

Six week old female nude Balb/C athyrnic nude mice were injected sub-cutaneously in the right flank with 5×10⁶ SKOV3-ipl cells. Five weeks post cell injection mice received, 50 μg of DNA (666μl) (pCMV Luc) formulated in various DLPD formulations as described below prepared at 75.0 μg DNA/ml were injected via the tail vein. Immediately after DLPD injection mice were subject to local ultrasound at the tumor site (1.5 W/cm² for 5 min) using a Sonitron 100 (Rich-Mar Corporation, Inola, Okla.). The mice were randomized into the following treatment groups (n=4-6 animal/group): (1); untreated animal, (2); vehicle (CHEMS:DOPE liposomes and protamine sulfate), (3); CHEMS:DOPE ALPD formulation, (4); CHEMS:DOPE:DSPE-PEG_{5K} ALPD formulation, (4); CHEMS:DOPE DSPE-PEG_{5K}-Folate ALPD formulation. Mean diameter and Zeta potentials are reported in Table 29. Selected tissues were harvested 16 h post ALPD administration and processed for luciferase expression as previously described.

As demonstrated in Table 30, the addition of 10 mol % DSPE-PEG_{5K}-Folate (Targeted- Shielded DLPDs) formulation results in an increase in the luciferase expression at the tumor site by 2-fold versuses 10 mol % DSPE-PEG_{5K} DLPD (Shielded DLPD) and by 5 fold over unmodified base DLPD formulation. Gene expression in other tissue was minimal.

### EXAMPLE 56

### Intravenous and Intratumoral Injection of Tumor-Bearing Mice with Anionic LPDs Containing Nucleic Acid Encoding a Therapeutic Gene: Non-Targeted and Targeted

Mice are injected with 5×10⁶ MDA-MB-231 tumor cell subcutaneously in the right flank. Five days after tumor innoculation animals are treated with Anionic LPDs (DLPDs) formulations as described below:

| Formulation | DNA μg |
|---|---|
| 1.) Untreated | 0 |
| 2.) Vehicle (CHEMS:DOPE:PEG) | 0 |
| 3.) DCC-TK (*Intratumoral) | 25 |
| 4.) LPD-TK + 10% PEG-Folate | 50 |
| 5.) CHEMS:DOPE (3:7) | 50 |
| 6.) CHEMS:DOPE:DSPE:PEG (3:6:1) | 50 |
| 7.) CHEMS;DOPE:DSPE:PEG: FOLATE (3:6:1) | 50 |
| 8.) NC12-DOPE (1:1) | 50 |
| 9.) NC12-DOPE:DSPE:PEG (1:0.8:0.2) | 50 |
| 10.) NC12-DOPE:DSPE:PEG: FOLATE (1:0.8:0.2) | 50 |

DC-Chol (DCC) liposomes are prepared as described in Yoo et al., Clinical Cancer Research 7:1237-1245, 2001, and LPDs are prepared as described above with DOTAP:CHOL, DOTAP:CHOL:DSPE-PEG, or DOTAP:CHOL:DSPE:PEG-FOLATE at a 12:1:1 ratio of lipid:protamine:DNA. DLPDs are prepared as described above with NC12-DOPE, NC12-DOPE:DSPE-PEG, and NC12-DOPE:DSPE-PEG-FOLATE. with 50 μg of plasmid DNA encoding a therapeutic gene (e.g., pk2 CMV TK 1) for all groups except Group 3 which is formulated with 25 μg as described previously. Injections of control, DCC, LPD or DLPD formulations are performed once per week for three weeks and are given intravenously for all groups except group 3, which received DCC injected intratumorally. Ganciclovir is administered intraperitoneally twice daily for two consecutive days beginning the day of the administration of the formulation for the three weeks the lipid formulations are administered at a dose of 100 mg/kg. Group 1 is untreated and receives no ganciclovir. Group 2 is the control group and receives vehicle (CHEMS:DOPE-PEG) and ganciclovir. All groups in the study are evaluated daily for survival and weekly for tumor growth as measured using calipers as performed in the art (individual animal tumor volumes are measured as well as means, medians and standard deviations for each group) and body weights. Tumor growth is determined by weekly caliper measurement of the tumor. The tumor volume (mm³) is calculated by multiplying the length, width, and the depth of each tumor and then dividing by 2 [(L ×W×D)/2]. In accordance with good animal practices known in the art, animals are removed from the study once the tumor size reaches 10% of total body weight or animals appear moribund.

TABLE 1
Zeta-Potential and Mean LPD Diameter in 5% Dextrose USP.

| | | nmol lipid: | Mean | | Zeta |
|---|---|---|---|---|---|
| | Lipid | μg protamine: | Diameter | | potential |
| LPD Lipid Formulation | mol ratio | μg DNA ratio | (nm) | polydispersity | (mVolt)* |
| DOTAP:CHOL | 1:1 | 12:0.97:1 | 228.0 | 0.334 | 44.6 +/− 6.0 |
| DOTAP:CHOL:DSPE- | 1:0.8:0.2 | 12:0.97:1 | 169.0 | 0.322 | 41.2 +/− 4.8 |
| PEG_{5K} | | | | | |
| DOTAP:CHOL:DSPE- | 1:0.8:0.2 | 12:0.97:1 | 177.4 | 0.521 | 25.9 +/− 0.8 |
| PEG_{5K}-RGD | | | | | |
| DOTAP:CHOL:DSPE- | 1:0.8:0.2 | 12:0.97:1 | 188.3 | 0.324 | 38.6 +/− 0.5 |
| PEG_{5K}-LHRH | | | | | |

*SD for the zeta potential was calculated based on five readings from the same sample.

TABLE 2
Effect of Addition of 10 mol % DSPE-PEG_{5K} to LPD on the
Achievable DNA Concentration.

| Final | Volume (ml) of | Volume | Volume | | |
|---|---|---|---|---|---|
| conc of | Protamine/ | (ml) of | (ml) | Size | |
| DNA: | DNA @ 333 μg | Liposome | of 5% | After | poly- |
| (μg/ml) | DNA/ml | (@ 6 μM) | Dextrose | 1 hour | dispersity |
| 150 | 0.225 | 0.075 | 0.200 | 256.7 | 0.77 |
| 200 | 0.300 | 0.100 | 0.100 | 244.3 | 0.61 |
| 250 | 0.420 | 0.140 | 0 | 250.2* | 0.67 |

*LPDs aggregated 24 h later

TABLE 3
FACS Analysis Representing the Percentage of Integrin or
LHRH Receptor Expression in 4 Different Cell Lines.

| | | MDA- | MDA- | MDA- | SKOV3- | | |
|---|---|---|---|---|---|---|---|
| | Clone # | MB-231* | MB-231** | MB-231 | IP1 | H-69 | LL/2 |
| Mab anti-αVβ5 | AB1961F | 41.0% | 23.7% | 41.5% | 79.9% | 2.4% | 58% |
| Mab anti-αVβ3 | AB1976H | 75.5% | 54.56 | 79.9% | 56.5% | 27.7% | 30.2% |
| Human anti-LHRH | A9E4 | 55.0% | 28.17% | 8.1% | 89.4% | 1.1% | 1.4% |
| receptor | | | | | | | |

Mab = mouse monoclonal antibody IgG₁:
*cells isolated from tumor bearing mice;
**cells cultured in vitro and detached using trypsin; MDA-MB-231 cells cultured in vitro and detached using EDTA. Typically, negative controls for all cell lines evaluated showed less than 5% positive cells.

TABLE 4
FACS Analysis Representing the Percentage of Cell Binding for
Di-I Labeled Targeted LPDs After 1 h Incubation With Cells.

| | Lipid mol | MDA- | MDA- | SKOV3- | | |
|---|---|---|---|---|---|---|
| LPD Lipid Formulation | ratio | MB-231* | MB-231 | IP1 | H-69 | LL/2 |
| DOTAP:CHOL | 1:1 | 72.8% | 83.0% | 54.9% | 60.5% | 40.3% |
| DOTAP:CHOL:DSPE-PEG_{5K} | 1:0.8:0.2 | 34.0% | 27.1% | 17.3% | 15.2% | 1.5% |
| DOTAP:CHOL:DSPE-PEG_{5K}-RGD | 1:0.8:0.2 | 45.5% | 43.2% | 36.0% | 14.5% | n.d. |
| DOTAP:CHOL:DSPE-PEG_{5K}-LHRH | 1:0.8:0.2 | 90.3% | 95.8% | 49.5% | 49.5% | 49.1% |

n.d. = not determined;
*data from cells isolated from tumor bearing mice. Typically, negative controls for all cell lines evaluated showed less than 2% positive cells.

TABLE 5
FACS Analysis Representing The Mean Fluorescent Intensity Of
Cells After 1 H Incubation At 37° C. With DiI Labeled LPDs.

| | Lipid mol | MDA- | MDA- | SKOV3- | | |
|---|---|---|---|---|---|---|
| LPD Lipid Formulation | ratio | MB-231* | MB-231 | IP1 | H-69 | LL/2 |
| DOTAP:CHOL | 1:1 | 214.9 | 33.5 | 22.6 | 77.0 | 96.6 |
| DOTAP:CHOL:DSPE-PEG_{5K} | 1:0.8:0.2 | 32.4 | 21.6 | 15.1 | 20.3 | 56.2 |
| DOTAP:CHOL:DSPE-PEG_{5K}-RGD | 1:0.8:0.2 | 93.5 | 27.3 | 19.5 | 40.25 | n.d. |
| DOTAP:CHOL:DSPE-PEG_{5K}-LHRH | 1:0.8:0.2 | 265.4 | 44.0 | 23.5 | 60.3 | 99.6 |

n.d. = not determined;
*data from cells isolated from tumor bearing mice. Typically, negative controls for all cell lines evaluated showed a mean fluorescent intensity less than 6.0.

TABLE 6
Mean LPD Diameter in 5% Dextrose USP.

| | Unimodal Mean |
|---|---|
| Formulation | Diameter (nm) |
| Lipid + Protamine | 182.8 |
| Protamine compacted DNA | 204.6 |
| Naked Plasmid DNA | 175.9 |
| LPD (DOTAP/CHOL) | 212.9 |
| LPD + 10% DSPE-PEG_{5K} | 136.4 |
| LPD + 10% DOPE-094 | 959.0 |
| LPD + 10% DSPE-PEG_{5K}-LHRH | 150.4 |
| LPD + 10% DSPE-PEG_{5K}-LHRH + | 423.4 |
| 10% DOPE-094 | |
| Low EU LPD | 223.4 |
| Lipid (DOTAP/CHOL) + DNA | 630.1 |

TABLE 7
Fold transfection increase in HepSK1 cells over the base LPD
formulation with increasing percentage of lipid-Elan094.

| | Percentage lipid-Elan094 | | | | | |
|---|---|---|---|---|---|---|
| | Base | | | | | |
| LPD-ligand | formulation | 1% | 2% | 5% | 10% | 20% |
| DOTAP:Chol:Elan218* | 1 | 2 | 2 | 2 | 3 | 4 |
| DOTAP:Chol:Elan219** | 1 | 5 | 4 | 2 | 1.5 | 1.5 |
| DOTAP:Chol:DMPE- | 1 | 7 | 30 | 120 | 295 | 254 |
| PEG-Elan218* | | | | | | |
| DOTAP:Chol:DMPE- | 1 | 2 | 20 | NA | NA | NA |
| PEG-Elan219** | | | | | | |

*Elan218 = cholesteryl-succinyl-Elan094
**Elan219 = DOPE-succinyl-Elan094

TABLE 8
Fold transfection increase in MD-MBA-231 cells over the base LPD
formulation with increasing percentage of lipid-Elan094.

| | Percentage lipid-Elan094 | | | | | |
|---|---|---|---|---|---|---|
| | Base | | | | | |
| LPD-ligand | formulation | 1% | 2% | 5% | 10% | 20% |
| DOTAP:Chol:Elan218* | 1 | 2 | 1.5 | 1 | 1 | 0.6 |
| DOTAP:Chol:Elan219** | 1 | 9 | 20 | 1.2 | 0.8 | 0.5 |
| DOTAP:Chol:DMPE- | 1 | 3 | 3 | 12 | 17 | 11 |
| PEG-Elan218* | | | | | | |
| DOTAP:Chol:DMPE- | 1 | 4 | 7 | 23 | 20 | 16 |
| PEG-Elan219** | | | | | | |

*Elan218 = cholesteryl-succinyl-Elan094
**Elan219 = DOPE-succinyl-Elan094

TABLE 9
Biophysical Properties of Anionic DLPD Formulations.

| | | (−) lipid | | | | | |
|---|---|---|---|---|---|---|---|
| | | prot | | | | Zeta | Zeta |
| DLPD Lipid | Molar | excess | | Size | | potential** | potential** |
| Formulation | ratio | ratio | DNA:Protratio | (nm) | Polydispersity | pH 4.5 | pH 7.5 |
| DOTAP:CHOL | 1:1 | n.a. | 2:1 | 109.1 | 0.610 | 34.6 | 35.0 |
| DOPS:CHOL | 5.5:4.5 | 6:1 | 2:1 | 193.4 | 0.131 | −38.4 | −45.5 |
| DOPG:CHOL | 5.5:4.5 | 6:1 | 2:1 | 196.0 | 0.282 | −49.9 | −43.7 |
| CHEMS:DOPE | 3:7 | 4:1 | 2:1 | 140.4 | −0.012 | 26.0 | −42.0 |
| CHEMS:DOPE:DSPE- | 3:6:1 | 4:1 | 2:1 | 125.6 | 0.344 | −14.9 | −35.1 |
| PEG_{5K} | | | | | | | |
| CHEMS:DOPE:DSPE- | 3:6:1 | 4:1 | 2:1 | 596.4 | 0.972 | N.D. | N.D. |
| PEG_{5K}-RGD | | | | | | | |
| CHEMS:DOPE:DSPE- | 3:6:1 | 4:1 | 2:1 | 906.7 | 1.611 | N.D. | N.D. |
| PEG_{5K}-LHRH | | | | | | | |
| DOPS:CHOL | 5.5:4.5 | 6:1 | 2:1 | 216.2 | −0.038 | N.D. | N.D. |
| DOPS:CHOL:DSPE- | 5.5:3.5:1 | 6:1 | 2:1 | 201.2 | 0.251 | N.D. | N.D. |
| PEG_{5K} | | | | | | | |
| DOPS:CHOL:DSPE- | 5.5:3.5:1 | 6:1 | 2:1 | 614.0 | 1.050 | N.D. | N.D. |
| PEG_{5K}-RGD | | | | | | | |
| DOPS:CHOL:DSPE- | 5.5:3.5:1 | 6:1 | 2:1 | 579.7 | 0.535 | N.D. | N.D. |
| PEG_{5K}-LHRH | | | | | | | |

N.D. = not determined.;
Prot = protamine sulfate USP;
Polydis = size polydispersity;
**SD = ±5 mV for zeta potential

TABLE 10
Effect Of DNA Concentration On DLPD Mean Diameter

| | | | DOPS:CHOL:D | | | | DOPS:CHOL:DS | |
|---|---|---|---|---|---|---|---|---|
| DNA Conc. | DOPS:CHOL | | SPE-PEG_{5K} | | DOPG:CHOL | | PE-PEG_{5K} | |
| (μg/ml) | Size (nm) | poly | Size (nm) | poly | Size (nm) | poly | Size (nm) | poly |
| 75.0 | 156.3 | 0.259 | 147.5 | 0.694 | 159.3 | 0.388 | 197.5 | 0.526 |
| 100.0 | 211.9 | 0.253 | 179.1 | 0.626 | 211.4 | 0.528 | 179.0 | 0.486 |
| 125.0 | 253.6 | 0.137 | 178.1 | 0.412 | 260.7 | 0.445 | 193.8 | 0.26 |

| | | | | | | |
|---|---|---|---|---|---|---|
| 150.0 | aggregate | 216.3 | 0.163 | 296.0 | 0.425 | aggregate |

TABLE 11
FACS Analysis Representing The Percentage Of Cell Binding
For DLPD Di-I Labeled
After 1 H Incubation With MDA-MB-231 cells.

| Lipid mol | | |
|---|---|---|
| ratio | | MDA-MB-231 |
| 1:1 | DOTAP:CHOL | 72.8% |
| 55:45 | DOPS:CHOL | 14.32% |
| 55:45 | DOPG:CHOL | 35.35% |
| 3:7 | CHEMS:DOPE | 31.97% |

Typically negative control for all cell lines evaluated using non-labeled liposomes shown less than 2% positive cells.

TABLE 12
Biophysical Proprieties of Anionic DLPDs

| | | (−) lipid | | | | | |
|---|---|---|---|---|---|---|---|
| | | prot | | | | Zeta | Zeta |
| | Molar | excess | DNA: | Size | | potential | potential |
| | ratio | ratio | Prot ratio | (nm) | Polydis | pH 4.5 | pH 7.5 |
| DOTAP:CHOL | 1:1 | n.a. | 2:1 | 109.1 | 0.610 | 34.6 | 35.0 |
| DOPS:CHOL | 5.5:4.5 | 6:1 | 2:1 | 193.4 | 0.131 | −38.4 | −45.5 |
| DOPG:CHOL | 5.5:4.5 | 6:1 | 2:1 | 196.0 | 0.282 | −49.9 | −43.7 |
| CHEMS:DOPE | 3:7 | 4:1 | 2:1 | 140.4 | −0.012 | 26.0 | −42.0 |
| CHEMS:DOPE:DSPE- | 3:6:1 | 4:1 | 2:1 | 125.6 | 0.344 | −14.9 | −35.1 |
| PEG_{5K} | | | | | | | |
| CHEMS:DOPE:DSPE- | 3:6:1 | 4:1 | 2:1 | 596.4 | 0.972 | N.D. | N.D. |
| PEG_{5K}-RGD | | | | | | | |
| CHEMS:DOPE:DSPE- | 3:6:1 | 4:1 | 2:1 | 906.7 | 1.611 | N.D. | N.D. |
| PEG_{5K}-LHRH | | | | | | | |
| DOPS:CHOL | 5.5:4.5 | 6:1 | 2:1 | 216.2 | −0.038 | N.D. | N.D. |
| DOPS:CHOL:DSPE- | 5.5:3.5:1 | 6:1 | 2:1 | 201.2 | 0.251 | N.D. | N.D. |
| PEG_{5K} | | | | | | | |
| DOPS:CHOL:DSPE- | 5.5:3.5:1 | 6:1 | 2:1 | 614.0 | 1.050 | N.D. | N.D. |
| PEG_{5K}-RGD | | | | | | | |
| DOPS:CHOL:DSPE- | 5.5:3.5:1 | 6:1 | 2:1 | 579.7 | 0.535 | N.D. | N.D. |
| PEG_{5K}-LHRH | | | | | | | |

N.D. = not determined.;
Prot = protamine sulfate USP;
Polydis = size polydispersity.
CHEMS formulations can not be generated using choesterol as helper lipid, in these particular experiments DOPE was selected because its fusogenic capacity. Control cationic LPDs were prepared at a 6:1 nmol DOTAP:ug DNA and 2:1 protamine DNA ratio

TABLE 13
DNA concentration titration in anionic DLPD: effect of DNA
concentration on the DLPD mean diameter.

| DNA | DOPS:CHOL | | DOPS:CHOL:DSPE- | | | | DOPS:CHOL:DSPE- | |
|---|---|---|---|---|---|---|---|---|
| Conc. | Size | | PEG_{5K} | | DOPG:CHOL: | | PEG_{5K} | |
| (ug/ml) | (nm) | poly | Size (nm) | poly | Size (nm) | poly | Size (nm) | poly |
| 75.0 | 156.3 | 0.259 | 147.5 | 0.694 | 159.3 | 0.388 | 197.5 | 0.526 |
| 100.0 | 211.9 | 0.253 | 179.1 | 0.626 | 211.4 | 0.528 | 179.0 | 0.486 |
| 125.0 | 253.6 | 0.137 | 178.1 | 0.412 | 260.7 | 0.445 | 193.8 | 0.26 |

| | | | | | | |
|---|---|---|---|---|---|---|
| 150.0 | aggregate | 216.3 | 0.163 | 296.0 | 0.425 | aggregate |

TABLE 14
FACS analysis representing the percentage of
cell binding for anionic DLPD Di-I labeled after
1 h incubation with MDA-MB-231 cells.

| Lipid mol | | |
|---|---|---|
| ratio | | MDA-MB-231 |
| 1:1 | DOTAP:CHOL | 72.8% |
| 55:45 | DOPS:CHOL | 14.32% |
| 55:45 | DOPG:CHOL | 35.35% |
| 3:7 | CHEMS:DOPE | 31.97% |

TABLE 15
Anionic DLPD mean diameter in an unimodal mode and
polydispersity for NC₁₂-DOPE:CHOL,
NC₁₂-DOPE:DOPE and NC₁₂-
DOPE:DOPC formulations.

| | size | |
|---|---|---|
| | (nm) | poly |
| NC12-DOPE:CHOL 4:1 | 162.7 | −0.093 |
| NC12-DOPE:CHOL 6:1 | 193.0 | −0.052 |
| NC12-DOPE:CHOL 8:1 | 130.7 | 0.427 |
| NC12-DOPE:DOPE 4:1 | 143.3 | 0.162 |
| NC12-DOPE:DOPE 6:1 | 113.6 | 0.476 |
| NC12-DOPE:DOPE 8:1 | 168.2 | 0.120 |
| NC12-DOPE:DOPC 4:1 | 126.8 | 0.227 |
| NC12-DOPE:DOPC 6:1 | 116.4 | 0.479 |
| NC12-DOPE:DOPC 8:1 | 115.6 | 0.698 |

TABLE 16
Cationic polymer condensed DNA particles mean diameter in
unimodal mode and particles polydispersity

| | size | |
|---|---|---|
| | (nm) | poly |
| Protamine DNA 2:1 | 152.5 | 0.250 |
| PEI DNA 2:1 | 183.3 | 1.463 |
| Eudragit EPO DNA 2:1 | 141.2 | 0.601 |
| Eudragit E100 DNA 2:1 | 174.0 | 0.160 |
| PMOETMAB DNA 2:1 | 329.5 | 0.077 |
| Spermidine DNA 2:1 | 2442.7 | −4.570 |
| Protamine DNA 3.5:1 | 127.4 | 0.288 |
| PEI DNA 3.5:1 | 680.2 | 2.223 |
| Eudragit EPO DNA 3.5:1 | 144.5 | −0.512 |
| Eudragit E100 DNA 3.5:1 | 76.1 | 3.528 |
| PMOETMAB DNA 3.5:1 | 246.4 | 0.347 |
| Spermidine DNA 3.5:1 | 1854.0 | −0.610 |

TABLE 17
DLPD mean diameter in an
unimodal mode and polydispersity values.

| | | size (nm) | poly |
|---|---|---|---|
| CHEMS:DOPE | Protamine DNA 2:1 | 111.6 | 0.296 |
| CHEMS:DOPE:DSPE-PEG_{5K} | | 119.6 | 0.743 |
| CHEMS:DOPE | PEI DNA 2:1 | Agg | Agg |
| CHEMS:DOPE:DSPE-PEG_{5K} | | 105.1 | 0.533 |
| CHEMS:DOPE | Eudragit EPO | 131.7 | 0.462 |
| | DNA 2:1 | | |
| CHEMS:DOPE:DSPE-PEG_{5K} | | 190.5 | 0.966 |
| CHEMS:DOPE | Eudragit E100 | Agg | Agg |
| | DNA 2:1 | | |
| CHEMS:DOPE:DSPE-PEG_{5K} | | Agg | Agg |
| CHEMS:DOPE | PMOETMAB | 202.0 | 0.903 |
| | DNA 2:1 | | |
| CHEMS:DOPE:DSPE-PEG_{5K} | | 280.4 | 0.976 |
| CHEMS:DOPE | Spermidine | 114.5 | 0.171 |
| | DNA 2:1 | | |
| CHEMS:DOPE:DSPE-PEG_{5K} | | 102.9 | 0.769 |
| CHEMS:DOPE | Protamine | 182.3 | 0.212 |
| | DNA 3.5:1 | | |
| CHEMS:DOPE:DSPE-PEG_{5K} | | 121.3 | 0.703 |
| CHEMS:DOPE | PEI DNA 3.5:1 | Agg | Agg |
| CHEMS:DOPE:DSPE-PEG_{5K} | | 108.4 | 0.296 |
| CHEMS:DOPE | Eudragit EPO | 162.3 | 0.393 |
| | DNA 3.5:1 | | |
| CHEMS:DOPE:DSPE-PEG_{5K} | | 158.7 | 0.882 |
| CHEMS:DOPE | Eudragit E100 | Agg | Agg |
| | DNA 3.5:1 | | |
| CHEMS:DOPE:DSPE-PEG_{5K} | | 98.5 | 0.678 |
| CHEMS:DOPE | PMOETMAB | 158.6 | 0.377 |
| | DNA 3.5:1 | | |
| CHEMS:DOPE:DSPE-PEG_{5K} | | 162.6 | 0.623 |
| CHEMS:DOPE | Spermidine | Agg | Agg |
| | DNA 3.5:1 | | |
| CHEMS:DOPE:DSPE-PEG_{5K} | | Agg | Agg |

DLPD were prepared at 38:2:1 of nanomoles of anionic lipid:μg Protamine:μg DNA ratio and DNA concentration of 75 μg/ml for CHEMS:DOPE and CHEMS:DOPE: 10 mol % DSPE-PEG_{5K} based formulation.
Agg = aggregation before dialysis.

TABLE 18

Condensed DNA mean diameter in an unimodal
mode and polydispersity.

| | |
|---|---|
| size | |
| (nm) | poly |

| | | |
|---|---|---|
| Protamine DNA 2:1 | 121.2 | −0.101 |
| PEI DNA 2:1 | 89.6 | 5.565 |
| Eudragit EPO DNA 2:1 | 158.6 | 0.249 |
| Eudragit E100 DNA 2:1 | 110.6 | 0.657 |
| PMOETMAB DNA 2:1 | 75.2 | 1.756 |
| RRRRRRRH (SEQ ID NO:44) DNA 2:1 | 111.5 | 0.148 |
| KHKHKHKHKGKHKHKHKHK | 25.1 | 9.265 |
| (SEQ ID NO:45) DNA 2:1 | | |

TABLE 19
DLPD mean diameter in an unimodal mode and polydispersity.

| | | size | | Zeta | Zeta |
|---|---|---|---|---|---|
| | | (nm) | poly | (mV) | Err |
| CHEMS:DOPE | Protamine | 180.9 | 0.373 | −47.6 | 0.7 |
| | DNA 2:1 | | | | |
| CHEMS:DOPE: 0.5% DSPE-PEG_{5K} | | 135.4 | 0.436 | −21.0 | 1.7 |
| CHEMS:DOPE: 0.5% DSPE-PEG_{5K}-Folate | | 152.6 | 0.475 | −21.9 | 0.3 |
| NC12DOPE:DOPE | | 181.1 | 0.479 | −45.3 | 2.0 |
| NC12DOPE:DOPE: 0.5% DSPE-PEG | | 164.1 | 0.616 | −47.7 | 1.0 |
| NC12DOPE:DOPE: 0.5% DSPE-PEG- | | 173.9 | 0.385 | −46.1 | 0.6 |
| Folate | | | | | |
| CHEMS:DOPE | PEI DNA | 222.1 | 0.180 | −46.2 | 0.4 |
| | 2:1 | | | | |
| CHEMS:DOPE: 0.5% DSPE-PEG_{5K} | | 114.2 | 0.270 | −21.2 | 0.5 |
| CHEMS:DOPE: 0.5% DSPE-PEG_{5K}-Folate | | 154.0 | 0.127 | −28.0 | 0.6 |
| NC12DOPE:DOPE | | 204.1 | 0.194 | −49.9 | 0.6 |
| NC12DOPE:DOPE: 0.5% DSPE-PEG | | 178.5 | 0.374 | −40.3 | 0.5 |
| NC12DOPE:DOPE: 0.5% DSPE-PEG- | | 192.2 | 0.482 | −37.4 | 0.4 |
| Folate | | | | | |
| CHEMS:DOPE | Eudragit | 193.4 | 0.390 | −40.0 | 1.0 |
| | EPO DNA | | | | |
| | 2:1 | | | | |
| CHEMS:DOPE: 0.5% DSPE-PEG_{5K} | | 159.0 | 0.322 | −19.0 | 0.7 |
| CHEMS:DOPE: 0.5% DSPE-PEG_{5K}-Folate | | 256.3 | 0.078 | −18.8 | 0.2 |
| NC12DOPE:DOPE | | 248.0 | 0.796 | −52.1 | 0.3 |
| NC12DOPE:DOPE: 0.5% DSPE-PEG | | 117.1 | 2.932 | −48.1 | 0.7 |
| NC12DOPE:DOPE: 0.5% DSPE-PEG- | | 240.6 | 0.579 | −48.1 | 0.7 |
| Folate | | | | | |
| CHEMS:DOPE | Eudragit | 191.5 | 0.325 | −40.8 | 1.1 |
| | E100 DNA | | | | |
| | 2:1 | | | | |
| CHEMS:DOPE: 0.5% DSPE-PEG_{5K} | | 1639. | 0.240 | −19.9 | 0.5 |
| CHEMS:DOPE: 0.5% DSPE-PEG_{5K}-Folate | | 253.7 | 0.013 | −18.4 | 0.7 |
| NC12DOPE:DOPE | | 259.8 | 0.93 | −54.5 | 2.1 |
| NC12DOPE:DOPE: 0.5% DSPE-PEG | | 213.6 | 0.738 | −48.0 | 0.9 |
| NC12DOPE:DOPE: 0.5% DSPE-PEG- | | 239.5 | 0.835 | −48.0 | 0.9 |
| Folate | | | | | |
| CHEMS:DOPE | PMOETM | 209.1 | 0.666 | −42.9 | 0.8 |
| | AB DNA | | | | |
| | 2:1 | | | | |
| CHEMS:DOPE: 0.5% DSPE-PEG_{5K} | | 278.0 | 0.635 | −15.3 | 0.8 |
| CHEMS:DOPE: 0.5% DSPE-PEG_{5K}-Folate | | 112.0 | 0.660 | −19.2 | 1.0 |
| NC12DOPE:DOPE | | 239.8 | 0.546 | −49.3 | 0.5 |
| NC12DOPE:DOPE: 0.5% DSPE-PEG | | 207.7 | 0.524 | −35.2 | 0.7 |
| NC12DOPE:DOPE: 0.5% DSPE-PEG- | | 259.3 | 0.553 | −40.8 | 0.8 |
| Folate | | | | | |
| CHEMS:DOPE | RRRRRRRH | 203.3 | 0.506 | −38.5 | 1.2 |
| | (SEQ ID | | | | |
| | NO: 44) | | | | |
| | DNA 2:1 | | | | |
| CHEMS:DOPE: 0.5% DSPE-PEG_{5K} | | 145.5 | 0.626 | −14.7 | 0.7 |
| CHEMS:DOPE: 0.5% DSPE-PEG_{5K}-Folate | | 173.3 | 0.802 | −16.5 | 0.4 |
| NC12DOPE:DOPE | | 199.1 | 0.378 | −40.7 | 1.4 |
| NC12DOPE:DOPE: 0.5% DSPE-PEG | | 204.5 | 0.855 | −36.6 | 0.9 |
| NC12DOPE:DOPE: 0.5% DSPE-PEG- | | 197.6 | 0.662 | −39.0 | 0.3 |
| Folate | | | | | |
| CHEMS:DOPE | KHKHKHKH | 192.1 | 0.460 | −36.6 | 0.6 |
| | KGKHKHKH | | | | |
| | KHK | | | | |
| | (SEQ ID | | | | |
| | NO: 45) | | | | |
| CHEMS:DOPE: 0.5% DSPE-PEG_{5K} | | 153.8 | 1.055 | −14.1 | 0.6 |
| CHEMS:DOPE: 0.5% DSPE-PEG_{5K}-Folate | | 132.8 | 0.760 | −18.6 | 0.9 |
| NC12DOPE:DOPE | | 203.5 | 0.527 | −49.5 | 2.5 |
| NC12DOPE:DOPE: 0.5% DSPE-PEG | | 187.9 | 0.610 | −39.5 | 1.4 |
| NC12DOPE:DOPE: 0.5% DSPE-PEG- | | 187.6 | 0.462 | −38.0 | 1.4 |
| Folate | | | | | |
| Table 19 legend: | | | | | |
| DLPD were prepared at 129 nanomoles total lipid 2 μg cationic polymer: 1 μg DNA ratio for CHEMS:DOPE and 156 nanomoles total lipid 2 μg cationic polymer: 1 μg DNA For the NC₁₂-DOPE:DOPE formulation. The DNA concentration for both DLPD formulation was 75 ug/ml. Zeta potential measurements were realized in 20 mM HEPES buffer pH 7.5 SD for the zeta potential have been calculated based on five readings from the same sample. DSPE-PEG_{5K} or DSPE-PEG_{5K}- | | | | | |
| # Folate were incorporated at 0.5 mole % into the LPD formulation. | | | | | |

TABLE 20
Effect of pH on anionic DLPD zeta potential in HEPES 20 mM,
DLPD formulated with PEI compacted DNA at 2:1 μg; μg ratio.

| | Size | | Zeta | Err |
|---|---|---|---|---|
| | nm | poly | (mV) | Zeta |
| CHEMS:DOPE at pH 7.5 | 222.1 | 0.180 | −41.6 | 0.7 |
| CHEMS:DOPE at pH 4.2 | n.d. | n.d. | +17.1 | 0.3 |
| NC12-DOPE:DOPE at pH 7.5 | 204.1 | 0.194 | −46.4 | 1.0 |
| NC12-DOPE:DOPE at pH 4.2 | n.d. | n.d. | −43.4 | 0.9 |

TABLE 21
Incorporation of PPAA into LPD
Effect on particle mean diameter in an unimodal mode and zeta potential.

| | PPAA/ | | | Zeta | Zeta | |
|---|---|---|---|---|---|---|
| | DNA | LPD | | (mV) | (mV) | Trans |
| | charge ratio | (nm) | poly | pH 7.5 | pH 4.2 | enhan* |
| DOTAP:CHOL LPD 12:1:1 no | 0 | 186.1 | 0.44 | +30.3 +/− 0.5 | 36.0 +/− 1.1 | 1.0 |
| PPAA | | | | | | |
| PPAA added to compacted DNA | | | | | | |
| DOTAP:CHOL LPD 12:1:1 0.75 μg | 0.25 (+) | Agg. | Agg. | Agg. | Agg. | n.d. |
| PPAA/μg DNA | | | | | | |
| DOTAP:CHOL LPD 12:1:1 1.5 μg | 0.5 (+) | Agg. | Agg. | Agg. | Agg. | n.d. |
| PPAA/μg DNA | | | | | | |
| DOTAP:CHOL LPD 12:1:1 3 μg | neutral | 421.0 | −0.06 | −22.4 +/− 0.9 | +19.5 +/− 1.3 | 10.2 |
| PPAA/μg DNA | | | | | | |
| DOTAP:CHOL LPD 12:1:1 6 μg | 1 (−) | 472.1 | 0.089 | −33.8 +/− 0.8 | +3.5 +/− 0.4 | 0.85 |
| PPAA/μg DNA | | | | | | |
| DOTAP:CHOL LPD 12:1:1 9 μg | 2 (−) | 519.2 | 0.541 | −35.2 +/− 1.1 | −9.8 +/− 0.8 | 0.18 |
| PPAA/μg DNA | | | | | | |
| DOTAP:CHOL LPD 12:2:1 1 μg | 0.066 (+) | Agg. | Agg. | Agg. | Agg. | n.d. |
| PPAA/μg DNA | | | | | | |
| DOTAP:CHOL LPD 12:2:1 3.75 μug | 0.25 (+) | Agg. | Agg. | Agg. | Agg. | n.d. |
| PPAA/μg DNA | | | | | | |
| DOTAP:CHOL LPD 12:2:1 7.5 μg | 0.5 (+) | 428.7 | 0.033 | −33.4 +/− 0.1 | +14.9 +/− 0.8 | 0.04 |
| PPAA/μg DNA | | | | | | |
| DOTAP:CHOL LPD 12:2:1 15 μg | 0 | 289.7 | 0.322 | −34.4 +/− 0.9 | +13.4 +/− 0.7 | 0.00 |
| PPAA/ug DNA | | | | | | |
| DOTAP:CHOL LPD 12:2:1 30 μg | 1 (−) | Agg. | Agg. | Agg. | Agg. | n.d. |
| PPAA/μg DNA | | | | | | |
| DOTAP:CHOL LPD 12:2:1 45 μg | 2 (−) | n.d. | n.d. | n.d. | n.d. | n.d. |
| PPAA/μg DNA | | | | | | |
| PPAA added to complete LPD | | | | | | |
| DOTAP:CHOL LPD 12:1:1 no | 0 | 183.2 | 0.434 | +19.5 +/− 1.0 | +39.5 +/− 1.3 | 1.0 |
| PPAA | | | | | | |
| DOTAP:CHOL LPD 12:1:1 0.75 μg | 0.25 (+) | Agg. | Agg. | Agg. | Agg. | n.d. |
| PPAA/μg DNA | | | | | | |
| DOTAP:CHOL LPD 12:1:1 1.5 μg | 0.5 (+) | Agg. | Agg. | Agg. | Agg. | n.d. |
| PPAA/μg DNA | | | | | | |
| DOTAP:CHOL LPD 12:1:1 3 μg | neutral | Agg. | Agg. | Agg. | Agg. | n.d. |
| PPAA/μg DNA | | | | | | |
| DOTAP:CHOL LPD 12:1:1 6 μg | 1 (−) | 412.4 | 0.220 | −34.1 +/− 0.5 | +9.2 +/− 1.2 | 4.88 |
| PPAA/μg DNA | | | | | | |
| DOTAP:CHOL LPD 12:1:1 9 μg | 2 (−) | 438.6 | 0.313 | −36.5 +/− 0.6 | −5.9 +/− 0.3 | 0.39 |
| PPAA/μg DNA | | | | | | |
| DOTAP:CHOL LPD 12:2:1 1 μg | 0.066 (+) | 3758.1 | −46.5 | +23.3 +/− 1.3 | 40.2 +/− 1.5 | 2.96 |
| PPAA/μg DNA | | | | | | |
| DOTAP:CHOL LPD 12:2:1 3.75 μg | 0.25 (+) | 389.7 | −0.42 | −22.2 +/− 1.3 | +33.0 +/− 1.1 | 11.3 |
| PPAA/μg DNA | | | | | | |
| DOTAP:CHOL LPD 12:2:1 7.5 μg | 0.5 (+) | 396.7 | 0.256 | −33.7 +/− 0.7 | +17.5 +/− 0.5 | 2.24 |
| PPAA/μg DNA | | | | | | |
| DOTAP:CHOL LPD 12:2:1 15 μg | 0 | 309.7 | 0.187 | −36.6 +/− 0.7 | −9.3 +/− 0.3 | 0.22 |
| PPAA/μg DNA | | | | | | |
| DOTAP:CHOL LPD 12:2:1 30 μg | 1 (−) | n.d. | n.d. | n.d. | n.d. | n.d. |
| PPAA/μg DNA | | | | | | |
| DOTAP:CHOL LPD 12:2:1 45 μg | 2 (−) | n.d. | n.d. | n.d. | n.d. | n.d. |
| PPAA/μg DNA | | | | | | |

LPD were prepared at 12:1:1 ratio (nmol lipid; μg Protamine:μg DNA). Final DNA concentration in LPD formulation was 150 μg DNA/ml.
SD for the zeta potential have been calculated based on five readings from the same sample.
Agg. = LPD aggregation.
n.d. = not determined.

TABLE 22
Effect on Particle Mean Diameter of Incorporation of PPAA into LPDs.

| | LPD | | | | Zeta | Zeta |
|---|---|---|---|---|---|---|
| | (nm) | | | | (mV) | (mV) |
| | 20 mM | | LPD | | pH 7.2 | pH 4.2 |
| | HEPES | | (nm) | | 20 mM | 20 mM |
| | pH 7.2 | poly | serum | poly | HEPES | HEPES |
| Serum alone | | | 345.7 | 0.837 | −24.0 +/− 0.7 | |
| DOTAP:CHOL LPD 12:1:1 no | 200.9 | 0.711 | 982.0 | 0.744 | 18.8 +/− 1 | 54.7 +/− 2.1 |
| PPAA | | | | | | |
| DOTAP:CHOL:10% DSPE- | 127.6 | 0.557 | 262.3 | 0.856 | 2.9 +/− 0.6 | 10.5 +/− 1.3 |
| PEG_{5K} LPD 12:1:1 no PPAA | | | | | | |
| DOTAP:CHOL:10% DSPE- | 152.1 | 0.364 | 286.4 | 0.761 | −0.2 +/− 0.4 | 4.9 +/− 0.7 |
| PEG_{5K}-Folate LPD 12:1:1 no | | | | | | |
| PPAA | | | | | | |
| DOTAP:CHOL:2% DSPE- | 143.8 | 0.122 | 255.8 | 0.720 | 1.9 +/− 0.3 | 8.8 +/− 0.9 |
| PEG_{5K} LPD 12:1:1 no PPAA | | | | | | |
| DOTAP:CHOL:2% DSPE- | 153.5 | 0.414 | 282.5 | 0.685 | 0.8 +/− 0.8 | 9.1 +/− 0.8 |
| PEG_{5K}-Folate LPD 12:1:1 no | | | | | | |
| PPAA | | | | | | |
| PPAA added to compacted | | | | | | |
| DNA | | | | | | |
| DOTAP:CHOL LPD 12:1:1 3 μg | 823.5 | 0.647 | 859.9 | 0.133 | −27.2 +/− 1.0 | 33.9 +/− 0.8 |
| PPAA/μg DNA | | | | | | |
| DOTAP:CHOL:10% DSPE- | 265.4 | 0.780 | 272.7 | 0.683 | −8.4 +/− 0.8 | 2.9 +/− 0.4 |
| PEG_{5K} LPD 12:1:1 3 μg | | | | | | |
| PPAA/μg DNA | | | | | | |
| DOTAP:CHOL:10% DSPE- | 273.4 | 0.523 | 296.6 | 0.819 | −12.5 +/− 0.6 | 2.6 +/− 0.5 |
| PEG_{5K}-Folate LPD 12:1:1 3 μg | | | | | | |
| PPAA/μg DNA | | | | | | |
| DOTAP:CHOL:2% DSPE- | 224.7 | 0.215 | 284.7 | 0.793 | −8.1 +/− 0.4 | 5.7 +/− 0.7 |
| PEG_{5K} LPD 12:1:1 3 μg | | | | | | |
| PPAA/μg DNA | | | | | | |
| DOTAP:CHOL:2% DSPE- | 319.1 | 0.211 | 403.9 | 0.751 | −11.4 +/− 0.4 | 7.4 +/− 0.6 |
| PEG_{5K}-Folate LPD 12:1:1 3 μg | | | | | | |
| PPAA/μg DNA | | | | | | |

SD for the zeta potential have been calculated based on five readings from the same sample. All zeta potential measurement were realized in absence of serum.

TABLE 23
FACS analysis representing the mean fluorescent intensity of KB cells
after 1 h incubation at 37° C. with targeted LPD Di-I labeled.

| | | |
|---|---|---|
| | | 3 ug |
| | | PPAA/ug |
| Lipid mol | | protamine- |
| ratio | No PPAA | DNA |

| | | | |
|---|---|---|---|
| DOTAP:CHOL | 1:1 | 1232.13 | 467.21 |
| DOTAP:CHOL:DSPE-PEG_{5K} | 1:0.98:0.02 | 988.44 | 218.28 |
| DOTAP:CHOL:DSPE-PEG_{5K}- | 1:0.98:0.02 | 1173.47 | 504.77 |
| Folate | | | |

n.d. = not determined, Typically negative control for all cell line evaluated were showing less than 2% positive cells.

TABLE 24
Mean Diamter and Zeta Potential for PPAA-containing LPDs with
Various % Ratios of DSPE-PEG

| | | | Zeta | Zeta |
|---|---|---|---|---|
| | | | (mV) pH 7.5 | (mV) pH 4.2 |
| | LPD | | 20 mM | 20 mM |
| | (nm) | poly | HEPES | HEPES |
| Protamine:DNA 1:1 | 78.6 | 0.267 | 13.0 +/− 1.8 | 8.5 +/− 0.5 |
| Protamine:DNA+ PPAA 1:1:3 | 992.6 | 1.882 | −23.3 +/− 0.9 | −8.4 +/− 0.6 |
| DOTAP:CHOL LPD 12:1:1 no PPAA | 223.3 | 0.902 | 31.8 +/− 1.6 | 35.0 +/− 0.9 |
| DOTAP:CHOL:2% DSPE-PEG_{5K} LPD | 130.7 | 0.285 | 3.3 +/− 1.5 | 5.2 +/− 0.6 |
| 12:1:1 no PPAA | | | | |
| DOTAP:CHOL:2% DSPE-PEG_{5K}-Folate | 172.0 | 0.241 | 5.5 +/− 0.5 | 6.9 +/− 0.4 |
| LPD 12:1:1 no PPAA | | | | |
| DOTAP:CHOL:8% DSPE-PEG_{2K}+ 2% | 132.9 | 0.137 | n.d. | n.d. |
| DSPE-PEG_{5K}-folate LPD 12:1:1 no PPAA | | | | |
| DOTAP:CHOL:5% DSPE-PEG_{2K}+ 2% | 131.6 | 0.267 | n.d. | n.d. |
| DSPE-PEG_{5K}-folate LPD 12:1:1 no PPAA | | | | |
| DOTAP:CHOL:2% DSPE-PEG_{2K}+ 2% | 179.5 | 0.571 | n.d. | n.d. |
| DSPE-PEG_{5K}-folate LPD 12:1:1 no PPAA | | | | |
| DOTAP:CHOL:8% DSPE-PEG_{2K}+ 2% | 165.3 | 0.260 | n.d. | n.d. |
| DSPE-PEG_{5K} LPD 12:1:1 no PPAA | | | | |
| DOTAP:CHOL:5% DSPE-PEG_{2K}+ 2% | 248.1 | 0.780 | n.d. | n.d. |
| DSPE-PEG_{5K} LPD 12:1:1 no PPAA | | | | |
| DOTAP:CHOL:2% DSPE-PEG_{2K}+ 2% | 152.0 | 0.309 | n.d. | n.d. |
| DSPE-PEG_{5K} LPD 12:1:1 no PPAA | | | | |
| DOTAP:CHOL:2% DSPE-PEG_{2K}+ 8% | 122.0 | 0.365 | n.d. | n.d. |
| DSPE-PEG_{5K}-Folate LPD 12:1:1 no PPAA | | | | |
| DOTAP:CHOL:2% DSPE-PEG_{2K}+ 8% | 147.1 | 0.416 | n.d. | n.d. |
| DSPE-PEG_{5K} LPD 12:1:1 no PPAA | | | | |
| PPAA added to compacted DNA | | | | |
| DOTAP:CHOL LPD 12:1:1 3 μg PPAA/μg | 501.5 | −.0547 | −24.5 +/− 0.3 | 42.6 +/− 4.2 |
| protamine-DNA | | | | |
| DOTAP:CHOL:2% DSPE-PEG_{5K} LPD | 217.7 | 0.652 | −15.5 +/− 1.1 | 6.4 +/− 0.7 |
| 12:1:1 3 μg PPAA/μg protamine-DNA | | | | |
| DOTAP:CHOL:2% DSPE-PEG_{5K}-Folate | 320.3 | 0.402 | −12.1 +/− 1.9 | 8.03 +/− 0.3 |
| LPD 12:1:1 3 μg PPAA/μg protamine- | | | | |
| DNA | | | | |
| DOTAP:CHOL:8% DSPE- | 210.6 | 0.367 | −12.5 +/− 0.6 | 5.3 +/− 0.8 |
| PEG_{2K}+ 2% DSPE-PEG_{5K}-folate | | | | |
| LPD 12:1:1 3 μg PPAA/μg protamineDNA | | | | |
| DOTAP:CHOL:5% DSPE- | 213.7 | 0.588 | −11.6 +/− 0.3 | 5.6 +/− 0.5 |
| PEG_{2K}+ 2% DSPE-PEG_{5K}-folate | | | | |
| LPD 12:1:1 3 μg PPAA/μg protamineDNA | | | | |
| DOTAP:CHOL:2% DSPE-PEG_{2K} 2% | 401.4 | 0.505 | −9.5 +/− 0.4 | 10.0 +/− 0.7 |
| DSPE-PEG_{5K}-folate | | | | |
| LPD 12:1:1 3 μg PPAA/μg protamineDNA | | | | |
| DOTAP:CHOL:8% DSPE-PEG_{2K}+ 2% | 287.3 | 0.782 | −13.2 +/− 0.7 | 6.6 +/− 0.6 |
| DSPE-PEG_{5K} LPD 12:1:1 3 μg PPAA/μg | | | | |
| protamine-DNA | | | | |
| DOTAP:CHOL:5% DSPE-PEG_{2K}+ 2% | 342.0 | 0.536 | −11.9 +/− 0.9 | 6.3 +/− 0.3 |
| DSPE-PEG_{5K} LPD 12:1:1 3 μg PPAA/μg | | | | |
| protamine-DNA | | | | |
| DOTAP:CHOL:2% DSPE-PEG_{2K}+ 2% | 240.7 | 0.458 | −10.9 +/− 0.6 | 4.4 +/− 0.4 |
| DSPE-PEG_{5K} LPD 12:1:1 3 μg PPAA/μg | | | | |
| protamine-DNA | | | | |
| DOTAP:CHOL:2% DSPE-PEG_{2K}+ 8% | 247.4 | 0.466 | −10.8 +/− 0.3 | 6.8 +/− 0.7 |
| DSPE-PEG_{5K}-Folate | | | | |
| LPD 12:1:1 3 μg PPAA/μg protamine-DNA | | | | |
| DOTAP:CHOL:2% DSPE-PEG_{2K}+ 8% | 264.9 | 0.713 | −13.1 +/− 1.0 | 3.9 +/− 0.4 |
| DSPE-PEG_{5K} | | | | |
| LPD 12:1:1 3 μg PPAA/μg protamineDNA | | | | |

Agg = LPD were aggregated,
n.d. = not determined.
*= Transfection enhancement over the same LPD formulation with out PPAA, transfection were realized in KB cells

TABLE 25
LPD mean diameter in an unimodal mode and Polydispersity.
LPD were prepared at 12:1:1 ratio DNA concentration of 150 μg
DNA/ml and 1.8 mM lipid concentration.

| | | LPD | |
|---|---|---|---|
| # | | (nm) | poly |
| 1 | DOTAP:CHOL + 3.0 μg PPAA/μg DNA | 438.0 | 0.830 |
| 2 | DOTAP:CHOL:DSPE-PEG_{5K} 2% + 3.0 μg | 278.3 | 0.368 |
| | PPAA/μg DNA | | |
| 3 | DOTAP:CHOL:DSPE-PEG_{5K} 10% + 3.0 μg | 252.9 | 0.478 |
| | PPAA/μg DNA | | |
| 4 | DOTAP:CHOL:DSPE-PEG_{5K}-Folate 2% + 3.0 μg | 305.8 | 0.215 |
| | PPAA/μg DNA | | |
| 5 | DOTAP:CHOL:DSPE-PEG_{5K}-Folate 10% + 3.0 μg | 293.0 | 0.532 |
| | PPAA/μg DNA | | |
| 6 | DOTAP:CHOL + 0.0 μg PPAA/μg DNA | 215.7 | 0.341 |
| 7 | DOTAP:CHOL:DSPE-PEG_{5K} 2% + 0.0 μg | 203.2 | 0.361 |
| | PPAA/μg DNA | | |
| 8 | DOTAP:CHOL:DSPE-PEG_{5K} 10% + 0.0 μg | 276.6 | 0.746 |
| | PPAA/μg DNA | | |
| 9 | DOTAP:CHOL:DSPE-PEG_{5K}-Folate 2% + 0.0 μg | 209.9 | 0.163 |
| | PPAA/μg DNA | | |
| 10 | DOTAP:CHOL:DSPE-PEG_{5K}-Folate 10% + 0.0 μg | 222.7 | 0.454 |
| | PPAA/μg DNA | | |
| 11 | DOTAP:CHOL + 2.5 μg PPAA/μg DNA | 509.8 | 0.690 |
| 12 | DOTAP:CHOL:DSPE-PEG_{5K} 2% + 2.5 μg | 268.2 | 0.199 |
| | PPAA/μg DNA | | |
| 13 | DOTAP:CHOL:DSPE-PEG_{5K} 10% + 2.5 μg | 252.8 | 0.359 |
| | PPAA/μg DNA | | |
| 14 | DOTAP:CHOL:DSPE-PEG_{5K}-Folate 2% + 2.5 μg | 290.7 | 0.293 |
| | PPAA/μg DNA | | |
| 15 | DOTAP:CHOL:DSPE-PEG_{5K}-Folate 10% + 2.5 μg | 240.6 | 0.370 |
| | PPAA/μg DNA | | |
| 16 | DOTAP:CHOL + 2.0 μg PPAA/μg DNA | 819.1 | 0.181 |
| 17 | DOTAP:CHOL:DSPE-PEG_{5K} 2% + 2.0 μg | 717.5 | 0.115 |
| | PPAA/μg DNA | | |
| 18 | DOTAP:CHOL:DSPE-PEG_{5K} 10% + 2.0 μg | 563.4 | 0.498 |
| | PPAA/μg DNA | | |
| 19 | DOTAP:CHOL:DSPE-PEG_{5K}-Folate 2% + 2.0 μg | 1245.4 | 0.984 |
| | PPAA/μg DNA | | |
| 20 | DOTAP:CHOL:DSPE-PEG_{5K}-Folate 10% + 2.0 μg | 328.0 | 0.358 |
| | PPAA/μg DNA | | |
| 21 | DOTAP:CHOL + 1.5 μg PPAA/μg DNA | 1352.1 | 1.318 |
| 22 | DOTAP:CHOL:DSPE-PEG_{5K} 2% + 1.5 μg | 629.9 | 0.695 |
| | PPAA/μg DNA | | |
| 23 | DOTAP:CHOL:DSPE-PEG_{5K} 10% + 1.5 μg | 366.2 | 0.053 |
| | PPAA/μg DNA | | |
| 24 | DOTAP:CHOL:DSPE-PEG_{5K}-Folate 2% + 1.5 μg | 1833.2 | 1.734 |
| | PPAA/μg DNA | | |
| 25 | DOTAP:CHOL:DSPE-PEG_{5K}-Folate 10% + 1.5 μg | 612.6 | 0.906 |
| | PPAA/μg DNA | | |
| 26 | DOTAP:CHOL + 1.0 μg PPAA/μg DNA | 1208.3 | 1.199 |
| 27 | DOTAP:CHOL:DSPE-PEG_{5K} 2% + 1.0 μg | 686.1 | 0.069 |
| | PPAA/μg DNA | | |
| 28 | DOTAP:CHOL:DSPE-PEG_{5K} 10% + 1.0 μg | 253.7 | 0.488 |
| | PPAA/μg DNA | | |
| 29 | DOTAP:CHOL:DSPE-PEG_{5K}-Folate 2% + 1.0 μg | 1005.1 | 1.256 |
| | PPAA/μg DNA | | |
| 30 | DOTAP:CHOL:DSPE-PEG_{5K}-Folate 10% + 1.0 μg | 402.9 | 0.325 |
| | PPAA/μg DNA | | |

TABLE 26
Effect of DSPE-PEG_{5K} and DSPE-PEG_{5K}-Folate Incorporation into
LPD Formulations on in vitro Complement Activation*

| Formulation | % CH50 Decrease | % Opsonization Level^{&} |
|---|---|---|
| LPD-PEG_{5K}, 2% | 88.7 ± 1.2 | 93.0 ± 2.2 |
| LPD-PEG_{5K}-folate, 2% | 96.5 ± 0.9 | 103.6 ± 1.7 |
| LPD-PEG_{5K}, 10% | 57.3 ± 12.4 | 60.7 ± 12.7 |
| LPD-PEG_{5K}-folate, 10% | 71.9 ± 9.6 | 77.1 ± 11.1 |

*N = 3
^{&}Unmodified LPDs defined as 100% opsonization.

TABLE 27
In vivo Tumor Progression for Mice treated with PEGylated LPD
formulations of Thymidine Kinase as a Model Therapeutic

| Group | Formulation | d56 Median Tumor Size |
|---|---|---|
| 1 | Untreated (no ganciclovir) | 1303 |
| 2 | Vehicle + Ganciclovir | 1250 |
| 3 | DCC-TK (*Intratumoral) | 402 |
| 4 | LPD-TK | 1250 |
| 5 | LPD-TK + 10% PEG | 616 |
| 6 | LPD-null + 10% PEG | 971 |
| 7 | DCC-TK (*Intratumoral) | 255 |
| 8 | LPD-TK | 782 |
| 9 | LPD-TK + 10% PEG | 345 |
| 10 | LPD-null + 10% PEG | 820 |

TABLE 28
In vivo Tumor Progression for Mice Treated with Folate-Targeted
PEGylated LPD formulations of Thymidine Kinase as a Model
Therapeutic

| Group | Formulation | d77 Median Tumor Size |
|---|---|---|
| 1 | Untreated (no ganciclovir) | 221 |
| 2 | Vehicle + Ganciclovir | 224 |
| 3 | DCC-TK (*Intratumoral) | 0 |
| 4 | LPD-TK | 42 |
| 5 | LPD-TK + 10% PEG | 109 |
| 6 | LPD-TK + 10% PEG | 37 |
| 7 | LPD-TK + 10% PEG | 59 |
| 8 | LPD-TK + 10% PEG-Folate | 18 |
| 9 | LPD-null + 10% PEG | 23 |
| 10 | LPD-null + 10% PEG-Folate | 142 |

TABLE 29
Size Data for Anionic LPDs for In vivo Biodistribution

| Formulation | nm | Polydispersity |
|---|---|---|
| CHEMS:DOPE (3:7) | 122.0 | 0.47 |
| CHEMS:DOPE:DSPE:PEG (3:6:1) | 141.2 | 0.59 |
| CHEMS:DOPE:DSPE:PEG:FOLATE (3:6:1) | 306.7 | 1.39 |

TABLE 30
CHEMS:DOPE Gene Expression in mice bearing SKOV3-ip-1
xenograft tumor model 16 h following intraveneous injection of
ALPD containing 75 μg DNA/mouse.

| | | N = 5 | N = 6 |
|---|---|---|---|
| | N = 4 | Chems: | Chems: |
| | Chems: | DOPE:DSPE- | DOPE:DSPE- |
| RLU/mg | DOPE(3:7) | PEG(3:6:1) | PEG-Folate(3:6:1) |
| Tumor | 1175 | 3045 | 5896 |
| Lung | 1639 | 3049 | 1020 |
| Heart | 740 | 2828 | 1415 |
| Liver | 176899 | 322169 | 391 |
| Spleen | 1753 | 2600 | 389 |

1. A lipid-nucleic acid complex comprising a compacted nucleic acid, a polycation, a targeting factor, and a lipid, wherein:
a) the targeting factor increases cellular bioavailability of the nucleic acid by a means other than interaction with a specific outer cell surface membrane receptor;
b) the complex does not comprise a protamine or a salt thereof; and
c) the mean diameter of the complex is greater than about 100 nm and less than 400 nm.

2. The complex of claim 1, wherein the targeting factor is a membrane disruptive polymer.

3. The complex of claim 1, wherein the mean diameter of the complex is about 300 nm or less.

4. The complex of claim 1, wherein the mean diameter of the complex is about 200 nm or less.

5. The complex of claim 1, further comprising a shielding agent.

6. The complex of claim 5, wherein the shielding agent increases circulatory half life of the complex, reduces binding of serum components to the complex, or reduces complement opsonization of the complex.

7. The complex of claim 5, wherein the shielding agent comprises polyethylene glycol (PEG).

8. The complex of claim 5, wherein the shielding agent is PEG.

9. The complex of claim 5, wherein the shielding agent comprises a pegylated lipid.

10. The complex of claim 1, wherein the polycation is a synthetic polycation, a polycationic polypeptide or salt thereof.

11. The complex of claim 10, wherein the polycation is a synthetic polycation.

12. The complex of claim 11, wherein the synthetic polycation is selected from the group consisting of polycationic methacryloxy polymers, polycationic methacrylate polymers and polycationic poly(alkenylimines).

13. The complex of 12, wherein the polycationic methacrylate polymer is comprised of dimethylamino methacrylate.

14. The complex of claim 11, wherein the synthetic polycation is selected from the group consisting of polyethyleneimine (PEI), poly(2-methacryloxyethyltrimethyl ammonium bromide) (PMOETMAB), and a co-polymer of dimethylamino methacrylate and methacrylic ester.

15. The complex of claim 1, wherein the targeting factor is a membrane disruptive synthetic polymer.

16. The complex of claim 1, wherein the targeting factor functions to increase cellular bioavailability by increasing transcription of the nucleic acid of the complex, by increasing uptake of the nucleic acid into the cell, by increasing uptake into a cellular compartment, by increasing exit of the nucleic acid from a cellular compartment, or by increasing transport of nucleic acid across a cell membrane.

17. The complex of claim 1, wherein the targeting factor is a membrane translocating peptide (MTLP).

18. The complex of 14, wherein the membrane translocating peptide is selected from the group consisting of H₂N-KKAAAVLLPVLLAAP-COOH (Elan094) (SEQ ID NO:2), H₂N-KKKAAAVLLPVLLAAP (ZElan094) (SEQ ID NO:3), H₂N-kkkaavllpvllaap (ZElan207) (SEQ ID NO:4), and H₂N-KKKAAAVLLPVLLAAPREDL (ZElan094R) (SEQ ID NO:5).

19. The complex of claim 1, wherein the targeting factor comprises a nuclear. localization sequence.

20. The complex of claim 19, wherein the nuclear localization sequence is SV 40 NLS.

21. The complex of claim 1, further comprising a co-lipid.

22. The complex of claim 1, wherein the targeting factor is conjugated to a PEG moiety.

23. The complex of any one of claims 1 to 17, or 19 to 22, wherein the lipid is a cationic lipid.

24. The complex of claim 23, wherein the cationic lipid is 1,2-bis(oleoyloxy)-3-trimethylammoniopropane (DOTAP).

25. The complex of claim 23, wherein the cationic lipid is DOTAP.

26. The complex of claim 23, wherein the co-lipid is selected from the group consisting of cholesterol, diphytanoyl phosphatidylethanolamine (DPHPE), dioleoyl phosphatidylethanolamine (DOPE), dioleoyl phosphatidylcholine (DOPC), dilauryl phosphatidylethanolamine (DLPE), 1,2-distearoyl-sn-glycero-3-phosphatidylethanolamine (DSPE), and dimyristoyl phosphatidylethanolamine (DPME).

27. A lipid-nucleic acid complex comprising a compacted nucleic acid and at least one lipid species that is fusogenic, wherein:
a) the complex has an aqueous core; and
b) the mean diameter of the complex is greater than about 100 nm and less than 400 nm.

28. A lipid-nucleic acid complex comprising a compacted nucleic acid, a polycation, a targeting factor and at least one lipid species, wherein:
a) the at least one lipid species is an anionic lipid;
b) the complex has an aqueous core;
c) the complex comprises at least one fusogenic moiety;
d) the mean diameter of the complex is greater than about 100 nm and less than 400 nm; and,
wherein the complex does not comprise protamine or a salt thereof.

29. The complex of claim 27, wherein the mean diameter of the complex is greater than about 100 nm and less than 200 nm.

30. The complex of claim 27, wherein the mean diameter of the complex is determined by incubation in 50% serum in buffer for about 1 hour.

31. The complex of claim 27, wherein the complex has reduced binding to complement C3A and C5A.

32. The complex of any one of claims 27-31, wherein the fusogenic lipid is a cone forming lipid.

33. The complex of claim 27, wherein the cone forming lipid is dioleoyl phosphatidylethanolamine (DOPE), 1,2-dioleoyl-sn-glycero-3-[phospho-L-serine] (DOPS), or N,N dioleyl-N,N-dimethyl-1,6-hexanediammonium chloride (TODMAC6).

34. The complex of claim 27, wherein the fusogenic lipid is pH sensitive.

35. The complex of claim 34, wherein the lipid is anionic at physiological pH, and fusogenicity is increased at about pH 5.5 to about pH 4.5 relative to physiological pH.

36. The complex of claim 35, wherein at about pH 4.5 the lipid is neutral or cationic.

37. The complex of claim 35, wherein the lipid is cholesteryl hemisuccinate (CHEMS) or 1,2-dioleoyl-sn-glycero-3-[phosphoethanolamine-N-dodecanoyl] (NC₁₂-DOPE).

38. The complex of claim 27, wherein the lipid is neutral or cationic.

39. The complex of claim 27, wherein the polycation is selected from the group consisting of synthetic polycations, polycationic polypeptides, and salts thereof.

40. The complex of claim 39, wherein the polycation is a synthetic polycation.

41. The complex of claim 40, wherein the synthetic polycation is selected from the group consisting of polycationic methacryloxypolymers, polycationic methacrylate polymers and polycationic poly(alkenylimines).

42. The complex of claim 41, wherein the synthetic polycationic methacrylate polymer is a polymer comprising dimethylamino methacrylate.

43. The complex of claim 40 is a synthetic polycation selected from the group consisting of polyethyleneimine (PEI), poly(2-methacryloxyethyltrimethyl ammonium bromide) (PMOETMAB), and a co-polymer of dimethylamino methacrylate and methacrylic ester.

44. A complex according to any one of claims 39-43, wherein the complex further comprises at least one co-lipid.

45. The complex of claim 44, wherein the complex comprises 1,2-distearoyl-sn-glycero-3-phosphotidylethanolamine (DSPE).

46. The complex of claim 27, wherein the complex further comprises at least one targeting factor that increases cellular bioavailability of the nucleic acid.

47. The complex of claim 46, wherein the presence of the targeting factor results in an increase in transcription of the nucleic acid, an increase in the uptake of nucleic acid into the cell, an increase in the uptake of nucleic acid into a cellular compartment, an increase in an exit of the nucleic acid from a cellular compartment, or an increase in transport of the nucleic acid across a membrane.

48. The complex of claim 46, wherein the targeting factor is selected from the group consisting of folate, insulin, an Arg-Gly-Asp (RGD) peptide, luteinizing hormone releasing hormone (LHRH), a membrane translocating peptide (MTLP) and a compound comprising a nuclear localization sequence.

49. The complex of claim 46, wherein the targeting factor is selected from the group consisting of galactose-H₂N-KKAAAVLLPVLLAAP-COOH (Elan094) (SEQ ID NO:2), galactose-H₂N-KKKAAAVLLPVLLAAP (ZElan094) (SEQ ID NO:3), galactose-H₂N-kkkaavllpvllaap (ZElan207) (SEQ ID NO:8), and galactose-H₂N-KKKAAAVLLPVLLAAPREDL (ZElan094R) (SEQ ID NO:5).

50. The complex of claim 27, wherein the lipid undergoes a structural change between physiologic pH and pH about 4.5 resulting in increased fusogenicity.

51. The complex of claim 1, wherein the complex is shielded.

52. The complex of claim 27, wherein the complex is shielded.

53. The complex of claim 51, further comprising a compound containing polyethylene glycol moieties.

54. The complex of claim 52, further comprising a compound containing polyethylene glycol moieties.

55. The complex of claim 53, wherein the compound is a pegylated lipid.

56. The complex of claim 54, wherein the compound is a pegylated lipid.

57. A method for preparing a lipid-nucleic acid complex comprising a compacted nucleic acid and at least one lipid species that is fusogenic, comprising:
a) mixing an aqueous micelle mixture comprising a lipid and at least one lipophilic surfactant with a nucleic acid mixture comprising a nucleic acid, wherein the lipid has or assumes fusogenic characteristics, and wherein at least one of the mixtures contains a component that causes the nucleic acid to compact; and
b) after the mixing removing the lipophilic surfactant from mixture resulting from step a).

58. The method of claim 57, further comprising including at least one targeting agent in at least one of the mixtures of step a).

59. A lipid-nucleic acid complex prepared by the method of claim 57.

60. A lipid-nucleic acid complex prepared by the method of claim 58.

61. A complex according to any one of claims 27-31, 33-43, 46-48, or 50-56, prepared by the method of claim 57.

62. A complex according to claim 44 prepared by the method of claim 57.

63. A complex according to claim 45 prepared by the method of claim 57.

64. A complex according to any one of claims 46 to 48 prepared by the method of claim 58.

65. A method of delivering a nucleic acid to a cell comprising contacting the cell with a complex according to any one of claims 1-17, 19-22, 27-31, 33-43, 46-48, or 50-56.

66. A method of delivering a nucleic acid to a cell comprising contacting the cell with a complex according to claim 23.

67. A method of delivering a nucleic acid to a cell comprising contacting the cell with a complex according to claim 24.

68. A method of delivering a nucleic acid to a cell comprising contacting the cell with a complex according to claim 25.

69. A method of delivering a nucleic acid to a cell comprising contacting the cell with a complex according to claim 26.

70. A method of delivering a nucleic acid to a cell comprising contacting the cell with a complex according to claim 44.

71. A method according to claim 65, wherein the delivery is in vivo to an individual.

72. A method according to claim 66, wherein the delivery is in vivo to an individual.

73. A method according to claim 67, wherein the delivery is in vivo to an individual.

74. A method according to claim 68, wherein the delivery is in vivo to an individual.

75. A method according to claim 69, wherein the delivery is in vivo to an individual.

76. A method according to claim 70, wherein the delivery is in vivo to an individual.

77. A method according to claim 71 wherein the delivery is intravenous.

78. A method according to claim 72 wherein the delivery is intravenous.

79. A method according to claim 73 wherein the delivery is intravenous.

80. A method according to claim 74 wherein the delivery is intravenous.

81. A method according to claim 75 wherein the delivery is intravenous.

82. A method according to claim 76 wherein the delivery is intravenous.

83. A method according to claim 71 wherein the individual is a human.

84. A method according to claim 72 wherein the individual is a human.

85. A method according to claim 73 wherein the individual is a human.

86. A method according to claim 74 wherein the individual is a human.

87. A method according to claim 75 wherein the individual is a human.

88. A method according to claim 76 wherein the individual is a human.

89. A method according to claim 77 wherein the individual is a human.

90. A method according to claim 78 wherein the individual is a human.

91. A method according to claim 79 wherein the individual is a human.

92. A method according to claim 80 wherein the individual is a human.

93. A method according to claim 81 wherein the individual is a human.

94. A method according to claim 82 wherein the individual is a human.
